(19) United States
(12) Patent Application Publication
(10) Pub. No.: US 2022/0364092 A1
(43) Pub. Date: Nov. 17, 2022

(54) TREM COMPOSITIONS FOR CON-RARE CODONS AND RELATED USES

(71) Applicant: FLAGSHIP PIONEERING, INC., Cambridge, MA (US)

(72) Inventors: Christine Elizabeth Hajdin, Boston, MA (US); David Arthur Berry, Newton, MA (US); Theonie Anastassiadis, Boston, MA (US); Noubar Boghos Afeyan, Lexington, MA (US)

(21) Appl. No.: 17/774,410

(22) PCT Filed: Nov. 4, 2020
(86) PCT No.: PCT/US2020/058948

§ 371 Date: May 4, 2022

Related U.S. Application Data
(60) Provisional application No. 62/930,361, filed on Nov. 4, 2019.

(51) Int. Cl. C12N 15/113; C12P 19/34

(52) U.S. Cl. CPC C12N 15/113; C12P 19/34

(57) ABSTRACT

The invention relates generally to uses of tRNA-based effector molecules (TREMs) corresponding to con-rare codons and methods of making the same.

### CROSS-REFERENCE TO RELATED APPLICATIONS

This application claims priority to U.S. Provisional Application 62/930,361 filed on Nov. 4, 2019, the entire contents of which is hereby incorporated by reference.

### BACKGROUND

Transfer RNAS (tRNAs) are molecules which possess a number of functions including the initiation and elongation of proteins.

### SUMMARY

The inventors have discovered that a TREM composition can be used to modulate a production parameter of an RNA, or a protein encoded by an RNA, wherein the RNA has a contextually-rare codon (“con-rare codon”). In an aspect, provided herein is a method of modulating a production parameter of an RNA, or a protein encoded by an RNA, in a target cell or tissue, comprising providing, e.g., administering, to the target cell or tissue, or contacting the target cell or tissue with, an effective amount of a tRNA effector molecule (TREM) (e.g., a TREM composition comprising a TREM), which TREM corresponds to a contextually-rare codon (“con-rare codon”) of the RNA, thereby modulating the production parameter of the RNA, or protein encoded by the RNA in the target cell or tissue.

In an embodiment, the target cell or tissue is obtained from a subject. In an embodiment, the method comprises administering the TREM composition to a subject. In an embodiment, the method comprises contacting the TREM composition with the target tissue or cell ex vivo. In an embodiment, the method comprises introducing the ex vivo-contacted target tissue or cell into a subject, e.g., an allogeneic or autologous subject.

In an embodiment, the production parameter comprises an expression parameter or a signaling parameter, e.g., as described herein. In an embodiment, the production parameter of the RNA is modulated, e.g., an RNA that can be translated into a polypeptide, e.g., a messenger RNA. In an embodiment, the production parameter of the RNA is increased or decreased. In an embodiment, the production parameter of the protein encoded by the RNA is modulated. In an embodiment, the production parameter of the protein is increased or decreased.

In an embodiment, the target cell or tissue comprises or is associated with, or correlated (negatively or positively) with, an unwanted characteristic or a selected characteristic. In an embodiment, the target cell or tissue comprises or is associated with, or correlated (negatively or positively) with, a disease or disorder. In an embodiment, the disease or disorder comprises a cancer. In an embodiment, the target cell or tissue is characterized by unwanted proliferation, e.g., benign or malignant proliferation. In some embodiments, the target cell or tissue is a cancer cell.

In an embodiment, the disease or disorder comprises a haploinsufficiency disorder, e.g., a disease in which an allele of a gene has a loss-of-function lesion, e.g., a total loss of function lesion. Exemplary haploinsufficiency disorders include GLUT1 deficiency syndrome 1, GLUT1 deficiency syndrome 2, a disorder caused by a GATA2 mutation (e.g., GATA2 deficiency; monocyte, B and NK lymphocyte deficiency; Emberger syndrome; monocytopenia and Mycobacterium avium complex/dendritic cell), Coffin-Siris syndrome 2, Charcot-Marie-Tooth disease, Robinow syndrome, Takenouchi-Kosaki syndrome, chromosome 1p35 deletion syndrome, chromosome 2p12-p11.2 deletion syndrome, WHIM syndrome, Mowat-Wilson syndrome, and Dravet syndrome.

In an embodiment, the target cell or tissue comprises a metabolic state or condition.

In an embodiment, the target cell or tissue comprises or is associated with a genetic event, e.g., a mutation, e.g., a point mutation, a rearrangement, a translocation, an insertion, or a deletion. In an embodiment, the genetic event comprises a single nucleotide polymorphism (SNP) or other marker. In an embodiment, the genetic event is associate with, or correlated (negatively or positively) with, a disease or disorder or a predisposition to a disease or disorder.

In an embodiment, the target cell or tissue comprises or is associated with, or correlated (negatively or positively) with, a pattern of gene expression, e.g., unwanted or insufficient expression of a gene.

In an embodiment, the target cell or tissue comprises or is associated with an epigenetic event, e.g., histone modification, e.g., an epigenetic event which is correlated (negatively or positively) with a disease or disorder or a predisposition to a disease or disorder.

In an embodiment, the target cell or tissue comprises a product, e.g., a nucleic acid (e.g., an RNA), protein, lipid, or sugar, associated with, or correlated (negatively or positively) with, a disorder or disease. In an embodiment, the cell or tissue produces a product, e.g., a nucleic acid (e.g., an RNA), protein, lipid, or sugar, the presence thereof is associated with, or correlated (negatively or positively) with, an unwanted state, e.g., a disease or disorder.

In an embodiment, the cell or tissue fails to produce, or fails to produce a sufficient amount of, a product, e.g., a nucleic acid (e.g., an RNA), protein, lipid, or sugar, and the absence or insufficient amount of such product is associated with, or correlated (negatively or positively) with, an unwanted state, e.g., a disease or disorder.

In an embodiment, the target cell or tissue comprises a certain developmental stage, e.g., embryonic, fetal, immature, mature, or senescent. In an embodiment, the target cell or a cell in the target tissue comprises a stage in the cell cycle, e.g., G0, G1, S, G2, or M. In an embodiment, the target cell or tissue is non-proliferative or quiescent. In an embodiment, the target cell or tissue is proliferative. In an embodiment the cell or tissue comprises a hematopoietic cell or tissue, e.g., a fibroblast. In an embodiment the cell or tissue comprises a hepatic cell or tissue. In an embodiment the cell or tissue comprises a renal cell or tissue. In an embodiment the cell or tissue comprises a neural cell or tissue, e.g., a neuron. In an embodiment the cell or tissue comprises a muscle cell or tissue. In an embodiment the cell or tissue comprises a skin cell or tissue.

In another aspect, the disclosure provides a method of determining the presence of a nucleic acid sequence, e.g., a DNA or RNA, having a contextually-rare codon (“con-rare codon nucleic acid sequence”), comprising: acquiring knowledge of the presence of the con-rare codon nucleic acid sequence in a sample from a subject, e.g., a target cell or tissue sample, wherein responsive to the acquisition of knowledge of the presence of the con-rare codon nucleic acid sequence: (1) the subject is classified as being a candidate to receive administration of an effective amount of a composition comprising a tRNA effector molecule (TREM) which corresponds to a contextually-rare codon (“con-rare codon”) of the nucleic acid sequence; or (2) the subject is identified as likely to respond to a treatment comprising the composition comprising the TREM.

In yet another aspect, provided herein is a method of treating a subject having a disease associated with a contextually-rare codon (“con-rare codon”), comprising: acquiring knowledge of the presence of a nucleic acid sequence, e.g., a DNA or RNA, having the con-rare codon (“con-rare codon nucleic acid sequence”) in a target cell or tissue sample from the subject; and administering to the subject an effective amount of a composition comprising a tRNA effector molecule (TREM) which corresponds to the con-rare codon of the nucleic acid sequence, thereby treating the disease in the subject.

In an embodiment, administering comprises providing to the target cell or tissue, or contacting the target cell or tissue with, an effective amount of a tRNA effector molecule (TREM) (e.g., a TREM composition comprising a TREM), which TREM corresponds to a contextually-rare codon (“con-rare codon”) of the RNA,

In an aspect, the disclosure provides a method of providing a tRNA effector molecule (TREM) to a subject, comprising: providing, e.g., administering, to the subject, an effective amount of a TREM, e.g., a TREM composition comprising a TREM, which TREM corresponds to a contextually-rare codon (“con-rare codon”) for a nucleic acid sequence in a target cell or tissue in the subject, thereby providing a TREM to the subject.

In an embodiment, administering comprises providing to the target cell or tissue, or contacting the target cell or tissue with, an effective amount of a tRNA effector molecule (TREM) (e.g., a TREM composition comprising a TREM), which TREM corresponds to a contextually-rare codon (“con-rare codon”) of the RNA,

In another aspect, provided herein is a method of manufacturing a tRNA effector molecule (TREM) composition comprising:

identifying a TREM corresponding to a contextually-rare (con-rare) codon;

combining the TREM with a component, e.g., a carrier or excipient. thereby manufacturing a TREM composition.

In an embodiment of any of the methods provided herein, the method comprises acquiring a value for a con-rare codon in the nucleic acid sequence, e.g., DNA or RNA, wherein the value is a function of one or more of the following factors, e.g., by evaluating or determining one or more of the following factors:

(1) the sequence of the codon;

(2) the availability of a corresponding tRNA, e.g., charged tRNA, for that con-rare codon in a target cell or tissue, e.g., one or more iso-acceptor tRNA molecules;

(3) the expression profile (or proteomic properties) of the target cell or tissue (e.g., the abundance of expression of other proteins which include the con-rare codon);

(4) the proportion of the tRNAs corresponding to the con-rare codon which are charged;

(5) the iso-decoder isotype of the tRNA corresponding to the con-rare codon; and

(6) a target cell or tissue characterization selected from:
(i) the presence or absence of an unwanted characteristic, e.g., the target cell or tissue is associated with a disorder or disease;
(ii) the presence or absence of unwanted proliferation in the target cell or tissue:
(iii) the presence or absence of a preselected genetic event, e.g., and event associated with a disorder or disease, in the nucleic acid of the target cell or tissue.

In an embodiment, (1) comprises determining the presence or absence of a con-rare codon.

In an embodiment, a determination of the availability of a tRNA comprises acquiring a measure of one, two, three or all of the following parameters:

(a) level of a tRNA corresponding to the con-rare codon (“con-rare codon tRNA”) compared to a tRNA corresponding to a different codon;

(b) function, e.g., polypeptide chain elongation function, of a con-rare codon tRNA compared to a tRNA corresponding to a different codon;

(c) modification, e.g., aminoacylation or post-transcriptional modification, of a con-rare codon tRNA compared to a tRNA corresponding to a different codon;

(d) sequence of a con-rare codon tRNA; and/or

(e) a value for proteome codon count-tRNA frequency (PCC-tF).

In an embodiment, a measure of availability (e.g., level) of a con-rare codon tRNA comprises a measure of the con-rare codon tRNA that is charged, e.g., aminoacylated, compared to: (1) the proportion of the con-rare codon tRNA that is not charged; or (2) the proportion of charged tRNA corresponding to a different codon.

In an embodiment of any of the methods provided herein, at least 10%, 20%, 30%, 40%, 50%, 60%, 70%, 80%, 85%, 90%, 95%, 99%, or 100% (by weight or number) of the TREMs in the TREM composition correspond to a con-rare codon. In an embodiment, the TREM composition comprises TREMs that correspond to a plurality of con-rare codons. In an embodiment, the TREM composition comprises: a first TREM which corresponds to a first con-rare codon; and an additional TREM which corresponds to a different con-rare codon.

In an embodiment of any of the methods provided herein, the TREM composition (e.g., composition comprising a TREM corresponding to a con-rare codon) is made by a method comprising:
(a) providing a host cell, comprising exogenous nucleic acid, e.g., a DNA or RNA, encoding a TREM under conditions sufficient to express the TREM; and
(b) purifying the expressed TREM from the host cell culture to produce a TREM composition, thereby making a TREM composition.

In an embodiment, the TREM composition (e.g., composition comprising a TREM corresponding to a con-rare codon), is a pharmaceutical composition comprising a TREM.

In an embodiment, the TREM composition (e.g., composition comprising a TREM corresponding to a con-rare codon), comprises a pharmaceutical excipient. In an embodiment, the TREM composition comprises a TREM fragment, e.g., as described herein.

In an embodiment, the TREM composition (e.g., composition comprising a TREM corresponding to a con-rare codon), comprises one or more, e.g., a plurality, of TREMs.

Disclosed herein, inter alia, is a method of modulating the expression of an RNA or a protein encoded by the RNA, in a target cell or tissue, which has a contextually rare codon (con-rare codon), and a method of identifying a con-rare codon. An RNA having a con-rare codon can have reduced expression, e.g., reduced expression of the protein encoded by said RNA, compared to, e.g., an RNA that does not have a con-rare codon. In an embodiment, the expression of a nucleic acid, e.g., RNA, or protein encoded by said nucleic acid, e.g., RNA (in a target cell or tissue) having a con-rare codon is modulated by providing to said target cell or tissue, an effective amount of a tRNA effector molecule (TREM), e.g., a TREM composition comprising a TREM, which TREM corresponds to a con-rare codon of the nucleic acid, e.g., RNA. In an embodiment, providing (e.g., administering) the TREM composition corresponding to the con-rare codon can result in an increase in a production parameter, e.g., expression parameter or signaling parameter, of the nucleic acid, e.g., RNA, or protein encoded by said nucleic acid, e.g., RNA, having the con-rare codon.

Methods disclosed herein comprise identifying a con-rare codon. In an embodiment, a con-rare codon is a codon that is limiting for a production parameter, e.g., an expression parameter or a signaling parameter, for a nucleic acid sequence having said con-rare codon or for a product of the nucleic acid, e.g., an RNA or a protein. In an embodiment, identification of a con-rare codon comprises evaluating contextual rareness (con-rarity) which is a function of normalized proteome codon count and tRNA availability in a specific or selected target tissue or cell. The specific or selected target tissue or cell exists in a particular context which may be, e.g., a cell or tissue type in a particular developmental stage, a cell or tissue type in a particular disease state, a cell present in a particular extracellular milieu, a cell which has undergone a change (e.g., differentiation, proliferation or activation); a cell with finite proliferative capacity (e.g., a primary cell); a cell with unlimited proliferative capacity (e.g., an immortalized cell); a cell with differential potential (e.g., a totipotent cell, a multipotent cell or a pluripotent cell); a differentiated cell; a somatic cell; a germline cell; or a cell with preselected level of RNA or protein expression. For example, the specific or selected target tissue or cell is specific for a particular tissue, e.g., a tissue formed by a germ layer, e.g., mesoderm, ectoderm or endoderm.

In an embodiment, contextual rareness (con-rarity) is a measure that is contextually dependent on tRNA availability or activity levels in a specific or selected target tissue or cell. Normalized proteome codon count is a function of codon count per nucleic acid sequence, e.g., gene, and the expression profile (or proteomic properties) of a target tissue or cell. In an embodiment, a tRNA corresponding to a con-rare codon is less available in amount or activity compared to the demand of said tRNA based on the codon count per nucleic acid sequence, e.g., gene, and thus the codon corresponding to said tRNA may be categorized as a con-rare codon.

For example, in a specific or selected cell where (on average) codon X appears Y times for every 100 codons associated with the cells' proteome, codon X is a con-rare codon if less than 10Y, 5Y, Y, 0.5Y, 0.2Y, or 0.1Y % of the existing, functionally available, temporally available, or translationally-competent tRNAs in that same cell correspond to codon X. In an embodiment, the level is Y. As another example, in a specific or selected cell where (on average) codon X appears 3 times for every 100 codons associated with the cells' proteome, codon X is a con-rare codon if less than 3% of the existing, functionally available, temporally available, or translationally-competent tRNAs in that same cell correspond to codon X.

In an embodiment, con-rarity takes into account both the supply of tRNAs corresponding to the codon and the demand placed on that supply in the context of a specific or selected cell or tissue.

Methods disclosed here comprise a TREM composition, and uses thereof, having a TREM which corresponds to a con-rare codon. Such TREM compositions can be used to modulate a production parameter, e.g., the production of a protein, in a specific or selected target or cell.

Methods described herein allow for the administration of a TREM composition having a TREM which corresponds to a con-rare codon to modulate a production parameter in vivo, of an RNA, or protein encoded by the RNA (heterologous or endogenous) in a subject, or in a target tissue or cell. Methods described herein also allow for the administration of a TREM composition which corresponds to a con-rare codon to modulate a production parameter in vitro, of an RNA, or protein encoded by an RNA having a con-rare codon.

The approach can take into account a number of factors, including, the availability, e.g., abundance, of a tRNA corresponding to a con-rare codon in the target tissue or cell; or the demand placed on a tRNA by the codons of other expressed nucleic acid sequences (other than the RNA whose production parameter is modulated) in the target tissue or cell. E.g., selection of a TREM can take into account the expression profile (or proteomic properties) in the target cell or tissue of nucleic acid sequences having a con-rare codon, and the frequency or proportion of appearance of the con-rare codon in nucleic acid sequences having a con-rare codon.

As disclosed herein, tRNA-based effector molecules (TREMs) are complex molecules which can mediate a variety of cellular processes. Compositions comprising a TREM or pharmaceutical compositions comprising a TREM can be administered to cells, tissues or subjects to modulate a production parameter of an RNA, or a protein encoded by an RNA, e.g., in vitro or in vivo. Also disclosed herein are methods of treating or preventing a disorder, or a symptom of a disorder (e.g., a disorder associated with a con-rare codon) by administering compositions comprising a TREM or pharmaceutical compositions comprising a TREM. Further disclosed herein are compositions comprising a TREM, or pharmaceutical compositions comprising a TREM, preparations, and methods of making the same.

Additional features of any of the aforesaid compositions (e.g., TREM composition or pharmaceutical composition comprising a TREM); methods of using said compositions and/or methods of making the same include one or more of the following enumerated embodiments.

Those skilled in the art will recognize or be able to ascertain using no more than routine experimentation, many equivalents to the specific embodiments of the invention described herein. Such equivalents are intended to be encompassed by the following enumerated embodiments.

### Enumerated Embodiments

E1. A method of modulating a production parameter of an RNA, or a protein encoded by an RNA, in a target cell or tissue, comprising:

providing, e.g., administering, to the target cell or tissue, or contacting the target cell or tissue with, an effective amount of a tRNA effector molecule (TREM) (e.g., a TREM composition comprising a TREM), which TREM corresponds to a contextually-rare codon (“con-rare codon”) of the RNA,

thereby modulating the production parameter of the RNA, or protein encoded by the RNA in the target cell or tissue.

E2. The method of embodiment E1, wherein the target cell or tissue is obtained from a subject.
E3. The method of embodiment E1, comprising administering the TREM composition to a subject.
E4. The method of embodiment E1, comprising contacting the TREM composition with the target tissue or cell ex vivo.
E5. The method of embodiment E4, comprising introducing the ex vivo-contacted target tissue or cell into a subject, e.g., an allogeneic or autologous subject.
E6. The method of any one of the preceding embodiments, wherein the target cell or tissue is a specific or selected target cell or tissue, e.g., a cell or tissue type in a particular developmental stage; a cell or tissue type in a particular disease state; or a cell present in a particular extracellular milieu.
E7. The method of any one of the preceding embodiments, wherein the target cell or tissue comprises or is associated, or correlated (negatively or positively) with, an unwanted characteristic or a selected characteristic.
E8. The method of any one of the preceding embodiments, wherein the target cell or tissue comprises or is associated, or correlated (negatively or positively) with, a disease or disorder.
E9. The method of embodiment of E8, wherein the disease or disorder comprises a cancer or a haploinsufficiency disorder.
E10. The method of any one of the preceding embodiments, wherein the target cell or tissue is characterized by unwanted proliferation, e.g., benign or malignant proliferation.
E11. The method of any one of the preceding embodiments, wherein the target cell or tissue comprises or is associated with a genetic event, e.g., a mutation, e.g., a point mutation, a rearrangement, a translocation, an insertion, or a deletion.
E12. The method of any one of preceding embodiments, wherein the target cell or tissue comprises or is associated with an epigenetic event, e.g., histone modification, e.g., an epigenetic event which is correlated (negatively or positively) with a disease or disorder or a predisposition to a disease or disorder.
E13. The method of any one of the preceding embodiments, wherein the target cell or tissue comprises a product, e.g., a nucleic acid (e.g., a RNA), protein, lipid, or sugar, associated with, or correlated (negatively or positively) with, a disorder or disease.
E14. The method of embodiments of E12 or E13, wherein the disease or disorder comprises a cancer or a haploinsufficiency disorder.
E15. The method of any one of the preceding embodiments, wherein the production parameter comprises an expression parameter or a signaling parameter, e.g., as described herein.
E16. The method of any one of the preceding embodiments, wherein the production parameter of the RNA is modulated, e.g., an RNA that can be translated into a polypeptide, e.g., a messenger RNA.
E17. The method of embodiment E7, wherein the production parameter of the RNA is increased or decreased.
E18. The method of any one of the preceding embodiments, wherein the production parameter of the protein encoded by the RNA is modulated.
E19. The method of embodiment E18, wherein the production parameter of the protein is increased.
E20. The method of embodiment E18, wherein the production parameter of the protein is decreased.
E21. A method of determining the presence of a nucleic acid sequence, e.g., a DNA or RNA, having a contextually-rare codon (“con-rare codon nucleic acid sequence”), comprising:

acquiring knowledge of the presence of the con-rare codon nucleic acid sequence in a sample from a subject, e.g., a target cell or tissue sample,

wherein responsive to the acquisition of knowledge of the presence of the con-rare codon nucleic acid sequence:

(1) the subject is classified as being a candidate to receive administration of an effective amount of a composition comprising a tRNA effector molecule (TREM) which corresponds to a contextually-rare codon (“con-rare codon”) of the nucleic acid sequence; or

(2) the subject is identified as likely to respond to a treatment comprising the composition comprising the TREM.

E22. A method of treating a subject having a disease associated with a contextually-rare codon (“con-rare codon”), comprising:

acquiring knowledge of the presence of a nucleic acid sequence, e.g., a DNA or RNA, having the con-rare codon (“con-rare codon nucleic acid sequence”) in a target cell or tissue sample from the subject; and

administering to the subject an effective amount of a composition comprising a tRNA effector molecule (TREM) which corresponds to the con-rare codon of the nucleic acid sequence,

thereby treating the disease in the subject.

E23. A method of providing a tRNA effector molecule (TREM) to a subject, comprising:

providing, e.g., administering, to the subject, an effective amount of a TREM, e.g., a TREM composition comprising a TREM, which TREM corresponds to a contextually-rare codon (“con-rare codon”) for a nucleic acid sequence in a target cell or tissue in the subject,

thereby providing a TREM to the subject.

E24. A method of manufacturing a tRNA effector molecule (TREM) composition comprising:

identifying a TREM corresponding to a contextually-rare (con-rare) codon;

combining the TREM with a component, e.g., a carrier or excipient. thereby manufacturing a TREM composition.

E25. The method of any one of the preceding embodiments, wherein the method comprises acquiring a value for a con-rare codon in the nucleic acid sequence, e.g., DNA or RNA, wherein the value is a function of one or more of the following factors, e.g., by evaluating or determining one or more of the following factors:

(1) the sequence of the codon;

(2) the availability of a corresponding tRNA, e.g., charged tRNA, for that con-rare codon in a target cell or tissue, e.g., one or more iso-acceptor tRNA molecules;

(3) the expression profile (or proteomic properties) of the target cell or tissue (e.g., the abundance of expression of other proteins which include the con-rare codon);

(4) the proportion of the tRNAs corresponding to the con-rare codon which are charged; and

(5) the iso-decoder isotype of the tRNA corresponding to the con-rare codon.

E26. The method of embodiment E25, wherein (1) comprises determining the presence or absence of a con-rare codon.
E27. The method of embodiment E25, wherein a determination of the availability of a tRNA comprises acquiring a measure of one, two, three or all of the following parameters:

(a) level of a tRNA corresponding to the con-rare codon (“con-rare codon tRNA”) compared to a tRNA corresponding to a different codon;

(b) function, e.g., polypeptide chain elongation function, of a con-rare codon tRNA compared to a tRNA corresponding to a different codon;

(c) modification, e.g., aminoacylation or post-transcriptional modification, of a con-rare codon tRNA compared to a tRNA corresponding to a different codon; and/or

(d) sequence of a con-rare codon tRNA.

E28. The method of embodiment E27, wherein a measure of availability (e.g., level) of a con-rare codon tRNA comprises a measure of the con-rare codon tRNA that is charged, e.g., aminoacylated, compared to: (1) the proportion of the con-rare codon tRNA that is not charged; or (2) the proportion of charged tRNA corresponding to a different codon.
E29. The method of any one of embodiments E25-E28, wherein responsive to said value, the target cell, or tissue, is identified as having a nucleic acid sequence having a con-rare codon (“con-rare codon nucleic acid sequence”) or an RNA having a con-rare codon (“con-rare codon RNA”).
E30. The method of any one of embodiments E25-E29, wherein responsive to said value, the RNA is identified as, an RNA having a con-rare codon.
E31. The method of any one of embodiments E1-E24, wherein the target cell or tissue is identified as having an RNA having a con-rare codon.
E32. The method of any one of embodiments E1-E24, wherein the nucleic acid sequence, e.g., DNA or RNA, is identified as, a nucleic acid sequence having a con-rare codon (“con-rare codon nucleic acid sequence”) or an RNA having a con-rare codon (“con-rare codon RNA”).
E33. The method of any one of the preceding embodiments, wherein the nucleic acid sequence (e.g., DNA or RNA) is, or is identified as, a nucleic acid sequence (e.g., DNA or RNA) having a plurality of con-rare codons.
E34. The method of any one of the preceding embodiments, wherein the nucleic acid sequence (e.g., DNA or RNA) is, or is identified as, a nucleic acid sequence (e.g., DNA or RNA) having a plurality of occurrences of a con-rare codon.
E35. The method of any one of the preceding embodiments, wherein the nucleic acid, e.g., RNA is, or is identified as, a nucleic acid, e.g., RNA, having a first tRNA which corresponds to a first con-rare codon; and an additional tRNA, e.g., a second tRNA, which corresponds to a different, e.g., a second, con-rare codon.
E36. The method of any one of the preceding embodiments, wherein the nucleic acid sequence (e.g., DNA or RNA) is, or is identified as, a nucleic acid sequence (e.g., DNA or RNA), having multiple occurrences of the first tRNA which corresponds to a first con-rare codon.
E37. The method of embodiment E35 or E36, wherein the nucleic acid sequence (e.g., DNA or RNA) is, or is identified as, a nucleic acid sequence (e.g., DNA or RNA) having multiple occurrences of the additional tRNA, e.g., a second tRNA, which corresponds to a different, e.g., a second, con-rare codon.
E38. The method of any one of the preceding embodiments, wherein modulation of a production parameter of the con-rare codon RNA comprises increasing a production parameter, e.g., an expression parameter or signaling parameter of the protein encoded by the con-rare codon RNA, e.g., increasing the expression level of the protein encoded by the con-rare codon RNA.
E39. The method of any one of the preceding embodiments, wherein modulation of a production parameter of the con-rare codon RNA comprises decreasing a production parameter, e.g., an expression parameter or signaling parameter, of the protein encoded by the con-rare codon RNA, e.g., decreasing the expression level of the protein encoded by the con-rare codon RNA.
E40. The method of any one of embodiments E25-E28, wherein a determination of the expression profile (or proteome codon count) of the target cell or tissue, comprises a measure of:
(a) the abundance (e.g., expression) of proteins in a target cell or tissue; and
(b) a protein codon count for expressed proteins in a target cell or tissue.
   E41. The method of any one of the preceding embodiments, wherein the con-rare codon is other than the initiator methionine codon (iMet).
   E42. The method of any one of the preceding embodiments, wherein the target cell or tissue is identified as comprising a con-rare-codon nucleic acid, e.g., RNA.
   E43. The method of any one of the preceding embodiments, wherein, the con-rare codon meets a reference value for one or more of the following:

(1) the sequence of the codon;

(2) the availability of a corresponding tRNA, e.g., charged tRNA, for that con-rare codon in a target cell or tissue, e.g., one or more iso-acceptor tRNA molecules;

(3) the expression profile (or proteomic properties) of the target cell or tissue (e.g., the abundance of expression of other proteins which include the con-rare codon);

(4) the proportion of the tRNAs corresponding to the con-rare codon which are charged; and

(5) the iso-decoder isotype of the tRNA corresponding to the con-rare codon;

E44. The method of embodiment E43, wherein the con-rare-codon meets a reference value for two of (1)-(5).
E45. The method of embodiment E43, wherein the con-rare-codon meets a reference value for three of (1)-(5).
E46. The method of embodiment E43, wherein the con-rare-codon meets a reference value for four of (1)-(5).
E47. The method of embodiment E43, wherein the con-rare-codon meets a reference value for all of (1)-(5).
E48. The method of embodiment E43, wherein the con-rare-codon meets a reference value for (1).
E49. The method of embodiment E43, wherein the con-rare-codon meets a reference value for (2).
E50. The method of embodiment E43, wherein the con-rare-codon meets a reference value for (3).
E51. The method of embodiment E43, wherein the con-rare-codon meets a reference value for (4).
E52. The method of embodiment E43, wherein the con-rare-codon meets a reference value for (5).
E53. The method of embodiment E43, wherein the reference value is a pre-determined or pre-selected reference value.
E54. The method of embodiment E43, wherein the reference value is determined according to a method described herein.
E55. The method of any of the preceding embodiments, wherein at least 10%, 20%, 30%, 40%, 50%, 60%, 70%, 80%, 85%, 90%, 95%, 99%, or 100% (by weight or number) of the TREMs in the TREM composition correspond to a con-rare codon.
E56. The method of any of the preceding embodiments, wherein the TREM composition comprises TREMs that correspond to a plurality of con-rare codons.
E57. The method of any of the preceding embodiments, wherein the TREM composition comprises: a first TREM which corresponds to a first con-rare codon; and an additional TREM which corresponds to a different con-rare codon.
E58. The method of any of the preceding embodiments, wherein the TREM composition comprises: a first TREM which corresponds to a first con-rare codon; and a second TREM which corresponds to a second con-rare codon.
E59. The method of any of the preceding embodiments, wherein the TREM composition comprises: a first TREM which corresponds to a first con-rare codon; a second TREM which corresponds to a second con-rare codon; and a third TREM which corresponds to a third con-rare codon.
E60. The method of any of the preceding embodiments, wherein the TREM composition comprises: a first TREM which corresponds to a first con-rare codon; a second TREM which corresponds to a second con-rare codon; a third TREM which corresponds to a third con-rare codon; and a fourth TREM which corresponds to a fourth con-rare codon.
E61. The method of any of the preceding embodiments, wherein the TREM composition comprises: a first TREM which corresponds to a first con-rare codon; a second TREM which corresponds to a second con-rare codon; a third TREM which corresponds to a third con-rare codon; a fourth TREM which corresponds to a fourth con-rare codon; and a fifth TREM which corresponds to a fifth con-rare codon.
E62. The method of any one of embodiments E56-E61, wherein at least 10%, 20%, 30%, 40%, 50%, 60%, 70%, 80%, 85%, 90%, 95%, 99%, or 100% (by weight or number) of the TREMs in the TREM composition correspond to the first con-rare codon.
E63. The method of any one of embodiments E56-E62, wherein at least 10%, 20%, 30%, 40%, 50%, 60%, 70%, 80%, 85%, 90%, 95%, 99%, or 100% (by weight or number) of the TREMs in the TREM composition correspond to the additional, e.g., second, third, fourth or fifth, con-rare codon.
E64. The method of any of the preceding embodiments, wherein at least 10%, 20%, 30%, 40%, 50%, 60%, 70%, 80%, 85%, 90%, 95%, 99%, or 100% (by weight or number) of the TREMs in the composition are charged.
E65. The method of any of the preceding embodiments, wherein the TREM composition comprises a first TREM which corresponds to a first con-rare codon and an additional TREM, e.g., a second, third, fourth, or fifth TREM, which corresponds to a different, e.g., second, third, fourth, or fifth, con-rare codon, and wherein at least 10%, 20%, 30%, 40%, 50%, 60%, 70%, 80%, 85%, 90%, 95%, 99%, or 100% (by weight or number) of the first TREM in the composition is charged.
E66. The method of embodiment E65, wherein at least 10%, 20%, 30%, 40%, 50%, 60%, 70%, 80%, 85%, 90%, 95%, 99%, or 100% (by weight or number) of the additional TREM e.g., second, third, fourth, or fifth TREM, in the composition is charged.
E67. The method of any of the preceding embodiments, wherein at least 10%, 20%, 30%, 40%, 50%, 60%, 70%, 80%, 85%, 90%, 95%, 99%, or 100% (by weight or number) of the TREMs in the preparation are of the same iso-decoder isotype.
E68. The method of any one of embodiments E1-E56, wherein the TREM composition comprises: a first TREM which corresponds to a first con-rare codon; and an additional TREM which corresponds to the first con-rare codon, e.g., the first TREM and the additional TREM are of the same iso-decoder isotype.
E69. The method of any one of embodiments E1-E56 or E68, wherein the TREM composition comprises: a first TREM which corresponds to a first con-rare codon; and a second TREM which corresponds to the first con-rare codon, e.g., the first TREM and the second TREM are of the same iso-decoder isotype.
E70. The method of any one of embodiments E1-E56 or E68-E69, wherein at least 10%, 20%, 30%, 40%, 50%, 60%, 70%, 80%, 85%, 90%, 95%, 99%, or 100% (by weight or number) of the TREMs in the TREM composition correspond to the first con-rare codon.
E71. The method of any one of embodiments E1-E56 or E68-E70, wherein at least 10%, 20%, 30%, 40%, 50%, 60%, 70%, 80%, 85%, 90%, 95%, 99%, or 100% (by weight or number) of the TREMs in the TREM composition correspond to the additional, e.g., second or third, con-rare codon, e.g., the first TREM and the additional TREM are of the same iso-decoder isotype.
E72. The method of any one of embodiments E1-E56 or E68-E71, wherein at least 10%, 20%, 30%, 40%, 50%, 60%, 70%, 80%, 85%, 90%, 95%, 99%, or 100% (by weight or number) of the TREMs in the composition are charged.
E73. The method of any one of embodiments E1-E56 or E68-E72, wherein the TREM composition comprises a first TREM which corresponds to a first con-rare codon, and an additional TREM, e.g., a second or third TREM, which corresponds to the first con-rare codon, and wherein at least 10%, 20%, 30%, 40%, 50%, 60%, 70%, 80%, 85%, 90%, 95%, 99%, or 100% (by weight or number) of the first TREM in the composition is charged.
E74. The method of embodiment E73, wherein at least 10%, 20%, 30%, 40%, 50%, 60%, 70%, 80%, 85%, 90%, 95%, 99%, or 100% (by weight or number) of the additional TREM e.g., second or third TREM, in the composition is charged.
E75. The method of any of the preceding embodiments, wherein the cell is a host cell.
E76. The method of any of the preceding embodiments, wherein the cell is a mammalian cell, e.g., a human cell, a murine cell, or a rodent cell.
E77. The method of any of the preceding embodiments, wherein the cell is a non-mammalian cell, e.g., a bacterial cell, an insect cell or a yeast cell.
E78. The method of any of the preceding embodiments, wherein the cell is a host cell chosen from: a HeLa cell, a HEK293T cell (e.g., a Freestyle 293-F cell), a HT-1080 cell, a PER.C6 cell, a HKB-11 cell, a CAP cell, a HuH-7 cell, a BHK 21 cell, an MRC-S cell, a MDCK cell, a VERO cell, a WI-38 cell, or a Chinese Hamster Ovary (CHO) cell.
E79. The method of any of the preceding embodiments, wherein the cell comprises an exogenous nucleic acid sequence.
E80. The method of any of the preceding embodiments, wherein the cell is autologous to the exogenous nucleic acid sequence.
E81. The method of any of the preceding embodiments, wherein the cell is allogeneic to the exogenous nucleic acid sequence.
E82. The method of any one of embodiments E79-E81, wherein the exogenous nucleic acid sequence (e.g., DNA or RNA) comprises a con-rare codon.
E83. The method of any one of embodiments E79-E82, wherein administration of a TREM composition corresponding to the con-rare codon to the cell, modulates a production parameter, e.g., expression parameter or signaling parameter, of a product, e.g., RNA or polypeptide, of the exogenous nucleic sequence.
E84. The method of any one of embodiments E79-E83, wherein administration of a TREM composition corresponding to the con-rare codon to the cell, increases a production parameter, e.g., expression parameter or signaling parameter, of a product, e.g., RNA or polypeptide, of the exogenous nucleic sequence.
E85. The method of any one of embodiments E79-E84, wherein administration of a TREM composition corresponding to the con-rare codon, to the cell decreases a production parameter, e.g., expression parameter or signaling parameter, of a product, e.g., RNA or polypeptide, of the exogenous nucleic sequence.
E86. The method of any of the preceding embodiments, wherein the modulation, increase or decrease in production parameter, is compared to an otherwise similar cell, which: (1) is not contacted with the TREM composition; (2) does not comprise an exogenous nucleic acid sequence; or (3) comprises an exogenous nucleic acid sequence which does not comprise a con-rare codon.
E87. A method of modulating a production parameter of an RNA, or a protein encoded by the RNA, in a cell, comprising:

optionally, acquiring knowledge of the presence of an RNA having a contextually-rare codon (“con-rare codon RNA”) in the cell,

providing to the cell an effective amount of a tRNA corresponding to the con-rare codon RNA,

thereby modulating the production parameter of the RNA, or the protein encoded by the RNA in the cell.

E88. A method of modulating a production parameter of an RNA, or a protein encoded by an RNA, in a cell, comprising:

optionally, acquiring knowledge of the presence of an RNA having a contextually-rare codon (“con-rare codon RNA”) in the cell,

modulating a culture parameter such that a production parameter of the RNA or protein encoded by the RNA is modulated.

E89. The method of any one of embodiments E87-E88, wherein acquiring knowledge of the con-rare codon RNA comprises acquiring a value for a con-rare codon in the RNA, wherein the value is a function of one or more of the following factors, e.g., by evaluating or determining one or more of the following factors:

(1) the sequence of the codon;

(2) the availability of a corresponding tRNA, e.g., charged tRNA, for that con-rare codon in a target cell or tissue, e.g., one or more iso-acceptor tRNA molecules;

(3) the expression profile (or proteomic properties) of the target cell or tissue (e.g., the abundance of expression of other proteins which include the con-rare codon);

(4) the proportion of the tRNAs corresponding to the con-rare codon which are charged;

(5) the iso-decoder isotype of the tRNA corresponding to the con-rare codon;

E90. The method of any one of embodiments E87-E89, wherein modulating a culture parameter comprises any one or all of the following:

(i) changing the amount of time a cell is cultured, e.g., increasing or decreasing the time;
(ii) changing the density of cells in the culture, e.g., increasing or decreasing the cell density;
(iii) changing a component of the culture, e.g., adding or removing or changing the concentration of a media component, a nutrient, a supplement, a pH modulator;
(iv) culturing the cell with one or more additional components, e.g., a cell or a purified cell component (e.g., a tRNA), a cell lysate;
(v) changing the temperature at which the cell is cultured, e.g., increasing or decreasing the temperature; or
(vi) changing the size of the vessel in which the cell is cultured in, e.g., increasing or decreasing the size of the vessel.
   E91. The method of any one of embodiments E87-E90, wherein the cell is a host cell.
   E92. The method of any one of embodiments E87-E91, wherein the cell is a mammalian cell, e.g., a human cell, a murine cell, or a rodent cell.
   E93. The method of any one of embodiments E87-E92, wherein the cell is a non-mammalian cell, e.g., a bacterial cell, an insect cell or a yeast cell.
   E94. The method of any one of embodiments E87-E93, wherein the cell is a host cell chosen from: a HeLa cell, a HEK293T cell (e.g., a Freestyle 293-F cell), a HT-1080 cell, a PER.C6 cell, a HKB-11 cell, a CAP cell, a HuH-7 cell, a BHK 21 cell, an MRC-S cell, a MDCK cell, a VERO cell, a WI-38 cell, or a Chinese Hamster Ovary (CHO) cell.
   E95. The method of any one of embodiments E87-E94, wherein the cell comprises an exogenous nucleic acid sequence.
   E96. The method of any one of embodiments E87-E95, wherein the cell is autologous to the exogenous nucleic acid sequence.
   E97. The method of any one of embodiments E87-E95, wherein the cell is allogeneic to the exogenous nucleic acid sequence.
   E98. The method of any one of embodiments E87-E97, wherein the exogenous nucleic acid sequence comprises a con-rare codon.
   E99. The method of any one of the preceding embodiments, wherein the TREM composition was made by a method comprising:
(a) providing a host cell, comprising exogenous nucleic acid, e.g., a DNA or RNA, encoding a TREM under conditions sufficient to express the TREM; and
(b) purifying the expressed TREM from the host cell culture to produce a TREM composition, thereby making a TREM composition.
   E100. The method of embodiment E99, further comprising making the TREM composition by a method comprising:
(a) providing a host cell, comprising exogenous nucleic acid, e.g., a DNA or RNA, encoding a TREM under conditions sufficient to express the TREM; and
(b) purifying the expressed TREM from the host cell culture to produce a TREM composition, thereby making a TREM composition.
   E101. The method of any one of the preceding embodiments, wherein the TREM composition is a pharmaceutical composition comprising a TREM.
   E102. The method of any one of the preceding embodiments, wherein the TREM composition comprises a pharmaceutical excipient.
   E103. The method of any one of embodiments E100-E102, comprising introducing the exogenous DNA or RNA into the mammalian host cell.
   E104. The method of any one of embodiments E100-E103, wherein the nucleic acid comprises a DNA, which upon transcription, expresses a TREM.
   E105. The method of any one of embodiments E100-E103, wherein the nucleic acid comprises an RNA, which upon reverse transcription, results in a DNA which can be transcribed to provide the TREM.
   E106. The method of any one of the preceding embodiments, wherein the TREM composition comprises a TREM fragment, e.g., as described herein.
   E107. The method of any one of embodiments E100-E106, wherein the host cell is a mammalian cell.
   E108. The method of any one of embodiments E100-E107, wherein the host cell comprises a cell selected from a HEK293T cell (e.g., a Freestyle 293-F cell), a HT-1080 cell, a PER.C6 cell, a HKB-11 cell, a CAP cell, a HuH-7 cell, a BHK 21 cell, an MRC-S cell, a MDCK cell, a VERO cell, a WI-38 cell, a Chinese Hamster Ovary (CHO) cell, or a MCF7 cell.
   E109. The method of any one of embodiments E100-E106, wherein the host cell is a non-mammalian cell, e.g., a bacterial cell, a yeast cell or an insect cell.
   E110. The method of any one of the preceding embodiments, wherein the TREM is a GMP-grade composition comprising a recombinant TREM (e.g., a TREM composition made in compliance with cGMP, and/or in accordance with similar requirements) comprising an RNA sequence at least 80% identical to an RNA sequence encoded by a DNA sequence listed in Table 1, or a fragment or functional fragment thereof.
   E111. The method of any one of the preceding embodiments, wherein the TREM comprises one or more post-transcriptional modifications listed in Table 2.
   E112. The method of embodiment E110-E111, wherein the composition comprising a recombinant TREM is at least 0.5 g, 1 g, 2 g, 3 g, 4 g, 5 g, 6 g, 7 g, 8 g, 9 g, 10 g, 15 g, 20 g, 30 g, 40 g, 50 g, 100 g, 200 g, 300 g, 400 g or 500 g.
   E113. The method of embodiment E110-E111, wherein the composition comprising a recombinant TREM is between 0.5 g to 500 g, between 0.5 g to 400 g, between 0.5 g to 300 g, between 0.5 g to 200 g, between 0.5 g to 100 g, between 0.5 g to 50 g, between 0.5 g to 40 g, between 0.5 g to 30 g, between 0.5 g to 20 g, between 0.5 g to 10 g, between 0.5 g to 9 g, between 0.5 g to 8 g, between 0.5 g to 7 g, between 0.5 g to 6 g, between 0.5 g to 5 g, between 0.5 g to 4 g, between 0.5 g to 3 g, between 0.5 g to 2 g, between 0.5 g to 1 g, between 1 g to 500 g, between 2 g to 500 g, between 5 g to 500 g, between 10 g to 500 g, between 20 g to 500 g, between 30 g to 500 g, between 40 g to 500 g, between 50 g to 500 g, between 100 g to 500 g, between 200 g to 500 g, between 300 g to 500 g, or between 400 g to 500 g.
   E114. The method of any one of the preceding embodiments, wherein the TREM composition comprises one or more, e.g., a plurality, of TREMs.
   E115. The method of any one of the preceding embodiments, wherein the TREM composition (or an intermediate in the production of a TREM composition) comprises one or more of the following characteristics:
(i) purity of at least 30%, 40%, 50%, 60%, 70%, 80%, 85%, 90%, 91%, 92%, 93%, 94%, 95%, 96%, 97%, 98%, or 99%;
(ii) host cell protein (HCP) contamination of less than 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
(iii) host cell protein (HCP) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, or 100 ng, per milligram (mg) of the TREM composition;
(iv) DNA, e.g., host cell DNA, of less than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
(v) Fragments of less than 0.1%, 1%, 2%, 3%, 4%, 5%, 6%, 7%, 8%, 9% or 10%;
(vi) low levels or absence of endotoxins, e.g., as measured by the Limulus amebocyte lysate (LAL) test;
(vii) in-vitro translation activity, e.g., as measured by an assay described in Example 8; (viii) TREM concentration of at least 0.1 ng/mL, 0.5 ng/mL, 1 ng/mL, 5 ng/mL, 10 ng/mL, 50 ng/mL, 0.1 ug/mL, 0.5 ug/mL, 1 ug/mL, 2 ug/mL, 5 ug/mL, 10 ug/mL, 20 ug/mL, 30 ug/mL, 40 ug/mL, 50 ug/mL, 60 ug/mL, 70 ug/mL, 80 ug/mL, 100 ug/mL, 200 ug/mL, 300 ug/mL, 500 ug/mL, 1000 ug/mL, 5000 ug/mL, 10,000 ug/mL, or 100,000 ug/mL;
(ix) sterility, e.g., as per cGMP guidelines for sterile drug products, e.g., the composition or preparation supports the growth of fewer than 100 viable microorganisms as tested under aseptic conditions, the composition or preparation meets the standard of USP <71>, and/or the composition or preparation meets the standard of USP <85>; or (x) viral contamination, e.g., the composition or preparation has an absence of or an undetectable level of viral contamination.
   E116. The method of any one of the preceding embodiments, wherein the TREM composition is contacted in vitro with a target cell or tissue.
   E117. The method of any one of the preceding embodiments, wherein the TREM composition is contacted ex vivo with a target cell or tissue, and optionally, the contacted cell or tissue is introduced, e.g., administered, into a subject, e.g., the subject from which the cell or tissue came, or a different subject.
   E118. The method of any one of the preceding embodiments, wherein the method is an in vivo method, e.g., a subject, or a tissue or cell of a subject, is contacted with the TREM composition in vivo.
   E119. The method of any one of the preceding embodiments, wherein the TREM composition is administered with a delivery agent, e.g., a liposome, a polymer (e.g., a polymer conjugate), a particle, a microsphere, microparticle, or a nanoparticle.
   E120. The method of any one of the preceding embodiments, wherein the TREM enhances:

(a) the stability of a product, e.g., a protein, and/or

(b) ribosome occupancy of a product.

E121. The method of any one of the preceding embodiments, wherein the TREM:
modulates ribosome occupancy;
modulates protein translation or stability;
modulates mRNA stability;
modulates protein folding or structure;
modulates protein transduction or compartmentalization;
modulates codon usage;
modulates cell fate; or
modulates a signaling pathway, e.g., a cellular signaling pathway.
   E122. The method of any one of the preceding embodiments, wherein the TREM comprises a post-transcriptional modification from Table 2.
   E123. The method of any one of the preceding embodiments, wherein the TREM comprises cognate adaptor function, and wherein the TREM mediates acceptance and incorporation of an amino acid associated in nature with the anti-codon of the TREM in the initiation or elongation of a peptide chain.
   E124. The method of any one of the preceding embodiments, wherein the TREM comprises an RNA sequence at least 80% identical to an RNA sequence of a tRNA which occurs naturally.
   E125. The method of any one of the preceding embodiments, wherein the TREM comprises an RNA sequence at least 80% identical to an RNA encoded by a DNA sequence listed in Table 1, or a fragment or functional fragment thereof.
   E126. The method of any one of the preceding embodiments, wherein the TREM comprises an RNA sequence encoded by a DNA sequence listed in Table 1, or a fragment thereof.
   E127. The method of any one of the preceding embodiments, wherein the TREM comprises an RNA sequence at least XX % identical to an RNA sequence encoded by a DNA sequence listed in Table 1, or a fragment thereof, wherein XX is selected from 80, 85, 90, 95, 96, 97, 98, or 99.
   E128. The method of embodiment E127, wherein XX is 80.
   E129. The method of embodiment E127, wherein XX is 85.
   E130. The method of embodiment E127, wherein XX is 90.
   E131. The method of embodiment E127, wherein XX is 95.
   E132. The method of embodiment E127, wherein XX is 97.
   E133. The method of embodiment E127, wherein XX is 98.
   E134. The method of embodiment E127, wherein XX is 99.
   E135. The method of any one of embodiments E127-E134, wherein the DNA sequence is SEQ ID NO:1 or a fragment thereof, or SEQ ID NO:2 or a fragment thereof, or SEQ ID NO: 3 or a fragment thereof, or SEQ ID NO:4 or a fragment thereof, or SEQ ID NO: 5 or a fragment thereof, or SEQ ID NO: 6 or a fragment thereof, or SEQ ID NO: 7 or a fragment thereof, or SEQ ID NO:8 or a fragment thereof, or SEQ ID NO: 9 or a fragment thereof, or SEQ ID NO:10 or a fragment thereof, or SEQ ID NO: 11 or a fragment thereof, or SEQ ID NO:12 or a fragment thereof, or SEQ ID NO: 13 or a fragment thereof, or SEQ ID NO: 14 or a fragment thereof, or SEQ ID NO: 15 or a fragment thereof, or SEQ ID NO: 16 or a fragment thereof, or SEQ ID NO: 17 or a fragment thereof, or SEQ ID NO: 18 or a fragment thereof, or SEQ ID NO: 19 or a fragment thereof, or SEQ ID NO: 20 or a fragment thereof, or SEQ ID NO: 21 or a fragment thereof, or SEQ ID NO: 22 or a fragment thereof, or SEQ ID NO: 23 or a fragment thereof, or SEQ ID NO: 24 or a fragment thereof, or SEQ ID NO: 25 or a fragment thereof, or SEQ ID NO: 26 or a fragment thereof, or SEQ ID NO: 27 or a fragment thereof, or SEQ ID NO: 28 or a fragment thereof, or SEQ ID NO: 29 or a fragment thereof, or SEQ ID NO: 30 or a fragment thereof, or SEQ ID NO: 31 or a fragment thereof, or SEQ ID NO: 32 or a fragment thereof, or SEQ ID NO: 33 or a fragment thereof, or SEQ ID NO: 34 or a fragment thereof, or SEQ ID NO: 35 or a fragment thereof, or SEQ ID NO: 36 or a fragment thereof, or SEQ ID NO: 37 or a fragment thereof, or SEQ ID NO: 38 or a fragment thereof, or SEQ ID NO: 39 or a fragment thereof, or SEQ ID NO: 40 or a fragment thereof, or SEQ ID NO: 41 or a fragment thereof, or SEQ ID NO: 42 or a fragment thereof, or SEQ ID NO: 43 or a fragment thereof, or SEQ ID NO: 44 or a fragment thereof, or SEQ ID NO: 45 or a fragment thereof, or SEQ ID NO: 46 or a fragment thereof, or SEQ ID NO: 47 or a fragment thereof, or SEQ ID NO: 48 or a fragment thereof, or SEQ ID NO: 49 or a fragment thereof, or SEQ ID NO: 50 or a fragment thereof, or SEQ ID NO: 51 or a fragment thereof, or SEQ ID NO: 52 or a fragment thereof, or SEQ ID NO: 53 or a fragment thereof, or SEQ ID NO: 54 or a fragment thereof, or SEQ ID NO: 55 or a fragment thereof, or SEQ ID NO: 56 or a fragment thereof, or SEQ ID NO: 57 or a fragment thereof, or SEQ ID NO: 58 or a fragment thereof, or SEQ ID NO: 59 or a fragment thereof, or SEQ ID NO: 60 or a fragment thereof, or SEQ ID NO: 61 or a fragment thereof, or SEQ ID NO: 62 or a fragment thereof, or SEQ ID NO: 63 or a fragment thereof, or SEQ ID NO: 64 or a fragment thereof, or SEQ ID NO: 65 or a fragment thereof, or SEQ ID NO: 66 or a fragment thereof, or SEQ ID NO: 67 or a fragment thereof, or SEQ ID NO: 68 or a fragment thereof, or SEQ ID NO: 69 or a fragment thereof, or SEQ ID NO: 70 or a fragment thereof, } or SEQ ID NO: 71 or a fragment thereof, or SEQ ID NO: 72 or a fragment thereof, or SEQ ID NO: 73 or a fragment thereof, or SEQ ID NO: 74 or a fragment thereof, or SEQ ID NO: 75 or a fragment thereof, or SEQ ID NO: 76 or a fragment thereof, or SEQ ID NO: 77 or a fragment thereof, or SEQ ID NO: 78 or a fragment thereof, or SEQ ID NO: 79 or a fragment thereof, or SEQ ID NO: 80 or a fragment thereof, or SEQ ID NO: 81 or a fragment thereof, or SEQ ID NO: 82 or a fragment thereof, or SEQ ID NO: 83 or a fragment thereof, or SEQ ID NO: 84 or a fragment thereof, or SEQ ID NO: 85 or a fragment thereof, or SEQ ID NO: 86 or a fragment thereof, or SEQ ID NO: 87 or a fragment thereof, or SEQ ID NO: 88 or a fragment thereof, or SEQ ID NO: 89 or a fragment thereof, or SEQ ID NO: 90 or a fragment thereof, or SEQ ID NO: 91 or a fragment thereof, or SEQ ID NO: 92 or a fragment thereof, or SEQ ID NO: 93 or a fragment thereof, or SEQ ID NO: 94 or a fragment thereof, or SEQ ID NO: 95 or a fragment thereof, or SEQ ID NO: 96 or a fragment thereof, or SEQ ID NO: 97 or a fragment thereof, or SEQ ID NO: 98 or a fragment thereof, or SEQ ID NO: 99 or a fragment thereof, or SEQ ID NO: 100 or a fragment thereof, or SEQ ID NO: 101 or a fragment thereof, or SEQ ID NO: 102 or a fragment thereof, or SEQ ID NO: 103 or a fragment thereof, or SEQ ID NO: 104 or a fragment thereof, or SEQ ID NO: 105 or a fragment thereof, or SEQ ID NO: 106 or a fragment thereof, or SEQ ID NO: 107 or a fragment thereof, or SEQ ID NO: 108 or a fragment thereof, or SEQ ID NO:109 or a fragment thereof, or SEQ ID NO: 110 or a fragment thereof, or SEQ ID NO: 111 or a fragment thereof, or SEQ ID NO: 112 or a fragment thereof, or SEQ ID NO: 113 or a fragment thereof, or SEQ ID NO: 114 or a fragment thereof, or SEQ ID NO: 115 or a fragment thereof, or SEQ ID NO: 116 or a fragment thereof, or SEQ ID NO: 117 or a fragment thereof, or SEQ ID NO: 118 or a fragment thereof, or SEQ ID NO: 119 or a fragment thereof, or SEQ ID NO: 120 or a fragment thereof, or SEQ ID NO: 121 or a fragment thereof, or SEQ ID NO: 122 or a fragment thereof, or SEQ ID NO: 123 or a fragment thereof, or SEQ ID NO: 124 or a fragment thereof, or SEQ ID NO: 125 or a fragment thereof, or SEQ ID NO: 126 or a fragment thereof, or SEQ ID NO: 127 or a fragment thereof, or SEQ ID NO: 128 or a fragment thereof, or SEQ ID NO: 129 or a fragment thereof, or SEQ ID NO: 130 or a fragment thereof, or SEQ ID NO: 131 or a fragment thereof, or SEQ ID NO: 132 or a fragment thereof, or SEQ ID NO: 133 or a fragment thereof, or SEQ ID NO: 134 or a fragment thereof, or SEQ ID NO: 135 or a fragment thereof, or SEQ ID NO:136 or a fragment thereof, or SEQ ID NO: 137 or a fragment thereof, or SEQ ID NO: 138 or a fragment thereof, or SEQ ID NO: 139 or a fragment thereof, or SEQ ID NO: 140 or a fragment thereof, or SEQ ID NO: 141 or a fragment thereof, or SEQ ID NO: 142 or a fragment thereof, or SEQ ID NO: 143 or a fragment thereof, or SEQ ID NO: 144 or a fragment thereof, or SEQ ID NO: 145 or a fragment thereof, or SEQ ID NO: 146 or a fragment thereof, or SEQ ID NO: 147 or a fragment thereof, or SEQ ID NO: 148 or a fragment thereof, or SEQ ID NO: 149 or a fragment thereof, or SEQ ID NO: 150 or a fragment thereof, or SEQ ID NO: 151 or a fragment thereof, or SEQ ID NO: 152 or a fragment thereof, or SEQ ID NO: 153 or a fragment thereof, or SEQ ID NO: 154 or a fragment thereof, or SEQ ID NO: 155 or a fragment thereof, or SEQ ID NO: 156 or a fragment thereof, or SEQ ID NO: 157 or a fragment thereof, or SEQ ID NO: 158 or a fragment thereof, or SEQ ID NO: 159 or a fragment thereof, or SEQ ID NO: 160 or a fragment thereof, or SEQ ID NO: 161 or a fragment thereof, or SEQ ID NO: 162 or a fragment thereof, or SEQ ID NO: 163 or a fragment thereof, or SEQ ID NO: 164 or a fragment thereof, or SEQ ID NO: 165 or a fragment thereof, or SEQ ID NO: 166 or a fragment thereof, or SEQ ID NO: 167 or a fragment thereof, or SEQ ID NO: 168 or a fragment thereof, or SEQ ID NO: 169 or a fragment thereof, or SEQ ID NO: 170 or a fragment thereof, or SEQ ID NO: 171 or a fragment thereof, or SEQ ID NO: 172 or a fragment thereof, or SEQ ID NO: 173 or a fragment thereof, or SEQ ID NO: 174 or a fragment thereof, or SEQ ID NO: 175 or a fragment thereof, or SEQ ID NO: 176 or a fragment thereof, or SEQ ID NO: 177 or a fragment thereof, or SEQ ID NO: 178 or a fragment thereof, or SEQ ID NO: 179 or a fragment thereof, or SEQ ID NO: 180 or a fragment thereof, or SEQ ID NO: 181 or a fragment thereof, or SEQ ID NO: 182 or a fragment thereof, or SEQ ID NO: 183 or a fragment thereof, or SEQ ID NO: 184 or a fragment thereof, or SEQ ID NO: 185 or a fragment thereof, or SEQ ID NO: 186 or a fragment thereof, or SEQ ID NO: 187 or a fragment thereof, or SEQ ID NO: 188 or a fragment thereof, or SEQ ID NO: 189 or a fragment thereof, or SEQ ID NO: 190 or a fragment thereof, or SEQ ID NO: 191 or a fragment thereof, or SEQ ID NO: 192 or a fragment thereof, or SEQ ID NO: 193 or a fragment thereof, or SEQ ID NO: 194 or a fragment thereof, or SEQ ID NO: 195 or a fragment thereof, or SEQ ID NO: 196 or a fragment thereof, or SEQ ID NO: 197 or a fragment thereof, or SEQ ID NO: 198 or a fragment thereof, or SEQ ID NO: 199 or a fragment thereof, or SEQ ID NO: 200 or a fragment thereof, or SEQ ID NO: 201 or a fragment thereof, or SEQ ID NO: 202 or a fragment thereof, or SEQ ID NO: 203 or a fragment thereof, or SEQ ID NO: 204 or a fragment thereof, or SEQ ID NO: 205 or a fragment thereof, or SEQ ID NO: 206 or a fragment thereof, or SEQ ID NO: 207 or a fragment thereof, or SEQ ID NO: 208 or a fragment thereof, or SEQ ID NO: 209 or a fragment thereof, or SEQ ID NO: 210 or a fragment thereof, or SEQ ID NO: 211 or a fragment thereof, or SEQ ID NO: 212 or a fragment thereof, or SEQ ID NO: 213 or a fragment thereof, or SEQ ID NO: 214 or a fragment thereof, or SEQ ID NO: 215 or a fragment thereof, or SEQ ID NO: 216 or a fragment thereof, or SEQ ID NO: 217 or a fragment thereof, or SEQ ID NO: 218 or a fragment thereof, or SEQ ID NO: 219 or a fragment thereof, or SEQ ID NO: 220 or a fragment thereof, or SEQ ID NO: 221 or a fragment thereof, or SEQ ID NO: 222 or a fragment thereof, or SEQ ID NO: 223 or a fragment thereof, or SEQ ID NO: 224 or a fragment thereof, or SEQ ID NO: 225 or a fragment thereof, or SEQ ID NO: 226 or a fragment thereof, or SEQ ID NO: 227 or a fragment thereof, or SEQ ID NO: 228 or a fragment thereof, or SEQ ID NO: 229 or a fragment thereof, or SEQ ID NO: 230 or a fragment thereof, or SEQ ID NO: 231 or a fragment thereof, or SEQ ID NO: 232 or a fragment thereof, or SEQ ID NO: 233 or a fragment thereof, or SEQ ID NO: 234 or a fragment thereof, or SEQ ID NO: 235 or a fragment thereof, or SEQ ID NO: 236 or a fragment thereof, or SEQ ID NO: 237 or a fragment thereof, or SEQ ID NO: 238 or a fragment thereof, or SEQ ID NO: 239 or a fragment thereof, or SEQ ID NO: 240 or a fragment thereof, or SEQ ID NO: 241 or a fragment thereof, or SEQ ID NO: 242 or a fragment thereof, or SEQ ID NO: 243 or a fragment thereof, or SEQ ID NO: 244 or a fragment thereof, or SEQ ID NO: 245 or a fragment thereof, or SEQ ID NO: 246 or a fragment thereof, or SEQ ID NO: 247 or a fragment thereof, or SEQ ID NO: 248 or a fragment thereof, or SEQ ID NO: 249 or a fragment thereof, or SEQ ID NO: 250 or a fragment thereof, or SEQ ID NO: 251 or a fragment thereof, or SEQ ID NO: 252 or a fragment thereof, or SEQ ID NO: 253 or a fragment thereof, or SEQ ID NO: 254 or a fragment thereof, or SEQ ID NO: 255 or a fragment thereof, or SEQ ID NO: 256 or a fragment thereof, or SEQ ID NO: 257 or a fragment thereof, or SEQ ID NO: 258 or a fragment thereof, or SEQ ID NO: 259 or a fragment thereof, or SEQ ID NO: 260 or a fragment thereof, or SEQ ID NO: 261 or a fragment thereof, or SEQ ID NO: 262 or a fragment thereof, or SEQ ID NO: 263 or a fragment thereof, or SEQ ID NO: 264 or a fragment thereof, or SEQ ID NO: 265 or a fragment thereof, or SEQ ID NO: 266 or a fragment thereof, or SEQ ID NO: 267 or a fragment thereof, or SEQ ID NO: 268 or a fragment thereof, or SEQ ID NO: 269 or a fragment thereof, or SEQ ID NO: 270 or a fragment thereof, or SEQ ID NO: 271 or a fragment thereof, or SEQ ID NO: 272 or a fragment thereof, or SEQ ID NO: 273 or a fragment thereof, or SEQ ID NO: 274 or a fragment thereof, or SEQ ID NO: 275 or a fragment thereof, or SEQ ID NO: 276 or a fragment thereof, or SEQ ID NO: 277 or a fragment thereof, or SEQ ID NO: 278 or a fragment thereof, or SEQ ID NO: 279 or a fragment thereof, or SEQ ID NO: 280 or a fragment thereof, or SEQ ID NO: 281 or a fragment thereof, or SEQ ID NO: 282 or a fragment thereof, or SEQ ID NO: 283 or a fragment thereof, or SEQ ID NO: 284 or a fragment thereof, or SEQ ID NO: 285 or a fragment thereof, or SEQ ID NO: 286 or a fragment thereof, or SEQ ID NO: 287 or a fragment thereof, or SEQ ID NO: 288 or a fragment thereof, or SEQ ID NO: 289 or a fragment thereof, or SEQ ID NO: 290 or a fragment thereof, or SEQ ID NO: 291 or a fragment thereof, or SEQ ID NO: 292 or a fragment thereof, or SEQ ID NO: 293 or a fragment thereof, or SEQ ID NO: 294 or a fragment thereof, or SEQ ID NO: 295 or a fragment thereof, or SEQ ID NO: 296 or a fragment thereof, or SEQ ID NO: 297 or a fragment thereof, or SEQ ID NO: 298 or a fragment thereof, or SEQ ID NO: 299 or a fragment thereof, or SEQ ID NO: 300 or a fragment thereof, or SEQ ID NO: 301 or a fragment thereof, or SEQ ID NO: 302 or a fragment thereof, or SEQ ID NO: 303 or a fragment thereof, or SEQ ID NO: 304 or a fragment thereof, or SEQ ID NO: 305 or a fragment thereof, or SEQ ID NO: 306 or a fragment thereof, or SEQ ID NO: 307 or a fragment thereof, or SEQ ID NO: 308 or a fragment thereof, or SEQ ID NO: 309 or a fragment thereof, or SEQ ID NO: 310 or a fragment thereof, or SEQ ID NO: 311 or a fragment thereof, or SEQ ID NO: 312 or a fragment thereof, or SEQ ID NO: 313 or a fragment thereof, or SEQ ID NO: 314 or a fragment thereof, or SEQ ID NO: 315 or a fragment thereof, or SEQ ID NO: 316 or a fragment thereof, or SEQ ID NO: 317 or a fragment thereof, or SEQ ID NO: 318 or a fragment thereof, or SEQ ID NO: 319 or a fragment thereof, or SEQ ID NO: 320 or a fragment thereof, or SEQ ID NO: 321 or a fragment thereof, or SEQ ID NO: 322 or a fragment thereof, or SEQ ID NO: 323 or a fragment thereof, or SEQ ID NO: 324 or a fragment thereof, or SEQ ID NO: 325 or a fragment thereof, or SEQ ID NO: 326 or a fragment thereof, or SEQ ID NO: 327 or a fragment thereof, or SEQ ID NO: 328 or a fragment thereof, or SEQ ID NO: 329 or a fragment thereof, or SEQ ID NO: 330 or a fragment thereof, or SEQ ID NO: 331 or a fragment thereof, or SEQ ID NO: 332 or a fragment thereof, or SEQ ID NO: 333 or a fragment thereof, or SEQ ID NO: 334 or a fragment thereof, or SEQ ID NO: 335 or a fragment thereof, or SEQ ID NO: 336 or a fragment thereof, or SEQ ID NO: 337 or a fragment thereof, or SEQ ID NO: 338 or a fragment thereof, or SEQ ID NO: 339 or a fragment thereof, or SEQ ID NO: 340 or a fragment thereof, or SEQ ID NO: 341 or a fragment thereof, or SEQ ID NO: 342 or a fragment thereof, or SEQ ID NO: 343 or a fragment thereof, or SEQ ID NO: 344 or a fragment thereof, or SEQ ID NO: 345 or a fragment thereof, or SEQ ID NO: 346 or a fragment thereof, or SEQ ID NO: 347 or a fragment thereof, or SEQ ID NO: 348 or a fragment thereof, or SEQ ID NO: 349 or a fragment thereof, or SEQ ID NO: 350 or a fragment thereof, or SEQ ID NO: 351 or a fragment thereof, or SEQ ID NO: 352 or a fragment thereof, or SEQ ID NO: 353 or a fragment thereof, or SEQ ID NO: 354 or a fragment thereof, or SEQ ID NO: 355 or a fragment thereof, or SEQ ID NO: 356 or a fragment thereof, or SEQ ID NO: 357 or a fragment thereof, or SEQ ID NO: 358 or a fragment thereof, or SEQ ID NO: 359 or a fragment thereof, or SEQ ID NO: 360 or a fragment thereof, or SEQ ID NO: 361 or a fragment thereof, or SEQ ID NO: 362 or a fragment thereof, or SEQ ID NO: 363 or a fragment thereof, or SEQ ID NO: 364 or a fragment thereof, or SEQ ID NO: 365 or a fragment thereof, or SEQ ID NO: 366 or a fragment thereof, or SEQ ID NO: 367 or a fragment thereof, or SEQ ID NO: 368 or a fragment thereof, or SEQ ID NO: 369 or a fragment thereof, or SEQ ID NO: 370 or a fragment thereof, or SEQ ID NO: 371 or a fragment thereof, or SEQ ID NO: 372 or a fragment thereof, or SEQ ID NO: 373 or a fragment thereof, or SEQ ID NO: 374 or a fragment thereof, or SEQ ID NO: 375 or a fragment thereof, or SEQ ID NO: 376 or a fragment thereof, or SEQ ID NO: 377 or a fragment thereof, or SEQ ID NO: 378 or a fragment thereof, or SEQ ID NO: 379 or a fragment thereof, or SEQ ID NO: 380 or a fragment thereof, or SEQ ID NO: 381 or a fragment thereof, or SEQ ID NO: 382 or a fragment thereof, or SEQ ID NO: 383 or a fragment thereof, or SEQ ID NO: 384 or a fragment thereof, or SEQ ID NO: 385 or a fragment thereof, or SEQ ID NO: 386 or a fragment thereof, or SEQ ID NO: 387 or a fragment thereof, or SEQ ID NO: 388 or a fragment thereof, or SEQ ID NO: 389 or a fragment thereof, or SEQ ID NO: 390 or a fragment thereof, or SEQ ID NO: 391 or a fragment thereof, or SEQ ID NO: 392 or a fragment thereof, or SEQ ID NO: 393 or a fragment thereof, or SEQ ID NO: 394 or a fragment thereof, or SEQ ID NO: 395 or a fragment thereof, or SEQ ID NO: 396 or a fragment thereof, or SEQ ID NO: 397 or a fragment thereof, or SEQ ID NO: 398 or a fragment thereof, or SEQ ID NO: 399 or a fragment thereof, or SEQ ID NO: 400 or a fragment thereof, or SEQ ID NO: 401 or a fragment thereof, or SEQ ID NO: 402 or a fragment thereof, or SEQ ID NO: 403 or a fragment thereof, or SEQ ID NO: 404 or a fragment thereof, or SEQ ID NO: 405 or a fragment thereof, or SEQ ID NO: 406 or a fragment thereof, or SEQ ID NO: 407 or a fragment thereof, or SEQ ID NO: 408 or a fragment thereof, or SEQ ID NO: 409 or a fragment thereof, or SEQ ID NO: 410 or a fragment thereof, or SEQ ID NO: 411 or a fragment thereof, or SEQ ID NO: 412 or a fragment thereof, or SEQ ID NO: 413 or a fragment thereof, or SEQ ID NO: 414 or a fragment thereof, or SEQ ID NO: 415 or a fragment thereof, or SEQ ID NO: 416 or a fragment thereof, or SEQ ID NO: 417 or a fragment thereof, or SEQ ID NO: 418 or a fragment thereof, or SEQ ID NO: 419 or a fragment thereof, or SEQ ID NO: 420 or a fragment thereof, or SEQ ID NO: 421 or a fragment thereof, or SEQ ID NO: 422 or a fragment thereof, or SEQ ID NO: 423 or a fragment thereof, or SEQ ID NO: 424 or a fragment thereof, or SEQ ID NO: 425 or a fragment thereof, or SEQ ID NO: 426 or a fragment thereof, or SEQ ID NO: 427 or a fragment thereof, or SEQ ID NO:428 or a fragment thereof, or SEQ ID NO: 429 or a fragment thereof, or SEQ ID NO: 430 or a fragment thereof, or SEQ ID NO: 431 or a fragment thereof, or SEQ ID NO: 432 or a fragment thereof, or SEQ ID NO: 433 or a fragment thereof, or SEQ ID NO: 434 or a fragment thereof, or SEQ ID NO: 435 or a fragment thereof, or SEQ ID NO: 436 or a fragment thereof, or SEQ ID NO: 437 or a fragment thereof, or SEQ ID NO: 438 or a fragment thereof, or SEQ ID NO: 439 or a fragment thereof, or SEQ ID NO: 440 or a fragment thereof, or SEQ ID NO: 441 or a fragment thereof, or SEQ ID NO: 442 or a fragment thereof, or SEQ ID NO: 443 or a fragment thereof, or SEQ ID NO: 444 or a fragment thereof, or SEQ ID NO: 445 or a fragment thereof, or SEQ ID NO: 446 or a fragment thereof, or SEQ ID NO: 447 or a fragment thereof, or SEQ ID NO: 448 or a fragment thereof, or SEQ ID NO: 449 or a fragment thereof, or SEQ ID NO: 450 or a fragment thereof, or SEQ ID NO: 451 or a fragment thereof.
   E136. The method of any one of the preceding embodiments, wherein the TREM comprises a consensus sequence of Formula I zzz,

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein:
(i) zzz indicates any of the twenty amino acids;
(ii) Formula I corresponds to all species; and
(iii) x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1- 24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271).
   E137. The method of any one of embodiments E1-E135, wherein the TREM comprises a consensus sequence of Formula II zzz,

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂
wherein:
   (i) zzz indicates any of the twenty amino acids;
   (ii) Formula II corresponds to mammals; and
   (iii) x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1- 24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271).
      E138. The method of any one of embodiments E1-E135, wherein the TREM comprises a consensus sequence of Formula IIII zzz,

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂
wherein:
   (i) zzz indicates any of the twenty amino acids;
   (ii) Formula III corresponds to humans; and
   (iii) x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1- 24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271).
      E139. The method of any one of embodiments E136-E138, wherein ZZZ indicates any of the following amino acids: alanine, arginine, asparagine, aspartate, cysteine, glutamine, glutamate, glycine, histidine, isoleucine, methionine, leucine, lysine, phenylalanine, proline, serine, threonine, tryptophan, tyrosine, or valine.
      E140. The method of any one of embodiments E136-E139, comprising a property selected from the following:
a) under physiological conditions residue R₀ forms a linker region, e.g., a Linker 1 region;
b) under physiological conditions residues R₁—R₂—R₃—R₄—R₅—R₆—R₇ and residues R₆₅—R₆₆—R₆₇—R₆₈—R₆₉—R₇₀—R₇₁ form a stem region, e.g., an AStD stem region;
c) under physiological conditions residues R₈—R₉ forms a linker region, e.g., a Linker 2 region;
d) under physiological conditions residues —R₁₀—R₁₁—R₁₂—R₁₃—R₁₄R₁₅—R₁₆—R₁₇—R₁₈—R₁₉—R₂₀—R₂₁—R₂₂—R₂₃—R₂₄—R₂₅—R₂₆—R₂₇—R₂₈ form a stem-loop region, e.g., a D arm Region; e) under physiological conditions residue —R₂₉ forms a linker region, e.g., a Linker 3 Region;
f) under physiological conditions residues —R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆ form a stem-loop region, e.g., an AC arm region;
g) under physiological conditions residue —[R₄₇]ₓ₁ comprises a variable region;
h) under physiological conditions residues —R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄ form a stem-loop region, e.g., a T arm Region; or
i) under physiological conditions residue R₇₂ forms a linker region, e.g., a Linker 4 region.
   E141. The method of embodiment E140, comprising any one of properties (a)-(i).
   E142. The method of embodiment E140, comprising any two of properties (a)-(i).
   E143. The method of embodiment E140, comprising any three of properties (a)-(i).
   E144. The method of embodiment E140, comprising any four of properties (a)-(i).
   E145. The method of embodiment E140, comprising any five of properties (a)-(i).
   E146. The method of embodiment E140, comprising any six of properties (a)-(i).
   E147. The method of embodiment E140, comprising any seven of properties (a)-(i).
   E148. The method of embodiment E140, comprising all of properties (a)-(i).
   E149. The method of embodiment E140, wherein the TREM comprises a variable region at position R₄₇.
   E150. The method of embodiment E140, wherein the variable region is 1-271 residues in length (e.g. 1-250, 1-225, 1-200, 1-175, 1-150, 1-125, 1-100, 1-75, 1-50, 1-40, 1-30, 1-29, 1-28, 1-27, 1-26, 1-25, 1-24, 1-23, 1-22, 1-21, 1-20, 1-19, 1-18, 1-17, 1-16, 1-15, 1-14, 1-13, 1-12, 1-11, 1-10, 10-271, 20-271, 30-271, 40-271, 50-271, 60-271, 70-271, 80-271, 100-271, 125-271, 150-271, 175-271, 200-271, 225-271, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 110, 125, 150, 175, 200, 225, 250, or 271 residues).
   E151. The method of embodiment E140, wherein the variable region the variable region comprises any one, all or a combination of Adenine, Cytosine, Guanine or Uracil.
   E152. The method of embodiment E140, wherein the variable region comprises a ribonucleic acid (RNA) sequence encoded by a deoxyribonucleic acid (DNA) sequence disclosed in Table 3, e.g., any one of SEQ ID NOs: 452-561 disclosed in Table 3.
   E153. A method of making a tRNA effector molecule (TREM), comprising:
(a) providing a host cell, comprising exogenous nucleic acid, e.g., a DNA or RNA, encoding a TREM under conditions sufficient to express the TREM, and
(b) purifying the expressed TREM from the host cell culture to produce a TREM composition, thereby making a TREM composition.
   E154. The method of embodiment E153, wherein the TREM composition comprises a TREM fragment, e.g., as described herein.
   E155. The method of embodiment E154, wherein the TREM fragment is produced in vivo, in the host cell.

E156. The method of embodiment E154, wherein the TREM fragment is produced by fragmenting an expressed TREM after production of the TREM by the cell, e.g., a TREM produced by the host cell is fragmented after release or purification from the host cell, e.g., the TREM is fragmented ex vivo.

E157. The method of any one of embodiments E153-E156, wherein the method results in an increase, e.g., at least a 2.2, 2.5, 3, 4, 5, 6, 7, 8, 9, 10, or 20-fold increase in the production of total of endogenous tRNA and TREM in the host cell (e.g., as measured by an assay described in any of Examples 9-13), e.g., as compared with a reference cell, e.g., a similar cell but not engineered or modified to express a TREM.
E158. The method of embodiment E157, wherein method results in an increase in TREM production and/or tRNA production between 2.2 to 20-fold, between 2.2 to 15-fold, between 2.2 to 10-fold, between 2.2 to 9-fold, between 2.2 to 8-fold, between 2.2 to 7-fold, between 2.2 to 6-fold, between 2.2 to 5-fold, between 2.2 to 4-fold, between 2.2 to 3-fold, between 2.2 to 2.5-fold, between 2.5 to 20-fold, between 3 to 20-fold, between 4 to 20-fold, between 5 to 20-fold, between 6 to 20-fold, between 7 to 20-fold, between 8 to 20-fold, between 9 to 20-fold, between 10 to 20-fold, or between 15 to 20-fold.
E159. The method of any one of embodiments E153-E158, wherein the method results in a detectable level of TREM in the host cell, e.g., as measured by an assay described in any of Examples 9-13.
E160. The method of any one of embodiments E153-E159, wherein the host cell is capable of a post-transcriptional modification, of the TREM.
E161. The method of any one of embodiments E153-E160, wherein the host cell is capable of a post-transcriptional modification, of the TREM, e.g., a post-transcriptional modification selected from Table 2.
E162. The method of any one of embodiments E153-E161, wherein the host cell has been modified to modulate, e.g., increase, its ability to provide a post-transcriptional modification, of the TREM, e.g., a post-transcriptional modification selected from Table 2, e.g., the host cell has been modified to provide for, an increase, or decrease in, the expression of a gene, e.g., a gene encoding an enzyme from Table 2, or a gene encoding an enzyme having nuclease activity (e.g., endonuclease activity or ribonuclease activity), e.g., or one or more of Dicer, Angiogenin, RNaseA, RNaseP, RNaseZ, Rny1 or PrrC.
E163. The method of any one of embodiments E153-E162, wherein the host cell is a mammalian cell capable of a post-transcriptional modification, of the TREM, e.g., a post-transcriptional modification selected from Table 2.
E164. The method of any one of embodiments E153-E163, wherein the host cell comprises a HeLa cell, a HEK293 cell, a HT-1080 cell, a PER.C6 cell, a HKB-11 cell, a CAP cell or a HuH-7 cell.
E165. The method of any one of embodiments E153-E164, wherein the host cell has increased expression of an oncogene, e.g., Ras, c-myc or c-jun.
E166. The method of any one of embodiments E153-E165, wherein the host cell has decreased expression of a tumor suppressor, e.g., p53 or Rb.
E167. The method of any one of embodiments E153-E166, wherein the host cell has increased expression of RNA Polymerase III (RNA Pol III).
E168. The method of any one of embodiments E153-E167, wherein the host cell is a non-mammalian host cell.
E169. The method of any one of embodiments E153-E168, wherein the host cell is a bacterial cell, e.g., an E. coli cell, or a yeast cell.
E170. The method of any one of embodiments E153-E169, further comprising measuring one or more of the following characteristics of the TREM composition (or an intermediate in the production of a TREM composition):
(i) purity of at least 30%, 40%, 50%, 60%, 70%, 80%, 85%, 90%, 91%, 92%, 93%, 94%, 95%, 96%, 97%, 98%, or 99%;
(ii) host cell protein (HCP) contamination of less than 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
(iii) host cell protein (HCP) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, or 100 ng, per milligram (mg) of the TREM composition;
(iv) DNA, e.g., host cell DNA, of less than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
(v) fragments of less than 0.1%, 1%, 2%, 3%, 4%, 5%, 6%, 7%, 8%, 9% or 10%;
(vi) low levels or absence of endotoxins, e.g., as measured by the Limulus amebocyte lysate (LAL) test;
(vii) in-vitro translation activity, e.g., as measured by an assay described in Example 8; (viii) TREM concentration of at least 0.1 ng/mL, 0.5 ng/mL, 1 ng/mL, 5 ng/mL, 10 ng/mL, 50 ng/mL, 0.1 ug/mL, 0.5 ug/mL, 1 ug/mL, 2 ug/mL, 5 ug/mL, 10 ug/mL, 20 ug/mL, 30 ug/mL, 40 ug/mL, 50 ug/mL, 60 ug/mL, 70 ug/mL, 80 ug/mL, 100 ug/mL, 200 ug/mL, 300 ug/mL, 500 ug/mL, 1000 ug/mL, 5000 ug/mL, 10,000 ug/mL, or 100,000 ug/mL;
(ix) sterility, e.g., as per cGMP guidelines for sterile drug products, e.g., the composition or preparation supports the growth of fewer than 100 viable microorganisms as tested under aseptic conditions, the composition or preparation meets the standard of USP <71>, and/or the composition or preparation meets the standard of USP <85>; or
(x) viral contamination, e.g., the composition or preparation has an absence of or an undetectable level of viral contamination.
   E171. The method of embodiment E170, further comprising, comparing the measured value with a reference value or a standard.
   E172. The method of embodiment E170, further comprising, in response to the comparison, modulating the TREM composition to:
(i) increase the purity of the composition;
(ii) decrease the amount of HCP in the composition;
(iii) decrease the amount of DNA in the composition;
(iv) decrease the amount of fragments in the composition;
(v) decrease the amount of endotoxins in the composition;
(vi) increase the in vitro translation activity of the composition;
(vii) increase the TREM concentration of the composition; or
(viii) increase the sterility of the composition.
   E173. The method of any one of embodiments E153-E172, wherein the TREM was purified from host cells cultured in a bioreactor.
   E174. The bioreactor of embodiment E173,
(i) comprising at least 1×10⁷, 1×10⁸, 1×10⁹, 1×10¹⁰, 1×10¹¹, 1×10¹², 1×10¹³, or 1×10¹⁴ host cells;
(ii) comprising between 100 mL and 100 liters of culture medium, e.g., at least 100 mL, 250 mL, 500 mL, 750 mL, 1 liter, 2 liters, 3 liters, 4 liters, 5 liters, 6 liters, 7 liters, 8 liters, 9 liters, 10 liters, 15 liters, 20 liters, 25 liters, 30 liters, 40 liters, 50 liters, 60 liters, 70 liters, 80 liters, 90 liters, or 100 liters of culture medium;
(iii) wherein the bioreactor is selected from a continuous flow bioreactor, a batch process bioreactor, a perfusion bioreactor, and a fed batch bioreactor; or
(iv) wherein the bioreactor is held under conditions sufficient to express the TREM.
   E175. The method of any one of embodiments E153-E174, wherein the TREM is encoded by, or expressed from, a nucleic acid sequence comprising:
(i) a control region sequence;
(ii) a sequence encoding a modified TREM;
(iii) a sequence encoding more than one TREM; or
(iv) a sequence other than a tRNA^{MET} sequence.
   E176. The method of embodiment E175, wherein the nucleic acid sequence comprises a promoter sequence.
   E177. The method of embodiment E175 or E176, wherein the nucleic acid sequence comprises a promoter sequence that comprises an RNA polymerase III (Pol III) recognition site, e.g., a Pol III binding site, e.g., a U6 promoter sequence or fragment thereof.

Other features, objects, and advantages of the invention will be apparent from the description and from the claims.

Unless otherwise defined, all technical and scientific terms used herein have the same meaning as commonly understood by one of ordinary skill in the art to which this invention pertains. All publications, patent applications, patents, and other references mentioned herein are incorporated by reference in their entirety. In addition, the materials, methods, and examples are illustrative only and not intended to be limiting.

### BRIEF DESCRIPTION OF THE DRAWINGS

The following detailed description of the disclosure may be better understood when read in conjunction with the appended drawings. It should be understood, however, that the disclosure is not limited to the precise arrangement and instrumentalities of the embodiments shown in the drawings.

FIGS. 1A-1B are images depicting the tRNA levels in HEK293T cells as quantified by Oxford Nanopore sequencing, as described in Example 1. FIG. 1A depicts tRNA profiling by Nanopore sequencing, wherein each line in the graph demonstrates a different sample preparation method. FIG. 1B depicts the levels of tRNA in normal cells compared to cells overexpressing the iMet tRNA.

FIG. 2 depicts the contextual rarity of tRNAs in HEK293T cells. The x axis shows the tRNA frequency in HEK293T cells as determined by tRNA quantification and the y axis shows the HEK293T proteome codon count as determined by the sum of all protein codon counts multiplied by the protein's respective abundance.

FIGS. 3A-3B show an exemplary method of TREM purification. FIG. 3A depicts the tRNA isolation method used for tRNA enrichment and isolation from cells. A phenol-chloroform (P/C) extraction is first used to remove cellular materials. The RNA fraction is flowed through a column, such as an miRNeasy column, to enrich for RNAs over 200 nucleotides and by a LiCl precipitation that serves to remove large RNAs. The material is then run through a G25 column to end up with the final enriched tRNA fraction. FIG. 3B shows that the purification method described in FIG. 3A results in a tRNA fraction that contains less RNA contaminants than a Trizol RNA extraction purification method.

FIGS. 4A-4B show that the tRNA purification method results shows that the tRNA purification method results in a tRNA elution (lane 3) without contaminating RNA of different sizes. In addition, FIG. 4 shows that engineering 293T cells to overexpress initiator methionine leads to more tRNA expression in the input (compare lanes 1 to 4). 293T iMet are 293T cells engineered to overexpress a plasmid which comprises the initiator methionine gene. Lanes 1: input from 293T parental cell purification, 2: flow through from 293T parental cell purification, 3: elution from 293T parental cell purification, 4: input from 293T iMet cell purification 5: flow through from 293T iMet cell purification 6: elution from 293T iMet cell purification.

FIG. 5 is a set of images depicting that two Cy3-labeled TREMs (Cy3-iMet-1 and Cy3-iMet-2) can be delivered via liposome transfection to cells, namely to U20S, HeLa, and H2199 cell lines.

FIGS. 6A-6C are graphs showing an increase in cell growth in three cells lines after transfection with a TREM corresponding to the initiator methionine (iMet), as described in Example 9. FIG. 6A is a graph showing increased % cellular confluency (a measure of cell growth) of U20S cells transfected with Cy3-labeled iMet-CAT-TREM or transfected with a Cy3-labeled non-targeted control. FIG. 6B is a graph showing increased % cellular confluency (a measure of cell growth) of H1299 cells transfected with Cy3-labeled iMet-CAT-TREM or transfected with a Cy3-labeled non-targeted control. FIG. 6C is a graph showing increased % cellular confluency (a measure of cell growth) of Hela cells transfected with Cy3-labeled iMet-CAT-TREM or transfected with a Cy3-labeled non-targeted control.

FIG. 7 is a graph depicting the results of a translational suppression assay, in which an exemplary TREM is transfected at increasing doses in mammalian cells encoding a NanoLuc reporter containing a TGA stop codon, which leads to increased bioluminescence as a readout of stop codon readthrough.

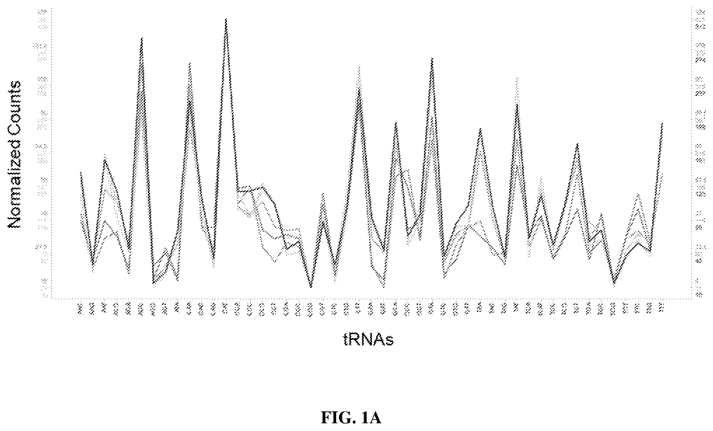

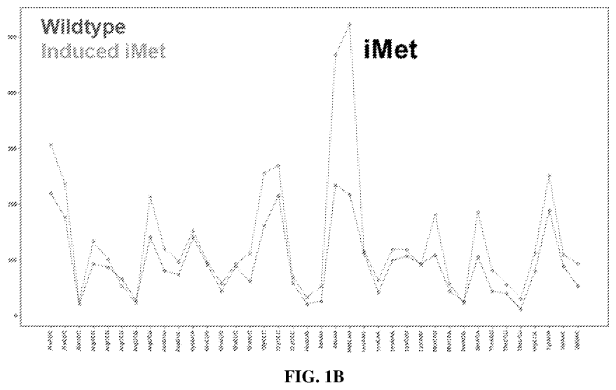

### DETAILED DESCRIPTION OF CERTAIN EMBODIMENTS

The present disclosure features, inter alia, methods of using tRNA-based effector molecules (TREMs) to modulate tRNA pools in a cell or a subject. Also disclosed herein are methods of treating a disorder or ameliorating a symptom of a disorder by administering a TREM composition comprising a TREM or a pharmaceutical composition comprising a TREM. As disclosed herein tRNA-based effector molecules (TREMs) are complex molecules which can mediate a variety of cellular processes. Pharmaceutical compositions comprising a TREM can be administered to a cell, a tissue, or to a subject to modulate these functions.

### Definitions

As used herein, the articles “a” and “an” refer to one or to more than one (e.g., to at least one) of the grammatical object of the article.

A “contextually rare codon” or “con-rare codon”, as those terms are used herein, refer to a codon which, in a target cell or tissue, is limiting for a production parameter, e.g., an expression parameter, for a nucleic acid sequence having a con-rare codon (“con-rare codon nucleic acid sequence”), e.g., because the availability of a tRNA corresponding to the con-rare codon is limiting for a production parameter. Contextual rareness or con-rarity can be identified or evaluated by determining if the addition of a tRNA corresponding to a con-rare codon modulates, typically increases, a production parameter for a nucleic acid sequence, e.g., gene. Contextual rareness or con-rarity can be identified or evaluated by whether a codon satisfies a reference value for proteome codon count-tRNA frequency (PCC-tF, as described herein). By way of example, the method of Example 3, can be used, or adapted to be used, to evaluate con-rarity. Con-rarity as a property of a codon, is a function of, and can be identified or evaluated on the basis of, one, two, three, four, five, six, or all of the following factors:

(1) the sequence of the con-rare codon, or candidate con-rare codon;

(2) the availability of a corresponding tRNA for the con-rare, or candidate con-rare, -codon in a target cell or tissue Availability as a parameter can comprise or be a function of, one or both of the observed or predicted abundance or availability of a tRNA that corresponds to the con-rare codon. In an embodiment, abundance can be evaluated by quantifying tRNAs present in a target cell or tissue. See, e.g., Example 1;

(3) the contextual demand (the demand in a target cell or tissue) for a tRNA, e.g., a con-rare tRNA, or a candidate con-rare tRNA. This can be identified or evaluated by use of a parameter, a contextual demand-parameter, which comprises or is a function of, the demand or usage of a con-rare tRNA by one, some, or all of the nucleic acid sequences having con-rare codons in a target tissue or cell, e.g., the other nucleic acid sequences in a target cell or tissue which have a con-rare codon. A demand parameter can comprise of, or be a function of one or more, or all of:
(a) the expression profile (or proteomic properties) in the target cell or tissue (e.g., the abundance of expression) of one, some, or all of the nucleic acid sequences in the target cell or tissue which have a con-rare codon (e.g., for one or more, a subset of, or all of the expressed con-rare codon nucleic acid sequences in the target cell or tissue). In an embodiment, the expression profile (or proteomic properties) can be evaluated by evaluating proteins expressed in a target cell or tissue. See, e.g., Example 2;
(b) a measure which comprises or is a function of the frequency or proportion of appearance of the con-rare codon in an expressed nucleic acid sequence (e.g., for one or more, a subset of, or all of the expressed con-rare codon nucleic acid sequences in the target cell or tissue); or
(c) a parameter that is a function of (3)(a) and (3)(b);

(4) a parameter (or use-parameter) related to the con-rare codon usage in a con-rare codon nucleic acid sequence, and can include one or more of:
(a) the expression profile (or proteomic properties) in the target cell or tissue (e.g., the abundance of expression) of one, some, or all of the nucleic acid sequences in the target cell or tissue which have a con-rare codon, or a candidate nucleic acid sequence having a con-rare codon, (e.g., for one or more, a subset of, or all of the expressed con-rare codon nucleic acid sequences(s) in the target cell or tissue). In an embodiment, the expression profile (or proteomic properties) can be evaluated by evaluating proteins expressed in a target cell or tissue. See, e.g., Example 2;
(b) a measure which comprises or is a function of the frequency or proportion of appearance of the con-rare codon in a nucleic acid sequence having a con-rare codon (e.g., for one or more, a subset of, or all of the expressed con-rare codon nucleic acid sequence(s) in the target cell or tissue); or
(c) a parameter that is a function of (4)(a) and (4)(b);

(5) the proportion of the tRNAs corresponding to the con-rare codon which are charged;

(6) the iso-decoder isotype of the tRNA corresponding to the con-rare codon; and

(7) one or more post-transcriptional modifications of the con-rare tRNA, or candidate con-rare tRNA.

In an embodiment, a con-optimized nucleic acid sequence has one less or one more con-rare codon than a reference sequence, e.g., a parental sequence, a naturally occurring sequence, a wildtype sequence, or a conventionally optimized sequence.

In an embodiment, con-rarity can be identified or evaluated by: (i) direct determination of whether a con-rare codon or candidate con-rare codon is limiting for a production parameter, e.g., in an assay analogous to that of Example 3; (ii) whether a con-rare or candidate con-rare codon meets a predetermined value, e.g., a standard or reference value (e.g., as described herein), of one or more, or all of factors (1)-(7); or (i) and (ii).

In an embodiment, con-rarity can be identified or evaluated by a production parameter, e.g., an expression parameter or a signaling parameter, e.g., as described herein.

In an embodiment, con-rarity is a function of normalized proteome codon count and tRNA abundance in a target tissue or cell. In an embodiment, con-rarity is a measure of codon frequency that is contextually dependent on tRNA abundance levels in a target tissue or cell.

Thus, the identification of a codon as a con-rare codon can involve a multi-parameter function of (1)-(7). In an embodiment, the con-rare codon meets a reference value for at least one of (1)-(7). In an embodiment, the con-rare codon meets a reference value for at least one of (1)-(7). In an embodiment, the con-rare codon meets a reference value for at least two of (1)-(7). In an embodiment, the con-rare codon meets a reference value for at least three of (1)-(7). In an embodiment, the con-rare codon meets a reference value for at least four of (1)-(7). In an embodiment, the con-rare codon meets a reference value for at least five of (1)-(7). In an embodiment, the con-rare codon meets a reference value for at least six of (1)-(7). In an embodiment, the con-rare codon meets a reference value for at all of (1)-(7). In an embodiment the reference value is a pre-determined or pre-selected value, e.g., as described herein.

In an embodiment, the identity of a con-rare codon is the DNA sequence which encodes for the codon in the nucleic acid sequence, e.g., gene.

In an embodiment, a con-rare codon is other than an iMet codon.

The methods disclosed herein, e.g., in the examples, provided herein, can be used to identify and test candidate con-rare codons.

In an embodiment, a con-rare codon is a function of the prevalence of the codon in the open reading frame (ORF) of protein coding genes in an organism, e.g., the proteome.

The availability, e.g., abundance, of tRNAs that correspond to a con-rare codon can be measured using an assay known in the art or as described herein, e.g., Nanopore sequencing, e.g., as described in Example 1. In an embodiment, a con-rare codon nucleic acid sequence has a low abundance of a tRNA corresponding to the con-rare codon, e.g., as compared to the abundance of a tRNA corresponding to a different/second codon.

The expression profile or proteomic property of a target cell or tissue refers to the protein expression, e.g., level of protein expression, from all of the protein coding genes in a target cell or tissue. The expression profile or proteomic property of a target cell or tissue can be measured using an assay known in the art or as described herein, e.g., a mass spectrometry based method, e.g., a SILAC based method as described in Example 2. In an embodiment, a protein coding gene in a target cell or tissue is a function of tissue or cell type specific regulation, e.g., a promoter element, an enhancer element, epigenetic regulation, and/or transcription factor control.

A “contextually-modified nucleic acid sequence” (sometimes referred to herein as a “con-modified nucleic acid sequence”) refers to a nucleic acid sequence in which the con-rarity of a codon of the con-modified nucleic acid sequence has been altered. E.g., a con-rare codon is replaced with a con-abundant codon and/or a con-abundant codon is replaced with a con-rare codon. In an embodiment, the con-modified nucleic acid sequence has one more or one less, e.g., two more or two lesser, con-rare codons, than a reference nucleic acid sequence. In an embodiment, the con-modified nucleic acid sequence has a codon with con-rarity that differs from the con-rarity of the corresponding codon in a reference nucleic acid sequence.

The reference nucleic acid sequence can be, e.g., any selected sequence, a parental sequence, a starting sequence, a wildtype or naturally occurring sequence that encodes the same amino acid at the corresponding codon, a wildtype or naturally occurring sequence that encodes the same polypeptide, or a conventionally codon-optimized sequence. In an embodiment, the reference nucleic acid sequence encodes the same polypeptide sequence as the con-modified nucleic acid sequence. In an embodiment, the reference nucleic acid sequence encodes a polypeptide sequence that differs from the con-modified nucleic acid sequence at a position other than the con-rare modified sequence. In an embodiment, a con-modified nucleic acid sequence results in a different production parameter, e.g., an expression parameter or signaling parameter, compared to that seen with expression of a reference nucleic acid sequence.

In an embodiment, a con-modified nucleic acid sequence refers to a nucleic acid sequence which has one more or one less, e.g., two more or two lesser, con-rare codons, than a reference sequence, wherein the con-modified nucleic acid sequence encodes a polypeptide that comprises the reference sequence.

A “contextually-rare tRNA” or “con-rare tRNA,” is a tRNA that corresponds to a con-rare codon.

A “contextually-abundant codon” or “con-abundant codon” as those terms are used herein, refer to a codon other than a con-rare codon.

A “con-rare codon nucleic acid sequence,” or a “nucleic acid sequence having a con-rare codon” as those terms are used herein, refer to a nucleic acid sequence, e.g., DNA, or RNA, or gene, comprising a con-rare codon. In an embodiment, in such con-rare codon nucleic acid sequences, modulation of a production parameter, e.g., an expression parameter or signaling parameter, can be mediated by altering the availability, e.g., abundance of a con-rare tRNA. In an embodiment, the con-rare codon is in a translated region of the con-rare codon nucleic acid sequence, e.g., in an open reading frame (ORF) or coding sequence (CDS).

A “con-rare codon RNA,” as that term is used herein, refers to an RNA sequence comprising a con-rare codon. In an embodiment, a con-rare codon RNA comprises a messenger RNA or an RNA that can be translated into a polypeptide or protein. In an embodiment, a con-rare codon RNA is transcribed from a complementary DNA sequence which comprises said con-rare codon. In an embodiment, the con-rare codon RNA is transcribed in vivo. In an embodiment, the con-rare codon RNA is transcribed in vitro.

A “codon-value” as that term is used herein, is a function of the con-rarity of a sequence-codon in a sequence. Con-rarity of a codon is a function of one or more factors as described in the definition of “con-rare codon” above. In an embodiment, a codon-value is the identity of a codon, e.g., a replacement codon selected to replace the sequence-codon. In an embodiment, when the replacement codon is a con-abundant codon, the sequence codon is a con-rare codon.

In an embodiment, when the replacement codon is a con-rare codon, the sequence-codon is a con-abundant codon.

A “sequence-codon” as that term is used herein, refers to a codon in a nucleic acid sequence for which a codon-value is acquired.

A “production parameter,” refers to an expression parameter and/or a signaling parameter. In an embodiment a production parameter is an expression parameter. An expression parameter includes an expression parameter of a polypeptide or protein encoded by the con-rare codon nucleic acid sequence; or an expression parameter of an RNA, e.g., messenger RNA, encoded by the con-rare codon nucleic acid sequence. In an embodiment, an expression parameter can include:

(a) protein translation;

(b) expression level (e.g., of polypeptide or protein, or mRNA);

(c) post-translational modification of polypeptide or protein;

(d) folding (e.g., of polypeptide or protein, or mRNA),

(e) structure (e.g., of polypeptide or protein, or mRNA),

(f) transduction (e.g., of polypeptide or protein),

(g) compartmentalization (e.g., of polypeptide or protein, or mRNA),

(h) incorporation (e.g., of polypeptide or protein, or mRNA) into a supermolecular structure, e.g., incorporation into a membrane, proteasome, or ribosome,

(i) incorporation into a multimeric polypeptide, e.g., a homo or heterodimer, and/or

(j) stability.

In an embodiment, a production parameter is a signaling parameter. A signaling parameter can include:

(1) modulation of a signaling pathway, e.g., a cellular signaling pathway which is downstream or upstream of the protein encoded by the con-rare codon nucleic acid sequence;

(2) cell fate modulation;

(3) ribosome occupancy modulation;

(4) protein translation modulation;

(5) mRNA stability modulation;

(6) protein folding and structure modulation;

(7) protein transduction or compartmentalization modulation; and/or

(8) protein stability modulation.

“Acquire” or “acquiring” as the terms are used herein, refer to obtaining possession of a value, e.g., a numerical value, by “directly acquiring” or “indirectly acquiring” the physical entity or value. “Directly acquiring” refers to performing a process (e.g., performing an analytical method) to obtain the value. “Indirectly acquiring” refers to receiving the value from another party or source (e.g., a third party laboratory that directly acquired the or value).

A “cognate adaptor function TREM,” as that term is used herein, refers to a TREM which mediates initiation or elongation with the AA (the cognate AA) associated in nature with the anti-codon of the TREM.

A “decreased expression,” as that term is used herein, refers to a decrease in comparison to a reference, e.g., in the case where altered control region, or addition of an agent, results in a decreased expression of the subject product, it is decreased relative to an otherwise similar cell without the alteration or addition.

An “exogenous nucleic acid,” as that term is used herein, refers to a nucleic acid sequence that is not present in or differs by at least one nucleotide from the closest sequence in a reference cell, e.g., a cell into which the exogenous nucleic acid is introduced. In an embodiment, an exogenous nucleic acid comprises a nucleic acid that encodes a TREM.

An “exogenous TREM,” as that term is used herein, refers to a TREM that:

(a) differs by at least one nucleotide or one post transcriptional modification from the closest sequence tRNA in a reference cell, e.g., a cell into which the exogenous nucleic acid is introduced;

(b) has been introduced into a cell other than the cell in which it was transcribed;

(c) is present in a cell other than one in which it naturally occurs; or

(d) has an expression profile, e.g., level or distribution, that is non-wildtype, e.g., it is expressed at a higher level than wildtype. In an embodiment, the expression profile can be mediated by a change introduced into a nucleic acid that modulates expression or by addition of an agent that modulates expression of the RNA molecule. In an embodiment an exogenous TREM comprises 1, 2, 3 or 4 of properties (a)-(d).

A “GMP-grade composition,” as that term is used herein, refers to a composition in compliance with current good manufacturing practice (cGMP) guidelines, or other similar requirements. In an embodiment, a GMP-grade composition can be used as a pharmaceutical product.

As used herein, the terms “increasing” and “decreasing” refer to modulation that results in, respectively, greater or lesser amounts of function, expression, or activity of a particular metric relative to a reference. For example, subsequent to administration to a cell, tissue or subject of a TREM described herein, the amount of a marker of a metric (e.g., protein translation, mRNA stability, protein folding) as described herein may be increased or decreased by at least 5%, 10%, 15%, 20%, 25%, 30%, 35%, 40%, 45%, 50%, 55%, 60%, 65%, 70%, 75%, 80%, 85%, 90%, 95% or 98%, 2×, 3×, 5×, 10× or more relative to the amount of the marker prior to administration or relative to the effect of a negative control agent. The metric may be measured subsequent to administration at a time that the administration has had the recited effect, e.g., at least 12 hours, 24 hours, one week, one month, 3 months, or 6 months, after a treatment has begun.

An “increased expression,” as that term is used herein, refers to an increase in comparison to a reference, e.g., in the case where altered control region, or addition of an agent, results in an increased expression of the subject product, it is increased relative to an otherwise similar cell without the alteration or addition.

An “isoacceptor,” as that term is used herein, refers to a plurality of tRNA molecule or TREMs wherein each molecule of the plurality comprises a different naturally occurring anticodon sequence and each molecule of the plurality mediates the incorporation of the same amino acid and that amino acid is the amino acid that naturally corresponds to the anticodons of the plurality.

A “non-cognate adaptor function TREM,” as that term is used herein, refers to a TREM which mediates initiation or elongation with an AA (a non-cognate AA) other than the AA associated in nature with the anticodon of the TREM. In an embodiment, a non-cognate adaptor function TREM is also referred to as a mischarged TREM (mTREM).

A “non-naturally occurring sequence,” as that term is used herein, refers to a sequence wherein an Adenine is replaced by a residue other than an analog of Adenine, a Cytosine is replaced by a residue other than an analog of Cytosine, a Guanine is replaced by a residue other than an analog of Guanine, and a Uracil is replaced by a residue other than an analog of Uracil. An analog refers to any possible derivative of the ribonucleotides, A, G, C or U. In an embodiment, a sequence having a derivative of any one of ribonucleotides A, G, C or U is a non-naturally occurring sequence.

An “oncogene,” as that term is used herein, refers to a gene that modulates one or more cellular processes including: cell fate determination, cell survival and genome maintenance. In an embodiment, an oncogene provides a selective growth advantage to the cell in which it is present, e.g., deregulated, e.g., genetically deregulated (e.g., mutated or amplified) or epigenetically deregulated. Exemplary oncogenes include, Myc (e.g., c-Myc, N-Myc or L-Myc), c-Jun, Wnt, or RAS.

A “pharmaceutical composition,” as that term is used herein, refers to a composition that is suitable for pharmaceutical use. Typically, a pharmaceutical composition comprises a pharmaceutical excipient. In an embodiment, a pharmaceutical composition can comprise a TREM (a pharmaceutical composition comprising a TREM). In an embodiment the TREM will be the only active ingredient in a pharmaceutical composition comprising a TREM. In embodiments a pharmaceutical composition, e.g., a pharmaceutical composition comprising a TREM, is free, substantially free, or has less than a pharmaceutically acceptable amount, of host cell proteins, DNA, e.g., host cell DNA, endotoxins, and bacteria. In an embodiment, a pharmaceutical composition, e.g., a pharmaceutical composition comprising a TREM, is a GMP-grade composition in compliance with current good manufacturing practice (cGMP) guidelines, or other similar requirements. In an embodiment, a pharmaceutical composition, e.g., a pharmaceutical composition comprising a TREM is sterile, e.g., the composition or preparation supports the growth of fewer than 100 viable microorganisms as tested under aseptic conditions, the composition or preparation meets the standard of USP <71>, and/or the composition or preparation meets the standard of USP <85>.

A “post-transcriptional processing,” as that term is used herein, with respect to a subject molecule, e.g., a TREM, RNA or tRNAs, refers to a covalent modification of the subject molecule. In an embodiment, the covalent modification occurs post-transcriptionally. In an embodiment, the covalent modification occurs co-transcriptionally. In an embodiment the modification is made in vivo, e.g., in a cell used to produce a TREM. In an embodiment the modification is made ex vivo, e.g., it is made on a TREM isolated or obtained from the cell which produced the TREM. In an embodiment, the post-transcriptional modification is selected from a post-transcriptional modification listed in Table 2.

A “recombinant TREM,” as that term is used herein, refers to a TREM that was expressed in a cell modified by human intervention, having a modification that mediates the production of the TREM, e.g., the cell comprises an exogenous sequence encoding the TREM, or a modification that mediates expression, e.g., transcriptional expression or post-transcriptional modification, of the TREM. A recombinant TREM can have the same, or a different, sequence, set of post-transcriptional modifications, or tertiary structure, as a reference tRNA, e.g., a native tRNA.

A “synthetic TREM,” as that term is used herein, refers to a TREM which was synthesized other than in a cell having an endogenous nucleic acid encoding the TREM, e.g., by cell-free solid phase synthesis. A synthetic TREM can have the same, or a different, sequence, set of post-transcriptional modifications, or tertiary structure, as a native tRNA.

A “TREM expressed in a heterologous cell,” as that term is used herein, refers to a TREM made under non-native conditions. E.g., a TREM, i) made in a cell that, differs, e.g., genetically, metabolically (e.g., has a different profile of gene expression or has a different level of a cellular component, e.g., an absorbed nutrient), or epigenetically, from a naturally occurring cell; ii) made in a cell that, is cultured under conditions, e.g., nutrition, pH, temperature, cell density, or stress conditions, that are different from native conditions (native conditions are the conditions under which a cell makes a tRNA in nature); or iii) was made in a cell at a level, at a rate, or at a concentration, or was localized in a compartment or location, that differs from a reference, e.g., at a level, at a rate, or at a concentration, or was localized in a compartment or location, that differs from that which occurs under native conditions. A TREM expressed in a heterologous cell can have the same, or a different, sequence, set of post-transcriptional modifications, or tertiary structure, as a native tRNA.

A “tRNA”, as that term is used herein, refers to a naturally occurring transfer ribonucleic acid in its native state.

A “tRNA-based effector molecule” or “TREM,” as that term is used herein, refers to an RNA molecule comprising a structure or property from (a)-(v) below, and which is a recombinant TREM, a synthetic TREM, or a TREM expressed from a heterologous cell. A TREM can have a plurality (e.g., 2, 3, 4, 5, 6, 7, 8, 9) of the structures and functions of (a)-(v).

In an embodiment, a TREM is non-native, as evaluated by structure or the way in which it was made.

In an embodiment, a TREM comprises one or more of the following structures or properties:

(a′) an optional linker region of a consensus sequence provided in the “Consensus Sequence” section, e.g., a Linker 1 region;

(a) an amino acid attachment domain that binds an amino acid, e.g., an acceptor stem domain (AStD), wherein an AStD comprises sufficient RNA sequence to mediate, e.g., when present in an otherwise wildtype tRNA, acceptance of an amino acid, e.g., its cognate amino acid or a non-cognate amino acid, and transfer of the amino acid (AA) in the initiation or elongation of a polypeptide chain. Typically, the AStD comprises a 3′-end adenosine (CCA) for acceptor stem charging which is part of synthetase recognition. In an embodiment the AStD has at least 75, 80, 85, 85, 90, 95, or 100% identity with a naturally occurring AStD, e.g., an AStD encoded by a nucleic acid in Table 1. In an embodiment, the TREM can comprise a fragment or analog of an AStD, e.g., an AStD encoded by a nucleic acid in Table 1, which fragment in embodiments has AStD activity and in other embodiments does not have AStD activity. (One of ordinary skill can determine the relevant corresponding sequence for any of the domains, stems, loops, or other sequence features mentioned herein from a sequence encoded by a nucleic acid in Table 1. E.g., one of ordinary skill can determine the sequence which corresponds to an AStD from a tRNA sequence encoded by a nucleic acid in Table 1.) In an embodiment the AStD falls under the corresponding sequence of a consensus sequence provided in the “Consensus Sequence” section, or differs from the consensus sequence by no more than 1, 2, 5, or 10 positions;

In an embodiment, the AStD comprises residues R₁—R₂—R₃—R₄—R₅—R₆—R₇ and residues R₆₅—R₆₆—R₆₇—R₆₈—R₆₉—R₇₀—R₇₁ of Formula I zzz, wherein ZZZ indicates any of the twenty amino acids;

In an embodiment, the AStD comprises residues R₁—R₂—R₃—R₄—R₅—R₆—R₇ and residues R₆₅—R₆₆—R₆₇—R₆₈—R₆₉—R₇₀—R₇₁ of Formula II zzz, wherein ZZZ indicates any of the twenty amino acids;

In an embodiment, the AStD comprises residues R₁—R₂—R₃—R₄—R₅—R₆—R₇ and residues R₆₅—R₆₆—R₆₇—R₆₈—R₆₉—R₇₀—R₇₁ of Formula IIII zzz, wherein ZZZ indicates any of the twenty amino acids;

(a′-1) a linker comprising residues R₈—R₉ of a consensus sequence provided in the “Consensus Sequence” section, e.g., a Linker 2 region;

(b) a dihydrouridine hairpin domain (DHD), wherein a DHD comprises sufficient RNA sequence to mediate, e.g., when present in an otherwise wildtype tRNA, recognition of aminoacyl-tRNA synthetase, e.g., acts as a recognition site for aminoacyl-tRNA synthetase for amino acid charging of the TREM. In embodiments, a DHD mediates the stabilization of the TREM's tertiary structure. In an embodiment the DHD has at least 75, 80, 85, 85, 90, 95, or 100% identity with a naturally occurring DHD, e.g., a DHD encoded by a nucleic acid in Table 1. In an embodiment, the TREM can comprise a fragment or analog of a DHD, e.g., a DHD encoded by a nucleic acid in Table 1, which fragment in embodiments has DHD activity and in other embodiments does not have DHD activity.

In an embodiment the DHD falls under the corresponding sequence of a consensus sequence provided in the “Consensus Sequence” section, or differs from the consensus sequence by no more than 1, 2, 5, or 10 positions;

In an embodiment, the DHD comprises residues R₁₀—R₁₁—R₁₂—R₁₃—R₁₄R₁₅—R₁₆—R₁₇—R₁₈—R₁₉—R₂₀—R₂₁—R₂₂—R₂₃—R₂₄—R₂₅—R₂₆—R₂₇—R₂₈ of Formula I zzz, wherein ZZZ indicates any of the twenty amino acids;

In an embodiment, the DHD comprises residues R₁₀—R₁₁—R₁₂—R₁₃—R₁₄R₁₅—R₁₆—R₁₇—R₁₈—R₁₉—R₂₀—R₂₁—R₂₂—R₂₃—R₂₄—R₂₅—R₂₆—R₂₇—R₂₈ of Formula II zzz, wherein ZZZ indicates any of the twenty amino acids;

In an embodiment, the DHD comprises residues R₁₀—R₁₁—R₁₂—R₁₃—R₁₄R₁₅—R₁₆—R₁₇—R₁₈—R₁₉—R₂₀—R₂₁—R₂₂—R₂₃—R₂₄—R₂₅—R₂₆—R₂₇—R₂₈ of Formula IIII zzz, wherein ZZZ indicates any of the twenty amino acids;

(b′-1) a linker comprising residue R₂₉ of a consensus sequence provided in the “Consensus Sequence” section, e.g., a Linker 3 region;

(c) an anticodon that binds a respective codon in an mRNA, e.g., an anticodon hairpin domain (ACHD), wherein an ACHD comprises sufficient sequence, e.g., an anticodon triplet, to mediate, e.g., when present in an otherwise wildtype tRNA, pairing (with or without wobble) with a codon; In an embodiment the ACHD has at least 75, 80, 85, 85, 90, 95, or 100% identity with a naturally occurring ACHD, e.g., an ACHD encoded by a nucleic acid in Table 1. In an embodiment, the TREM can comprise a fragment or analog of an ACHD, e.g., an ACHD encoded by a nucleic acid in Table 1, which fragment in embodiments has ACHD activity and in other embodiments does not have ACHD activity.

In an embodiment the ACHD falls under the corresponding sequence of a consensus sequence provided in the “Consensus Sequence” section, or differs from the consensus sequence by no more than 1, 2, 5, or 10 positions;

In an embodiment, the ACHD comprises residues —R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆ of Formula I zzz, wherein ZZZ indicates any of the twenty amino acids;

In an embodiment, the ACHD comprises residues —R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆ of Formula II zzz, wherein ZZZ indicates any of the twenty amino acids;

In an embodiment, the ACHD comprises residues —R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆ of Formula III zzz, wherein ZZZ indicates any of the twenty amino acids;

(d) a variable loop domain (VLD), wherein a VLD comprises sufficient RNA sequence to mediate, e.g., when present in an otherwise wildtype tRNA, recognition of aminoacyl-tRNA synthetase, e.g., acts as a recognition site for aminoacyl-tRNA synthetase for amino acid charging of the TREM. In embodiments, a VLD mediates the stabilization of the TREM's tertiary structure. In an embodiment, a VLD modulates, e.g., increases, the specificity of the TREM, e.g., for its cognate amino acid, e.g., the VLD modulates the TREM's cognate adaptor function. In an embodiment the VLD has at least 75, 80, 85, 85, 90, 95, or 100% identity with a naturally occurring VLD, e.g., a VLD encoded by a nucleic acid in Table 1. In an embodiment, the TREM can comprise a fragment or analog of a VLD, e.g., a VLD encoded by a nucleic acid in Table 1, which fragment in embodiments has VLD activity and in other embodiments does not have VLD activity.

In an embodiment the VLD falls under the corresponding sequence of a consensus sequence provided in the “Consensus Sequence” section.

In an embodiment, the VLD comprises residue —[R₄₇]ₓ₁ of a consensus sequence provided in the “Consensus Sequence” section, wherein x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271);

(e) a thymine hairpin domain (THD), wherein a THD comprises sufficient RNA sequence, to mediate, e.g., when present in an otherwise wildtype tRNA, recognition of the ribosome, e.g., acts as a recognition site for the ribosome to form a TREM-ribosome complex during translation. In an embodiment the THD has at least 75, 80, 85, 85, 90, 95, or 100% identity with a naturally occurring THD, e.g., a THD encoded by a nucleic acid in Table 1. In an embodiment, the TREM can comprise a fragment or analog of a THD, e.g., a THD encoded by a nucleic acid in Table 1, which fragment in embodiments has THD activity and in other embodiments does not have THD activity.

In an embodiment the THD falls under the corresponding sequence of a consensus sequence provided in the “Consensus Sequence” section, or differs from the consensus sequence by no more than 1, 2, 5, or 10 positions;

In an embodiment, the THD comprises residues —R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄ of Formula I zzz, wherein ZZZ indicates any of the twenty amino acids;

In an embodiment, the THD comprises residues —R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄ of Formula II zzz, wherein ZZZ indicates any of the twenty amino acids;

In an embodiment, the THD comprises residues —R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄ of Formula III zzz, wherein ZZZ indicates any of the twenty amino acids;

(e′ 1) a linker comprising residue R₇₂ of a consensus sequence provided in the “Consensus Sequence” section, e.g., a Linker 4 region;

(f) under physiological conditions, it comprises a stem structure and one or a plurality of loop structures, e.g., 1, 2, or 3 loops. A loop can comprise a domain described herein, e.g., a domain selected from (a)-(e). A loop can comprise one or a plurality of domains. In an embodiment, a stem or loop structure has at least 75, 80, 85, 85, 90, 95, or 100% identity with a naturally occurring stem or loop structure, e.g., a stem or loop structure encoded by a nucleic acid in Table 1. In an embodiment, the TREM can comprise a fragment or analog of a stem or loop structure, e.g., a stem or loop structure encoded by a nucleic acid in Table 1, which fragment in embodiments has activity of a stem or loop structure, and in other embodiments does not have activity of a stem or loop structure;

(g) a tertiary structure, e.g., an L-shaped tertiary structure;

(h) adaptor function, i.e., the TREM mediates acceptance of an amino acid, e.g., its cognate amino acid and transfer of the AA in the initiation or elongation of a polypeptide chain;

(i) cognate adaptor function wherein the TREM mediates acceptance and incorporation of an amino acid (e.g., cognate amino acid) associated in nature with the anti-codon of the TREM to initiate or elongate a polypeptide chain;

(j) non-cognate adaptor function, wherein the TREM mediates acceptance and incorporation of an amino acid (e.g., non-cognate amino acid) other than the amino acid associated in nature with the anti-codon of the TREM in the initiation or elongation of a polypeptide chain;

(k) a regulatory function, e.g., an epigenetic function (e.g., gene silencing function or signaling pathway modulation function), cell fate modulation function, mRNA stability modulation function, protein stability modulation function, protein transduction modulation function, or protein compartmentalization function;

(l) a structure which allows for ribosome binding;

(m) a post-transcriptional modification, e.g., it comprises one or more modifications from Table 2, e.g., 1, 2, 3, 4, 5, 6, 7, 8, 9, 10, 11, 12, 13, 14, or 15 modifications listed in Table 2;

(n) the ability to inhibit a functional property of a tRNA, e.g., any of properties (h)-(k) possessed by a tRNA;

(o) the ability to modulate cell fate;

(p) the ability to modulate ribosome occupancy;

(q) the ability to modulate protein translation;

(r) the ability to modulate mRNA stability;

(s) the ability to modulate protein folding and structure;

(t) the ability to modulate protein transduction or compartmentalization;

(u) the ability to modulate protein stability; or

(v) the ability to modulate a signaling pathway, e.g., a cellular signaling pathway.

In an embodiment, a TREM comprises a full-length tRNA molecule or a fragment thereof.

In an embodiment, a TREM comprises the following properties: (a)-(e).

In an embodiment, a TREM comprises the following properties: (a) and (c).

In an embodiment, a TREM comprises the following properties: (a), (c) and (h).

In an embodiment, a TREM comprises the following properties: (a), (c), (h) and (b).

In an embodiment, a TREM comprises the following properties: (a), (c), (h) and (e).

In an embodiment, a TREM comprises the following properties: (a), (c), (h), (b) and (e).

In an embodiment, a TREM comprises the following properties: (a), (c), (h), (b), (e) and (g).

In an embodiment, a TREM comprises the following properties: (a), (c), (h) and (m).

In an embodiment, a TREM comprises the following properties: (a), (c), (h), (m), and (g).

In an embodiment, a TREM comprises the following properties: (a), (c), (h), (m) and (b).

In an embodiment, a TREM comprises the following properties: (a), (c), (h), (m) and (e).

In an embodiment, a TREM comprises the following properties: (a), (c), (h), (m), (g), (b) and (e).

In an embodiment, a TREM comprises the following properties: (a), (c), (h), (m), (g), (b), (e) and (q).

In an embodiment, a TREM comprises:

(i) an amino acid attachment domain that binds an amino acid (e.g., an AStD, as described in (a) herein; and

(ii) an anticodon that binds a respective codon in an mRNA (e.g., an ACHD, as described in (c) herein).

In an embodiment the TREM comprises a flexible RNA linker which provides for covalent linkage of (i) to (ii).

In an embodiment, the TREM mediates protein translation.

In an embodiment a TREM comprises a linker, e.g., an RNA linker, e.g., a flexible RNA linker, which provides for covalent linkage between a first and a second structure or domain. In an embodiment, an RNA linker comprises at least 1, 2, 3, 4, 5, 6, 7, 8, 9, 10, 11, 12, 13, 14 or 15 ribonucleotides. A TREM can comprise one or a plurality of linkers, e.g., in embodiments a TREM comprising (a), (b), (c), (d) and (e) can have a first linker between a first and second domain, and a second linker between a third domain and another domain.

In an embodiment, a TREM comprises an RNA sequence at least 60, 65, 70, 75, 80, 85, 90, 95, 96, 97, 98 or 99% identical with, or which differs by no more than 1, 2, 3, 4, 5, 10, 15, 20, 25, or 30 ribonucleotides from, an RNA sequence encoded by a DNA sequence listed in Table 1, or a fragment or functional fragment thereof. In an embodiment, a TREM comprises an RNA sequence encoded by a DNA sequence listed in Table 1, or a fragment or functional fragment thereof. In an embodiment, a TREM comprises an RNA sequence encoded by a DNA sequence at least 60, 65, 70, 75, 80, 85, 90, 95, 96, 97, 98 or 99% identical with a DNA sequence listed in Table 1, or a fragment or functional fragment thereof. In an embodiment, a TREM comprises a TREM domain, e.g., a domain described herein, comprising at least 60, 65, 70, 75, 80, 85, 90, 95, 96, 97, 98, or 99% identical with, or which differs by no more than 1, 2, 3, 4, 5, 10, or 15, ribonucleotides from, an RNA encoded by a DNA sequence listed in Table 1, or a fragment or a functional fragment thereof. In an embodiment, a TREM comprises a TREM domain, e.g., a domain described herein, comprising an RNA sequence encoded by DNA sequence listed in Table 1, or a fragment or functional fragment thereof. In an embodiment, a TREM comprises a TREM domain, e.g., a domain described herein, comprising an RNA sequence encoded by DNA sequence at least 60, 65, 70, 75, 80, 85, 90, 95, 96, 97, 98 or 99% identical with a DNA sequence listed in Table 1, or a fragment or functional fragment thereof.

In an embodiment, a TREM is 76-90 nucleotides in length. In embodiments, a TREM or a fragment or functional fragment thereof is between 10-90 nucleotides, between 10-80 nucleotides, between 10-70 nucleotides, between 10-60 nucleotides, between 10-50 nucleotides, between 10-40 nucleotides, between 10-30 nucleotides, between 10-20 nucleotides, between 20-90 nucleotides, between 20-80 nucleotides, 20-70 nucleotides, between 20-60 nucleotides, between 20-50 nucleotides, between 20-40 nucleotides, between 30-90 nucleotides, between 30-80 nucleotides, between 30-70 nucleotides, between 30-60 nucleotides, or between 30-50 nucleotides.

In an embodiment, a TREM is aminoacylated, e.g., charged, with an amino acid by an aminoacyl tRNA synthetase.

In an embodiment, a TREM is not charged with an amino acid, e.g., an uncharged TREM (uTREM).

In an embodiment, a TREM comprises less than a full length tRNA. In embodiments, a TREM can correspond to a naturally occurring fragment of a tRNA, or to a non-naturally occurring fragment. Exemplary fragments include: TREM halves (e.g., from a cleavage in the ACHD, e.g., in the anticodon sequence, e.g., 5′ halves or 3′ halves); a 5′ fragment (e.g., a fragment comprising the 5′ end, e.g., from a cleavage in a DHD or the ACHD); a 3′ fragment (e.g., a fragment comprising the 3′ end, e.g., from a cleavage in the THD); or an internal fragment (e.g., from a cleavage in one or more of the ACHD, DHD or THD).

A “TREM composition,” as that term is used herein, refers to a composition comprising a plurality of TREMs. A TREM composition can comprise one or more species of TREMs. In an embodiment, the composition comprises only a single species of TREM. In an embodiment, the TREM composition comprises a first TREM species and a second TREM species. In an embodiment, the TREM composition comprises X TREM species, wherein X=2, 3, 4, 5, 6, 7, 8, 9, or 10. In an embodiment, the TREM has at least 70, 75, 80, 85, 90, or 95, or has 100%, identity with a sequence encoded by a nucleic acid in Table 1. A TREM composition can comprise one or more species of TREMs. In an embodiment, the TREM composition is purified from cell culture. In an embodiment the cell culture from which the TREM is purified comprises at least 1×10⁷ host cells, 1×10⁸ host cells, 1×10⁹ host cells, 1×10¹⁰ host cells, 1×10¹¹ host cells, 1×10¹² host cells, 1×10¹³ host cells, or 1×10¹⁴ host cells. In an embodiment, the TREM composition is at least 10, 20, 30, 40, 50, 60, 70, 80, 90, 95 or 99% dry weight TREMs (for a liquid composition dry weight refers to the weight after removal of substantially all liquid, e.g., after lyophilization). In an embodiment, the composition is a liquid. In an embodiment, the composition is dry, e.g., a lyophilized material. In an embodiment, the composition is a frozen composition. In an embodiment, the composition is sterile. In an embodiment, the composition comprises at least 0.5 g, 1.0 g, 5.0 g, 10 g, 15 g, 25 g, 50 g, 100 g, 200 g, 400 g, or 500 g (e.g., as determined by dry weight) of TREM.

A “tumor suppressor,” as that term is used herein, refers to a gene that modulates one or more cellular processes including: cell fate determination, cell survival and genome maintenance. In an embodiment, a tumor suppressor provides a selective growth advantage to the cell in which it is deregulated, e.g., genetically deregulated (e.g., mutated or deleted) or epigenetically deregulated. Exemplary tumor suppressors include p53 or Rb.

“Pairs with” or “pairing,” as those terms are used herein, refer to the correspondence of a codon with an anticodon and includes fully complementary codon:anticodon pairs as well as “wobble” pairing, in which the third position need not be complementary. Fully complementary pairing refers to pairing of all three positions of the codon with the corresponding anticodon according to Watson-Crick base pairing. Wobble pairing refers to complementary pairing of the first and second positions of the codon with the corresponding anticodon according to Watson-Crick base pairing, and flexible pairing at the third position of the codon with the corresponding anticodon.

The terms modified, replace, derived and similar terms, when used or applied in reference to a product, refer only to the end product or structure of the end product, and are not restricted by any method of making or manufacturing the product, unless expressly provided as such in this disclosure.

Headings, titles, subtitles, numbering or other alpha/numeric hierarchies are included merely for ease of reading and absent explicit language to the contrary do not indicate order of performance, order of importance, magnitude or other value.

### Contextually-Rare Codons (“Con-Rare Codons”)

Disclosed herein is the observation that a production parameter of an RNA, or a protein encoded by an RNA having a con-rare codon, can be modulated by administration of a TREM composition comprising a TREM corresponding to said con-rare codon. Accordingly, this disclosure provides, inter alia, methods of identifying a contextually rare codon (“con-rare codon”), compositions of TREMs corresponding to a con-rare codon and uses of said TREM compositions.

A con-rare codon is a codon that is limiting for a production parameter, e.g., an expression parameter or a signaling parameter, for a nucleic acid sequence, e.g., a DNA or an RNA, or a protein encoded by a nucleic acid sequence, e.g., a DNA or an RNA. Contextual rareness or con-rarity can be identified or evaluated by determining if the addition of a tRNA corresponding to a con-rare codon modulates, typically increases, a production parameter for a target nucleic acid sequence, e.g., target, e.g., gene. In an embodiment, con-rarity as a property of a codon, is a function of, one, two, three, four, all of the following factors:

(1) the sequence of the codon;

(2) the availability of a corresponding tRNA, e.g., charged tRNA, for that con-rare codon in a target cell or tissue, e.g., one or more iso-acceptor tRNA molecules;

(3) the expression profile (or proteomic properties) of the target cell or tissue (e.g., the abundance of expression of other proteins which include the con-rare codon);

(4) the proportion of the tRNAs corresponding to the con-rare codon which are charged; and

(5) the iso-decoder isotype of the tRNA corresponding to the con-rare codon.

In an embodiment, con-rarity is a function of normalized proteome codon count and tRNA abundance in a target tissue or cell. In an embodiment, con-rarity is a measure of codon frequency that is contextually dependent on tRNA abundance levels in a target tissue or cell. In an embodiment, con-rarity can be identified or evaluated by a production parameter, e.g., an expression parameter or a signaling parameter, e.g., as described herein.

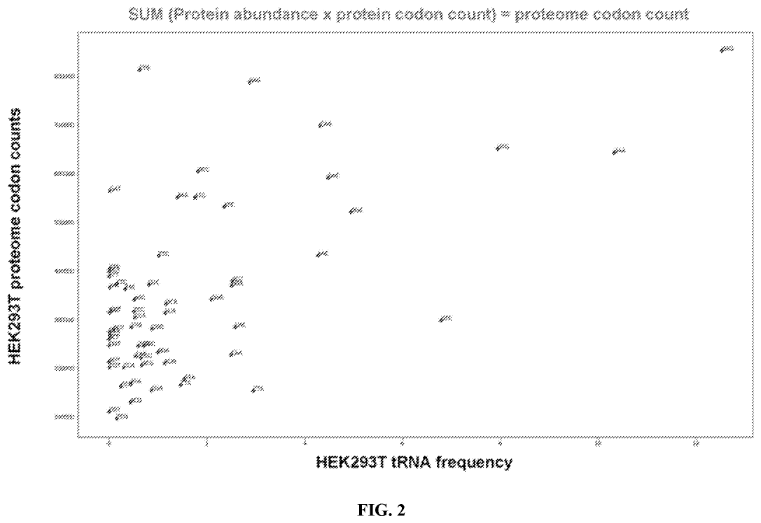

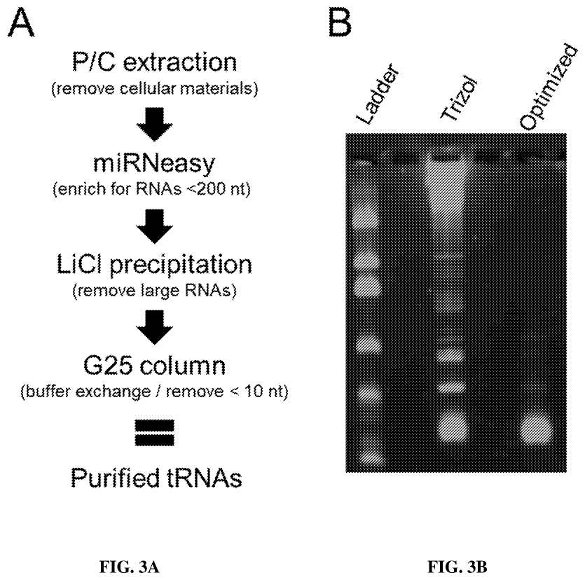

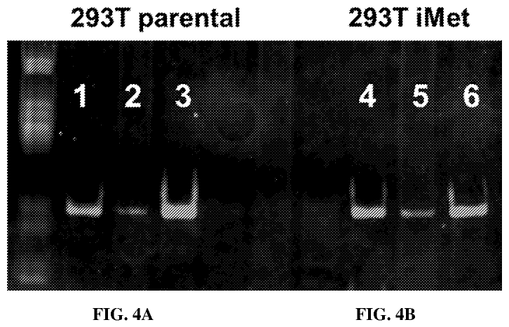

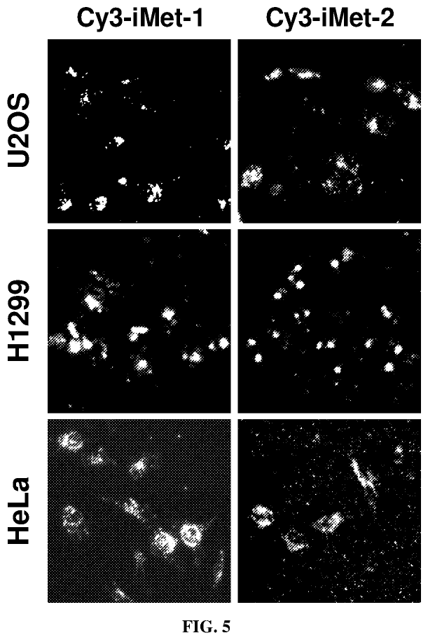

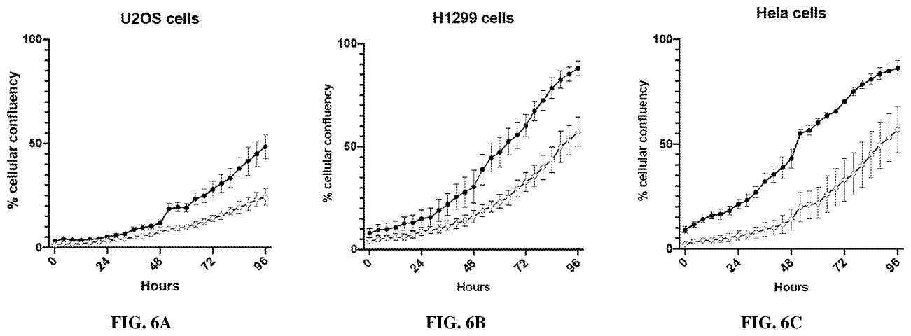

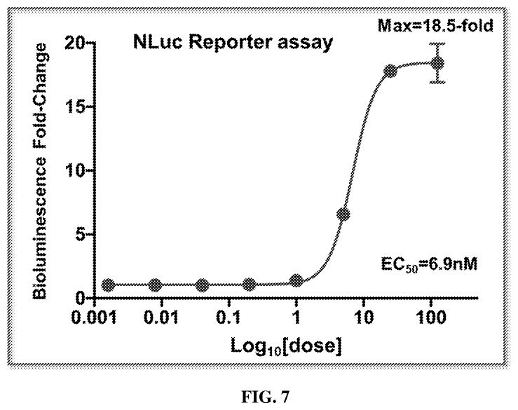

An exemplary method of evaluating con-rarity and identifying a con-rare codon is provided in Example 3, or for example, in FIG. 2.

### Exemplary Reference Values for Evaluating Con-Rarity

In an embodiment, contextual rareness or con-rarity can be identified or evaluated by whether a codon satisfies a reference value for proteome codon count-tRNA frequency (PCC-tF, as described herein).

In an embodiment, con-rarity is a function of normalized proteome codon count and the tRNA profile, e.g., as described herein. In an embodiment, con-rarity is determined by dividing the normalized proteome codon count by the tRNA profile determined by Nanopore or other tRNA sequencing experiment. This provides a measure of codon usage that is contextually dependent on the tRNA profile, e.g., tRNA abundance levels.

In an embodiment, a codon is determined to be contextually rare (con-rare) if the con-rarity meets a reference value, e.g., a pre-determined or pre-selected reference value, e.g., a threshold, e.g., an internal threshold, e.g., as described herein. In an embodiment, the reference value is a value under which e.g., 1.5× sigma of the normally fit distribution to that codon frequency.

In an embodiment, a codon is con-rare if the value of a normalized proteome codon count divided by the tRNA profile value for a particular tRNA meets a reference value, e.g., a pre-determined or pre-selected reference value, e.g., a threshold, e.g., an internal threshold.

In an embodiment, a codon is con-rare if the value of a normalized proteome codon count divided by the tRNA profile value for a particular tRNA is in the top 5%, 10%, 20%, 30%, or 40% of values for normalized proteome codon count divided by the tRNA profile value for all codons measured, e.g., wherein all 64 codons are measured. In an embodiment, a codon is con-rare if the value of a normalized proteome codon count divided by the tRNA profile value for a particular tRNA is in the top 5% of values for normalized proteome codon count divided by the tRNA profile value for all codons measured. In an embodiment, a codon is con-rare if the value of a normalized proteome codon count divided by the tRNA profile value for a particular tRNA is in the top 10% of values for normalized proteome codon count divided by the tRNA profile value for all codons measured. In an embodiment, a codon is con-rare if the value of a normalized proteome codon count divided by the tRNA profile value for a particular tRNA is in the top 20% of values for normalized proteome codon count divided by the tRNA profile value for all codons measured. In an embodiment, a codon is con-rare if the value of a normalized proteome codon count divided by the tRNA profile value for a particular tRNA is in the top 30% of values for normalized proteome codon count divided by the tRNA profile value for all codons measured. In an embodiment, a codon is con-rare if the value of a normalized proteome codon count divided by the tRNA profile value for a particular tRNA is in the top 40% of values for normalized proteome codon count divided by the tRNA profile value for all codons measured.

In an embodiment, a codon is con-rare if for the value of a normalized proteome codon count divided by the tRNA profile value for a particular tRNA, the value for the normalized proteome codon count is below the value for all codons measured and the value for tRNA profile, is above the value for all codons measured, e.g., wherein all 64 codons are measured.

In an embodiment, a codon is a con-rare codon if it is in the upper left quadrant of a plot of normalized proteome codon count (y-axis) vs tRNA profile (x-axis), with equal number of codons in each quadrant, e.g., wherein all 64 codons are measured.

In an embodiment, a codon is a con-rare codon if it is in a quadrant other than the lower right quadrant of a plot of normalized proteome codon count (y-axis) vs tRNA profile (x-axis), with equal number of codons in each quadrant, e.g., wherein all 64 codons are measured.

### Proteome Codon Count-tRNA Frequency (PCC-tF)

In another aspect, proteome codon count (for a selected codon) can be used in conjunction with tRNA frequency (for tRNAs having the selected codon) to provide a measure of con-rarity for the selected codon. This parameter is referred to herein as proteome codon count-tRNA frequency, or PCC-tF. Proteome codon count can serve as a measure of “demand” for a tRNA having a selected codon. tRNA frequency can serve as a measure of “supply” for a tRNA having a selected codon.

Proteome codon count, as used herein, refers to the sum (for all of the proteins of a set of reference proteins in a target cell (or tissue)) of the number of times the codon is used in a protein of the reference set multiplied by the value of that protein's abundance. Proteome codon count can be expressed as Σ(protein abundance×protein codon count)_{R1-Rn}, wherein R is the set of proteins. Typically the reference set is all of the proteins expressed in a target cell (or tissue) or a portion of the proteins expressed in a target cell, e.g., all proteins for which the abundance of the protein is greater than 1%, 5%, 10%, 15%, 20%, 25%, 30%, 35%, 40%, 45%, 50%, 55%, 60%. 65%, 70%, 75%, 80%, 85%, 90%, 95% or more by number or molecular weight of all the proteins expressed in the target cell (or tissue) or all of the proteins detectable by a method to determine proteomic quantification, e.g., mass spectrometry.

tRNA frequency for a selected target cell (or tissue) can be determined, by way of example, by sequencing methods.

Con-rarity, (or an element of con-rarity, where other elements contribute to the overall determination of con-rarity), for a codon can be defined or evaluated by a function of a codon's proteome codon count and its cognate tRNA frequency in a target cell (or tissue), e.g, by the a function of the ratio of one to the other (PCC-tF). In an embodiment, the function is the ratio of tRNA frequency to proteome codon count. If increasing tRNA frequency is plotted on the x axis and increasing proteome codon count is plotted on the Y axis (see, e.g., FIG. 2) then in an embodiment, the tendency toward the upper left quadrant is associated with relatively greater con-rarity and the tendency toward the bottom right quadrant is associated with relatively lessor con-rarity.

Con-rarity, (or an element of con-rarity), for a codon can be defined or evaluated by the codon satisfying a reference value for proteome codon count and satisfying a reference value for tRNA frequency in a target cell (or tissue), or for satisfying a reference value for PCC-tF.

The range of values for proteome codon count for a set of reference proteins can be divided into subranges, e.g., into quartiles, quintiles, deciles, or percentiles. Likewise, the range of values for tRNA frequency (for a selected codon) can divided into subranges, e.g., into quartiles, quintiles, deciles, or percentiles. In an embodiment, con-rarity (or an element of con-rarity) can be defined or evaluated as a codon which meets a selected reference for proteome codon count and meets a selected reference for tRNA frequency.

In an embodiment, a codon is con-rare (or satisfies an element of con-rarity) if the codon falls within a selected subrange or set of subranges for proteome codon count and has a codon frequency of less than a reference value or which falls into a selected subrange or set of subranges for frequency, or has a value for PCC-tF corresponding to satisfying such selected subranges or sets of subranges.

In an embodiment, a codon is con-rare (or satisfies an element of con-rarity) if it is in fifth decile or above for proteome codon count and in the fifth decile, or lower, for tRNA frequency, or has a value for PCC-tF corresponding to satisfying such selected subranges or sets of subranges.

In an embodiment, a codon is con-rare (or satisfies an element of con-rarity) if it is in fourth decile or above for proteome codon count and in the fourth decile, or lower, for tRNA frequency, or has a value for PCC-tF corresponding to satisfying such selected subranges or sets of subranges.

In an embodiment, a codon is con-rare (or satisfies an element of con-rarity) if it is in third decile or above for proteome codon count and in the third decile, or lower, for tRNA frequency, or has a value for PCC-tF corresponding to satisfying such selected subranges or sets of subranges.

In an embodiment, a codon is con-rare (or satisfies an element of con-rarity) if it is in second decile or above for proteome codon count and in the second decile, or lower, for tRNA frequency, or has a value for PCC-tF corresponding to satisfying such selected subranges or sets of subranges.

In an embodiment, a codon is con-rare (or satisfies an element of con-rarity) if it is in first decile or above for proteome codon count and in the first decile, or lower, for tRNA frequency, or has a value for PCC-tF corresponding to satisfying such selected subranges or sets of subranges.

Methods of Modulating a Production Parameter of an RNA, or a Protein Encoded by an RNA Having a Con-Rare Codon with a TREM Composition

A production parameter of an RNA, or a protein encoded by an RNA having a con-rare codon, can be modulated by administration of a TREM composition comprising a TREM corresponding to said con-rare codon.

In an aspect, provided herein is a method of method of modulating a production parameter of an RNA, or a protein encoded by an RNA, in a target cell or tissue, comprising:

providing, e.g., administering, to the target cell or tissue, or contacting the target cell or tissue with, an effective amount of a tRNA effector molecule (TREM) (e.g., a TREM composition comprising a TREM), which TREM corresponds to a contextually-rare codon (“con-rare codon”) of the RNA,

thereby modulating the production parameter of the RNA, or protein encoded by the RNA in the target cell or tissue.

The TREM composition can be administered to the subject or the target cell or tissue can be contacted ex vivo with the TREM composition. In an embodiment, the target cell or tissue which has been contacted ex vivo with the TREM composition can be introduced into a subject, e.g., an allogeneic subject or an autologous subject.

Modulation of a production parameter of an RNA, or a protein encoded by an RNA having a con-rare codon by administration of a TREM composition (e.g., comprising a TREM corresponding to the con-rare codon) comprises modulation of an expression parameter or a signaling parameter, e.g., as described herein.

For example, administration of a TREM composition to a target cell or tissue can result in an increase or decrease in any one or more of the following expression parameters for the con-rare codon RNA:

(a) protein translation;

(b) expression level (e.g., of polypeptide or protein, or mRNA);

(c) post-translational modification of polypeptide or protein;

(d) folding (e.g., of polypeptide or protein, or mRNA),

(e) structure (e.g., of polypeptide or protein, or mRNA),

(f) transduction (e.g., of polypeptide or protein),

(g) compartmentalization (e.g., of polypeptide or protein, or mRNA),

(h) incorporation (e.g., of polypeptide or protein, or mRNA) into a supermolecular structure, e.g., incorporation into a membrane, proteasome, or ribosome,

(i) incorporation into a multimeric polypeptide, e.g., a homo or heterodimer, and/or

(j) stability.

As another example, administration of a TREM composition to a target cell or tissue can result in an increase or decrease in any one or more of the following signaling parameters for the con-rare codon RNA:

(1) modulation of a signaling pathway, e.g., a cellular signaling pathway which is downstream or upstream of the protein encoded by the con-rare codon RNA;

(2) cell fate modulation;

(3) ribosome occupancy modulation;

(4) protein translation modulation;

(5) mRNA stability modulation;

(6) protein folding and structure modulation;

(7) protein transduction or compartmentalization modulation; and/or

(8) protein stability modulation.

A production parameter (e.g., an expression parameter and/or a signaling parameter) may be modulated, e.g., by at least 5% (e.g., at least 10%, 15%, 20%, 25%, 30%, 40%. 50%. 60%. 70%, 80%, 90%, 100%, 150%, 200% or more) compared to a reference nucleic acid sequence, e.g., parental, wildtype or conventionally optimized nucleic acid sequence.

### Host Cells

A host cell is a cell (e.g., a cultured cell) that can be used for expression and/or purification of a TREM. In an embodiment, a host cell comprises a mammalian cell or a non-mammalian cell. In an embodiment, a host cell comprises a mammalian cell, e.g., a human cell, or a rodent cell. In an embodiment, a host cell comprises a HeLa cell, a HEK293T cell (e.g., a Freestyle 293-F cell), a HT-1080 cell, a PER.C6 cell, a HKB-11 cell, a CAP cell, a HuH-7 cell, a BHK 21 cell, an MRC-S cell, a MDCK cell, a VERO cell, a WI-38 cell, or a Chinese Hamster Ovary (CHO) cell. In an embodiment, a host cell comprises a cancer cell, e.g., a solid tumor cell (e.g., a breast cancer cell (e.g., a MCF7 cell), a pancreatic cell line (e.g. a MIA PaCa-2 cell), a lung cancer cell, or a prostate cancer cell, or a hematological cancer cell). In an embodiment, a host cell is a primary cell, e.g., a cell that has not been immortalized or a cell with a finite proliferation capacity. In an embodiment, a host cell is a cell derived from a subject, e.g., a patient.

In an embodiment, a host cell comprises a non-mammalian cell, e.g., a bacterial cell, a yeast cell or an insect cell. In an embodiment, a host cell comprises a bacterial cell, e.g., an E. coli cell. In an embodiment, a host cell comprises a yeast cell, e.g., a S. cerevisiae cell. In an embodiment, a host cell comprises an insect cell, e.g., a Sf-9 cell or a Hi5 cell.

In an embodiment, a host cell comprises a cell that expresses one or more tissue specific tRNAs. For example, a host cell can comprise a cell derived from a tissue associated with expression of a tRNA, e.g., a tissue specific tRNA. In an embodiment, a host cell that expresses a tissue specific tRNA is modified to express a TREM, or a fragment thereof.

In an embodiment, a host cell is a cell that can be maintained under conditions that allow for expression of a TREM.

In an embodiment, a host cell is capable of post-transcriptionally modifying the TREM, e.g., adding a post-transcriptional modification selected from Table 2. In an embodiment, a host cell expresses (e.g., naturally or heterologously) an enzyme listed in Table 2. In an embodiment, a host cell expresses (e.g., naturally or heterologously) an enzyme, e.g., an enzyme having nuclease activity (e.g., endonuclease activity or ribonuclease activity), e.g., or one or more of Dicer, Angiogenin, RNaseA, RNaseP, RNaseZ, Rny1 or PrrC.

### Method of Culturing Host Cell

A host cell can be cultured in a medium that promotes growth, e.g., proliferation or hyperproliferation of the host cell. A host cell can be cultured in a suitable media, e.g., any of the following media: DMEM, MEM, MEM alpha, RPMI, F-10 media, F-12 media, DMEM/F-12 media, IMDM, Medium 199, Leibovitz L-15, McCoys's 5A, MDCB media, or CMRL media. In an embodiment the media is supplemented with glutamine. In an embodiment, the media is not supplemented with glutamine. In an embodiment, a host cell is cultured in media that has an excess of nutrients, e.g., is not nutrient limiting.

A host cell can be cultured in a medium comprising or supplemented with one or a combination of growth factors, cytokines or hormones, e.g., one or a combination of serum (e.g., fetal bovine serum (FBS)), HEPES, fibroblast growth factor (FGFs), epidermal growth factors (EGFs), insulin-like growth factors (IGFs), transforming growth factor beta (TGFb), platelet derived growth factor (PDGFs), hepatocyte growth factor (HGFs), or tumor necrosis factor (TNFs).

A host cell, e.g., a non-mammalian host cell, can be cultured in any of the following media: Luria Broth, YPD media or Grace's Medium.

A host cell can also be cultured under conditions that induce stress, e.g., cellular stress, osmotic stress, translational stress, or oncogenic stress. In an embodiment, a host cell expressing a TREM, cultured under conditions that induce stress (e.g., as described herein) results in a fragment of the TREM, e.g., as described herein.

A host cell can be cultured under nutrient limiting conditions, e.g., the host cell is cultured in media that has a limited amount of one or more nutrients. Examples of nutrients that can be limiting are amino acids, lipids, carbohydrates, hormones, growth factors or vitamins. In an embodiment, a host cell expressing a TREM, cultured in media that has a limited amount of one or more nutrients, e.g., the media is nutrient starved, results in a fragment of the TREM, e.g., as described herein. In an embodiment, a host cell expressing a TREM, cultured in media that has a limited amount of one or more nutrients, e.g., the media is nutrient starved, results in a TREM that is uncharged (e.g. a uTREM).

A host cell can comprise an immortalized cell, e.g., a cell which expresses one or more enzymes involved in immortalization, e.g., TERT. In an embodiment, a host cell can be propagated indefinitely.

A host cell can be cultured in suspension or as a monolayer. Host cell cultures can be performed in a cell culture vessel or a bioreactor. Cell culture vessels include a cell culture dish, plate or flask. Exemplary cell culture vessels include 35 mm, 60 mm, 100 mm, or 150 mm dishes, multi-well plates (e.g., 6-well, 12-well, 24-well, 48-well or 96 well plates), or T-25, T-75 or T-160 flasks.

In an embodiment, a host cell can be cultured in a bioreactor. A bioreactor can be, e.g., a continuous flow batch bioreactor, a perfusion bioreactor, a batch process bioreactor or a fed batch bioreactor. A bioreactor can be maintained under conditions sufficient to express the TREM. The culture conditions can be modulated to optimize yield, purity or structure of the TREM. In an embodiment, a bioreactor comprises at least 1×10⁷, 1×10⁸, 1×10⁹, 1×10¹⁰, 1×10¹¹, 1×10¹², 1×10¹³, or 1×10¹⁴ host cells.

In an embodiment, a bioreactor comprises between 1×10⁵ host cells/mL to 1×10⁹ host cells/mL, between 5×10⁵ host cells/mL to 1×10⁹ host cells/mL, between 1×10⁶ host cells/mL to 1×10⁹ host cells/mL; between 5×10⁶ host cells/mL to 1×10⁹ host cells/mL, between 1×10⁷ host cells/mL to 1×10⁹ host cells/mL, between 5×10⁷ host cells/mL to 1×10⁹ host cells/mL, between 1×10⁸ host cells/mL to 1×10⁹ host cells/mL, between 5×10⁸ host cells/mL to 1×10⁹ host cells/mL, between 1×10⁵ host cells/mL to 5×10⁸ host cells/mL, between 1×10⁵ host cells/mL to 1×10⁸ host cells/mL, between 1×10⁵ host cells/mL to 5×10⁷ host cells/mL, between 1×10⁵ host cells/mL to 1×10⁷ host cells/mL, between 1×10⁵ host cells/mL to 5×10⁶ host cells/mL, between 1×10⁵ host cells/mL to 1×10⁶ host cells/mL, or between 1×10⁵ host cells/mL to 5×10⁵ host cells/mL.

In an embodiment, a bioreactor is maintained under conditions that promote growth of the host cell, e.g., at a temperature (e.g., 37° C.) and gas concentration (e.g., 5% CO₂) that is permissive for growth of the host cell.

For example, in some aspects, a bioreactor unit can perform one or more, or all, of the following: feeding of nutrients and/or carbon sources, injection of suitable gas (e.g., oxygen), inlet and outlet flow of fermentation or cell culture medium, separation of gas and liquid phases, maintenance of temperature, maintenance of oxygen and C02 levels, maintenance of pH level, agitation (e.g., stirring), and/or cleaning/sterilizing. Exemplary bioreactor units, may contain multiple reactors within the unit, for example the unit can have 1, 2, 3, 4, 5, 10, 15, 20, 25, 30, 35, 40, 45, 50, 60, 70, 80, 90, or 100, or more bioreactors in each unit and/or a facility may contain multiple units having a single or multiple reactors within the facility. Any suitable bioreactor diameter can be used.

In an embodiment, the bioreactor can have a volume between about 100 mL and about 100 L. Non-limiting examples include a volume of 100 mL, 250 mL, 500 mL, 750 mL, 1 liter, 2 liters, 3 liters, 4 liters, 5 liters, 6 liters, 7 liters, 8 liters, 9 liters, 10 liters, 15 liters, 20 liters, 25 liters, 30 liters, 40 liters, 50 liters, 60 liters, 70 liters, 80 liters, 90 liters, 100 liters. Additionally, suitable reactors can be multi-use, single-use, disposable, or non-disposable and can be formed of any suitable material including metal alloys such as stainless steel (e.g., 316L or any other suitable stainless steel) and Inconel, plastics, and/or glass. In some embodiments, suitable reactors can be round, e.g., cylindrical. In some embodiments, suitable reactors can be square, e.g., rectangular. Square reactors may in some cases provide benefits over round reactors such as ease of use (e.g., loading and setup by skilled persons), greater mixing and homogeneity of reactor contents, and lower floor footprint.

### Method of Modifying Host Cells

A host cell can be modified to optimize the production of a TREM, e.g., to have optimized TREM yield, purity, structure (e.g., folding), or stability. In an embodiment, a host cell can be modified (e.g., using a method described herein), to increase or decrease the expression of a desired molecule, e.g., gene, which optimizes production of the TREM, e.g., optimizes yield, purity, structure or stability of the TREM. In an embodiment, a host cell can be epigenetically modified, e.g., using a method described herein, to increase or decrease the expression of a desired gene, which optimizes production.

In an embodiment, a host cell can be modified to increase or decrease the expression of an oncogene (e.g., as described herein), a tumor suppressor (e.g., as described herein) or a molecule involved in tRNA or TREM modulation (e.g., a gene involved in tRNA or TREM transcription, processing, modification, stability or folding). Exemplary oncogenes include Myc (e.g., c-Myc, N-Myc or L-Myc), c-Jun, Wnt, or RAS. Exemplary tumor suppressors include p53 or Rb. Exemplary molecules involved in tRNA or TREM modulation include: RNA Polymerase III (Pol III) and Pol III accessory molecules (e.g., TFIIIB); Maf1, Trm1, Mck1 or Kns 1; enzymes involved in tRNA or TREM modification, e.g., genes listed in Table 2; or molecules with nuclease activity, e.g., or one or more of Dicer, Angiogenin, RNaseA, RNaseP, RNaseZ, Rny1 or PrrC.

In an embodiment, a host cell can be modified by: transfection (e.g., transient transfection or stable transfection); transduction (e.g., viral transduction, e.g., lentiviral, adenoviral or retroviral transduction); electroporation; lipid-based delivery of an agent (e.g., liposomes), nanoparticle based delivery of an agent; or other methods known in the art.

In an embodiment, a host cell can be modified to increase the expression of, e.g., overexpress, a desired molecule, e.g., a gene (e.g., an oncogene, or a gene involved in tRNA or TREM modulation (e.g., a gene encoding an enzyme listed in Table 2, or a gene encoding an enzyme having nuclease activity (e.g., endonuclease activity or ribonuclease activity), e.g., or one or more of Dicer, Angiogenin, RNaseA, RNaseP, RNaseZ, Rny1 or PrrC. Exemplary methods of increasing the expression of a gene include: (a) contacting the host cell with a nucleic acid (e.g., DNA, or RNA) encoding the gene; (b) contacting the host cell with a peptide that expresses the target protein; (c) contacting the host cell with a molecule (e.g., a small RNA (e.g., a micro RNA, or a small interfering RNA) or a low molecular weight compound) that modulates, e.g., increases the expression of the target gene; or (d) contacting the host cell with a gene editing moiety (e.g., a zinc finger nuclease (ZFN) or a Cas9/CRISPR molecule) that inhibits (e.g., mutates or knocks-out) the expression of a negative regulator of the target gene. In an embodiment, a nucleic acid encoding the gene, or a plasmid containing a nucleic acid encoding the gene can be introduced into the host cell by transfection or electroporation. In an embodiment, a nucleic acid encoding a gene can be introduced into the host cell by contacting the host cell with a virus (e.g., a lentivirus, adenovirus or retrovirus) expressing the gene.

In an embodiment, a host cell can be modified to decrease the expression of, e.g., minimize the expression, of a desired molecule, e.g., a gene (e.g., a tumor suppressor, or a gene involved in tRNA or TREM modulation). Exemplary methods of decreasing the expression of a gene include: (a) contacting the host cell with a nucleic acid (e.g., DNA, or RNA) encoding an inhibitor of the gene (e.g., a dominant negative variant or a negative regulator of the gene or protein encoded by the gene); (b) contacting the host cell with a peptide that inhibits the target protein; (c) contacting the host cell with a molecule (e.g., a small RNA (e.g., a micro RNA, or a small interfering RNA) or a low molecular weight compound) that modulates, e.g., inhibits the expression of the target gene; or (d) contacting the host cell with a gene editing moiety (e.g., a zinc finger nuclease (ZFN) or a Cas9/CRISPR molecule) that inhibits (e.g., mutates or knocks-out) the expression of the target gene. In an embodiment, a nucleic acid encoding an inhibitor of the gene, or a plasmid containing a nucleic acid encoding an inhibitor of the gene can be introduced into the host cell by transfection or electroporation. In an embodiment, a nucleic acid encoding an inhibitor of the gene can be introduced into the host cell by contacting the host cell with a virus (e.g., a lentivirus, adenovirus or retrovirus) expressing the inhibitor of the gene.

In an embodiment, a host cell (e.g., a host cell described herein) is modified (e.g., by transfection with a nucleic acid), to express, e.g., overexpress, an oncogene, e.g., an oncogene described herein, e.g., c-Myc.

In an embodiment, a host cell (e.g., a host cell described herein) is modified (e.g., by transfection with a nucleic acid), to repress, e.g., downregulate, expression of a tumor suppressor, e.g., a tumor suppressor described herein, e.g., p53 or Rb.

In an embodiment, a host cell (e.g., a HEK293T cell) is modified (e.g., using a CRISPR/Cas9 molecule) to inhibit, e.g., knockout, expression of a gene that modulates a tRNA or TREM, e.g., Maf1. In an embodiment, a host cell (e.g., a HEK293T cell) is modified to overexpress a gene that modulates a tRNA or TREM, e.g., Trm1.

In an embodiment, a host cell (e.g., a HEK293T cell) is modified to overexpress a gene that modulates a tRNA or TREM, e.g., Trm1, and to overexpress an oncogene, e.g., an oncogene described herein, e.g., c-Myc.

### TREM

A “tRNA-based effector molecule” or “TREM” refers to an RNA molecule comprising one or more of the properties described herein. A TREM can be charged with an amino acid, e.g., a cognate amino acid; charged with a non-cognate amino acid (e.g., a mischarged TREM (mTREM); or not charged with an amino acid, e.g., an uncharged TREM (uTREM).

In an embodiment, a TREM described herein is a TREM that corresponds to a con-rare codon in a nucleic acid sequence, e.g., DNA or RNA. A nucleic acid sequence having a con-rare codon or an RNA having a con-rare codon can be identified by any of the methods disclosed herein. A tRNA corresponding to the con-rare codon (con-rare tRNA) and/or a TREM corresponding to the con-rare codon can also be determined by any of the methods disclosed herein.

In an embodiment, a TREM (e.g., a TREM corresponding to a con-rare codon) comprises a ribonucleic acid (RNA) sequence encoded by a deoxyribonucleic acid (DNA) sequence disclosed in Table 1, e.g., any one of SEQ ID NOs: 1-451 disclosed in Table 1. In an embodiment, a TREM comprises an RNA sequence at least 60%, 65%, 70%, 75%, 80%, 82%, 85%, 87%, 88%, 90%, 92%, 95%, 96%, 97%, 98%, or 99% identical to an RNA sequence encoded by a DNA sequence provided in Table 1, e.g., any one of SEQ ID NOs: 1-451 disclosed in Table 1. In an embodiment, a TREM comprises an RNA sequence encoded by a DNA sequence at least 60%, 65%, 70%, 75%, 80%, 82%, 85%, 87%, 88%, 90%, 92%, 95%, 96%, 97%, 98%, or 99% identical to a DNA sequence provided in Table 1, e.g., any one of SEQ ID NOs: 1-451 disclosed in Table 1.

In an embodiment, a TREM (e.g., a TREM corresponding to a con-rare codon) comprises at least 30 consecutive nucleotides of an RNA sequence encoded by a DNA sequence disclosed in Table 1, e.g., at least 30 consecutive nucleotides of an RNA sequence encoded by any one of SEQ ID NOs: 1-451 disclosed in Table 1. In an embodiment, a TREM comprises at least 30 consecutive nucleotides of an RNA sequence at least 60%, 65%, 70%, 75%, 80%, 82%, 85%, 87%, 88%, 90%, 92%, 95%, 96%, 97%, 98%, or 99% identical to an RNA sequence encoded by a DNA sequence provided in Table 1, e.g., any one of SEQ ID NOs: 1-451 disclosed in Table 1. In an embodiment, a TREM comprises at least 30 consecutive nucleotides of an RNA sequence encoded by a DNA sequence at least 60%, 65%, 70%, 75%, 80%, 82%, 85%, 87%, 88%, 90%, 92%, 95%, 96%, 97%, 98%, or 99% identical to a DNA sequence provided in Table 1, e.g., any one of SEQ ID NOs: 1-451 disclosed in Table 1.

TABLE 1
List of tRNA sequences

| SEQ | | |
|---|---|---|
| ID | | |
| NO | tRNA name | tRNA sequence |
| 1 | Ala_AGC_chr6:28763741-28763812 (-) | GGGGGTATAGCTCAGTGGTAGAGCGCGTGCTTAGCATGCACGAGGTCC |
| | | TGGGTTCGATCCCCAGTACCTCCA |
| 2 | Ala_AGC_chr6:26687485-26687557 (+) | GGGGAATTAGCTCAAGTGGTAGAGCGCTTGCTTAGCACGCAAGAGGTA |
| | | GTGGGATCGATGCCCACATTCTCCA |
| 3 | Ala_AGC_chr6:26572092-26572164 (-) | GGGGAATTAGCTCAAATGGTAGAGCGCTCGCTTAGCATGCGAGAGGTA |
| | | GCGGGATCGATGCCCGCATTCTCCA |
| 4 | Ala_AGC_chr6:26682715-26682787 (+) | GGGGAATTAGCTCAAGTGGTAGAGCGCTTGCTTAGCATGCAAGAGGTA |
| | | GTGGGATCGATGCCCACATTCTCCA |
| 5 | Ala_AGC_chr6:26705606-26705678 (+) | GGGGAATTAGCTCAAGCGGTAGAGCGCTTGCTTAGCATGCAAGAGGTA |
| | | GTGGGATCGATGCCCACATTCTCCA |
| 6 | Ala_AGC_chr6:26673590-26673662 (+) | GGGGAATTAGCTCAAGTGGTAGAGCGCTTGCTTAGCATGCAAGAGGTA |
| | | GTGGGATCAATGCCCACATTCTCCA |
| 7 | Ala_AGC_chr14:89445442-89445514 (+) | GGGGAATTAGCTCAAGTGGTAGAGCGCTCGCTTAGCATGCGAGAGGTA |
| | | GTGGGATCGATGCCCGCATTCTCCA |
| 8 | Ala_AGC_chr6:58196623-58196695 (-) | GGGGAATTAGCCCAAGTGGTAGAGCGCTTGCTTAGCATGCAAGAGGTA |
| | | GTGGGATCGATGCCCACATTCTCCA |
| 9 | Ala_AGC_chr6:28806221-28806292 (-) | GGGGGTGTAGCTCAGTGGTAGAGCGCGTGCTTAGCATGCACGAGGCCC |
| | | CGGGTTCAATCCCCGGCACCTCCA |
| 10 | Ala_AGC_chr6:28574933-28575004 (+) | GGGGGTGTAGCTCAGTGGTAGAGCGCGTGCTTAGCATGTACGAGGTCC |
| | | CGGGTTCAATCCCCGGCACCTCCA |
| 11 | Ala_AGC_chr6:28626014-28626085 (-) | GGGGATGTAGCTCAGTGGTAGAGCGCATGCTTAGCATGCATGAGGTCC |
| | | CGGGTTCGATCCCCAGCATCTCCA |
| 12 | Ala_AGC_chr6:28678366-28678437 (+) | GGGGGTGTAGCTCAGTGGTAGAGCGCGTGCTTAGCATGCACGAGGCCC |
| | | TGGGTTCAATCCCCAGCACCTCCA |
| 13 | Ala_AGC_chr6:28779849-28779920 (-) | GGGGGTATAGCTCAGCGGTAGAGCGCGTGCTTAGCATGCACGAGGTCC |
| | | TGGGTTCAATCCCCAATACCTCCA |
| 14 | Ala_AGC_chr6:28687481-28687552 (+) | GGGGGTGTAGCTCAGTGGTAGAGCGCGTGCTTAGCATGCACGAGGCCC |
| | | CGGGTTCAATCCCTGGCACCTCCA |
| 15 | Ala_AGC_chr2:27274082-27274154 (+) | GGGGGATTAGCTCAAATGGTAGAGCGCTCGCTTAGCATGCGAGAGGTA |
| | | GCGGGATCGATGCCCGCATCCTCCA |
| 16 | Ala_AGC_chr6:26730737-26730809 (+) | GGGGAATTAGCTCAGGCGGTAGAGCGCTCGCTTAGCATGCGAGAGGTA |
| | | GCGGGATCGACGCCCGCATTCTCCA |
| 17 | Ala_CGC_chr6:26553731-26553802 (+) | GGGGATGTAGCTCAGTGGTAGAGCGCATGCTTCGCATGTATGAGGTCC |
| | | CGGGTTCGATCCCCGGCATCTCCA |
| 18 | Ala_CGC_chr6:28641613-28641684 (-) | GGGGATGTAGCTCAGTGGTAGAGCGCATGCTTCGCATGTATGAGGCCC |
| | | CGGGTTCGATCCCCGGCATCTCCA |
| 19 | Ala_CGC_chr2:157257281-157257352 | GGGGATGTAGCTCAGTGGTAGAGCGCGCGCTTCGCATGTGTGAGGTCC |
| | (+) | CGGGTTCAATCCCCGGCATCTCCA |
| 20 | Ala_CGC_chr6:28697092-28697163 (+) | GGGGGTGTAGCTCAGTGGTAGAGCGCGTGCTTCGCATGTACGAGGCCC |
| | | CGGGTTCGACCCCCGGCTCCTCCA |
| 21 | Ala_TGC_chr6:28757547-28757618 (-) | GGGGGTGTAGCTCAGTGGTAGAGCGCATGCTTTGCATGTATGAGGTCC |
| | | CGGGTTCGATCCCCGGCACCTCCA |
| 22 | Ala_TGC_chr6:28611222-28611293 (+) | GGGGATGTAGCTCAGTGGTAGAGCGCATGCTTTGCATGTATGAGGTCC |
| | | CGGGTTCGATCCCCGGCATCTCCA |
| 23 | Ala_TGC_chr5:180633868-180633939 | GGGGATGTAGCTCAGTGGTAGAGCGCATGCTTTGCATGTATGAGGCCC |
| | (+) | CGGGTTCGATCCCCGGCATCTCCA |
| 24 | Ala_TGC_chr12:125424512-125424583 | GGGGATGTAGCTCAGTGGTAGAGCGCATGCTTTGCACGTATGAGGCCC |
| | (+) | CGGGTTCAATCCCCGGCATCTCCA |
| 25 | Ala_TGC_chr6:28785012-28785083 (-) | GGGGGTGTAGCTCAGTGGTAGAGCGCATGCTTTGCATGTATGAGGCCT |
| | | CGGGTTCGATCCCCGACACCTCCA |
| 26 | Ala_TGC_chr6:28726141-28726212 (-) | GGGGGTGTAGCTCAGTGGTAGAGCACATGCTTTGCATGTGTGAGGCCC |
| | | CGGGTTCGATCCCCGGCACCTCCA |
| 27 | Ala_TGC_chr6:28770577-28770647 (-) | GGGGGTGTAGCTCAGTGGTAGAGCGCATGCTTTGCATGTATGAGGCCT |
| | | CGGTTCGATCCCCGACACCTCCA |
| 28 | Arg_ACG_chr6:26328368-26328440 (+) | GGGCCAGTGGCGCAATGGATAACGCGTCTGACTACGGATCAGAAGATT |
| | | CCAGGTTCGACTCCTGGCTGGCTCG |
| 29 | Arg_ACG_chr3:45730491-45730563 (-) | GGGCCAGTGGCGCAATGGATAACGCGTCTGACTACGGATCAGAAGATT |
| | | CTAGGTTCGACTCCTGGCTGGCTCG |
| 30 | Arg_CCG_chr6:28710729-28710801 (-) | GGCCGCGTGGCCTAATGGATAAGGCGTCTGATTCCGGATCAGAAGATT |
| | | GAGGGTTCGAGTCCCTTCGTGGTCG |
| 31 | Arg_CCG_chr17:66016013-66016085 (-) | GACCCAGTGGCCTAATGGATAAGGCATCAGCCTCCGGAGCTGGGGATT |
| | | GTGGGTTCGAGTCCCATCTGGGTCG |
| 32 | Arg_CCT_chr17:73030001-73030073 (+) | GCCCCAGTGGCCTAATGGATAAGGCACTGGCCTCCTAAGCCAGGGATT |
| | | GTGGGTTCGAGTCCCACCTGGGGTA |
| 33 | Arg_CCT_chr17:73030526-73030598 (-) | GCCCCAGTGGCCTAATGGATAAGGCACTGGCCTCCTAAGCCAGGGATT |
| | | GTGGGTTCGAGTCCCACCTGGGGTG |
| 34 | Arg_CCT_chr16:3202901-3202973 (+) | GCCCCGGTGGCCTAATGGATAAGGCATTGGCCTCCTAAGCCAGGGATT |
| | | GTGGGTTCGAGTCCCACCCGGGGTA |
| 35 | Arg_CCT_chr7:139025446-139025518 | GCCCCAGTGGCCTAATGGATAAGGCATTGGCCTCCTAAGCCAGGGATT |
| | (+) | GTGGGTTCGAGTCCCATCTGGGGTG |
| 36 | Arg_CCT_chr16:3243918-3243990 (+) | GCCCCAGTGGCCTGATGGATAAGGTACTGGCCTCCTAAGCCAGGGATT |
| | | GTGGGTTCGAGTTCCACCTGGGGTA |
| 37 | Arg_TCG_chr15:89878304-89878376 (+) | GGCCGCGTGGCCTAATGGATAAGGCGTCTGACTTCGGATCAGAAGATT |
| | | GCAGGTTCGAGTCCTGCCGCGGTCG |
| 38 | Arg_TCG_chr6:26323046-26323118 (+) | GACCACGTGGCCTAATGGATAAGGCGTCTGACTTCGGATCAGAAGATT |
| | | GAGGGTTCGAATCCCTCCGTGGTTA |
| 39 | Arg_TCG_chr17:73031208-73031280 (+) | GACCGCGTGGCCTAATGGATAAGGCGTCTGACTTCGGATCAGAAGATT |
| | | GAGGGTTCGAGTCCCTTCGTGGTCG |
| 40 | Arg_TCG_chr6:26299905-26299977 (+) | GACCACGTGGCCTAATGGATAAGGCGTCTGACTTCGGATCAGAAGATT |
| | | GAGGGTTCGAATCCCTTCGTGGTTA |
| 41 | Arg_TCG_chr6:28510891-28510963 (-) | GACCACGTGGCCTAATGGATAAGGCGTCTGACTTCGGATCAGAAGATT |
| | | GAGGGTTCGAATCCCTTCGTGGTTG |
| 42 | Arg_TCG_chr9:112960803-112960875 | GGCCGTGTGGCCTAATGGATAAGGCGTCTGACTTCGGATCAAAAGATT |
| | (+) | GCAGGTTTGAGTTCTGCCACGGTCG |
| 43 | Arg_TCT_chr1:94313129-94313213 (+) | GGCTCCGTGGCGCAATGGATAGCGCATTGGACTTCTAGAGGCTGAAGG |
| | | CATTCAAAGGTTCCGGGTTCGAGTCCCGGCGGAGTCG |
| 44 | Arg_TCT_chr17:8024243-8024330 (+) | GGCTCTGTGGCGCAATGGATAGCGCATTGGACTTCTAGTGACGAATAG |
| | | AGCAATTCAAAGGTTGTGGGTTCGAATCCCACCAGAGTCG |
| 45 | Arg_TCT_chr9:131102355-131102445 (-) | GGCTCTGTGGCGCAATGGATAGCGCATTGGACTTCTAGCTGAGCCTAG |
| | | TGTGGTCATTCAAAGGTTGTGGGTTCGAGTCCCACCAGAGTCG |
| 46 | Arg_TCT_chr11:59318767-59318852 (+) | GGCTCTGTGGCGCAATGGATAGCGCATTGGACTTCTAGATAGTTAGAG |
| | | AAATTCAAAGGTTGTGGGTTCGAGTCCCACCAGAGTCG |
| 47 | Arg_TCT_chr1:159111401-159111474 (-) | GTCTCTGTGGCGCAATGGACGAGCGCGCTGGACTTCTAATCCAGAGGT |
| | | TCCGGGTTCGAGTCCCGGCAGAGATG |
| 48 | Arg_TCT_chr6:27529963-27530049 (+) | GGCTCTGTGGCGCAATGGATAGCGCATTGGACTTCTAGCCTAAATCAA |
| | | GAGATTCAAAGGTTGCGGGTTCGAGTCCCTCCAGAGTCG |
| 49 | Asn_GTT_chr1:161510031-161510104 | GTCTCTGTGGCGCAATCGGTTAGCGCGTTCGGCTGTTAACCGAAAGGT |
| | (+) | TGGTGGTTCGATCCCACCCAGGGACG |
| 50 | Asn_GTT_chr1:143879832-143879905 (-) | GTCTCTGTGGCGCAATCGGCTAGCGCGTTTGGCTGTTAACTAAAAGGTT |
| | | GGCGGTTCGAACCCACCCAGAGGCG |
| 51 | Asn_GTT_chr1:144301611-144301684 | GTCTCTGTGGTGCAATCGGTTAGCGCGTTCCGCTGTTAACCGAAAGCTT |
| | (+) | GGTGGTTCGAGCCCACCCAGGGATG |
| 52 | Asn_GTT_chr1:149326272-149326345 (-) | GTCTCTGTGGCGCAATCGGCTAGCGCGTTTGGCTGTTAACTAAAAAGTT |
| | | GGTGGTTCGAACACACCCAGAGGCG |
| 53 | Asn_GTT_chr1:148248115-148248188 | GTCTCTGTGGCGCAATCGGTTAGCGCGTTCGGCTGTTAACCGAAAGGT |
| | (+) | TGGTGGTTCGAGCCCACCCAGGGACG |
| 54 | Asn_GTT_chr1:148598314-148598387 (-) | GTCTCTGTGGCGCAATCGGTTAGCGCATTCGGCTGTTAACCGAAAGGT |
| | | TGGTGGTTCGAGCCCACCCAGGGACG |
| 55 | Asn_GTT_chr1:17216172-17216245 (+) | GTCTCTGTGGCGCAATCGGTTAGCGCGTTCGGCTGTTAACCGAAAGAT |
| | | TGGTGGTTCGAGCCCACCCAGGGACG |
| 56 | Asn_GTT_chr1:16847080-16847153 (-) | GTCTCTGTGGCGCAATCGGTTAGCGCGTTCGGCTGTTAACTGAAAGGTT |
| | | GGTGGTTCGAGCCCACCCAGGGACG |
| 57 | Asn_GTT_chr1:149230570-149230643 (-) | GTCTCTGTGGCGCAATGGGTTAGCGCGTTCGGCTGTTAACCGAAAGGT |
| | | TGGTGGTTCGAGCCCATCCAGGGACG |
| 58 | Asn_GTT_chr1:148000805-148000878 | GTCTCTGTGGCGTAGTCGGTTAGCGCGTTCGGCTGTTAACCGAAAAGTT |
| | (+) | GGTGGTTCGAGCCCACCCAGGAACG |
| 59 | Asn_GTT_chr1:149711798-149711871 (-) | GTCTCTGTGGCGCAATCGGCTAGCGCGTTTGGCTGTTAACTAAAAGGTT |
| | | GGTGGTTCGAACCCACCCAGAGGCG |
| 60 | Asn_GTT_chr1:145979034-145979107 (-) | GTCTCTGTGGCGCAATCGGTTAGCGCGTTCGGCTGTTAACTGAAAGGTT |
| | | AGTGGTTCGAGCCCACCCGGGGACG |
| 61 | Asp_GTC_chr12:98897281-98897352 (+) | TCCTCGTTAGTATAGTGGTTAGTATCCCCGCCTGTCACGCGGGAGACCG |
| | | GGGTTCAATTCCCCGACGGGGAG |
| 62 | Asp_GTC_chr1:161410615-161410686 (-) | TCCTCGTTAGTATAGTGGTGAGTATCCCCGCCTGTCACGCGGGAGACC |
| | | GGGGTTCGATTCCCCGACGGGGAG |
| 63 | Asp_GTC_chr6:27551236-27551307(-) | TCCTCGTTAGTATAGTGGTGAGTGTCCCCGTCTGTCACGCGGGAGACC |
| | | GGGGTTCGATTCCCCGACGGGGAG |
| 64 | Cys_GCA_chr7:149007281-149007352 | GGGGGCATAGCTCAGTGGTAGAGCATTTGACTGCAGATCAAGAGGTCC |
| | (+) | CTGGTTCAAATCCAGGTGCCCCCT |
| 65 | Cys_GCA_chr7:149074601-149074672 (-) | GGGGGTATAGCTCAGGGGTAGAGCATTTGACTGCAGATCAAGAGGTCC |
| | | CTGGTTCAAATCCAGGTGCCCCCC |
| 66 | Cys_GCA_chr7:149112229-149112300 (-) | GGGGGTATAGCTTAGCGGTAGAGCATTTGACTGCAGATCAAGAGGTCC |
| | | CCGGTTCAAATCCGGGTGCCCCCT |
| 67 | Cys_GCA_chr7:149344046-149344117 (-) | GGGGGTATAGCTTAGGGGTAGAGCATTTGACTGCAGATCAAAAGGTCC |
| | | CTGGTTCAAATCCAGGTGCCCCTT |
| 68 | Cys_GCA_chr7:149052766-149052837 (-) | GGGGGTATAGCTCAGGGGTAGAGCATTTGACTGCAGATCAAGAGGTCC |
| | | CCAGTTCAAATCTGGGTGCCCCCT |
| 69 | Cys_GCA_chr17:37017937-37018008 (-) | GGGGGTATAGCTCAGGGGTAGAGCATTTGACTGCAGATCAAGAAGTCC |
| | | CCGGTTCAAATCCGGGTGCCCCCT |
| 70 | Cys_GCA_chr7:149281816-149281887 | GGGGGTATAGCTCAGGGGTAGAGCATTTGACTGCAGATCAAGAGGTCT |
| | (+) | CTGGTTCAAATCCAGGTGCCCCCT |
| 71 | Cys_GCA_chr7:149243631-149243 702 | GGGGGTATAGCTCAGGGGTAGAGCACTTGACTGCAGATCAAGAAGTCC |
| | (+) | TTGGTTCAAATCCAGGTGCCCCCT |
| 72 | Cys_GCA_chr7:149388272-149388343 (-) | GGGGATATAGCTCAGGGGTAGAGCATTTGACTGCAGATCAAGAGGTCC |
| | | CCGGTTCAAATCCGGGTGCCCCCC |
| 73 | Cys_GCA_chr7:149072850-149072921 (-) | GGGGGTATAGTTCAGGGGTAGAGCATTTGACTGCAGATCAAGAGGTCC |
| | | CTGGTTCAAATCCAGGTGCCCCCT |
| 74 | Cys_GCA_chr7:149310156-149310227(-) | GGGGGTATAGCTCAGGGGTAGAGCATTTGACTGCAAATCAAGAGGTCC |
| | | CTGATTCAAATCCAGGTGCCCCCT |
| 75 | Cys_GCA_chr4:124430005-124430076 (-) | GGGGGTATAGCTCAGTGGTAGAGCATTTGACTGCAGATCAAGAGGTCC |
| | | CCGGTTCAAATCCGGGTGCCCCCT |
| 76 | Cys_GCA_chr7:149295046-149295117 | GGGCGTATAGCTCAGGGGTAGAGCATTTGACTGCAGATCAAGAGGTCC |
| | (+) | CCAGTTCAAATCTGGGTGCCCCCT |
| 77 | Cys_GCA_chr7:149361915-149361986 | GGGGGTATAGCTCACAGGTAGAGCATTTGACTGCAGATCAAGAGGTCC |
| | (+) | CCGGTTCAAATCTGGGTGCCCCCT |
| 78 | Cys_GCA_chr7:149253802-149253871 | GGGCGTATAGCTCAGGGGTAGAGCATTTGACTGCAGATCAAGAGGTCC |
| | (+) | CCAGTTCAAATCTGGGTGCCCA |
| 79 | Cys_GCA_chr7:149292305-149292376 (-) | GGGGGTATAGCTCACAGGTAGAGCATTTGACTGCAGATCAAGAGGTCC |
| | | CCGGTTCAAATCCGGTTACTCCCT |
| 80 | Cys_GCA_chr7:149286164-149286235 (-) | GGGGGTATAGCTCAGGGGTAGAGCACTTGACTGCAGATCAAGAGGTCC |
| | | CTGGTTCAAATCCAGGTGCCCCCT |
| 81 | Cys_GCA_chr17:37025545-37025616 (-) | GGGGGTATAGCTCAGTGGTAGAGCATTTGACTGCAGATCAAGAGGTCC |
| | | CTGGTTCAAATCCGGGTGCCCCCT |
| 82 | Cys_GCA_chr15:80036997-80037069 (+) | GGGGGTATAGCTCAGTGGGTAGAGCATTTGACTGCAGATCAAGAGGTC |
| | | CCCGGTTCAAATCCGGGTGCCCCCT |
| 83 | Cys_GCA_chr3:131947944-131948015 (-) | GGGGGTGTAGCTCAGTGGTAGAGCATTTGACTGCAGATCAAGAGGTCC |
| | | CTGGTTCAAATCCAGGTGCCCCCT |
| 84 | Cys_GCA_chr1:93981834-93981906 (-) | GGGGGTATAGCTCAGGTGGTAGAGCATTTGACTGCAGATCAAGAGGTC |
| | | CCCGGTTCAAATCCGGGTGCCCCCT |
| 85 | Cys_GCA_chr14:73429679-73429750(+) | GGGGGTATAGCTCAGGGGTAGAGCATTTGACTGCAGATCAAGAGGTCC |
| | | CCGGTTCAAATCCGGGTGCCCCCT |
| 86 | Cys_GCA_chr3:131950642-131950713 (-) | GGGGGTATAGCTCAGGGGTAGAGCATTTGACTGCAGATCAAGAGGTCC |
| | | CTGGTTCAAATCCAGGTGCCCCCT |
| 87 | Gln_CTG_chr6:18836402-18836473 (+) | GGTTCCATGGTGTAATGGTTAGCACTCTGGACTCTGAATCCAGCGATCC |
| | | GAGTTCAAATCTCGGTGGAACCT |
| 88 | Gln_CTG_chr6:27515531-27515602 (-) | GGTTCCATGGTGTAATGGTTAGCACTCTGGACTCTGAATCCAGCGATCC |
| | | GAGTTCAAGTCTCGGTGGAACCT |
| 89 | Gln_CTG_chr1:145963304-145963375 | GGTTCCATGGTGTAATGGTGAGCACTCTGGACTCTGAATCCAGCGATC |
| | (+) | CGAGTTCGAGTCTCGGTGGAACCT |
| 90 | Gln_CTG_chr1:147737382-147737453 (-) | GGTTCCATGGTGTAATGGTAAGCACTCTGGACTCTGAATCCAGCGATC |
| | | CGAGTTCGAGTCTCGGTGGAACCT |
| 91 | Gln_CTG_chr6:27263212-27263283 (+) | GGTTCCATGGTGTAATGGTTAGCACTCTGGACTCTGAATCCGGTAATCC |
| | | GAGTTCAAATCTCGGTGGAACCT |
| 92 | Gln_CTG_chr6:27759135-27759206 (-) | GGCCCCATGGTGTAATGGTCAGCACTCTGGACTCTGAATCCAGCGATC |
| | | CGAGTTCAAATCTCGGTGGGACCC |
| 93 | Gln_CTG_chr1:147800937-147801008 | GGTTCCATGGTGTAATGGTAAGCACTCTGGACTCTGAATCCAGCCATCT |
| | (+) | GAGTTCGAGTCTCTGTGGAACCT |
| 94 | Gln_TTG_chr17:47269890-47269961 (+) | GGTCCCATGGTGTAATGGTTAGCACTCTGGACTTTGAATCCAGCGATCC |
| | | GAGTTCAAATCTCGGTGGGACCT |
| 95 | Gln_TTG_chr6:28557156-28557227 (+) | GGTCCCATGGTGTAATGGTTAGCACTCTGGACTTTGAATCCAGCAATCC |
| | | GAGTTCGAATCTCGGTGGGACCT |
| 96 | Gln_TTG_chr6:26311424-26311495 (-) | GGCCCCATGGTGTAATGGTTAGCACTCTGGACTTTGAATCCAGCGATC |
| | | CGAGTTCAAATCTCGGTGGGACCT |
| 97 | Gln_TTG_chr6:145503859-145503930 | GGTCCCATGGTGTAATGGTTAGCACTCTGGGCTTTGAATCCAGCAATCC |
| | (+) | GAGTTCGAATCTTGGTGGGACCT |
| 98 | Glu_CTC_chr1:145399233-145399304 (-) | TCCCTGGTGGTCTAGTGGTTAGGATTCGGCGCTCTCACCGCCGCGGCCC |
| | | GGGTTCGATTCCCGGTCAGGGAA |
| 99 | Glu_CTC_chr1:249168447-249168518 | TCCCTGGTGGTCTAGTGGTTAGGATTCGGCGCTCTCACCGCCGCGGCCC |
| | (+) | GGGTTCGATTCCCGGTCAGGAAA |
| 100 | Glu_TTC_chr2:131094701-131094772 (-) | TCCCATATGGTCTAGCGGTTAGGATTCCTGGTTTTCACCCAGGTGGCCC |
| | | GGGTTCGACTCCCGGTATGGGAA |
| 101 | Glu_TTC_chr13:45492062-45492133 (-) | TCCCACATGGTCTAGCGGTTAGGATTCCTGGTTTTCACCCAGGCGGCCC |
| | | GGGTTCGACTCCCGGTGTGGGAA |
| 102 | Glu_TTC_chr1:17199078-17199149 (+) | TCCCTGGTGGTCTAGTGGCTAGGATTCGGCGCTTTCACCGCCGCGGCCC |
| | | GGGTTCGATTCCCGGCCAGGGAA |
| 103 | Glu_TTC_chr1:16861774-16861845 (-) | TCCCTGGTGGTCTAGTGGCTAGGATTCGGCGCTTTCACCGCCGCGGCCC |
| | | GGGTTCGATTCCCGGTCAGGGAA |
| 104 | Gly_CCC_chr1:16872434-16872504 (-) | GCATTGGTGGTTCAGTGGTAGAATTCTCGCCTCCCACGCGGGAGACCC |
| | | GGGTTCAATTCCCGGCCAATGCA |
| 105 | Gly_CCC_chr2:70476123-70476193 (-) | GCGCCGCTGGTGTAGTGGTATCATGCAAGATTCCCATTCTTGCGACCCG |
| | | GGTTCGATTCCCGGGCGGCGCA |
| 106 | Gly_CCC_chr17:19764175-19764245 (+) | GCATTGGTGGTTCAATGGTAGAATTCTCGCCTCCCACGCAGGAGACCC |
| | | AGGTTCGATTCCTGGCCAATGCA |
| 107 | Gly_GCC_chr1:161413094-161413164 | GCATGGGTGGTTCAGTGGTAGAATTCTCGCCTGCCACGCGGGAGGCCC |
| | (+) | GGGTTCGATTCCCGGCCCATGCA |
| 108 | Gly_GCC_chr1:161493637-161493707 (-) | GCATTGGTGGTTCAGTGGTAGAATTCTCGCCTGCCACGCGGGAGGCCC |
| | | GGGTTCGATTCCCGGCCAATGCA |
| 109 | Gly_GCC_chr16:70812114-70812184 (-) | GCATTGGTGGTTCAGTGGTAGAATTCTCGCCTGCCACGCGGGAGGCCC |
| | | GGGTTTGATTCCCGGCCAGTGCA |
| 110 | Gly_GCC_chr1:161450356-161450426 | GCATAGGTGGTTCAGTGGTAGAATTCTTGCCTGCCACGCAGGAGGCCC |
| | (+) | AGGTTTGATTCCTGGCCCATGCA |
| 111 | Gly_GCC_chr16:70822597-70822667 (+) | GCATTGGTGGTTCAGTGGTAGAATTCTCGCCTGCCATGCGGGCGGCCG |
| | | GGCTTCGATTCCTGGCCAATGCA |
| 112 | Gly_TCC_chr19:4724082-4724153 (+) | GCGTTGGTGGTATAGTGGTTAGCATAGCTGCCTTCCAAGCAGTTGACC |
| | | CGGGTTCGATTCCCGGCCAACGCA |
| 113 | Gly_TCC_chr1:145397864-145397935 (-) | GCGTTGGTGGTATAGTGGTGAGCATAGCTGCCTTCCAAGCAGTTGACC |
| | | CGGGTTCGATTCCCGGCCAACGCA |
| 114 | Gly_TCC_chr17:8124866-8124937 (+) | GCGTTGGTGGTATAGTGGTAAGCATAGCTGCCTTCCAAGCAGTTGACC |
| | | CGGGTTCGATTCCCGGCCAACGCA |
| 115 | Gly_TCC_chr1:161409961-161410032 (-) | GCGTTGGTGGTATAGTGGTGAGCATAGTTGCCTTCCAAGCAGTTGACC |
| | | CGGGCTCGATTCCCGCCCAACGCA |
| 116 | His_GTG_chr1:145396881-145396952 (-) | GCCGTGATCGTATAGTGGTTAGTACTCTGCGTTGTGGCCGCAGCAACCT |
| | | CGGTTCGAATCCGAGTCACGGCA |
| 117 | His_GTG_chr1:149155828-149155899 (-) | GCCATGATCGTATAGTGGTTAGTACTCTGCGCTGTGGCCGCAGCAACC |
| | | TCGGTTCGAATCCGAGTCACGGCA |
| 118 | Ile_AAT_chr6:58149254-58149327 (+) | GGCCGGTTAGCTCAGTTGGTTAGAGCGTGGCGCTAATAACGCCAAGGT |
| | | CGCGGGTTCGATCCCCGTACGGGCCA |
| 119 | Ile_AAT_chr6:27655967-27656040 (+) | GGCCGGTTAGCTCAGTTGGTTAGAGCGTGGTGCTAATAACGCCAAGGT |
| | | CGCGGGTTCGATCCCCGTACTGGCCA |
| 120 | Ile_AAT_chr6:27242990-27243063 (-) | GGCTGGTTAGCTCAGTTGGTTAGAGCGTGGTGCTAATAACGCCAAGGT |
| | | CGCGGGTTCGATCCCCGTACTGGCCA |
| 121 | Ile_AAT_chr17:8130309-8130382 (-) | GGCCGGTTAGCTCAGTTGGTTAGAGCGTGGTGCTAATAACGCCAAGGT |
| | | CGCGGGTTCGAACCCCGTACGGGCCA |
| 122 | Ile_AAT_chr6:26554350-26554423 (+) | GGCCGGTTAGCTCAGTTGGTTAGAGCGTGGTGCTAATAACGCCAAGGT |
| | | CGCGGGTTCGATCCCCGTACGGGCCA |
| 123 | Ile_AAT_chr6:26745255-26745328 (-) | GGCCGGTTAGCTCAGTTGGTTAGAGCGTGGTGCTAATAACGCTAAGGT |
| | | CGCGGGTTCGATCCCCGTACTGGCCA |
| 124 | Ile_AAT_chr6:26721221-26721294 (-) | GGCCGGTTAGCTCAGTTGGTCAGAGCGTGGTGCTAATAACGCCAAGGT |
| | | CGCGGGTTCGATCCCCGTACGGGCCA |
| 125 | Ile_AAT_chr6:27636362-2763 643 5 (+) | GGCCGGTTAGCTCAGTCGGCTAGAGCGTGGTGCTAATAACGCCAAGGT |
| | | CGCGGGTTCGATCCCCGTACGGGCCA |
| 126 | Ile_AAT_chr6:27241739-27241812 (+) | GGCTGGTTAGTTCAGTTGGTTAGAGCGTGGTGCTAATAACGCCAAGGT |
| | | CGTGGGTTCGATCCCCATATCGGCCA |
| 127 | Ile_GAT_chrX:3756418-3756491 (-) | GGCCGGTTAGCTCAGTTGGTAAGAGCGTGGTGCTGATAACACCAAGGT |
| | | CGCGGGCTCGACTCCCGCACCGGCCA |
| 128 | Ile_TAT_chr19:39902808-39902900 (-) | GCTCCAGTGGCGCAATCGGTTAGCGCGCGGTACTTATATGACAGTGCG |
| | | AGCGGAGCAATGCCGAGGTTGTGAGTTCGATCCTCACCTGGAGCA |
| 129 | Ile_TAT_chr2:43037676-43037768 (+) | GCTCCAGTGGCGCAATCGGTTAGCGCGCGGTACTTATACAGCAGTACA |
| | | TGCAGAGCAATGCCGAGGTTGTGAGTTCGAGCCTCACCTGGAGCA |
| 130 | Ile_TAT_chr6:26988125-26988218 (+) | GCTCCAGTGGCGCAATCGGTTAGCGCGCGGTACTTATATGGCAGTATG |
| | | TGTGCGAGTGATGCCGAGGTTGTGAGTTCGAGCCTCACCTGGAGCA |
| 131 | Ile_TAT_chr6:27599200-27599293 (+) | GCTCCAGTGGCGCAATCGGTTAGCGCGCGGTACTTATACAACAGTATA |
| | | TGTGCGGGTGATGCCGAGGTTGTGAGTTCGAGCCTCACCTGGAGCA |
| 132 | Ile_TAT_chr6:28505367-28505460 (+) | GCTCCAGTGGCGCAATCGGTTAGCGCGCGGTACTTATAAGACAGTGCA |
| | | CCTGTGAGCAATGCCGAGGTTGTGAGTTCAAGCCTCACCTGGAGCA |
| 133 | Leu_AAG_chr5:180524474-180524555 (-) | GGTAGCGTGGCCGAGCGGTCTAAGGCGCTGGATTAAGGCTCCAGTCTC |
| | | TTCGGAGGCGTGGGTTCGAATCCCACCGCTGCCA |
| 134 | Leu_AAG_chr5:180614701-180614782 | GGTAGCGTGGCCGAGCGGTCTAAGGCGCTGGATTAAGGCTCCAGTCTC |
| | (+) | TTCGGGGGCGTGGGTTCGAATCCCACCGCTGCCA |
| 135 | Leu_AAG_chr6:28956779-28956860 (+) | GGTAGCGTGGCCGAGCGGTCTAAGGCGCTGGATTAAGGCTCCAGTCTC |
| | | TTCGGGGGCGTGGGTTCAAATCCCACCGCTGCCA |
| 136 | Leu_AAG_chr6:28446400-28446481 (-) | GGTAGCGTGGCCGAGTGGTCTAAGACGCTGGATTAAGGCTCCAGTCTC |
| | | TTCGGGGGCGTGGGTTTGAATCCCACCGCTGCCA |
| 137 | Leu_CAA_chr6:28864000-28864105 (-) | GTCAGGATGGCCGAGTGGTCTAAGGCGCCAGACTCAAGCTAAGCTTCC |
| | | TCCGCGGTGGGGATTCTGGTCTCCAATGGAGGCGTGGGTTCGAATCCC |
| 138 | Leu_CAA_chr6:28908830-28908934 (+) | GTCAGGATGGCCGAGTGGTCTAAGGCGCCAGACTCAAGCTTGGCTTCC |
| | | TCGTGTTGAGGATTCTGGTCTCCAATGGAGGCGTGGGTTCGAATCCCA |
| 139 | Leu_CAA_chr6:27573417-27573524 (-) | GTCAGGATGGCCGAGTGGTCTAAGGCGCCAGACTCAAGCTTACTGCTT |
| | | CCTGTGTTCGGGTCTTCTGGTCTCCGTATGGAGGCGTGGGTTCGAATCC |
| 140 | Leu_CAA_chr6:27570348-27570454 (-) | GTCAGGATGGCCGAGTGGTCTAAGGCGCCAGACTCAAGTTGCTACTTC |
| | | CCAGGTTTGGGGCTTCTGGTCTCCGCATGGAGGCGTGGGTTCGAATCC |
| 141 | Leu_CAA_chr1:249168054-249168159 | GTCAGGATGGCCGAGTGGTCTAAGGCGCCAGACTCAAGGTAAGCACCT |
| | (+) | TGCCTGCGGGCTTTCTGGTCTCCGGATGGAGGCGTGGGTTCGAATCCC |
| 142 | Leu_CAA_chr11:9296790-9296863 (+) | GCCTCCTTAGTGCAGTAGGTAGCGCATCAGTCTCAAAATCTGAATGGT |
| | | CCTGAGTTCAAGCCTCAGAGGGGGCA |
| 143 | Leu_CAA_chr1:161581736-161581819 (-) | GTCAGGATGGCCGAGCAGTCTTAAGGCGCTGCGTTCAAATCGCACCCT |
| | | CCGCTGGAGGCGTGGGTTCGAATCCCACTTTTGACA |
| 144 | Leu_CAG_chr1:161411323-161411405 | GTCAGGATGGCCGAGCGGTCTAAGGCGCTGCGTTCAGGTCGCAGTCTC |
| | (+) | CCCTGGAGGCGTGGGTTCGAATCCCACTCCTGACA |
| 145 | Leu_CAG_chr16:57333863-57333945 (+) | GTCAGGATGGCCGAGCGGTCTAAGGCGCTGCGTTCAGGTCGCAGTCTC |
| | | CCCTGGAGGCGTGGGTTCGAATCCCACTTCTGACA |
| 146 | Leu_TAA_chr6:144537684-144537766 | ACCAGGATGGCCGAGTGGTTAAGGCGTTGGACTTAAGATCCAATGGAC |
| | (+) | ATATGTCCGCGTGGGTTCGAACCCCACTCCTGGTA |
| 147 | Leu_TAA_chr6:27688898-27688980 (-) | ACCGGGATGGCCGAGTGGTTAAGGCGTTGGACTTAAGATCCAATGGGC |
| | | TGGTGCCCGCGTGGGTTCGAACCCCACTCTCGGTA |
| 148 | Leu_TAA_chr11:59319228-59319310 (+) | ACCAGAATGGCCGAGTGGTTAAGGCGTTGGACTTAAGATCCAATGGAT |
| | | TCATATCCGCGTGGGTTCGAACCCCACTTCTGGTA |
| 149 | Leu_TAA_chr6:27198334-27198416 (-) | ACCGGGATGGCTGAGTGGTTAAGGCGTTGGACTTAAGATCCAATGGAC |
| | | AGGTGTCCGCGTGGGTTCGAGCCCCACTCCCGGTA |
| 150 | Leu_TAG_chr17:8023632-8023713 (-) | GGTAGCGTGGCCGAGCGGTCTAAGGCGCTGGATTTAGGCTCCAGTCTC |
| | | TTCGGAGGCGTGGGTTCGAATCCCACCGCTGCCA |
| 151 | Leu_TAG_chr14:21093529-21093610 (+) | GGTAGTGTGGCCGAGCGGTCTAAGGCGCTGGATTTAGGCTCCAGTCTC |
| | | TTCGGGGGCGTGGGTTCGAATCCCACCACTGCCA |
| 152 | Leu_TAG_chr16:22207032-22207113 (-) | GGTAGCGTGGCCGAGTGGTCTAAGGCGCTGGATTTAGGCTCCAGTCAT |
| | | TTCGATGGCGTGGGTTCGAATCCCACCGCTGCCA |
| 153 | Lys_CTT_chr14:58706613-58706685 (-) | GCCCGGCTAGCTCAGTCGGTAGAGCATGGGACTCTTAATCCCAGGGTC |
| | | GTGGGTTCGAGCCCCACGTTGGGCG |
| 154 | Lys_CTT_chr19:36066750-36066822(+) | GCCCAGCTAGCTCAGTCGGTAGAGCATAAGACTCTTAATCTCAGGGTT |
| | | GTGGATTCGTGCCCCATGCTGGGTG |
| 155 | Lys_CTT_chr19:52425393-52425466 (-) | GCAGCTAGCTCAGTCGGTAGAGCATGAGACTCTTAATCTCAGGGTCAT |
| | | GGGTTCGTGCCCCATGTTGGGTGCCA |
| 156 | Lys_CTT_chr1:145395522-145395594 (-) | GCCCGGCTAGCTCAGTCGGTAGAGCATGAGACTCTTAATCTCAGGGTC |
| | | GTGGGTTCGAGCCCCACGTTGGGCG |
| 157 | Lys_CTT_chr16:3207406-3207478 (-) | GCCCGGCTAGCTCAGTCGGTAGAGCATGAGACCCTTAATCTCAGGGTC |
| | | GTGGGTTCGAGCCCCACGTTGGGCG |
| 158 | Lys_CTT_chr16:3241501-3241573 (+) | GCCCGGCTAGCTCAGTCGGTAGAGCATGGGACTCTTAATCTCAGGGTC |
| | | GTGGGTTCGAGCCCCACGTTGGGCG |
| 159 | Lys_CTT_ch16:3230555-3230627 (-) | GCCCGGCTAGCTCAGTCGATAGAGCATGAGACTCTTAATCTCAGGGTC |
| | | GTGGGTTCGAGCCGCACGTTGGGCG |
| 160 | Lys_CTT_chr1:55423542-55423614(-) | GCCCAGCTAGCTCAGTCGGTAGAGCATGAGACTCTTAATCTCAGGGTC |
| | | ATGGGTTTGAGCCCCACGTTTGGTG |
| 161 | Lys_CTT_chr16:3214939-3215011 (+) | GCCTGGCTAGCTCAGTCGGCAAAGCATGAGACTCTTAATCTCAGGGTC |
| | | GTGGGCTCGAGCTCCATGTTGGGCG |
| 162 | Lys_CTT_chr5:26198539-26198611 (-) | GCCCGACTACCTCAGTCGGTGGAGCATGGGACTCTTCATCCCAGGGTT |
| | | GTGGGTTCGAGCCCCACATTGGGCA |
| 163 | Lys_TTT_chr16:73512216-73512288 (-) | GCCTGGATAGCTCAGTTGGTAGAGCATCAGACTTTTAATCTGAGGGTC |
| | | CAGGGTTCAAGTCCCTGTTCAGGCA |
| 164 | Lys_TTT_chr12:27843306-27843378 (+) | ACCCAGATAGCTCAGTCAGTAGAGCATCAGACTTTTAATCTGAGGGTC |
| | | CAAGGTTCATGTCCCTTTTTGGGTG |
| 165 | Lys_TTT_chr11:122430655-122430727 | GCCTGGATAGCTCAGTTGGTAGAGCATCAGACTTTTAATCTGAGGGTC |
| | (+) | CAGGGTTCAAGTCCCTGTTCAGGCG |
| 166 | Lys_TTT_chr1:204475655-204475727 (+) | GCCCGGATAGCTCAGTCGGTAGAGCATCAGACTTTTAATCTGAGGGTC |
| | | CAGGGTTCAAGTCCCTGTTCGGGCG |
| 167 | Lys_TTT_chr6:27559593-27559665 (-) | GCCTGGATAGCTCAGTCGGTAGAGCATCAGACTTTTAATCTGAGGGTC |
| | | CAGGGTTCAAGTCCCTGTTCAGGCG |
| 168 | Lys_TTT_chr11:59323902-59323974 (+) | GCCCGGATAGCTCAGTCGGTAGAGCATCAGACTTTTAATCTGAGGGTC |
| | | CGGGGTTCAAGTCCCTGTTCGGGCG |
| 169 | Lys_TTT_chr6:27302769-27302841 (-) | GCCTGGGTAGCTCAGTCGGTAGAGCATCAGACTTTTAATCTGAGGGTC |
| | | CAGGGTTCAAGTCCCTGTCCAGGCG |
| 170 | Lys_TTT_chr6:28715521-28715593 (+) | GCCTGGATAGCTCAGTTGGTAGAACATCAGACTTTTAATCTGACGGTG |
| | | CAGGGTTCAAGTCCCTGTTCAGGCG |
| 171 | Met_CAT_chr8:124169470-124169542 (-) | GCCTCGTTAGCGCAGTAGGTAGCGCGTCAGTCTCATAATCTGAAGGTC |
| | | GTGAGTTCGATCCTCACACGGGGCA |
| 172 | Met_CAT_chr16:71460396-71460468 (+) | GCCCTCTTAGCGCAGTGGGCAGCGCGTCAGTCTCATAATCTGAAGGTC |
| | | CTGAGTTCGAGCCTCAGAGAGGGCA |
| 173 | Met_CAT_chr6:28912352-28912424 (+) | GCCTCCTTAGCGCAGTAGGCAGCGCGTCAGTCTCATAATCTGAAGGTC |
| | | CTGAGTTCGAACCTCAGAGGGGGCA |
| 174 | Met_CAT_chr6:26735574-26735646 (-) | GCCCTCTTAGCGCAGCGGGCAGCGCGTCAGTCTCATAATCTGAAGGTC |
| | | CTGAGTTCGAGCCTCAGAGAGGGCA |
| 175 | Met_CAT_chr6:26701712-26701784(+) | GCCCTCTTAGCGCAGCTGGCAGCGCGTCAGTCTCATAATCTGAAGGTC |
| | | CTGAGTTCAAGCCTCAGAGAGGGCA |
| 176 | Met_CAT_chr16:87417628-87417700 (-) | GCCTCGTTAGCGCAGTAGGCAGCGCGTCAGTCTCATAATCTGAAGGTC |
| | | GTGAGTTCGAGCCTCACACGGGGCA |
| 177 | Met_CAT_chr6:58168492-58168564 (-) | GCCCTCTTAGTGCAGCTGGCAGCGCGTCAGTTTCATAATCTGAAAGTCC |
| | | TGAGTTCAAGCCTCAGAGAGGGCA |
| 178 | Phe_GAA_chr6:28758499-28758571 (-) | GCCGAAATAGCTCAGTTGGGAGAGCGTTAGACTGAAGATCTAAAGGTC |
| | | CCTGGTTCGATCCCGGGTTTCGGCA |
| 179 | Phe_GAA_chr11:59333853-59333925 (-) | GCCGAAATAGCTCAGTTGGGAGAGCGTTAGACTGAAGATCTAAAGGTC |
| | | CCTGGTTCAATCCCGGGTTTCGGCA |
| 180 | Phe_GAA_chr6:28775610-28775682 (-) | GCCGAGATAGCTCAGTTGGGAGAGCGTTAGACTGAAGATCTAAAGGTC |
| | | CCTGGTTCAATCCCGGGTTTCGGCA |
| 181 | Phe_GAA_chr6:28791093-28791166 (-) | GCCGAAATAGCTCAGTTGGGAGAGCGTTAGACCGAAGATCTTAAAGGT |
| | | CCCTGGTTCAATCCCGGGTTTCGGCA |
| 182 | Phe_GAA_chr6:28731374-28731447 (-) | GCTGAAATAGCTCAGTTGGGAGAGCGTTAGACTGAAGATCTTAAAGTT |
| | | CCCTGGTTCAACCCTGGGTTTCAGCC |
| 183 | Pro_AGG_chr16:3241989-3242060 (+) | GGCTCGTTGGTCTAGGGGTATGATTCTCGCTTAGGATGCGAGAGGTCC |
| | | CGGGTTCAAATCCCGGACGAGCCC |
| 184 | Pro_AGG_chr1:167684725-167684796 (-) | GGCTCGTTGGTCTAGGGGTATGATTCTCGCTTAGGGTGCGAGAGGTCC |
| | | CGGGTTCAAATCCCGGACGAGCCC |
| 185 | Pro_CGG_chr1:167683962-167684033 | GGCTCGTTGGTCTAGGGGTATGATTCTCGCTTCGGGTGCGAGAGGTCC |
| | (+) | CGGGTTCAAATCCCGGACGAGCCC |
| 186 | Pro_CGG_chr6:27059521-27059592 (+) | GGCTCGTTGGTCTAGGGGTATGATTCTCGCTTCGGGTGTGAGAGGTCCC |
| | | GGGTTCAAATCCCGGACGAGCCC |
| 187 | Pro_TGG_chr14:21101165-21101236 (+) | GGCTCGTTGGTCTAGTGGTATGATTCTCGCTTTGGGTGCGAGAGGTCCC |
| | | GGGTTCAAATCCCGGACGAGCCC |
| 188 | Pro_TGG_chr11:75946869-75946940 (-) | GGCTCGTTGGTCTAGGGGTATGATTCTCGGTTTGGGTCCGAGAGGTCCC |
| | | GGGTTCAAATCCCGGACGAGCCC |
| 189 | Pro_TGG_chr5:180615854-180615925 (-) | GGCTCGTTGGTCTAGGGGTATGATTCTCGCTTTGGGTGCGAGAGGTCCC |
| | | GGGTTCAAATCCCGGACGAGCCC |
| 190 | SeC_TCA_chr19:45981859-45981945 (-) | GCCCGGATGATCCTCAGTGGTCTGGGGTGCAGGCTTCAAACCTGTAGC |
| | | TGTCTAGCGACAGAGTGGTTCAATTCCACCTTTCGGGCG |
| 191 | SeC_TCA_chr22:44546537-44546620 (+) | GCTCGGATGATCCTCAGTGGTCTGGGGTGCAGGCTTCAAACCTGTAGC |
| | | TGTCTAGTGACAGAGTGGTTCAATTCCACCTTTGTA |
| 192 | Ser_AGA_chr6:27509554-27509635 (-) | GTAGTCGTGGCCGAGTGGTTAAGGCGATGGACTAGAAATCCATTGGGG |
| | | TTTCCCCGCGCAGGTTCGAATCCTGCCGACTACG |
| 193 | Ser_AGA_chr6:26327817-26327898 (+) | GTAGTCGTGGCCGAGTGGTTAAGGCGATGGACTAGAAATCCATTGGGG |
| | | TCTCCCCGCGCAGGTTCGAATCCTGCCGACTACG |
| 194 | Ser_AGA_chr6:27499987-27500068 (+) | GTAGTCGTGGCCGAGTGGTTAAGGCGATGGACTAGAAATCCATTGGGG |
| | | TTTCCCCACGCAGGTTCGAATCCTGCCGACTACG |
| 195 | Ser_AGA_chr6:27521192-27521273 (-) | GTAGTCGTGGCCGAGTGGTTAAGGTGATGGACTAGAAACCCATTGGGG |
| | | TCTCCCCGCGCAGGTTCGAATCCTGCCGACTACG |
| 196 | Ser_CGA_chr17:8042199-8042280 (-) | GCTGTGATGGCCGAGTGGTTAAGGCGTTGGACTCGAAATCCAATGGGG |
| | | TCTCCCCGCGCAGGTTCGAATCCTGCTCACAGCG |
| 197 | Ser_CGA_chr6:27177628-27177709 (+) | GCTGTGATGGCCGAGTGGTTAAGGCGTTGGACTCGAAATCCAATGGGG |
| | | TCTCCCCGCGCAGGTTCAAATCCTGCTCACAGCG |
| 198 | Ser_CGA_chr6:27640229-27640310 (-) | GCTGTGATGGCCGAGTGGTTAAGGTGTTGGACTCGAAATCCAATGGGG |
| | | GTTCCCCGCGCAGGTTCAAATCCTGCTCACAGCG |
| 199 | Ser_CGA_chr12:56584148-56584229 (+) | GTCACGGTGGCCGAGTGGTTAAGGCGTTGGACTCGAAATCCAATGGGG |
| | | TTTCCCCGCACAGGTTCGAATCCTGTTCGTGACG |
| 200 | Ser_GCT_chr6:27065085-27065166 (+) | GACGAGGTGGCCGAGTGGTTAAGGCGATGGACTGCTAATCCATTGTGC |
| | | TCTGCACGCGTGGGTTCGAATCCCACCCTCGTCG |
| 201 | Ser_GCT_chr6:27265775-27265856 (+) | GACGAGGTGGCCGAGTGGTTAAGGCGATGGACTGCTAATCCATTGTGC |
| | | TCTGCACGCGTGGGTTCGAATCCCACCTTCGTCG |
| 202 | Ser_GCT_chr11:66115591-66115672 (+) | GACGAGGTGGCCGAGTGGTTAAGGCGATGGACTGCTAATCCATTGTGC |
| | | TTTGCACGCGTGGGTTCGAATCCCATCCTCGTCG |
| 203 | Ser_GCT_chr6:28565117-28565198 (-) | GACGAGGTGGCCGAGTGGTTAAGGCGATGGACTGCTAATCCATTGTGC |
| | | TCTGCACGCGTGGGTTCGAATCCCATCCTCGTCG |
| 204 | Ser_GCT_chr6:28180815-28180896 (+) | GACGAGGTGGCCGAGTGGTTAAGGCGATGGACTGCTAATCCATTGTGC |
| | | TCTGCACACGTGGGTTCGAATCCCATCCTCGTCG |
| 205 | Ser_GCT_chr6:26305718-26305801 (-) | GGAGAGGCCTGGCCGAGTGGTTAAGGCGATGGACTGCTAATCCATTGT |
| | | GCTCTGCACGCGTGGGTTCGAATCCCATCCTCGTCG |
| 206 | Ser_TGA_chr10:69524261-69524342 (+) | GCAGCGATGGCCGAGTGGTTAAGGCGTTGGACTTGAAATCCAATGGGG |
| | | TCTCCCCGCGCAGGTTCGAACCCTGCTCGCTGCG |
| 207 | Ser_TGA_chr6:27513468-27513549 (+) | GTAGTCGTGGCCGAGTGGTTAAGGCGATGGACTTGAAATCCATTGGGG |
| | | TTTCCCCGCGCAGGTTCGAATCCTGCCGACTACG |
| 208 | Ser_TGA_chr6:26312824-26312905 (-) | GTAGTCGTGGCCGAGTGGTTAAGGCGATGGACTTGAAATCCATTGGGG |
| | | TCTCCCCGCGCAGGTTCGAATCCTGCCGACTACG |
| 209 | Ser_TGA_chr6:27473607-27473688 (-) | GTAGTCGTGGCCGAGTGGTTAAGGCGATGGACTTGAAATCCATTGGGG |
| | | TTTCCCCGCGCAGGTTCGAATCCTGTCGGCTACG |
| 210 | Thr_AGT_chr17:8090478-8090551 (+) | GGCGCCGTGGCTTAGTTGGTTAAAGCGCCTGTCTAGTAAACAGGAGAT |
| | | CCTGGGTTCGAATCCCAGCGGTGCCT |
| 211 | Thr_AGT_chr6:26533145-26533218 (-) | GGCTCCGTGGCTTAGCTGGTTAAAGCGCCTGTCTAGTAAACAGGAGAT |
| | | CCTGGGTTCGAATCCCAGCGGGGCCT |
| 212 | Thr_AGT_chr6:28693795-28693868 (+) | GGCTCCGTAGCTTAGTTGGTTAAAGCGCCTGTCTAGTAAACAGGAGAT |
| | | CCTGGGTTCGACTCCCAGCGGGGCCT |
| 213 | Thr_AGT_chr6:27694473-27694546 (+) | GGCTTCGTGGCTTAGCTGGTTAAAGCGCCTGTCTAGTAAACAGGAGAT |
| | | CCTGGGTTCGAATCCCAGCGAGGCCT |
| 214 | Thr_AGT_chr17:8042770-8042843 (-) | GGCGCCGTGGCTTAGCTGGTTAAAGCGCCTGTCTAGTAAACAGGAGAT |
| | | CCTGGGTTCGAATCCCAGCGGTGCCT |
| 215 | Thr_AGT_chr6:27130050-27130123 (+) | GGCCCTGTGGCTTAGCTGGTCAAAGCGCCTGTCTAGTAAACAGGAGAT |
| | | CCTGGGTTCGAATCCCAGCGGGGCCT |
| 216 | Thr_CGT_chr6:28456770-28456843 (-) | GGCTCTATGGCTTAGTTGGTTAAAGCGCCTGTCTCGTAAACAGGAGAT |
| | | CCTGGGTTCGACTCCCAGTGGGGCCT |
| 217 | Thr_CGT_chr16:14379750-14379821 (+) | GGCGCGGTGGCCAAGTGGTAAGGCGTCGGTCTCGTAAACCGAAGATCA |
| | | CGGGTTCGAACCCCGTCCGTGCCT |
| 218 | Thr_CGT_chr6:28615984-28616057 (-) | GGCTCTGTGGCTTAGTTGGCTAAAGCGCCTGTCTCGTAAACAGGAGAT |
| | | CCTGGGTTCGAATCCCAGCGGGGCCT |
| 219 | Thr_CGT_chr17:29877093-29877164 (+) | GGCGCGGTGGCCAAGTGGTAAGGCGTCGGTCTCGTAAACCGAAGATCG |
| | | CGGGTTCGAACCCCGTCCGTGCCT |
| 220 | Thr_CGT_chr6:27586135-27586208 (+) | GGCCCTGTAGCTCAGCGGTTGGAGCGCTGGTCTCGTAAACCTAGGGGT |
| | | CGTGAGTTCAAATCTCACCAGGGCCT |
| 221 | Thr_TGT_chr6:28442329-28442402 (-) | GGCTCTATGGCTTAGTTGGTTAAAGCGCCTGTCTTGTAAACAGGAGAT |
| | | CCTGGGTTCGAATCCCAGTAGAGCCT |
| 222 | Thr_TGT_chr1:222638347-222638419 (+) | GGCTCCATAGCTCAGTGGTTAGAGCACTGGTCTTGTAAACCAGGGGTC |
| | | GCGAGTTCGATCCTCGCTGGGGCCT |
| 223 | Thr_TGT_chr14:21081949-21082021 (-) | GGCTCCATAGCTCAGGGGTTAGAGCGCTGGTCTTGTAAACCAGGGGTC |
| | | GCGAGTTCAATTCTCGCTGGGGCCT |
| 224 | Thr_TGT_chr14:21099319-21099391 (-) | GGCTCCATAGCTCAGGGGTTAGAGCACTGGTCTTGTAAACCAGGGGTC |
| | | GCGAGTTCAAATCTCGCTGGGGCCT |
| 225 | Thr_TGT_chr14:21149849-21149921 (+) | GGCCCTATAGCTCAGGGGTTAGAGCACTGGTCTTGTAAACCAGGGGTC |
| | | GCGAGTTCAAATCTCGCTGGGGCCT |
| 226 | Thr_TGT_chr5:180618687-180618758 (-) | GGCTCCATAGCTCAGGGGTTAGAGCACTGGTCTTGTAAACCAGGGTCG |
| | | CGAGTTCAAATCTCGCTGGGGCCT |
| 227 | Trp_CCA_chr17:8124187-8124258 (-) | GGCCTCGTGGCGCAACGGTAGCGCGTCTGACTCCAGATCAGAAGGTTG |
| | | CGTGTTCAAATCACGTCGGGGTCA |
| 228 | Trp_CCA_chr17:19411494-19411565 (+) | GACCTCGTGGCGCAATGGTAGCGCGTCTGACTCCAGATCAGAAGGTTG |
| | | CGTGTTCAAGTCACGTCGGGGTCA |
| 229 | Trp_CCA_chr6:26319330-26319401 (-) | GACCTCGTGGCGCAACGGTAGCGCGTCTGACTCCAGATCAGAAGGTTG |
| | | CGTGTTCAAATCACGTCGGGGTCA |
| 230 | Trp_CCA_chr12:98898030-98898101 (+) | GACCTCGTGGCGCAACGGTAGCGCGTCTGACTCCAGATCAGAAGGCTG |
| | | CGTGTTCGAATCACGTCGGGGTCA |
| 231 | Trp_CCA_chr7:99067307-99067378 (+) | GACCTCGTGGCGCAACGGCAGCGCGTCTGACTCCAGATCAGAAGGTTG |
| | | CGTGTTCAAATCACGTCGGGGTCA |
| 232 | Tyr_ATA_chr2:219110549-219110641 | CCTTCAATAGTTCAGCTGGTAGAGCAGAGGACTATAGCTACTTCCTCA |
| | (+) | GTAGGAGACGTCCTTAGGTTGCTGGTTCGATTCCAGCTTGAAGGA |
| 233 | Tyr_GTA_chr6:26569086-26569176 (+) | CCTTCGATAGCTCAGTTGGTAGAGCGGAGGACTGTAGTTGGCTGTGTC |
| | | CTTAGACATCCTTAGGTCGCTGGTTCGAATCCGGCTCGAAGGA |
| 234 | Tyr_GTA_chr2:27273650-27273738 (+) | CCTTCGATAGCTCAGTTGGTAGAGCGGAGGACTGTAGTGGATAGGGCG |
| | | TGGCAATCCTTAGGTCGCTGGTTCGATTCCGGCTCGAAGGA |
| 235 | Tyr_GTA_chr6:26577332-26577420 (+) | CCTTCGATAGCTCAGTTGGTAGAGCGGAGGACTGTAGGCTCATTAAGC |
| | | AAGGTATCCTTAGGTCGCTGGTTCGAATCCGGCTCGGAGGA |
| 236 | Tyr_GTA_chr14:21125623-21125716 (-) | CCTTCGATAGCTCAGCTGGTAGAGCGGAGGACTGTAGATTGTATAGAC |
| | | ATTTGCGGACATCCTTAGGTCGCTGGTTCGATTCCAGCTCGAAGGA |
| 237 | Tyr_GTA_chr8:67025602-67025694 (+) | CCTTCGATAGCTCAGCTGGTAGAGCGGAGGACTGTAGCTACTTCCTCA |
| | | GCAGGAGACATCCTTAGGTCGCTGGTTCGATTCCGGCTCGAAGGA |
| 238 | Tyr_GTA_chr8:67026223-67026311 (+) | CCTTCGATAGCTCAGCTGGTAGAGCGGAGGACTGTAGGCGCGCGCCCG |
| | | TGGCCATCCTTAGGTCGCTGGTTCGATTCCGGCTCGAAGGA |
| 239 | Tyr_GTA_chr14:21121258-21121351 (-) | CCTTCGATAGCTCAGCTGGTAGAGCGGAGGACTGTAGCCTGTAGAAAC |
| | | ATTTGTGGACATCCTTAGGTCGCTGGTTCGATTCCGGCTCGAAGGA |
| 240 | Tyr_GTA_chr14:21131351-21131444 (-) | CCTTCGATAGCTCAGCTGGTAGAGCGGAGGACTGTAGATTGTACAGAC |
| | | ATTTGCGGACATCCTTAGGTCGCTGGTTCGATTCCGGCTCGAAGGA |
| 241 | Tyr_GTA_chr14:21151432-21151520 (+) | CCTTCGATAGCTCAGCTGGTAGAGCGGAGGACTGTAGTACTTAATGTG |
| | | TGGTCATCCTTAGGTCGCTGGTTCGATTCCGGCTCGAAGGA |
| 242 | Tyr_GTA_chr6:26595102-26595190 (+) | CCTTCGATAGCTCAGCTGGTAGAGCGGAGGACTGTAGGGGTTTGAATG |
| | | TGGTCATCCTTAGGTCGCTGGTTCGAATCCGGCTCGGAGGA |
| 243 | Tyr_GTA_chr14:21128117-21128210 (-) | CCTTCGATAGCTCAGCTGGTAGAGCGGAGGACTGTAGACTGCGGAAAC |
| | | GTTTGTGGACATCCTTAGGTCGCTGGTTCAATTCCGGCTCGAAGGA |
| 244 | Tyr_GTA_chr6:26575798-26575887 (+) | CTTTCGATAGCTCAGTTGGTAGAGCGGAGGACTGTAGGTTCATTAAAC |
| | | TAAGGCATCCTTAGGTCGCTGGTTCGAATCCGGCTCGAAGGA |
| 245 | Tyr_GTA_chr8:66609532-66609619 (-) | TCTTCAATAGCTCAGCTGGTAGAGCGGAGGACTGTAGGTGCACGCCCG |
| | | TGGCCATTCTTAGGTGCTGGTTTGATTCCGACTTGGAGAG |
| 246 | Val_AAC_chr3:169490018-169490090 | GTTTCCGTAGTGTAGTGGTTATCACGTTCGCCTAACACGCGAAAGGTCC |
| | (+) | CCGGTTCGAAACCGGGCGGAAACA |
| 247 | Val_AAC_chr5:180615416-180615488 (-) | GTTTCCGTAGTGTAGTGGTCATCACGTTCGCCTAACACGCGAAAGGTC |
| | | CCCGGTTCGAAACCGGGCGGAAACA |
| 248 | Val_AAC_chr6:27618707-27618779 (-) | GTTTCCGTAGTGTAGTGGTTATCACGTTCGCCTAACACGCGAAAGGTCC |
| | | CTGGATCAAAACCAGGCGGAAACA |
| 249 | Val_AAC_chr6:27648885-27648957 (-) | GTTTCCGTAGTGTAGTGGTTATCACGTTCGCCTAACACGCGAAAGGTCC |
| | | GCGGTTCGAAACCGGGCGGAAACA |
| 250 | Val_AAC_chr6:27203288-27203360 (+) | GTTTCCGTAGTGTAGTGGTTATCACGTTTGCCTAACACGCGAAAGGTCC |
| | | CCGGTTCGAAACCGGGCAGAAACA |
| 251 | Val_AAC_chr6:28703206-28703277 (-) | GGGGGTGTAGCTCAGTGGTAGAGCGTATGCTTAACATTCATGAGGCTC |
| | | TGGGTTCGATCCCCAGCACTTCCA |
| 252 | Val_CAC_chr1:161369490-161369562 (-) | GTTTCCGTAGTGTAGTGGTTATCACGTTCGCCTCACACGCGAAAGGTCC |
| | | CCGGTTCGAAACCGGGCGGAAACA |
| 253 | Val_CAC_chr6:27248049-27248121 (-) | GCTTCTGTAGTGTAGTGGTTATCACGTTCGCCTCACACGCGAAAGGTCC |
| | | CCGGTTCGAAACCGGGCAGAAGCA |
| 254 | Val_CAC_chr19:4724647-4724719 (-) | GTTTCCGTAGTGTAGCGGTTATCACATTCGCCTCACACGCGAAAGGTCC |
| | | CCGGTTCGATCCCGGGCGGAAACA |
| 255 | Val_CAC_chr1:149298555-149298627 (-) | GTTTCCGTAGTGTAGTGGTTATCACGTTCGCCTCACACGCGAAAGGTCC |
| | | CCGGTTCGAAACTGGGCGGAAACA |
| 256 | Val_CAC_chr1:149684088-149684161 (-) | GTTTCCGTAGTGTAGTGGTTATCACGTTCGCCTCACACGCGTAAAGGTC |
| | | CCCGGTTCGAAACCGGGCGGAAACA |
| 257 | Val_CAC_chr6:27173867-27173939(-) | GTTTCCGTAGTGGAGTGGTTATCACGTTCGCCTCACACGCGAAAGGTC |
| | | CCCGGTTTGAAACCAGGCGGAAACA |
| 258 | Val_TAC_chr11:59318102-59318174 (-) | GGTTCCATAGTGTAGTGGTTATCACGTCTGCTTTACACGCAGAAGGTCC |
| | | TGGGTTCGAGCCCCAGTGGAACCA |
| 259 | Val_TAC_chr11:59318460-59318532 (-) | GGTTCCATAGTGTAGCGGTTATCACGTCTGCTTTACACGCAGAAGGTCC |
| | | TGGGTTCGAGCCCCAGTGGAACCA |
| 260 | Val_TAC_chr10:5895674-5895746 (-) | GGTTCCATAGTGTAGTGGTTATCACATCTGCTTTACACGCAGAAGGTCC |
| | | TGGGTTCAAGCCCCAGTGGAACCA |
| 261 | Val_TAC_chr6:27258405-27258477 (+) | GTTTCCGTGGTGTAGTGGTTATCACATTCGCCTTACACGCGAAAGGTCC |
| | | TCGGGTCGAAACCGAGCGGAAACA |
| 262 | iMet_CAT_chr1:153643726-153643797 | AGCAGAGTGGCGCAGCGGAAGCGTGCTGGGCCCATAACCCAGAGGTC |
| | (+) | GATGGATCGAAACCATCCTCTGCTA |
| 263 | iMet_CAT_chr6:27745664-27745735 (+) | AGCAGAGTGGCGCAGCGGAAGCGTGCTGGGCCCATAACCCAGAGGTC |
| | | GATGGATCTAAACCATCCTCTGCTA |
| 264 | Glu_TTC_chr1:16861773-16861845 (-) | TCCCTGGTGGTCTAGTGGCTAGGATTCGGCGCTTTCACCGCCGCGGCCC |
| | | GGGTTCGATTCCCGGTCAGGGAAT |
| 265 | Gly_CCC_chr1:17004765-17004836 (-) | GCGTTGGTGGTTTAGTGGTAGAATTCTCGCCTCCCATGCGGGAGACCC |
| | | GGGTTCAATTCCCGGCCACTGCAC |
| 266 | Gly_CCC_chr1:17053779-17053850 (+) | GGCCTTGGTGGTGCAGTGGTAGAATTCTCGCCTCCCACGTGGGAGACC |
| | | CGGGTTCAATTCCCGGCCAATGCA |
| 267 | Glu_TTC_chr1:17199077-17199149 (+) | GTCCCTGGTGGTCTAGTGGCTAGGATTCGGCGCTTTCACCGCCGCGGCC |
| | | CGGGTTCGATTCCCGGCCAGGGAA |
| 268 | Asn_GTT_chr1:17216171-17216245 (+) | TGTCTCTGTGGCGCAATCGGTTAGCGCGTTCGGCTGTTAACCGAAAGA |
| | | TTGGTGGTTCGAGCCCACCCAGGGACG |
| 269 | Arg_TCT_chr1:94313128-94313213 (+) | TGGCTCCGTGGCGCAATGGATAGCGCATTGGACTTCTAGAGGCTGAAG |
| | | GCATTCAAAGGTTCCGGGTTCGAGTCCCGGCGGAGTCG |
| 270 | Lys_CTT_chr1:145395521-145395594 (-) | GCCCGGCTAGCTCAGTCGGTAGAGCATGAGACTCTTAATCTCAGGGTC |
| | | GTGGGTTCGAGCCCCACGTTGGGCGC |
| 271 | His_GTG_chr1:145396880-145396952 (-) | GCCGTGATCGTATAGTGGTTAGTACTCTGCGTTGTGGCCGCAGCAACCT |
| | | CGGTTCGAATCCGAGTCACGGCAG |
| 272 | Gly_TCC_chr1:145397863-145397935 (-) | GCGTTGGTGGTATAGTGGTGAGCATAGCTGCCTTCCAAGCAGTTGACC |
| | | CGGGTTCGATTCCCGGCCAACGCAG |
| 273 | Glu_CTC_chr1:145399232-145399304 (-) | TCCCTGGTGGTCTAGTGGTTAGGATTCGGCGCTCTCACCGCCGCGGCCC |
| | | GGGTTCGATTCCCGGTCAGGGAAA |
| 274 | Gln_CTG_chr1:145963303-145963375 | AGGTTCCATGGTGTAATGGTGAGCACTCTGGACTCTGAATCCAGCGAT |
| | (+) | CCGAGTTCGAGTCTCGGTGGAACCT |
| 275 | Asn_GTT_chr1:148000804-148000878 | TGTCTCTGTGGCGTAGTCGGTTAGCGCGTTCGGCTGTTAACCGAAAAGT |
| | (+) | TGGTGGTTCGAGCCCACCCAGGAACG |
| 276 | Asn_GTT_chr1:148248114-148248188 | TGTCTCTGTGGCGCAATCGGTTAGCGCGTTCGGCTGTTAACCGAAAGG |
| | (+) | TTGGTGGTTCGAGCCCACCCAGGGACG |
| 277 | Asn_GTT_chr1:148598313-148598387 (-) | GTCTCTGTGGCGCAATCGGTTAGCGCATTCGGCTGTTAACCGAAAGGT |
| | | TGGTGGTTCGAGCCCACCCAGGGACGC |
| 278 | Asn_GTT_chr1:149230569-149230643 (-) | GTCTCTGTGGCGCAATGGGTTAGCGCGTTCGGCTGTTAACCGAAAGGT |
| | | TGGTGGTTCGAGCCCATCCAGGGACGC |
| 279 | Val_CAC_chr1:149294665-149294736 (-) | GCACTGGTGGTTCAGTGGTAGAATTCTCGCCTCACACGCGGGACACCC |
| | | GGGTTCAATTCCCGGTCAAGGCAA |
| 280 | Val_CAC_chr1:149298554-149298627 (-) | GTTTCCGTAGTGTAGTGGTTATCACGTTCGCCTCACACGCGAAAGGTCC |
| | | CCGGTTCGAAACTGGGCGGAAACAG |
| 281 | Gly_CCC_chr1:149680209-149680280 (-) | GCACTGGTGGTTCAGTGGTAGAATTCTCGCCTCCCACGCGGGAGACCC |
| | | GGGTTTAATTCCCGGTCAAGATAA |
| 282 | Val_CAC_chr1:149684087-149684161 (-) | GTTTCCGTAGTGTAGTGGTTATCACGTTCGCCTCACACGCGTAAAGGTC |
| | | CCCGGTTCGAAACCGGGCGGAAACAT |
| 283 | Met_CAT_chr1:153643725-153643797 | TAGCAGAGTGGCGCAGCGGAAGCGTGCTGGGCCCATAACCCAGAGGT |
| | (+) | CGATGGATCGAAACCATCCTCTGCTA |
| 284 | Val_CAC_chr1:161369489-161369562 (-) | GTTTCCGTAGTGTAGTGGTTATCACGTTCGCCTCACACGCGAAAGGTCC |
| | | CCGGTTCGAAACCGGGCGGAAACAA |
| 285 | Asp_GTC_chr1:161410614-161410686 (-) | TCCTCGTTAGTATAGTGGTGAGTATCCCCGCCTGTCACGCGGGAGACC |
| | | GGGGTTCGATTCCCCGACGGGGAGG |
| 286 | Gly_GCC_chr1:161413093-161413164 | TGCATGGGTGGTTCAGTGGTAGAATTCTCGCCTGCCACGCGGGAGGCC |
| | (+) | CGGGTTCGATTCCCGGCCCATGCA |
| 287 | Glu_CTC_chr1:161417017-161417089 (-) | TCCCTGGTGGTCTAGTGGTTAGGATTCGGCGCTCTCACCGCCGCGGCCC |
| | | GGGTTCGATTCCCGGTCAGGGAAG |
| 288 | Asp_GTC_chr1:161492934-161493006 | ATCCTTGTTACTATAGTGGTGAGTATCTCTGCCTGTCATGCGTGAGAGA |
| | (+) | GGGGGTCGATTCCCCGACGGGGAG |
| 289 | Gly_GCC_chr1:161493636-161493707 (-) | GCATTGGTGGTTCAGTGGTAGAATTCTCGCCTGCCACGCGGGAGGCCC |
| | | GGGTTCGATTCCCGGCCAATGCAC |
| 290 | Leu_CAG_chr1:161500131-161500214 (-) | GTCAGGATGGCCGAGCGGTCTAAGGCGCTGCGTTCAGGTCGCAGTCTC |
| | | CCCTGGAGGCGTGGGTTCGAATCCCACTCCTGACAA |
| 291 | Gly_TCC_chr1:161500902-161500974 | CGCGTTGGTGGTATAGTGGTGAGCATAGCTGCCTTCCAAGCAGTTGAC |
| | (+) | CCGGGTTCGATTCCCGGCCAACGCA |
| 292 | Asn_GTT_chr1:161510030-161510104 | CGTCTCTGTGGCGCAATCGGTTAGCGCGTTCGGCTGTTAACCGAAAGG |
| | (+) | TTGGTGGTTCGATCCCACCCAGGGACG |
| 293 | Glu_TTC_chr1:161582507-161582579 (+) | CGCGTTGGTGGTGTAGTGGTGAGCACAGCTGCCTTTCAAGCAGTTAAC |
| | | GCGGGTTCGATTCCCGGGTAACGAA |
| 294 | Pro_CGG_chr1:167683961-167684033 | CGGCTCGTTGGTCTAGGGGTATGATTCTCGCTTCGGGTGCGAGAGGTC |
| | (+) | CCGGGTTCAAATCCCGGACGAGCCC |
| 295 | Pro_AGG_chr1:167684724-167684796 (-) | GGCTCGTTGGTCTAGGGGTATGATTCTCGCTTAGGGTGCGAGAGGTCC |
| | | CGGGTTCAAATCCCGGACGAGCCCT |
| 296 | Lys_TTT_chr1:204475654-204475727 (+) | CGCCCGGATAGCTCAGTCGGTAGAGCATCAGACTTTTAATCTGAGGGT |
| | | CCAGGGTTCAAGTCCCTGTTCGGGCG |
| 297 | Lys_TTT_chr1:204476157-204476230(-) | GCCCGGATAGCTCAGTCGGTAGAGCATCAGACTTTTAATCTGAGGGTC |
| | | CAGGGTTCAAGTCCCTGTTCGGGCGT |
| 298 | Leu_CAA_chr1:249168053-249168159 | TGTCAGGATGGCCGAGTGGTCTAAGGCGCCAGACTCAAGGTAAGCACC |
| | (+) | TTGCCTGCGGGCTTTCTGGTCTCCGGATGGAGGCGTGGGTTCGAATCCC |
| 299 | Glu_CTC_chr1:249168446-249168518 | TTCCCTGGTGGTCTAGTGGTTAGGATTCGGCGCTCTCACCGCCGCGGCC |
| | (+) | CGGGTTCGATTCCCGGTCAGGAAA |
| 300 | Tyr_GTA_chr2:27273649-27273738 (+) | GCCTTCGATAGCTCAGTTGGTAGAGCGGAGGACTGTAGTGGATAGGGC |
| | | GTGGCAATCCTTAGGTCGCTGGTTCGATTCCGGCTCGAAGGA |
| 301 | Ala_AGC_chr2:27274081-27274154 (+) | CGGGGGATTAGCTCAAATGGTAGAGCGCTCGCTTAGCATGCGAGAGGT |
| | | AGCGGGATCGATGCCCGCATCCTCCA |
| 302 | Ile_TAT_chr2:43037675-43037768 (+) | AGCTCCAGTGGCGCAATCGGTTAGCGCGCGGTACTTATACAGCAGTAC |
| | | ATGCAGAGCAATGCCGAGGTTGTGAGTTCGAGCCTCACCTGGAGCA |
| 303 | Gly_CCC_chr2:70476122-70476193 (-) | GCGCCGCTGGTGTAGTGGTATCATGCAAGATTCCCATTCTTGCGACCCG |
| | | GGTTCGATTCCCGGGCGGCGCAT |
| 304 | Glu_TTC_chr2:131094700-131094772 (-) | TCCCATATGGTCTAGCGGTTAGGATTCCTGGTTTTCACCCAGGTGGCCC |
| | | GGGTTCGACTCCCGGTATGGGAAC |
| 305 | Ala_CGC_chr2:157257280-157257352 | GGGGGATGTAGCTCAGTGGTAGAGCGCGCGCTTCGCATGTGTGAGGTC |
| | (+) | CCGGGTTCAATCCCCGGCATCTCCA |
| 306 | Gly_GCC_chr2:157257658-157257729 (-) | GCATTGGTGGTTCAGTGGTAGAATTCTCGCCTGCCACGCGGGAGGCCC |
| | | GGGTTCGATTCCCGGCCAATGCAA |
| 307 | Arg_ACG_chr3:45730490-45730563 (-) | GGGCCAGTGGCGCAATGGATAACGCGTCTGACTACGGATCAGAAGATT |
| | | CTAGGTTCGACTCCTGGCTGGCTCGC |
| 308 | Val_AAC_chr3:169490017-169490090 | GGTTTCCGTAGTGTAGTGGTTATCACGTTCGCCTAACACGCGAAAGGT |
| | (+) | CCCCGGTTCGAAACCGGGCGGAAACA |
| 309 | Val_AAC_chr5:180596609-180596682 | AGTTTCCGTAGTGTAGTGGTTATCACGTTCGCCTAACACGCGAAAGGT |
| | (+) | CCCCGGTTCGAAACCGGGCGGAAACA |
| 310 | Leu_AAG_chr5:180614700-180614782 | AGGTAGCGTGGCCGAGCGGTCTAAGGCGCTGGATTAAGGCTCCAGTCT |
| | (+) | CTTCGGGGGCGTGGGTTCGAATCCCACCGCTGCCA |
| 311 | Val_AAC_chr5:180615415-180615488 (-) | GTTTCCGTAGTGTAGTGGTCATCACGTTCGCCTAACACGCGAAAGGTC |
| | | CCCGGTTCGAAACCGGGCGGAAACAT |
| 312 | Pro_TGG_chr5:180615853-180615925 (-) | GGCTCGTTGGTCTAGGGGTATGATTCTCGCTTTGGGTGCGAGAGGTCCC |
| | | GGGTTCAAATCCCGGACGAGCCCA |
| 313 | Thr_TGT_chr5:180618686-180618758 (-) | GGCTCCATAGCTCAGGGGTTAGAGCACTGGTCTTGTAAACCAGGGTCG |
| | | CGAGTTCAAATCTCGCTGGGGCCTG |
| 314 | Ala_TGC_chr5:180633867-180633939 | TGGGGATGTAGCTCAGTGGTAGAGCGCATGCTTTGCATGTATGAGGCC |
| | (+) | CCGGGTTCGATCCCCGGCATCTCCA |
| 315 | Lys_CTT_chr5:180634754-180634827 (+) | CGCCCGGCTAGCTCAGTCGGTAGAGCATGAGACTCTTAATCTCAGGGT |
| | | CGTGGGTTCGAGCCCCACGTTGGGCG |
| 316 | Val_AAC_chr5:180645269-180645342 (-) | GTTTCCGTAGTGTAGTGGTTATCACGTTCGCCTAACACGCGAAAGGTCC |
| | | CCGGTTCGAAACCGGGCGGAAACAA |
| 317 | Lys_CTT_chr5:180648978-180649051 (-) | GCCCGGCTAGCTCAGTCGGTAGAGCATGAGACTCTTAATCTCAGGGTC |
| | | GTGGGTTCGAGCCCCACGTTGGGCGT |
| 318 | Val_CAC_chr5:180649394-180649467 (-) | GTTTCCGTAGTGTAGTGGTTATCACGTTCGCCTCACACGCGAAAGGTCC |
| | | CCGGTTCGAAACCGGGCGGAAACAC |
| 319 | Met_CAT_chr6:26286753-26286825 (+) | CAGCAGAGTGGCGCAGCGGAAGCGTGCTGGGCCCATAACCCAGAGGT |
| | | CGATGGATCGAAACCATCCTCTGCTA |
| 320 | Ser_GCT_chr6:26305717-26305801 (-) | GGAGAGGCCTGGCCGAGTGGTTAAGGCGATGGACTGCTAATCCATTGT |
| | | GCTCTGCACGCGTGGGTTCGAATCCCATCCTCGTCGC |
| 321 | Gln_TTG_chr6:26311423-26311495 (-) | GGCCCCATGGTGTAATGGTTAGCACTCTGGACTTTGAATCCAGCGATC |
| | | CGAGTTCAAATCTCGGTGGGACCTG |
| 322 | Gln_TTG_chr6:26311974-26312046 (-) | GGCCCCATGGTGTAATGGTTAGCACTCTGGACTTTGAATCCAGCGATC |
| | | CGAGTTCAAATCTCGGTGGGACCTA |
| 323 | Ser_TGA_chr6:26312823-26312905 (-) | GTAGTCGTGGCCGAGTGGTTAAGGCGATGGACTTGAAATCCATTGGGG |
| | | TCTCCCCGCGCAGGTTCGAATCCTGCCGACTACGG |
| 324 | Met_CAT_chr6:26313351-26313423 (-) | AGCAGAGTGGCGCAGCGGAAGCGTGCTGGGCCCATAACCCAGAGGTC |
| | | GATGGATCGAAACCATCCTCTGCTAT |
| 325 | Arg_TCG_chr6:26323045-26323118 (+) | GGACCACGTGGCCTAATGGATAAGGCGTCTGACTTCGGATCAGAAGAT |
| | | TGAGGGTTCGAATCCCTCCGTGGTTA |
| 326 | Ser_AGA_chr6:26327816-26327898 (+) | TGTAGTCGTGGCCGAGTGGTTAAGGCGATGGACTAGAAATCCATTGGG |
| | | GTCTCCCCGCGCAGGTTCGAATCCTGCCGACTACG |
| 327 | Met_CAT_chr6:26330528-26330600 (-) | AGCAGAGTGGCGCAGCGGAAGCGTGCTGGGCCCATAACCCAGAGGTC |
| | | GATGGATCGAAACCATCCTCTGCTAG |
| 328 | Leu_CAG_chr6:26521435-26521518 (+) | CGTCAGGATGGCCGAGCGGTCTAAGGCGCTGCGTTCAGGTCGCAGTCT |
| | | CCCCTGGAGGCGTGGGTTCGAATCCCACTCCTGACA |
| 329 | Thr_AGT_chr6:26533144-26533218 (-) | GGCTCCGTGGCTTAGCTGGTTAAAGCGCCTGTCTAGTAAACAGGAGAT |
| | | CCTGGGTTCGAATCCCAGCGGGGCCTG |
| 330 | Arg_ACG_chr6:26537725-26537798 (+) | AGGGCCAGTGGCGCAATGGATAACGCGTCTGACTACGGATCAGAAGA |
| | | TTCCAGGTTCGACTCCTGGCTGGCTCG |
| 331 | Val_CAC_chr6:26538281-26538354 (+) | GGTTTCCGTAGTGTAGTGGTTATCACGTTCGCCTCACACGCGAAAGGTC |
| | | CCCGGTTCGAAACCGGGCGGAAACA |
| 332 | Ala_CGC_chr6:26553730-26553802 (+) | AGGGGATGTAGCTCAGTGGTAGAGCGCATGCTTCGCATGTATGAGGTC |
| | | CCGGGTTCGATCCCCGGCATCTCCA |
| 333 | Ile_AAT_chr6:26554349-26554423 (+) | TGGCCGGTTAGCTCAGTTGGTTAGAGCGTGGTGCTAATAACGCCAAGG |
| | | TCGCGGGTTCGATCCCCGTACGGGCCA |
| 334 | Pro_AGG_chr6:26555497-26555569 (+) | CGGCTCGTTGGTCTAGGGGTATGATTCTCGCTTAGGGTGCGAGAGGTC |
| | | CCGGGTTCAAATCCCGGACGAGCCC |
| 335 | Lys_CTT_chr6:26556773-26556846 (+) | AGCCCGGCTAGCTCAGTCGGTAGAGCATGAGACTCTTAATCTCAGGGT |
| | | CGTGGGTTCGAGCCCCACGTTGGGCG |
| 336 | Tyr_GTA_chr6:26569085-26569176 (+) | TCCTTCGATAGCTCAGTTGGTAGAGCGGAGGACTGTAGTTGGCTGTGT |
| | | CCTTAGACATCCTTAGGTCGCTGGTTCGAATCCGGCTCGAAGGA |
| 337 | Ala_AGC_chr6:26572091-26572164 (-) | GGGGAATTAGCTCAAATGGTAGAGCGCTCGCTTAGCATGCGAGAGGTA |
| | | GCGGGATCGATGCCCGCATTCTCCAG |
| 338 | Met_CAT_chr6:26766443-26766516 (+) | CGCCCTCTTAGCGCAGCGGGCAGCGCGTCAGTCTCATAATCTGAAGGT |
| | | CCTGAGTTCGAGCCTCAGAGAGGGCA |
| 339 | Ile_TAT_chr6:26988124-26988218 (+) | TGCTCCAGTGGCGCAATCGGTTAGCGCGCGGTACTTATATGGCAGTAT |
| | | GTGTGCGAGTGATGCCGAGGTTGTGAGTTCGAGCCTCACCTGGAGCA |
| 340 | His_GTG_chr6:27125905-27125977 (+) | TGCCGTGATCGTATAGTGGTTAGTACTCTGCGTTGTGGCCGCAGCAACC |
| | | TCGGTTCGAATCCGAGTCACGGCA |
| 341 | Ile_AAT_chr6:27144993-27145067 (-) | GGCCGGTTAGCTCAGTTGGTTAGAGCGTGGTGCTAATAACGCCAAGGT |
| | | CGCGGGTTCGATCCCCGTACGGGCCAC |
| 342 | Val_AAC_chr6:27203287-27203360 (+) | AGTTTCCGTAGTGTAGTGGTTATCACGTTTGCCTAACACGCGAAAGGTC |
| | | CCCGGTTCGAAACCGGGCAGAAACA |
| 343 | Val_CAC_chr6:27248048-27248121 (-) | GCTTCTGTAGTGTAGTGGTTATCACGTTCGCCTCACACGCGAAAGGTCC |
| | | CCGGTTCGAAACCGGGCAGAAGCAA |
| 344 | Asp_GTC_chr6:27447452-27447524 (+) | TTCCTCGTTAGTATAGTGGTGAGTATCCCCGCCTGTCACGCGGGAGACC |
| | | GGGGTTCGATTCCCCGACGGGGAG |
| 345 | Ser_TGA_chr6:27473606-27473688 (-) | GTAGTCGTGGCCGAGTGGTTAAGGCGATGGACTTGAAATCCATTGGGG |
| | | TTTCCCCGCGCAGGTTCGAATCCTGTCGGCTACGG |
| 346 | Gln_CTG_chr6:27487307-27487379 (+) | AGGTTCCATGGTGTAATGGTTAGCACTCTGGACTCTGAATCCAGCGAT |
| | | CCGAGTTCAAATCTCGGTGGAACCT |
| 347 | Asp_GTC_chr6:27551235-27551307 (-) | TCCTCGTTAGTATAGTGGTGAGTGTCCCCGTCTGTCACGCGGGAGACC |
| | | GGGGTTCGATTCCCCGACGGGGAGA |
| 348 | Val_AAC_chr6:27618706-27618779 (-) | GTTTCCGTAGTGTAGTGGTTATCACGTTCGCCTAACACGCGAAAGGTCC |
| | | CTGGATCAAAACCAGGCGGAAACAA |
| 349 | Ile_AAT_chr6:27655966-27656040 (+) | CGGCCGGTTAGCTCAGTTGGTTAGAGCGTGGTGCTAATAACGCCAAGG |
| | | TCGCGGGTTCGATCCCCGTACTGGCCA |
| 366 | Pro_AGG_chr7:128423503-128423575 | TGGCTCGTTGGTCTAGGGGTATGATTCTCGCTTAGGGTGCGAGAGGTC |
| | (+) | CCGGGTTCAAATCCCGGACGAGCCC |
| 367 | Arg_CCT_chr7:139025445-139025518 | AGCCCCAGTGGCCTAATGGATAAGGCATTGGCCTCCTAAGCCAGGGAT |
| | (+) | TGTGGGTTCGAGTCCCATCTGGGGTG |
| 368 | Cys_GCA_chr7:149388271-149388343 (-) | GGGGATATAGCTCAGGGGTAGAGCATTTGACTGCAGATCAAGAGGTCC |
| | | CCGGTTCAAATCCGGGTGCCCCCCC |
| 369 | Tyr_GTA_chr8:67025601-67025694 (+) | CCCTTCGATAGCTCAGCTGGTAGAGCGGAGGACTGTAGCTACTTCCTC |
| | | AGCAGGAGACATCCTTAGGTCGCTGGTTCGATTCCGGCTCGAAGGA |
| 370 | Tyr_GTA_chr8:67026222-67026311 (+) | CCCTTCGATAGCTCAGCTGGTAGAGCGGAGGACTGTAGGCGCGCGCCC |
| | | GTGGCCATCCTTAGGTCGCTGGTTCGATTCCGGCTCGAAGGA |
| 371 | Ala_AGC_chr8:67026423-67026496 (+) | TGGGGGATTAGCTCAAATGGTAGAGCGCTCGCTTAGCATGCGAGAGGT |
| | | AGCGGGATCGATGCCCGCATCCTCCA |
| 372 | Ser_AGA_chr8:96281884-96281966 (-) | GTAGTCGTGGCCGAGTGGTTAAGGCGATGGACTAGAAATCCATTGGGG |
| | | TCTCCCCGCGCAGGTTCGAATCCTGCCGACTACGG |
| 373 | Met_CAT_chr8:124169469-124169542 (-) | GCCTCGTTAGCGCAGTAGGTAGCGCGTCAGTCTCATAATCTGAAGGTC |
| | | GTGAGTTCGATCCTCACACGGGGCAC |
| 374 | Arg_TCT_chr9:131102354-131102445 (-) | GGCTCTGTGGCGCAATGGATAGCGCATTGGACTTCTAGCTGAGCCTAG |
| | | TGTGGTCATTCAAAGGTTGTGGGTTCGAGTCCCACCAGAGTCGA |
| 375 | Asn_GTT_chr10:22518437-22518511 (-) | GTCTCTGTGGCGCAATCGGTTAGCGCGTTCGGCTGTTAACCGAAAGGT |
| | | TGGTGGTTCGAGCCCACCCAGGGACGC |
| 376 | Ser_TGA_chr10:69524260-69524342 (+) | GGCAGCGATGGCCGAGTGGTTAAGGCGTTGGACTTGAAATCCAATGGG |
| | | GTCTCCCCGCGCAGGTTCGAACCCTGCTCGCTGCG |
| 377 | Val_TAC_chr11:59318101-59318174 (-) | GGTTCCATAGTGTAGTGGTTATCACGTCTGCTTTACACGCAGAAGGTCC |
| | | TGGGTTCGAGCCCCAGTGGAACCAT |
| 378 | Val_TAC_chr11:59318459-59318532 (-) | GGTTCCATAGTGTAGCGGTTATCACGTCTGCTTTACACGCAGAAGGTCC |
| | | TGGGTTCGAGCCCCAGTGGAACCAC |
| 379 | Arg_TCT_chr11:59318766-59318852 (+) | TGGCTCTGTGGCGCAATGGATAGCGCATTGGACTTCTAGATAGTTAGA |
| | | GAAATTCAAAGGTTGTGGGTTCGAGTCCCACCAGAGTCG |
| 380 | Leu_TAA_chr11:59319227-59319310 (+) | TACCAGAATGGCCGAGTGGTTAAGGCGTTGGACTTAAGATCCAATGGA |
| | | TTCATATCCGCGTGGGTTCGAACCCCACTTCTGGTA |
| 381 | Lys_TTT_chr11:59323901-59323974 (+) | GGCCCGGATAGCTCAGTCGGTAGAGCATCAGACTTTTAATCTGAGGGT |
| | | CCGGGGTTCAAGTCCCTGTTCGGGCG |
| 382 | Phe_GAA_chr11:59324969-59325042 (-) | GCCGAAATAGCTCAGTTGGGAGAGCGTTAGACTGAAGATCTAAAGGTC |
| | | CCTGGTTCGATCCCGGGTTTCGGCAG |
| 383 | Lys_TTT_chr11:59327807-59327880 (-) | GCCCGGATAGCTCAGTCGGTAGAGCATCAGACTTTTAATCTGAGGGTC |
| | | CAGGGTTCAAGTCCCTGTTCGGGCGG |
| 384 | Phe_GAA_chr11:59333852-59333925 (-) | GCCGAAATAGCTCAGTTGGGAGAGCGTTAGACTGAAGATCTAAAGGTC |
| | | CCTGGTTCAATCCCGGGTTTCGGCAG |
| 385 | Ser_GCT_chr11:66115590-66115672 (+) | GGACGAGGTGGCCGAGTGGTTAAGGCGATGGACTGCTAATCCATTGTG |
| | | CTTTGCACGCGTGGGTTCGAATCCCATCCTCGTCG |
| 386 | Pro_TGG_chr11:75946868-75946940 (-) | GGCTCGTTGGTCTAGGGGTATGATTCTCGGTTTGGGTCCGAGAGGTCCC |
| | | GGGTTCAAATCCCGGACGAGCCCC |
| 387 | Ser_CGA_chr12:56584147-56584229 (+) | AGTCACGGTGGCCGAGTGGTTAAGGCGTTGGACTCGAAATCCAATGGG |
| | | GTTTCCCCGCACAGGTTCGAATCCTGTTCGTGACG |
| 388 | Asp_GTC_chr12:98897280-98897352 (+) | CTCCTCGTTAGTATAGTGGTTAGTATCCCCGCCTGTCACGCGGGAGACC |
| | | GGGGTTCAATTCCCCGACGGGGAG |
| 389 | Trp_CCA_chr12:98898029-98898101 (+) | GGACCTCGTGGCGCAACGGTAGCGCGTCTGACTCCAGATCAGAAGGCT |
| | | GCGTGTTCGAATCACGTCGGGGTCA |
| 390 | Ala_TGC_chr12:125406300-125406372 (-) | GGGGATGTAGCTCAGTGGTAGAGCGCATGCTTTGCATGTATGAGGCCC |
| | | CGGGTTCGATCCCCGGCATCTCCAT |
| 391 | Phe_GAA_chr12:125412388-125412461 | GCCGAAATAGCTCAGTTGGGAGAGCGTTAGACTGAAGATCTAAAGGTC |
| | (-) | CCTGGTTCGATCCCGGGTTTCGGCAG |
| 392 | Ala_TGC_chr12:125424511-125424583 | AGGGGATGTAGCTCAGTGGTAGAGCGCATGCTTTGCACGTATGAGGCC |
| | (+) | CCGGGTTCAATCCCCGGCATCTCCA |
| 393 | Asn_GTT_chr13:31248100-31248174 (-) | GTCTCTGTGGCGCAATCGGTTAGCGCGTTCGGCTGTTAACCGAAAGGT |
| | | TGGTGGTTCGAGCCCACCCAGGGACGG |
| 394 | Glu_TTC_chr13:45492061-45492133 (-) | TCCCACATGGTCTAGCGGTTAGGATTCCTGGTTTTCACCCAGGCGGCCC |
| | | GGGTTCGACTCCCGGTGTGGGAAC |
| 395 | Thr_TGT_chr14:21081948-21082021 (-) | GGCTCCATAGCTCAGGGGTTAGAGCGCTGGTCTTGTAAACCAGGGGTC |
| | | GCGAGTTCAATTCTCGCTGGGGCCTG |
| 396 | Leu_TAG_chr14:21093528-21093610 (+) | TGGTAGTGTGGCCGAGCGGTCTAAGGCGCTGGATTTAGGCTCCAGTCT |
| | | CTTCGGGGGCGTGGGTTCGAATCCCACCACTGCCA |
| 397 | Thr_TGT_chr14:21099318-21099391 (-) | GGCTCCATAGCTCAGGGGTTAGAGCACTGGTCTTGTAAACCAGGGGTC |
| | | GCGAGTTCAAATCTCGCTGGGGCCTC |
| 398 | Pro_TGG_chr14:21101164-21101236 (+) | TGGCTCGTTGGTCTAGTGGTATGATTCTCGCTTTGGGTGCGAGAGGTCC |
| | | CGGGTTCAAATCCCGGACGAGCCC |
| 399 | Tyr_GTA_chr14:21131350-21131444 (-) | CCTTCGATAGCTCAGCTGGTAGAGCGGAGGACTGTAGATTGTACAGAC |
| | | ATTTGCGGACATCCTTAGGTCGCTGGTTCGATTCCGGCTCGAAGGAA |
| 400 | Thr_TGT_chr14:21149848-21149921 (+) | AGGCCCTATAGCTCAGGGGTTAGAGCACTGGTCTTGTAAACCAGGGGT |
| | | CGCGAGTTCAAATCTCGCTGGGGCCT |
| 401 | Tyr_GTA_chr14:21151431-21151520 (+) | TCCTTCGATAGCTCAGCTGGTAGAGCGGAGGACTGTAGTACTTAATGT |
| | | GTGGTCATCCTTAGGTCGCTGGTTCGATTCCGGCTCGAAGGA |
| 402 | Pro_TGG_chr14:21152174-21152246 (+) | TGGCTCGTTGGTCTAGGGGTATGATTCTCGCTTTGGGTGCGAGAGGTCC |
| | | CGGGTTCAAATCCCGGACGAGCCC |
| 403 | Lys_CTT_chr14:58706612-58706685 (-) | GCCCGGCTAGCTCAGTCGGTAGAGCATGGGACTCTTAATCCCAGGGTC |
| | | GTGGGTTCGAGCCCCACGTTGGGCGC |
| 404 | Ile_AAT_chr14:102783428-102783 502 | CGGCCGGTTAGCTCAGTTGGTTAGAGCGTGGTGCTAATAACGCCAAGG |
| | (+) | TCGCGGGTTCGATCCCCGTACGGGCCA |
| 405 | Glu_TTC_chr15:26327380-26327452 (-) | TCCCACATGGTCTAGCGGTTAGGATTCCTGGTTTTCACCCAGGCGGCCC |
| | | GGGTTCGACTCCCGGTGTGGGAAT |
| 406 | Ser_GCT_chr15:40886022-40886104 (-) | GACGAGGTGGCCGAGTGGTTAAGGCGATGGACTGCTAATCCATTGTGC |
| | | TCTGCACGCGTGGGTTCGAATCCCATCCTCGTCGA |
| 407 | His_GTG_chr15:45490803-45490875 (-) | GCCGTGATCGTATAGTGGTTAGTACTCTGCGTTGTGGCCGCAGCAACCT |
| | | CGGTTCGAATCCGAGTCACGGCAT |
| 408 | His_GTG_chr15:45493348-45493420 (+) | CGCCGTGATCGTATAGTGGTTAGTACTCTGCGTTGTGGCCGCAGCAAC |
| | | CTCGGTTCGAATCCGAGTCACGGCA |
| 409 | Gln_CTG_chr15:66161399-66161471 (-) | GGTTCCATGGTGTAATGGTTAGCACTCTGGACTCTGAATCCAGCGATCC |
| | | GAGTTCAAATCTCGGTGGAACCTG |
| 410 | Lys_CTT_chr15:79152903-79152976 (+) | TGCCCGGCTAGCTCAGTCGGTAGAGCATGGGACTCTTAATCCCAGGGT |
| | | CGTGGGTTCGAGCCCCACGTTGGGCG |
| 411 | Arg_TCG_chr15:89878303-89878376 (+) | GGGCCGCGTGGCCTAATGGATAAGGCGTCTGACTTCGGATCAGAAGAT |
| | | TGCAGGTTCGAGTCCTGCCGCGGTCG |
| 412 | Gly_CCC_chr16:686735-686806 (-) | GCGCCGCTGGTGTAGTGGTATCATGCAAGATTCCCATTCTTGCGACCCG |
| | | GGTTCGATTCCCGGGCGGCGCAC |
| 413 | Arg_CCG_chr16:3200674-3200747 (+) | GGGCCGCGTGGCCTAATGGATAAGGCGTCTGATTCCGGATCAGAAGAT |
| | | TGAGGGTTCGAGTCCCTTCGTGGTCG |
| 414 | Arg_CCT_chr16:3202900-3202973 (+) | CGCCCCGGTGGCCTAATGGATAAGGCATTGGCCTCCTAAGCCAGGGAT |
| | | TGTGGGTTCGAGTCCCACCCGGGGTA |
| 415 | Lys_CTT_chr16:3207405-3207478 (-) | GCCCGGCTAGCTCAGTCGGTAGAGCATGAGACCCTTAATCTCAGGGTC |
| | | GTGGGTTCGAGCCCCACGTTGGGCGT |
| 416 | Thr_CGT_chr16:14379749-14379821 (+) | AGGCGCGGTGGCCAAGTGGTAAGGCGTCGGTCTCGTAAACCGAAGATC |
| | | ACGGGTTCGAACCCCGTCCGTGCCT |
| 417 | Leu_TAG_chr16:22207031-22207113 (-) | GGTAGCGTGGCCGAGTGGTCTAAGGCGCTGGATTTAGGCTCCAGTCAT |
| | | TTCGATGGCGTGGGTTCGAATCCCACCGCTGCCAC |
| 418 | Leu_AAG_chr16:22308460-22308542 (+) | GGGTAGCGTGGCCGAGCGGTCTAAGGCGCTGGATTAAGGCTCCAGTCT |
| | | CTTCGGGGGCGTGGGTTCGAATCCCACCGCTGCCA |
| 419 | Leu_CAG_chr16:57333862-57333945 (+) | AGTCAGGATGGCCGAGCGGTCTAAGGCGCTGCGTTCAGGTCGCAGTCT |
| | | CCCCTGGAGGCGTGGGTTCGAATCCCACTTCTGACA |
| 420 | Leu_CAG_chr16:57334391-57334474 (-) | GTCAGGATGGCCGAGCGGTCTAAGGCGCTGCGTTCAGGTCGCAGTCTC |
| | | CCCTGGAGGCGTGGGTTCGAATCCCACTTCTGACAG |
| 421 | Met_CAT_chr16:87417627-87417700 (-) | GCCTCGTTAGCGCAGTAGGCAGCGCGTCAGTCTCATAATCTGAAGGTC |
| | | GTGAGTTCGAGCCTCACACGGGGCAG |
| 422 | Leu_TAG_chr17:8023631-8023713 (-) | GGTAGCGTGGCCGAGCGGTCTAAGGCGCTGGATTTAGGCTCCAGTCTC |
| | | TTCGGAGGCGTGGGTTCGAATCCCACCGCTGCCAG |
| 423 | Arg_TCT_chr17:8024242-8024330 (+) | TGGCTCTGTGGCGCAATGGATAGCGCATTGGACTTCTAGTGACGAATA |
| | | GAGCAATTCAAAGGTTGTGGGTTCGAATCCCACCAGAGTCG |
| 424 | Gly_GCC_chr17:8029063-8029134 (+) | CGCATTGGTGGTTCAGTGGTAGAATTCTCGCCTGCCACGCGGGAGGCC |
| | | CGGGTTCGATTCCCGGCCAATGCA |
| 425 | Ser_CGA_chr17:8042198-8042280 (-) | GCTGTGATGGCCGAGTGGTTAAGGCGTTGGACTCGAAATCCAATGGGG |
| | | TCTCCCCGCGCAGGTTCGAATCCTGCTCACAGCGT |
| 426 | Thr_AGT_chr17:8042769-8042843 (-) | GGCGCCGTGGCTTAGCTGGTTAAAGCGCCTGTCTAGTAAACAGGAGAT |
| | | CCTGGGTTCGAATCCCAGCGGTGCCTG |
| 427 | Trp_CCA_chr17:8089675-8089747 (+) | CGACCTCGTGGCGCAACGGTAGCGCGTCTGACTCCAGATCAGAAGGTT |
| | | GCGTGTTCAAATCACGTCGGGGTCA |
| 428 | Ser_GCT_chr17:8090183-8090265 (+) | AGACGAGGTGGCCGAGTGGTTAAGGCGATGGACTGCTAATCCATTGTG |
| | | CTCTGCACGCGTGGGTTCGAATCCCATCCTCGTCG |
| 429 | Thr_AGT_chr17:8090477-8090551 (+) | CGGCGCCGTGGCTTAGTTGGTTAAAGCGCCTGTCTAGTAAACAGGAGA |
| | | TCCTGGGTTCGAATCCCAGCGGTGCCT |
| 430 | Trp_CCA_chr17:8124186-8124258 (-) | GGCCTCGTGGCGCAACGGTAGCGCGTCTGACTCCAGATCAGAAGGTTG |
| | | CGTGTTCAAATCACGTCGGGGTCAA |
| 431 | Gly_TCC_chr17:8124865-8124937 (+) | AGCGTTGGTGGTATAGTGGTAAGCATAGCTGCCTTCCAAGCAGTTGAC |
| | | CCGGGTTCGATTCCCGGCCAACGCA |
| 432 | Asp_GTC_chr17:8125555-8125627 (-) | TCCTCGTTAGTATAGTGGTGAGTATCCCCGCCTGTCACGCGGGAGACC |
| | | GGGGTTCGATTCCCCGACGGGGAGA |
| 433 | Pro_CGG_chr17:8126150-8126222 (-) | GGCTCGTTGGTCTAGGGGTATGATTCTCGCTTCGGGTGCGAGAGGTCC |
| | | CGGGTTCAAATCCCGGACGAGCCCT |
| 434 | Thr_AGT_chr17:8129552-8129626 (-) | GGCGCCGTGGCTTAGTTGGTTAAAGCGCCTGTCTAGTAAACAGGAGAT |
| | | CCTGGGTTCGAATCCCAGCGGTGCCTT |
| 435 | Ser_AGA_chr17:8129927-8130009 (-) | GTAGTCGTGGCCGAGTGGTTAAGGCGATGGACTAGAAATCCATTGGGG |
| | | TCTCCCCGCGCAGGTTCGAATCCTGCCGACTACGT |
| 436 | Trp_CCA_chr17:19411493-19411565 (+) | TGACCTCGTGGCGCAATGGTAGCGCGTCTGACTCCAGATCAGAAGGTT |
| | | GCGTGTTCAAGTCACGTCGGGGTCA |
| 437 | Thr_CGT_chr17:29877092-29877164 (+) | AGGCGCGGTGGCCAAGTGGTAAGGCGTCGGTCTCGTAAACCGAAGATC |
| | | GCGGGTTCGAACCCCGTCCGTGCCT |
| 438 | Cys_GCA_chr17:37023897-37023969 (+) | AGGGGGTATAGCTCAGTGGTAGAGCATTTGACTGCAGATCAAGAGGTC |
| | | CCCGGTTCAAATCCGGGTGCCCCCT |
| 439 | Cys_GCA_chr17:37025544-37025616 (-) | GGGGGTATAGCTCAGTGGTAGAGCATTTGACTGCAGATCAAGAGGTCC |
| | | CTGGTTCAAATCCGGGTGCCCCCTC |
| 440 | Cys_GCA_chr17:37309986-37310058 (-) | GGGGGTATAGCTCAGTGGTAGAGCATTTGACTGCAGATCAAGAGGTCC |
| | | CCGGTTCAAATCCGGGTGCCCCCTC |
| 441 | Gln_TTG_chr17:47269889-47269961 (+) | AGGTCCCATGGTGTAATGGTTAGCACTCTGGACTTTGAATCCAGCGAT |
| | | CCGAGTTCAAATCTCGGTGGGACCT |
| 442 | Arg_CCG_chr17:66016012-66016085 (-) | GACCCAGTGGCCTAATGGATAAGGCATCAGCCTCCGGAGCTGGGGATT |
| | | GTGGGTTCGAGTCCCATCTGGGTCGC |
| 443 | Arg_CCT_chr17:73030000-73030073 (+) | AGCCCCAGTGGCCTAATGGATAAGGCACTGGCCTCCTAAGCCAGGGAT |
| | | TGTGGGTTCGAGTCCCACCTGGGGTA |
| 444 | Arg_CCT_chr17:73030525-73030598 (-) | GCCCCAGTGGCCTAATGGATAAGGCACTGGCCTCCTAAGCCAGGGATT |
| | | GTGGGTTCGAGTCCCACCTGGGGTGT |
| 445 | Arg_TCG_chr17:73031207-73031280 (+) | AGACCGCGTGGCCTAATGGATAAGGCGTCTGACTTCGGATCAGAAGAT |
| | | TGAGGGTTCGAGTCCCTTCGTGGTCG |
| 446 | Asn_GTT_chr19:1383561-1383635 (+) | CGTCTCTGTGGCGCAATCGGTTAGCGCGTTCGGCTGTTAACCGAAAGG |
| | | TTGGTGGTTCGAGCCCACCCAGGGACG |
| 447 | Gly_TCC_chr19:4724081-4724153 (+) | GGCGTTGGTGGTATAGTGGTTAGCATAGCTGCCTTCCAAGCAGTTGAC |
| | | CCGGGTTCGATTCCCGGCCAACGCA |
| 448 | Val_CAC_chr19:4724646-4724719 (-) | GTTTCCGTAGTGTAGCGGTTATCACATTCGCCTCACACGCGAAAGGTCC |
| | | CCGGTTCGATCCCGGGCGGAAACAG |
| 449 | Thr_AGT_chr19:33667962-33668036 (+) | TGGCGCCGTGGCTTAGTTGGTTAAAGCGCCTGTCTAGTAAACAGGAGA |
| | | TCCTGGGTTCGAATCCCAGCGGTGCCT |
| 450 | Ile_TAT_chr19:39902807-39902900 (-) | GCTCCAGTGGCGCAATCGGTTAGCGCGCGGTACTTATATGACAGTGCG |
| | | AGCGGAGCAATGCCGAGGTTGTGAGTTCGATCCTCACCTGGAGCAC |
| 451 | Gly_GCC_chr21:18827106-18827177 (-) | GCATGGGTGGTTCAGTGGTAGAATTCTCGCCTGCCACGCGGGAGGCCC |
| | | GGGTTCGATTCCCGGCCCATGCAG |

In an embodiment, a TREM (e.g., a TREM corresponding to a con-rare codon), e.g., an exogenous TREM, comprises 1, 2, 3, or 4 of the following properties:

(a) differs by at least one nucleotide or one post transcriptional modification from the closest sequence tRNA in a reference cell, e.g., a cell into which the exogenous nucleic acid is introduced;

(b) has been introduced into a cell other than the cell in which it was transcribed;

(c) is present in a cell other than one in which it naturally occurs; or

(d) has an expression profile, e.g., level or distribution, that is non-wildtype, e.g., it is expressed at a higher level than wildtype.

In an embodiment, the expression profile can be mediated by a change introduced into a nucleic acid that modulates expression, or by addition of an agent that modulates expression of the RNA molecule.

In an embodiment, a TREM (e.g., a TREM corresponding to a con-rare codon), e.g., an exogenous TREM comprises (a), (b), (c) and (d).

In an embodiment, a TREM (e.g., a TREM corresponding to a con-rare codon), e.g., an exogenous TREM comprises (a), (b) and (c).

In an embodiment, a TREM (e.g., a TREM corresponding to a con-rare codon), e.g., an exogenous TREM comprises (a), (b) and (d).

In an embodiment, a TREM (e.g., a TREM corresponding to a con-rare codon), e.g., an exogenous TREM comprises (a), (c) and (d).

In an embodiment, a TREM (e.g., a TREM corresponding to a con-rare codon), e.g., an exogenous TREM comprises (b), (c) and (d).

In an embodiment, a TREM (e.g., a TREM corresponding to a con-rare codon), e.g., an exogenous TREM comprises (a) and (d).

In an embodiment, a TREM (e.g., a TREM corresponding to a con-rare codon), e.g., an exogenous TREM comprises (c) and (d).

### TREM Fragments

In an embodiment, a TREM (e.g., a TREM corresponding to a con-rare codon) comprises a fragment (sometimes referred to herein as a TREM fragment), e.g., a fragment of a RNA encoded by a deoxyribonucleic acid sequence disclosed in Table 1. E.g., the TREM includes less than the full sequence of a tRNA, e.g., less than the full sequence of a tRNA with the same anticodon, from the same species as the subject being treated, or both. In an embodiment, the production of a TREM fragment, e.g., from a full length TREM or a longer fragment, can be catalyzed by an enzyme, e.g., an enzyme having nuclease activity (e.g., endonuclease activity or ribonuclease activity), e.g., Dicer, Angiogenin, RNaseP, RNaseZ, Rny1, or PrrC.

In an embodiment, a TREM fragment (e.g., a TREM fragment corresponding to a con-rare codon) can be produced in vivo, ex vivo or in vitro. In an embodiment, a TREM fragment is produced in vivo, in the host cell. In an embodiment, a TREM fragment is produced ex vivo. In an embodiment, a TREM fragment is produced in vitro, e.g., as described in Example 6. In an embodiment, the TREM fragment is produced by fragmenting an expressed TREM after production of the TREM by the cell, e.g., a TREM produced by the host cell is fragmented after release or purification from the host cell, e.g., the TREM is fragmented ex vivo or in vitro.

Exemplary TREM fragments include TREM halves (e.g., from a cleavage in the ACHD, e.g., 5′TREM halves or 3′ TREM halves), a 5′ fragment (e.g., a fragment comprising the 5′ end, e.g., from a cleavage in a DHD or the ACHD), a 3′ fragment (e.g., a fragment comprising the 3′ end of a TREM, e.g., from a cleavage in the THD), or an internal fragment (e.g., from a cleavage in one or more of the ACHD, DHD or THD).

In an embodiment, a TREM fragment (e.g., a TREM fragment corresponding to a con-rare codon) comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of an RNA sequence encoded by a DNA sequence provided in Table 1, e.g., any one of SEQ ID NOs: 1-451 disclosed in Table 1. In an embodiment, a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of an RNA sequence at least 80%, 85%, 90%, 95%, 96%, 97%, 98%, or 99% identical to an RNA sequence encoded by a DNA sequence provided in Table 1, e.g., any one of SEQ ID NOs: 1-451 disclosed in Table 1. In an embodiment, a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of an RNA sequence encoded by a DNA sequence at least 80%, 85%, 90%, 95%, 96%, 97%, 98%, or 99% identical to a DNA sequence provided in Table 1, e.g., any one of SEQ ID NOs: 1-451 disclosed in Table 1.

In an embodiment, a TREM fragment (e.g., a TREM corresponding to a con-rare codon) comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence encoded by a DNA sequence disclosed in Table 1, e.g., any one of SEQ ID NOs: 1-451 disclosed in Table 1. In an embodiment, a TREM fragment comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence which is at least 80%, 85%, 90%, 95%, 96%, 97%, 98%, 99% or 100% identical to an RNA sequence encoded by a DNA sequence provided in Table 1, e.g., any one of SEQ ID NOs: 1-451 disclosed in Table 1. In an embodiment, a TREM fragment comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence encoded by a DNA sequence with at least 80%, 82%, 85%, 87%, 88%, 90%, 92%, 95%, 96%, 97%, 98%, 99% or 100% identity to a DNA sequence provided in Table 1, e.g., any one of SEQ ID NOs: 1-451 disclosed in Table 1.

In an embodiment, a TREM fragment (e.g., a TREM corresponding to a con-rare codon) comprises a sequence of a length of between 10-90 ribonucleotides (rnt), between 10-80 rnt, between 10-70 rnt, between 10-60 rnt, between 10-50 rnt, between 10-40 rnt, between 10-30 rnt, between 10-20 rnt, between 20-90 rnt, between 20-80 rnt, 20-70 rnt, between 20-60 rnt, between 20-50 rnt, between 20-40 rnt, between 30-90 rnt, between 30-80 rnt, between 30-70 rnt, between 30-60 rnt, or between 30-50 rnt.

In an embodiment, a TREM fragment (e.g., a TREM corresponding to a con-rare codon) comprises a TREM structure, domain, or activity, e.g., as described herein above. In an embodiment, a TREM fragment comprises adaptor function, e.g., as described herein. In an embodiment, a TREM fragment comprises cognate adaptor function, e.g., as described herein. In an embodiment, a TREM fragment comprises non-cognate adaptor function, e.g., as described herein. In an embodiment, a TREM fragment comprises regulatory function, e.g., as described herein.

In an embodiment, a TREM fragment (e.g., a TREM corresponding to a con-rare codon) comprises translation inhibition function, e.g., displacement of an initiation factor, e.g., eIF4G.

In an embodiment, a TREM fragment (e.g., a TREM corresponding to a con-rare codon) comprises epigenetic function, e.g., epigenetic inheritance of a disorder, e.g., a metabolic disorder. In some embodiments, an epigenetic inheritance function can have a generational impact, e.g., as compared to somatic epigenetic regulation.

In an embodiment, a TREM fragment (e.g., a TREM corresponding to a con-rare codon) comprises retroviral regulation function, e.g., regulation of retroviral reverse transcription, e.g., HERV regulation.

In an embodiment, a TREM fragment (e.g., a TREM corresponding to a con-rare codon) comprises gene silencing function, e.g., by binding to AGO and/or PIWI.

In an embodiment, a TREM fragment (e.g., a TREM corresponding to a con-rare codon) comprises neuroprotectant function, e.g., by the sequestration of a translation initiation factor, e.g., in stress granules, to promote, e.g., motor neuron survival under cellular stress.

In an embodiment, a TREM fragment (e.g., a TREM corresponding to a con-rare codon) comprises anti-cancer function, e.g., by preventing cancer progression through the binding and/or sequestration of, e.g., metastatic transcript-stabilizing proteins.

In an embodiment, a TREM fragment (e.g., a TREM corresponding to a con-rare codon) comprises cell survival function, e.g., increased cell survival, by binding to, e.g., cytochrome c and/or cyt c ribonucleoprotein complex.

In an embodiment, a TREM fragment (e.g., a TREM corresponding to a con-rare codon) comprises ribosome biogenesis function, e.g., a TREM fragment can regulate ribosome biogenesis by, e.g., regulation of, e.g., binding to, an mRNA coding for ribosomal proteins.

### TREM Modifications

A TREM described herein (e.g., a TREM corresponding to a con-rare codon), can comprise a moiety, often referred to herein as a modification, e.g., a moiety described in Table 2. While the term modification as used herein should not generally be construed to be the product of any particular process, in embodiments, the formation of a modification can be mediated by an enzyme in Table 2. In embodiments, the modification is formed post-transcriptionally. In embodiments, the modification is formed co-transcriptionally. In an embodiment, the modification occurs in vivo, e.g., in the host cell.

In an embodiment, the modification is a modification listed in any of rows 1-62 of Table 2. In an embodiment, the modification is a modification listed in any of rows 1-62 of Table 2, and the formation of the modification is mediated by an enzyme in Table 2. In an embodiment the modification is selected from a row in Table 2 and the formation of the modification is mediated by an enzyme from the same row in Table 2.

TABLE 2
List of tRNA modifications and associated enzymes.

| | Short | | |
|---|---|---|---|
| | Name | Modification | Enzyme list |
| 1 | m1Am | 1,2'-O-dimethyladenosine | METTL3 |
| 2 | imG | wyosine | Trm5, Tywl, Tyw2, Tyw3, and Tyw4 |
| 3 | m5s2U | 5-methyl-2-thiouridine | TrmU |
| 4 | m6t6A | N6-methyl-N6-threonylcarbamoyladenosine | TRMO, TrmO |
| 5 | QtRNA | queuosine | TGTase |
| 6 | OHyW | hydroxywybutosine | Trm5, TYW1, TYW2, TYW3, TYW4 |
| 7 | io6A | N6-(cis-hydroxyisopentenyl)adenosine | TRIT1 |
| 8 | Gr(p) | 2'-O-ribosylguanosine (phosphate) | |
| 9 | ho5U | 5-hydroxyuridine | |
| 10 | ncm5Um | 5-carbamoylmethyl-2'-O-methyluridine | ELP1, ELP2, ELP3, ELP4, ELP5, ELP6, |
| | | | KTI111, KTI112, KTI113, Uba4, Urm1, Tum1, |
| | | | Ncs6, Ncs2, Trm9, Sit4, Isu1, Isu2, Sap185, |
| | | | Sap190 |
| 11 | OHyW* | hydroxywybutosine | wybutosine hydroxylases |
| 12 | acp3U | 3-(3-amino-3-carboxypropyl)uridine | |
| 13 | mcm5s2U | 5-methoxycarbonylmethyl-2-thiouridine | ALKBH8, Ncs6, Trm9, Ncs2, TrmU, |
| | | | CTU1, CTU2, ELP1, ELP2, ELP3, ELP4, ELP5, |
| | | | ELP6 |
| 14 | m5U | 5-methyluridine | Trm2 |
| 15 | D | dihydrouridine | DUS1, DUS2, DUS3, DUS4 |
| 16 | mcm5Um | 5-methoxycarbonylmethyl-2'-O- | ELP1, ELP2, ELP3, ELP4, ELP5, ELP6,Trm9, |
| | | methyluridine | ALKBH-MT, ? |
| 17 | m5C | 5-methylcytidine | Dnmt2, Dnmt2, EfmM, Nop2, Rcm1, Rlm1, |
| | | | RlmO, RsmB, RsmF, Trm4, nsun2 |
| 18 | ac4C | N4-acetylcytidine | NAT10, Rra1, TmcA |
| 19 | m1A | 1-methyladenosine | Bmt2, KamB, NpmA, Rrp8, TRMT10C, |
| | | | Trm61, TrmI, TrmK, Trmt61A, Trmt61B |
| 20 | tm5U | 5-taurinomethyluridine | MTU1 |
| 21 | m1G | 1-methylguanosine | AviRa, RImA(I), RlmA(II), TRM5, TRMT10A, |
| | | | TRMT10B, TRMT10C, Taw22, Trm10, Trm5, |
| | | | Trmb, TrmD |
| 22 | Cm | 2-O-methylcytidine | |
| 23 | m1I | 1-methylinosine | |
| 24 | Ar(p) | 2'O-ribosyladenosine (phosphate) | |
| 25 | galQtRNA | galactosyl-queuosine | |
| 26 | mcm5U | 5-methoxycarbonylmethyluridine | ALKBH8, Trm9, ELP1, ELP2, ELP3, ELP4, |
| | | | ELP5, ELP6 |
| 27 | m1Y | 1-methylpseudouridine | |
| 28 | Gm | 2'O-methylguanosine | MRM1, Mrm1, Nop1, RNMTL1, RlmB, Spb1, |
| | | | Trm3, Trm7, TrmH |
| 29 | manQtRNA | mannosyl-queuosine | Man/Gal-Q-transferase |
| 30 | yW | wybutosine | TYW1, 2, 3, 4 |
| 31 | f5C | 5-formylcytidine | MTU1 |
| 32 | tm5s2U | 5-taurinomethyl-2-thiouridine | TrmU |
| 33 | m2, 2G | N2,N2-dimethylguanosine | Trm1 |
| 34 | chm5U | 5-carboxyhydroxymethyluridine | |
| 35 | s2U | 2-thiouridine | MnmA, Mtu1, Ncs2, Ncs6, TrmU |
| 36 | mnm5s2U | 5-methylaminomethyl-2-thiouridine | MnmCD, MnmD, MnmA, Mtu1, TrmU |
| 37 | m6A | N6-methyladenosine | ErmAM, ErmBC, ErmC′, Ime4, METTL14, |
| | | | METTL3, RlmF, RlmJ, RsmA, TrmM |
| 38 | mchm5U | 5-(carboxyhydroxymethyl)uridine methyl | ALKBH8 |
| | | ester | |
| 39 | m2G | N2-methylguanosine | Trm112, Trm11 |
| 40 | cmnm5U | 5-carboxymethylaminomethyluridine | tRNA (cytidine(34)-2'-O)-methyltransferase |
| 41 | Ym | 2'O-methylpseudouridine | NEP1 |
| 42 | f5Cm | 5-formyl-2'-O-methylcytidine | |
| 43 | ncm5U | 5-carbamoylmethyluridine | ELP1, ELP2, ELP3, ELP4, ELP5, ELP6 |
| 44 | I | inosine | Tad1, Tad2, Tad3, TadA |
| 45 | g6A | N6-glycinylcarbamoyladenosine | METTL8 |
| 46 | cmnm5s2U | 5-carboxymethylaminomethyl-2-thiouridine | MnmA, Mtu1, TrmU, MnmE, MnmG, Mss1, |
| | | | Mto1 |
| 47 | Um | 2'O-methyluridine | AviRb, MRM2, Mrm2, Nop1, RlmE, Spb1, |
| | | | Trm44, TrmJ, TrmL, aTrm56 |
| 48 | Y | pseudouridine | Cbf5, Pus1, Pus10, Pus2, Pus3, Pus4, Pus5, |
| | | | Pus6, Pus7, Pus8, Pus9, RluA, RluB, RluC, |
| | | | RluD, RluE, RluF, TruA, TruB, TruC, TruD |
| 49 | ms2i6A | 2-methylthio-N6-isopentenyladenosine | MiaA |
| 50 | m3C | 3 -methylcytidine | Trm140, METTL2 and METTL6 |
| 51 | o2yW | peroxywybutosine | TRM5, TYW1, TYW2, TYW3, TYW4, TYW5, |
| | | | TRM4 |
| 52 | m5Um | 5,2'O-dimethyluridine | |
| 53 | ms2t6A | 2-methylthio-N6- | Yrdc/Sua5, MtaB/e-MtaB, SAM, “S” |
| | | threonylcarbamoyladenosine | |
| 54 | i6A | N6-isopentenyladenosine | MiaA, Mod5 |
| 55 | ms2io6A | 2-methylthio-N6-(cis-hydroxyisopentenyl) | MiaE |
| | | adenosine | |
| 56 | Am | 2_-O-methyladenosine | (2'-O-methyladenosine-N6-)-methyltransferase |
| 57 | m7G | 7-methylguanosine | Abd1, ArmA, Bud23, RlmKL, RmtB, RsmG, |
| | | | Sgm, TRMB, Trm8, TrmB, WDR4 |
| 58 | t6A | N6-threonylcarbamoyladenosine | Bud32, Gon7, Cgi121 |
| 59 | | N1-methy Iguanine | Trm10 |
| 60 | | N7-methy Iguanine | Trm8, Trm82 |
| 61 | | 2'-O methylribose | Trm3, Trm13, Trm44, Trm7, Trm732, Rtt10 |
| 62 | | Ribose 2'-O-ribosyl phosphate | Rit1 |

### TREM Fusion

In an embodiment, a TREM disclosed herein (e.g., a TREM corresponding to a con-rare codon), comprises an additional moiety, e.g., a fusion moiety. In an embodiment, the fusion moiety can be used for purification, to alter folding of the TREM, or as a targeting moiety. In an embodiment, the fusion moiety can comprise a tag, a linker, can be cleavable or can include a binding site for an enzyme. In an embodiment, the fusion moiety can be disposed at the N terminal of the TREM or at the C terminal of the TREM. In an embodiment, the fusion moiety can be encoded by the same or different nucleic acid molecule that encodes the TREM.

### TREM Consensus Sequence

In an embodiment, a TREM disclosed herein (e.g., a TREM corresponding to a con-rare codon), comprises a consensus sequence provided herein.

In an embodiment, a TREM disclosed herein (e.g., a TREM corresponding to a con-rare codon), comprises a consensus sequence of Formula I zzz, wherein zzz indicates any of the twenty amino acids and Formula I corresponds to all species.

In an embodiment, a TREM disclosed herein (e.g., a TREM corresponding to a con-rare codon), comprises a consensus sequence of Formula II zzz, wherein zzz indicates any of the twenty amino acids and Formula II corresponds to mammals.

In an embodiment, a TREM disclosed herein (e.g., a TREM corresponding to a con-rare codon), comprises a consensus sequence of Formula III zzz, wherein zzz indicates any of the twenty amino acids and Formula III corresponds to humans.

In an embodiment, a TREM disclosed herein (e.g., a TREM corresponding to a con-rare codon), comprises a property selected from the following:

a) under physiological conditions residue R₀ forms a linker region, e.g., a Linker 1 region;

b) under physiological conditions residues R₁—R₂—R₃—R₄—R₅—R₆—R₇ and residues R₆₅—R₆₆—R₆₇—R₆₈—R₆₉—R₇₀—R₇₁ form a stem region, e.g., an AStD stem region;

c) under physiological conditions residues R₈—R₉ forms a linker region, e.g., a Linker 2 region;

d) under physiological conditions residues —R₁₀—R₁₁—R₁₂—R₁₃—R₁₄R₁₅—R₁₆—R₁₇—R₁₈—R₁₉—R₂₀—R₂₁—R₂₂—R₂₃—R₂₄—R₂₅—R₂₆—R₂₇—R₂₈ form a stem-loop region, e.g., a D arm Region;

e) under physiological conditions residue —R₂₉ forms a linker region, e.g., a Linker 3 Region;

f) under physiological conditions residues —R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆ form a stem-loop region, e.g., an AC arm region;

g) under physiological conditions residue —[R₄₇]ₓ₁ comprises a variable region, e.g., as described herein;

h) under physiological conditions residues —R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄ form a stem-loop region, e.g., a T arm Region; or

i) under physiological conditions residue R₇₂ forms a linker region, e.g., a Linker 4 region.

### Alanine TREM Consensus Sequence

In an embodiment, a TREM disclosed herein comprises the sequence of Formula I_{ALA},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Ala is:
Rₒ=absent;
R₁₄, R₅₇=are independently A or absent;
R₂₆=A, C, G or absent;
R₅, R₆, R₁₅, R₁₆, R₂₁, R₃₀, R₃₁, R₃₂, R₃₄, R₃₇, R₄₁, R₄₂, R₄₃, R₄₄, R₄₅, R₄₈, R₄₉, R₅₀, R₅₈, R₅₉, R₆₃, R₆₄, R₆₆, R₆₇=are independently N or absent;
R₁₁, R₃₅, R₆₅=are independently A, C, U or absent;
R₁, R₉, R₂₀, R₃₈, R₄₀, R₅₁, R₅₂, R₅₆=are independently A, G or absent;
R₇, R₂₂, R₂₅, R₂₇, R₂₉, R₄₆, R₅₃, R₇₂=are independently A, G, U or absent;
R₂₄, R₆₉=are independently A, U or absent;
R₇₀, R₇₁=are independently C or absent;
R₃, R₄=are independently C, G or absent;
R₁₂, R₃₃, R₃₆, R₆₂, R₆₈=are independently C, G, U or absent;
R₁₃, R₁₇, R₂₈, R₃₉, R₅₅, R₆₀, R₆₁=are independently C, U or absent;
R₁₀, R₁₉, R₂₃=are independently G or absent;
R₂=G, U or absent;
R₈, R₁₈, R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{ALA},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Ala is:
Rₒ, R₁₈=are absent;
R₁₄, R₂₄, R₅₇=are independently A or absent;
R₁₅, R₂₆, R₆₄=are independently A, C, G or absent;
R₁₆, R₃₁, R₅₀, R₅₉=are independently N or absent;
R₁₁, R₃₂, R₃₇, R₄₁, R₄₃, R₄₅, R₄₉, R₆₅, R₆₆=are independently A, C, U or absent;
R₁, R₅, R₉, R₂₅, R₂₇, R₃₈, R₄₀, R₄₆, R₅₁, R₅₆=are independently A, G or absent;
R₇, R₂₂, R₂₉, R₄₂, R₄₄, R₅₃, R₆₃, R₇₂=are independently A, G, U or absent;
R₆, R₃₅, R₆₉=are independently A, U or absent;
R₅₅, R₆₀, R₇₀, R₇₁=are independently C or absent;
R₃=C, G or absent;
R₁₂, R₃₆, R₄₈=are independently C, G, U or absent;
R₁₃, R₁₇, R₂₈, R₃₀, R₃₄, R₃₉, R₅₈, R₆₁, R₆₂, R₆₇, R₆₈=are independently C, U or absent;
R₄, R₁₀, R₁₉, R₂₀, R₂₃, R₅₂=are independently G or absent;
R₂, R₈, R₃₃=are independently G, U or absent;
R₂₁, R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{ALA},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Ala is:
Rₒ, R₁₈=are absent;
R₁₄, R₂₄, R₅₇, R₇₂=are independently A or absent;
R₁₅, R₂₆, R₆₄=are independently A, C, G or absent;
R₁₆, R₃₁, R₅₀=are independently N or absent;
R₁₁, R₃₂, R₃₇, R₄₁, R₄₃, R₄₅, R₄₉, R₆₅, R₆₆=are independently A, C, U or absent;
R₅, R₉, R₂₅, R₂₇, R₃₈, R₄₀, R₄₆, R₅₁, R₅₆=are independently A, G or absent;
R₇, R₂₂, R₂₉, R₄₂, R₄₄, R₅₃, R₆₃=are independently A, G, U or absent;
R₆, R₃₅=are independently A, U or absent;
R₅₅, R₆₀, R₆₁, R₇₀, R₇₁=are independently C or absent;
R₁₂, R₄₈, R₅₉=are independently C, G, U or absent;
R₁₃, R₁₇, R₂₈, R₃₀, R₃₄, R₃₉, R₅₈, R₆₂, R₆₇, R₆₈=are independently C, U or absent;
R₁, R₂, R₃, R₄, R₁₀, R₁₉, R₂₀, R₂₃, R₅₂=are independently G or absent;
R₃₃, R₃₆=are independently G, U or absent;
R₈, R₂₁, R₅₄, R₆₉=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Arginine TREM Consensus Sequence

In an embodiment, a TREM disclosed herein comprises the sequence of Formula I _{ARG},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Arg is:
R₅₇=A or absent;
R₉,R₂₇=are independently A,C,G or absent;
R₁,R₂,R₃,R₄,R₅,R₆,R₇,R₁₁,R₁₂,R₁₆,R₂₁,R₂₂,R₂₃,R₂₅,R₂₆,R₂₉,R₃₀,R₃₁,R₃₂,R₃₃,R₃₄,R₃₇,R₄₂,R₄₄,R₄₅, R₄₆,R₄₈,R₄₉,R₅₀,R₅₁,R₅₈,R₆₂,R₆₃,R₆₄,R₆₅,R₆₆,R₆₇,R₆₈,R₆₉,R₇₀,R₇₁=are independently N or absent;
R₁₃,R₁₇,R₄₁=are independently A,C,U or absent;
R₁₉,R₂₀,R₂₄,R₄₀,R₅₆=are independently A,G or absent;
R₁₄,R₁₅,R₇₂=are independently A,G,U or absent;
R₁₈=A,U or absent;
R₃₈=C or absent;
R₃₅,R₄₃,R₆₁=are independently C,G,U or absent;
R₂₈,R₅₅,R₅₉,R₆₀=are independently C,U or absent;
Rₒ,R₁₀,R₅₂=are independently G or absent;
R₈,R₃₉=are independently G,U or absent;
R₃₆,R₅₃,R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{ARG},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₄—R₆₅—R₆₆—R₆₇—R₆₈—R₆₉—R₇₀—R₇₁—R₇₂
wherein the consensus for Arg is:
R₁₈=absent;
R₂₄,R₅₇=are independently A or absent;
R₄₁=A,C or absent;
R₃,R₇,R₃₄,R₅₀=are independently A,C,G or absent;
R₂,R₅,R₆,R₁₂,R₂₆,R₃₂,R₃₇,R₄₄,R₅₈,R₆₆,R₆₇,R₆₈,R₇₀=are independently N or absent;
R₄₉,R₇₁=are independently A,C,U or absent;
R₁,R₁₅,R₁₉,R₂₅,R₂₇,R₄₀,R₄₅,R₄₆,R₅₆,R₇₂=are independently A,G or absent;
R₁₄,R₂₉,R₆₃=are independently A,G,U or absent;
R₁₆,R₂₁=are independently A,U or absent;
R₃₈,R₆₁=are independently C or absent;
R₃₃,R₄₈=are independently C,G or absent;
R₄,R₉,R₁₁,R₄₃,R₆₂,R₆₄,R₆₉=are independently C,G,U or absent;
R₁₃,R₂₂,R₂₈,R₃₀,R₃₁,R₃₅,R₅₅,R₆₀,R₆₅=are independently C,U or absent;
Rₒ,R₁₀,R₂₀,R₂₃,R₅₁,R₅₂=are independently G or absent;
R₈,R₃₉,R₄₂=are independently G,U or absent;
R₁₇,R₃₆,R₅₃,R₅₄,R₅₉=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{ARG},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂
wherein the consensus for Arg is:
R₁₈=is absent;
R₁₅,R₂₁,R₂₄,R₄₁,R₅₇=are independently A or absent;
R₃₄,R₄₄=are independently A,C or absent;
R₃,R₅,R₅₈=are independently A,C,G or absent;
R₂,R₆,R₆₆,R₇₀=are independently N or absent;
R₃₇,R₄₉=are independently A,C,U or absent;
R₁,R₂₅,R₂₉,R₄₀,R₄₅,R₄₆,R₅₀=are independently A,G or absent;
R₁₄,R₆₃,R₆₈=are independently A,G,U or absent;
R₁₆=A,U or absent;
R₃₈,R₆₁=are independently C or absent;
R₇,R₁₁,R₁₂,R₂₆,R₄₈=are independently C,G or absent;
R₆₄,R₆₇,R₆₉=are independently C,G,U or absent;
R₄,R₁₃,R₂₂,R₂₈,R₃₀,R₃₁,R₃₅,R₄₃,R₅₅,R₆₀,R₆₂,R₆₅,R₇₁=are independently C,U or absent;
Rₒ,R₁₀,R₁₉,R₂₀,R₂₃,R₂₇,R₃₃,R₅₁,R₅₂,R₅₆,R₇₂=are independently G or absent;
R₈,R₉,R₃₂,R₃₉,R₄₂=are independently G,U or absent;
R₁₇,R₃₆,R₅₃,R₅₄,R₅₉=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Asparagine TREM Consensus Sequence

In an embodiment, a TREM disclosed herein comprises the sequence of Formula I _{ASN},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Asn is:
Rₒ,R₁₈=are absent;
R₄₁=A or absent;
R₁₄,R₄₈,R₅₆=are independently A,C,G or absent;
R₂,R₄,R₅,R₆,R₁₂,R₁₇,R₂₆,R₂₉,R₃,R₃₁,R₄₄,R₄₅,R₄₆,R₄₉,R₅₀,R₅₈,R₆₂,R₆₃,R₆₅,R₆₆,R₆₇,R₆₈,R₇₀,R₇₁=are independently N or absent;
R₁₁,R₁₃,R₂₂,R₄₂,R₅₈,R₅₉=are independently A,C,U or absent;
R₉,R₁₅,R₂₄,R₂₇,R₃₄,R₃₇,R₅₁,R₇₂=are independently A,G or absent;
R₁,R₇,R₂₅,R₆₉=are independently A,G,U or absent;
R₄₀,R₅₇=are independently A,U or absent;
R₆₀=C or absent;
R₃₃=C,G or absent;
R₂₁,R₃₂,R₄₃,R₆₄=are independently C,G,U or absent;
R₃,R₁₆,R₂₈,R₃₅,R₃₆,R₆₁=are independently C,U or absent;
R₁₀,R₁₉,R₂₀,R₅₂=are independently G or absent;
R₅₄=G,U or absent;
R₈,R₂₃,R₃₈,R₃₉,R₅₃=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{ASN},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Asn is:
R₀,R₁₈=are absent
R₂₄,R₄₁,R₄₆,R₆₂=are independently A or absent;
R₅₉=A,C or absent;
R₁₄,R₅₆,R₆₆=are independently A,C,G or absent;
R₁₇,R₂₉=are independently N or absent;
R₁₁,R₂₆,R₄₂,R₅₅=are independently A,C,U or absent;
R₁,R₉,R₁₂,R₁₅,R₂₅,R₃₄,R₃₇,R₄₈,R₅₁,R₆₇,R₆₈,R₆₉,R₇₀,R₇₂=are independently A,G or absent;
R₄₄,R₄₅,R₅₈=are independently A,G,U or absent;
R₄₀,R₅₇=are independently A,U or absent;
R₅,R₂₈,R₆₀=are independently C or absent;
R₃₃,R₆₅=are independently C,G or absent;
R₂₁,R₄₃,R₇₁=are independently C,G,U or absent;
R₃,R₆,R₁₃,R₂₂,R₃₂,R₃₅,R₃₆,R₆₁,R₆₃,R₆₄=are independently C,U or absent;
R₇,R₁₀,R₁₉,R₂₀,R₂₇,R₄₉,R₅₂=are independently G or absent;
R₅₄=G,U or absent;
R₂,R₄,R₅,R₁₆,R₂₃,R₃₀,R₃₁,R₃₈,R₃₉,R₅₀,R₅₃=are independently U or absent; [R₄₇]ₓi=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{ASN},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Asn is:
R₀,R₁₈=are absent
R₂₄,R₄₀,R₄₁,R₄₆,R₆₂=are independently A or absent;
R₅₉=A,C or absent;
R₁₄,R₅₆,R₆₆=are independently A,C,G or absent;
R₁₁,R₂₆,R₄₂,R₅₅=are independently A,C,U or absent;
R₁,R₉,R₁₂,R₁₅,R₃₄,R₃₇,R₄₈,R₅₁,R₆₇,R₆₈,R₆₉,R₇₀=are independently A,G or absent;
R₄₄,R₄₅,R₅₈=are independently A,G,U or absent;
R₅₇=A,U or absent;
R₅,R₂₈,R₆₀=are independently C or absent;
R₃₃,R₆₅=are independently C,G or absent;
R₁₇,R₂₁,R₂₉=are independently C,G,U or absent;
R₃,R₆,R₁₃,R₂₂,R₃₂,R₃₅,R₃₆,R₄₃,R₆₁,R₆₃,R₆₄,R₇₁=are independently C,U or absent;
R₇,R₁₀,R₁₉,R₂₀,R₂₅,R₂₇,R₄₉,R₅₂,R₇₂=are independently G or absent;
R₅₄=G,U or absent;
R₂,R₄,R₈,R₁₆,R₂₃,R₃₀,R₃₁,R₃₈,R₃₉,R₅₀,R₅₃=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Aspartate TREM Consensus Sequence

In an embodiment, a TREM disclosed herein comprises the sequence of Formula I _{ASP},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Asp is:
R₀=absent
R₂₄,R₇₁=are independently A,C or absent;
R₃₃,R₄₆=are independently A,C,G or absent;
R₂,R₃,R₄,R₅,R₆,R₁₂,R₁₆,R₂₂,R₂₆,R₂₉,R₃₁,R₃₂,R₄₄,R₄₈,R₄₉,R₅₈,R₆₃,R₆₄,R₆₆,R₆₇,R₆₈,R₆₉=are independently N or absent;
R₁₃,R₂₁,R₃₄,R₄₁,R₅₇,R₆₅=are independently A,C,U or absent;
R₉,R₁₀,R₁₄,R₁₅,R₂₀,R₂₇,R₃₇,R₄₀,R₅₁,R₅₆,R₇₂=are independently A,G or absent;
R₇,R₂₅,R₄₂=are independently A,G,U or absent;
R₃₉=C or absent;
R₅₀,R₆₂=are independently C,G or absent;
R₃₀,R₄₃,R₄₅,R₅₅,R₇₀=are independently C,G,U or absent;
R₈,R₁₁,R₁₇,R₁₈,R₂₈,R₃₅,R₅₃,R₅₉,R₆₀,R₆₁=are independently C,U or absent;
R₁₉,R₅₂=are independently G or absent;
R₁=G,U or absent;
R₂₃,R₃₆,R₃₈,R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{ASP},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Asp is:
R₀,R₁₇,R₁₈,R₂₃=are independently absent;
R₉,R₄₀=are independently A or absent; R₂₄,R₇₁=are independently A,C or absent;
R₆₇,R₆₈=are independently A,C,G or absent;
R₂,R₆,R₆₆=are independently N or absent;
R₅₇,R₆₃=are independently A,C,U or absent;
R₁₀,R₁₄,R₂₇,R₃₃,R₃₇,R₄₄,R₄₆,R₅₁,R₅₆,R₆₄,R₇₂=are independently A,G or absent;
R₇,R₁₂,R₂₆,R₆₅=are independently A,U or absent;
R₃₉,R₆₁,R₆₂=are independently C or absent;
R₃,R₃₁,R₄₅,R₇₀=are independently C,G or absent;
R₄,R₅,R₂₉,R₄₃,R₅₅=are independently C,G,U or absent;
R₈,R₁₁,R₁₃,R₃₀,R₃₂,R₃₄,R₃₅,R₄₁,R₄₈,R₅₃,R₅₉,R₆₀=are independently C,U or absent;
R₁₅,R₁₉,R₂₀,R₂₅,R₄₂,R₅₀,R₅₂=are independently G or absent;
R₁,R₂₂,R₄₉,R₅₈,R₆₉=are independently G,U or absent;
R₁₆,R₂₁,R₂₈,R₃₆,R₃₈,R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{ASP},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Asp is:
R₀,R₁₇,R₁₈,R₂₃=are absent
R₉,R₁₂,R₄₀,R₆₅,R₇₁=are independently A or absent;
R₂,R₂₄,R₅₇=are independently A,C or absent;
R₆,R₁₄,R₂₇,R₄₆,R₅₁,R₅₆,R₆₄,R₆₇,R₆₈=are independently A,G or absent;
R₃,R₃₁,R₃₅,R₃₉,R₆₁,R₆₂=are independently C or absent;
R₆₆=C,G or absent;
R₅,R₈,R₂₉,R₃₀,R₃₂,R₃₄,R₄₁,R₄₃,R₄₈,R₅₅,R₅₉,R₆₀,R₆₃=are independently C,U or absent;
R₁₀,R₁₅,R₁₉,R₂₀,R₂₅,R₃₃,R₃₇,R₄₂,R₄₄,R₄₅,R₄₉,R₅₀,R₅₂,R₆₉,R₇₀,R₇₂=are independently G or absent;
R₂₂,R₅₈=are independently G,U or absent;
R₁,R₄,R₇,R₁,R₁₃,R₁₆,R₂₁,R₂₆,R₂₈,R₃₆,R₃₈,R₅₃,R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Cysteine TREM Consensus Sequence

In an embodiment, a TREM disclosed herein comprises the sequence of Formula I _{CYS},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Cys is:
R₀=absent
R₁₄,R₃₉,R₅₇=are independently A or absent;
R₄₁=A,C or absent;
R₁₀,R₁₅,R₂₇,R₃₃,R₆₂=are independently A,C,G or absent;
R₃,R₄,R₅,R₆,R₁₂,R₁₃,R₁₆,R₂₄,R₂₆,R₂₉,R₃₀,R₃₁,R₃₂,R₃₄,R₄₂,R₄₄,R₄₅,R₄₆,R₄₈,R₄₉,R₅₈,R₆₃,R₆₄,R₆₆, R₆₇,R₆₈,R₆₉,R₇₀=are independently N or absent;
R₆₅=A,C,U or absent;
R₉,R₂₅,R₃₇,R₄₀,R₅₂,R₅₆=are independently A,G or absent;
R₇,R₂₀,R₅₁=are independently A,G,U or absent;
R₁₈,R₃₈,R₅₅=are independently C or absent;
R₂=C, G or absent;
R₂₁,R₂₈,R₄₃,R₅₀=are independently C,G,U or absent;
R₁₁,R₂₂,R₂₃,R₃₅,R₃₆,R₅₉,R₆₀,R₆₁,R₇₁,R₇₂=are independently C,U or absent; R₁,R₁₉=are independently G or absent;
R₁₇=G,U or absent;
R₈,R₅₃,R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{CYS},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Cys is:
R₀,R₁₈,R₂₃=are absent;
R₁₄,R₂₄,R₂₆,R₂₉,R₃₉,R₄₁,R₄₅,R₅₇=are independently A or absent;
R₄₄=A,C or absent;
R₂₇,R₆₂=are independently A,C,G or absent;
R₁₆=A,C,G,U or absent;
R₃₀,R₇₀=are independently A,C,U or absent;
R₅,R₇,R₉,R₂₅,R₃₄,R₃₇,R₄₀,R₄₆,R₅₂,R₅₆,R₅₈,R₆₆=are independently A,G or absent;
R₂₀,R₅₁=are independently A,G,U or absent;
R₃₅,R₃₈,R₄₃,R₅₅,R₆₉=are independently C or absent;
R₂,R₄,R₁₅=are independently C,G or absent;
R₁₃=C,G,U or absent;
R₆,R₁₁,R₂₈,R₃₆,R₄₈,R₄₉,R₅₀,R₆₀,R₆₁,R₆₇,R₆₈,R₇₁,R₇₂=are independently C,U or absent;
R₁,R₃,R₁₀,R₁₉,R₃₃,R₆₃=are independently G or absent;
R₈,R₁₇,R₂₁,R₆₄=are independently G,U or absent;
R₁₂,R₂₂,R₃₁,R₃₂,R₄₂,R₅₃,R₅₄,R₆₅=are independently U or absent;
R₅₉=U, or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{CYS},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Cys is:
R₀,R₁₈,R₂₃=are absent
R₁₄,R₂₄,R₂₆,R₂₉,R₃₄,R₃₉,R₄₁,R₄₅,R₅₇,R₅₈=are independently A or absent;
R₄₄,R₇₀=are independently A,C or absent;
R₆₂=A,C,G or absent;
R₁₆=N or absent;
R₅,R₇,R₉,R₂₀,R₄₀,R₄₆,R₅₁,R₅₂,R₅₆,R₆₆=are independently A,G or absent;
R₂₈,R₃₅,R₃₈,R₄₃,R₅₅,R₆₇,R₆₉=are independently C or absent;
R₄,R₁₅=are independently C,G or absent;
R₆,R₁₁,R₁₃,R₃₀,R₄₈,R₄₉,R₅₀,R₆₀,R₆₁,R₆₈,R₇₁,R₇₂=are independently C,U or absent;
R₁,R₂,R₃,R₁₀,R₁₉,R₂₅,R₂₇,R₃₃,R₃₇,R₆₃=are independently G or absent;
R₈,R₂₁,R₆₄=are independently G,U or absent;
R₁₂,R₁₇,R₂₂,R₃₁,R₃₂,R₃₆,R₄₂,R₅₃,R₅₄, R₅₉,R₆₅=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Glutamine TREM Consensus Sequence

In an embodiment, a TREM disclosed herein comprises the sequence of Formula I _{GLN},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Gln is:
Rₒ,R₁₈=are absent;
R₁₄,R₂₄,R₅₇=are independently A or absent;
R₉,R₂₆,R₂₇,R₃₃,R₅₆=are independently A,C,G or absent;
R₂,R₄,R₅,R₆,R₁₂,R₁₃,R₁₆,R₂₁,R₂₂,R₂,R₂₉,R₃₀,R₃₁,R₃₂,R₃₄,R₄₁,R₄₂,R₄₄,R₄₅,R₄₆,R₄₈,R₄₉,R₅₀,R₅₈,R₆₂,R₆₃,R₆₆,R₆₇,R₆₈,R₆₉,R₇₀=are independently N or absent;
R₁₇,R₂₃,R₄₃,R₆₅,R₇₁=are independently A,C,U or absent;
R₁₅,R₄₀,R₅₁,R₅₂=are independently A,G or absent;
R₁,R₇,R₇₂=are independently A,G,U or absent;
R₃,R₁₁,R₃₇,R₆₀,R₆₄=are independently C,G,U or absent;
R₂₈,R₃₅,R₅₅,R₅₉,R₆₁=are independently C,U or absent;
R₁₀,R₁₉,R₂₀=are independently G or absent;
R₃₉=G,U or absent;
R₈,R₃₆,R₃₈,R₅₃,R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{GLN},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Gln is:
R₀,R₁₈,R₂₃=are absent
R₁₄,R₂₄,R₅₇=are independently A or absent;
R₁₇,R₇₁=are independently A,C or absent;
R₂₅,R₂₆,R₃₃,R₄₄,R₄₆,R₅₆,R₆₉=are independently A,C,G or absent;
R₄,R₅,R₁₂,R₂₂,R₂₉,R₃₀,R₄₈,R₄₉,R₆₃,R₆₇,R₆₈=are independently N or absent;
R₃₁,R₄₃,R₆₂,R₆₅,R₇₀=are independently A,C,U or absent;
R₁₅,R₂₇,R₃₄,R₄₀,R₄₁,R₅₁,R₅₂=are independently A,G or absent;
R₂,R₇,R₂₁,R₄₅,R₅₀,R₅₈,R₆₆,R₇₂=are independently A,G,U or absent;
R₃,R₁₃,R₃₂,R₃₇,R₄₂,R₆₀,R₆₄=are independently C,G,U or absent;
R₆,R₁₁,R₂₈,R₃₅,R₅₅,R₅₉,R₆₁=are independently C,U or absent;
R₉,R₁₀,R₁₉,R₂₀=are independently G or absent;
R₁,R₁₆,R₃₉=are independently G,U or absent;
R₈,R₃₆,R₃₈,R₅₃,R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{GLN},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Gln is:
R₀,R₁₈,R₂₃=are absent
R₁₄,R₂₄,R₄₁,R₅₇=are independently A or absent;
R₁₇,R₇₁=are independently A,C or absent;
R₅,R₂₅,R₂₆,R₄₆,R₅₆,R₆₉=are independently A,C,G or absent;
R₄,R₂₂,R₂₉,R₃₀,R₄₈,R₄₉,R₆₃,R₆₈=are independently N or absent;
R₄₃,R₆₂,R₆₅,R₇₀=are independently A,C,U or absent;
R₁₅,R₂₇,R₃₃,R₃₄,R₄₀,R₅₁,R₅₂=are independently A,G or absent;
R₂,R₇,R₁₂,R₄₅,R₅₀,R₅₈,R₆₆=are independently A,G,U or absent;
R₃₁=A,U or absent;
R₃₂,R₄₄,R₆₀=are independently C,G or absent;
R₃,R₁₃,R₃₇,R₄₂,R₆₄,R₆₇=are independently C,G,U or absent;
R₆,R₁₁,R₂₈,R₃₅,R₅₅,R₅₉,R₆₁=are independently C,U or absent;
R₉,R₁₀,R₁₉,R₂₀=are independently G or absent;
R₁,R₂₁,R₃₉,R₇₂=are independently G,U or absent;
R₈,R₁₆,R₃₆,R₃₈,R₅₃,R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Glutamate TREM Consensus Sequence

In an embodiment, a TREM disclosed herein comprises the sequence of Formula I _{GLU},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Glu is:
Rₒ=absent;
R₃₄,R₄₃,R₆₈,R₆₉=are independently A,C,G or absent;
R₁,R₂,R₅,R₆,R₉,R₁₂,R₁₆,R₂₀,R₂₁,R₂₆,R₂₇,R₂₉,R₃₀,R₃₁,R₃₂,R₃₃,R₄₁,R₄₄,R₄₅,R₄₆,R₄₈,R₅₀,R₅₁,R₅₈,R₆₃, R₆₄,R₆₅,R₆₆,R₇₀,R₇₁=are independently N or absent;
R₁₃,R₁₇,R₂₃,R₆₁=are independently A,C,U or absent;
R₁₀,R₁₄,R₂₄,R₄₀,R₅₂,R₅₆=are independently A,G or absent;
R₇,R₁₅,R₂₅,R₆₇,R₇₂=are independently A,G,U or absent;
R₁₁,R₅₇=are independently A,U or absent;
R₃₉=C,G or absent;
R₃,R₄,R₂₂,R₄₂,R₄₉,R₅₅,R₆₂=are independently C,G,U or absent;
R₁₈,R₂₈,R₃₅,R₃₇,R₅₃,R₅₉,R₆₀=are independently C,U or absent;
R₁₉=G or absent;
R₈,R₃₆,R₃₈,R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{GLU},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Glu is:
R₀,R₁₈,R₂₃=are absent
R₁₇,R₄₀=are independently A or absent;
R₂₆,R₂₇,R₃₄,R₄₃,R₆₈,R₆₉,R₇₁=are independently A,C,G or absent;
R₁,R₂,R₅,R₁₂,R₂₁,R₃₁,R₃₃,R₄₁,R₄₅,R₄₈,R₅₁,R₅₈,R₆₆,R₇₀=are independently N or absent;
R₄₄,R₆₁=are independently A,C,U or absent;
R₉,R₁₄,R₂₄,R₂₅,R₅₂,R₅₆,R₆₃=are independently A,G or absent;
R₇,R₁₅,R₄₆,R₅₀,R₆₇,R₇₂=are independently A,G,U or absent;
R₂₉,R₅₇=are independently A,U or absent;
R₆₀=C or absent;
R₃₉=C,G or absent;
R₃,R₆,R₂₀,R₃₀,R₃₂,R₄₂,R₅₅,R₆₂,R₆₅=are independently C,G,U or absent;
R₄,R₈,R₁₆,R₂₈,R₃₅,R₃₇,R₄₉,R₅₃,R₅₉=are independently C,U or absent;
R₁₀,R₁₉=are independently G or absent;
R₂₂,R₆₄=are independently G,U or absent;
R₁₁,R₁₃,R₃₆,R₃₈,R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{GLU},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]Xi-R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Glu is:
R₀,R₁₇,R₁₈,R₂₃=are absent
R₁₄,R₂₇,R₄₀,R₇₁=are independently A or absent;
R₄₄=A,C or absent;
R₄₃=A,C,G or absent;
R₁,R₃₁,R₃₃,R₄₅,R₅₁,R₆₆=are independently N or absent;
R₂₁,R₄₁=are independently A,C,U or absent;
R₇,R₂₄,R₂₅,R₅₀,R₅₂,R₅₆,R₆₃,R₆₈,R₇₀=are independently A,G or absent;
R₅,R₄₆=are independently A,G,U or absent;
R₂₉,R₅₇,R₆₇,R₇₂=are independently A,U or absent;
R₂,R₃₉,R₆₀=are independently C or absent;
R₃,R₁₂,R₂₀,R₂₆,R₃₄,R₆₉=are independently C,G or absent;
R₆,R₃₀,R₄₂,R₄₅,R₆₅=are independently C,G,U or absent;
R₄,R₁₆,R₂₈,R₃₅,R₃₇,R₄₉,R₅₃,R₅₅,R₅₈,R₆₁,R₆₂=are independently C,U or absent;
R₉,R₁₀,R₁₉,R₆₄=are independently G or absent;
R₁₅,R₂₂,R₃₂=are independently G,U or absent;
R₈,R₁₁,R₁₃,R₃₆,R₃₈,R₅₄,R₅₉=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Glycine TREM Consensus Sequence

In an embodiment, a TREM disclosed herein comprises the sequence of Formula I _{GLY},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Gly is:
Rₒ=absent;
R₂₄=A or absent;
R₃,R₉,R₄₀,R₅₀,R₅₁=are independently A,C,G or absent;
R₄,R₅,R₆,R₇,R₁₂,R₁₆,R₂₁,R₂₂,R₂₆,R₂₉,R₃₀,R₃₁,R₃₂,R₃₃,R₃₄,R₄₁,R₄₂,R₄₃,R₄₄,R₄₅,R₄₆,R₄₈,R₄₉,R₅₈,R₆₃,R₆₄,R₆₅,R₆₆,R₆₇,R₆₈=are independently N or absent;
R₅₉=A,C,U or absent;
R₁,R₁₀,R₁₄,R₁₅,R₂₇,R₅₆=are independently A,G or absent;
R₂₀,R₂₅=are independently A,G,U or absent;
R₅₇,R₇₂=are independently A,U or absent;
R₃₈,R₃₉,R₆₀=are independently C or absent;
R₅₂=C,G or absent;
R₂,R₁₉,R₃₇,R₅₄,R₅₅,R₆₁,R₆₂,R₆₉,R₇₀=are independently C,G,U or absent;
R₁₁,R₁₃,R₁₇,R₂₈,R₃₅,R₃₆,R₇₁=are independently C,U or absent;
R₈,R₁₈,R₂₃,R₅₃=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{GLY},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Gly is:
R₀,R₁₈,R₂₃=are absent
R₂₄,R₂₇,R₄₀,R₇₂=are independently A or absent;
R₂₆=A,C or absent;
R₃,R₇,R₆₈=are independently A,C,G or absent;
R₅,R₃₀,R₄₁,R₄₂,R₄₄,R₄₉,R₆₇=are independently A,C,G,U or absent;
R₃₁,R₃₂,R₃₄=are independently A,C,U or absent;
R₉,R₁₀,R₁₄,R₁₅,R₃₃,R₅₀,R₅₆=are independently A,G or absent;
R₁₂,R₁₆,R₂₂,R₂₅,R₂₉,R₄₆=are independently A,G,U or absent;
R₅₇=A,U or absent;
R₁₇,R₃₈,R₃₉,R₆₀,R₆₁,R₇₁=are independently C or absent;
R₆,R₅₂,R₆₄,R₆₆=are independently C,G or absent;
R₂,R₄,R₃₇,R₄₈,R₅₅,R₆₅=are independently C,G,U or absent;
R₁₃,R₃₅,R₄₃,R₆₂,R₆₉=are independently C,U or absent;
R₁,R₁₉,R₂₀,R₅₁,R₇₀=are independently G or absent;
R₂₁,R₄₅,R₆₃=are independently G,U or absent;
R₈,R₁₁,R₂₈,R₃₆,R₅₃,R₅₄,R₅₈,R₅₉=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{GLY},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Gly is:
R₀,R₁₈,R₂₃=are absent
R₂₄,R₂₇,R₄₀,R₇₂=are independently A or absent;
R₂₆=A,C or absent;
R₃,R₇,R₄₉,R₆₈=are independently A,C,G or absent;
R₅,R₃₀,R₄₁,R₄₄,R₆₇=are independently N or absent;
R₃₁,R₃₂,R₃₄=are independently A,C,U or absent;
R₉,R₁₀,R₁₄,R₁₅,R₃₃,R₅₀,R₅₆=are independently A,G or absent;
R₁₂,R₂₅,R₂₉,R₄₂,R₄₆=are independently A,G,U or absent;
R₁₆,R₅₇=are independently A,U or absent;
R₁₇,R₃₈,R₃₉,R₆₀,R₆₁,R₇₁=are independently C or absent;
R₆,R₅₂,R₆₄,R₆₆=are independently C,G or absent;
R₃₇,R₄₈,R₆₅=are independently C,G,U or absent;
R₂,R₄,R₁₃,R₃₅,R₄₃,R₅₅,R₆₂,R₆₉=are independently C,U or absent;
R₁,R₁₉,R₂₀,R₅₁,R₇₀=are independently G or absent;
R₂₁,R₂₂,R₄₅,R₆₃=are independently G,U or absent;
R₈,R₁₁,R₂₈,R₃₆,R₅₃,R₅₄,R₅₈,R₅₉=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Histidine TREM Consensus Sequence

In an embodiment, a TREM disclosed herein comprises the sequence of Formula I _{HIS},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for His is:
R₂₃=absent;
R₁₄,R₂₄,R₅₇=are independently A or absent;
R₇₂=A,C or absent;
R₉,R₂₇,R₄₃,R₄₈,R₆₉=are independently A,C,G or absent;
R₃,R₄,R₅,R₆,R₁₂,R₂₅,R₂₆,R₂₉,R₃₀,R₃₁,R₃₄,R₄₂,R₄,R₄₆,R₄₉,R₅₀,R₅₈,R₆₂,R₆₃,R₆₆,R₆₇,R₆₈=are independently N or absent;
R₁₃,R₂₁,R₄₁,R₄₄,R₆₅=are independently A,C,U or absent;
R₄₀,R₅₁,R₅₆,R₇₀=are independently A,G or absent;
R₇,R₃₂=are independently A,G,U or absent;
R₅₅,R₆₀=are independently C or absent;
R₁₁,R₁₆,R₃₃,R₆₄=are independently C,G,U or absent;
R₂,R₁₇,R₂₂,R₂₈,R₃₅,R₅₃,R₅₉,R₆₁,R₇₁=are independently C,U or absent;
R₁,R₁₀,R₁₅,R₁₉,R₂₀,R₃₇,R₃₉,R₅₂=are independently G or absent;
Rₒ=G,U or absent;
R₈,R₁₈,R₃₆,R₃₈,R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{HIS},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for His is:
R₀,R₁₇,R₁₈,R₂₃=are absent;
R₇,R₁₂,R₁₄,R₂₄,R₂₇,R₄₅,R₅₇,R₅₈,R₆₃,R₆₇,R₇₂=are independently A or absent;
R₃=A,C,U or absent;
R₄,R₄₃,R₅₆,R₇₀=are independently A,G or absent;
R₄₉=A,U or absent;
R₂,R₂₈,R₃₀,R₄₁,R₄₂,R₄₄,R₄₈,R₅₅,R₆₀,R₆₆,R₇₁=are independently C or absent;
R₂₅=C,G or absent;
R₉=C,G,U or absent;
R₈,R₁₃,R₂₆,R₃₃,R₃₅,R₅₀,R₅₃,R₆₁,R₆₈=are independently C,U or absent;
R₁,R₆,R₁₀,R₁₅,R₁₉,R₂₀,R₃₂,R₃₄,R₃₇,R₃₉,R₄₀,R₄₆,R₅₁,R₅₂,R₆₂,R₆₄,R₆₉=are independently G or absent;
R₁₆=G,U or absent;
R₅,R₁₁,R₂₁,R₂₂,R₂₉,R₃₁,R₃₆,R₃₈,R₅₄,R₅₉,R₆₅=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{HIS},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for His is:
R₀,R₁₇,R₁₈,R₂₃=are absent
R₇,R₁₂,R₁₄,R₂₄,R₂₇,R₄₅,R₅₇,R₅₈,R₆₃,R₆₇,R₇₂=are independently A or absent;
R₃=A,C or absent;
R₄,R₄₃,R₅₆,R₇₀=are independently A,G or absent;
R₄₉=A,U or absent;
R₂,R₂₈,R₃₀,R₄₁,R₄₂,R₄₄,R₄₈,R₅₅,R₆₀,R₆₆,R₇₁=are independently C or absent;
R₈,R₉,R₂₆,R₃₃,R₃₅,R₅₀,R₆₁,R₆₈=are independently C,U or absent;
R₁,R₆,R₁₀,R₁₅,R₁₉,R₂₀,R₂₅,R₃₂,R₃₄,R₃₇,R₃₉,R₄₀,R₄₆,R₅₁,R₅₂,R₆₂,R₆₄,R₆₉=are independently G or absent;
R₅,R₁₁,R₁₃,R₁₆,R₂₁,R₂₂,R₂₉,R₃₁,R₃₆,R₃₈,R₅₃,R₅₄,R₅₉,R₆₅=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Isoleucine TREM Consensus Sequence

In an embodiment, a TREM disclosed herein comprises the sequence of Formula I _{ILE},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₂—R₃—R₄—R₆₅—R₆₆—R₇—R₆₈—R₆₉—R₇₀—R₇₁—R₇₂

wherein the consensus for Ile is:
R₂₃=absent;
R₃₈,R₄₁,R₅₇,R₇₂=are independently A or absent;
R₁,R₂₆=are independently A,C,G or absent;
R₃,R₄,R₆,R₁₆,R₃₁,R₃₂,R₃₄,R₃₇,R₄₂,R₄₃,R₄₄,R₄₅,R₄₆,R₄₈,R₄₉,R₅₀,R₅₈,R₅₉,R₆₂,R₆₃,R₆₄,R₆₆,R₆₇,R₆₈, R₆₉=are independently N or absent;
R₂₂,R₆₁,R₆₅=are independently A,C,U or absent;
R₉,R₁₄,R₁₅,R₂₄,R₂₇,R₄₀=are independently A,G or absent;
R₇,R₂₅,R₂₉,R₅₁,R₅₆=are independently A,G,U or absent;
R₁₈,R₅₄=are independently A,U or absent;
R₆₀=C or absent;
R₂,R₅₂,R₇₀=are independently C,G or absent;
R₅,R₁₂,R₂₁,R₃₀,R₃₃,R₇₁=are independently C,G,U or absent;
R₁₁,R₁₃,R₁₇,R₂₈,R₃₅,R₅₃,R₅₅=are independently C,U or absent;
R₁₀,R₁₉,R₂₀=are independently G or absent;
R₈,R₃₆,R₃₉=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{ILE},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Ile is:
R₀,R₁₈,R₂₃=are absent
R₂₄,R₃₈,R₄₀,R₄₁,R₅₇,R₇₂=are independently A or absent;
R₂₆,R₆₅=are independently A,C or absent;
R₅₈,R₅₉,R₆₇=are independently N or absent;
R₂₂=A,C,U or absent;
R₆,R₉,R₁₄,R₁₅,R₂₉,R₃₄,R₄₃,R₄₆,R₄₈,R₅₀,R₅₁,R₆₃,R₆₉=are independently A,G or absent;
R₃₇,R₅₆=are independently A,G,U or absent;
R₅₄=A,U or absent;
R₂₈,R₃₅,R₆₀,R₆₂,R₇₁=are independently C or absent;
R₂,R₅₂,R₇₀=are independently C,G or absent;
R₅=C,G,U or absent;
R₃,R₄,R₁₁,R₁₃,R₁₇,R₂₁,R₃₀,R₄₂,R₄₄,R₄₅,R₄₉,R₅₃,R₅₅,R₆₁,R₆₄,R₆₆=are independently C,U or absent;
R₁,R₁₀,R₁₉,R₂₀,R₂₅,R₂₇,R₃₁,R₆₈=are independently G or absent; R₇,R₁₂,R₃₂=are independently G,U or absent;
R₈,R₁₆,R₃₃,R₃₆,R₃₉=are independently U or absent; [R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{ILE},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Ile is:
R₀,R₁₈,R₂₃=are absent
R₁₄,R₂₄,R₃₈,R₄₀,R₄₁,R₅₇,R₇₂=are independently A or absent;
R₂₆,R₆₅=are independently A,C or absent;
R₂₂,R₅₉=are independently A,C,U or absent;
R₆,R₉,R₁₅,R₃₄,R₄₃,R₄₆,R₅₁,R₅₆,R₆₃,R₆₉=are independently A,G or absent;
R₃₇=A,G,U or absent;
R₁₃,R₂₈,R₃₅,R₄₄,R₅₅,R₆₀,R₆₂,R₇₁=are independently C or absent;
R₂,R₅,R₇₀=are independently C,G or absent;
R₅₈,R₆₇=are independently C,G,U or absent;
R₃,R₄,R₁₁,R₁₇,R₂₁,R₃₀,R₄₂,R₄₅,R₄₉,R₅₃,R₆₁,R₆₄,R₆₆=are independently C,U or absent;
R₁,R₁₀,R₁₉,R₂₀,R₂₅,R₂₇,R₂₉,R₃₁,R₃₂,R₄₈,R₅₀,R₅₂,R₆₈=are independently G or absent;
R₇,R₁₂=are independently G,U or absent;
R₈,R₁₆,R₃₃,R₃₆,R₃₉,R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Methionine TREM Consensus Sequence

In an embodiment, a TREM disclosed herein comprises the sequence of Formula I _{MET},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Met is:
R₀,R₂₃=are absent;
R₁₄,R₃₈,R₄₀,R₅₇=are independently A or absent;
R₆₀=A,C or absent;
R₃₃,R₄₈,R₇₀=are independently A,C,G or absent;
R₁,R₃,R₄,R₅,R₆,R₁₁,R₁₂,R₁₆,R₁₇,R₂₁,R₂₂,R₂₆,R₂₇,R₂₉,R₃₀,R₃₁,R₃₂,R₄₂,R₄₄,R₄₅,R₄₆,R₄₉,R₅₀,R₅₈,R₆₂, R₆₃,R₆₆,R₆₇,R₆₈,R₆₉,R₇₁=are independently N or absent;
R₁₈,R₃₅,R₄₁,R₅₉,R₆₅=are independently A,C,U or absent;
R₉,R₁₅,R₅₁=are independently A,G or absent;
R₇,R₂₄,R₂₅,R₃₄,R₅₃,R₅₆=are independently A,G,U or absent;
R₇₂=A,U or absent;
R₃₇=C or absent;
R₁₀,R₅₅=are independently C,G or absent;
R₂,R₁₃,R₂₈,R₄₃,R₆₄=are independently C,G,U or absent;
R₃₆,R₆₁=are independently C,U or absent;
R₁₉,R₂₀,R₅₂=are independently G or absent;
R₈,R₃₉,R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{MET},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Met is:
R₀,R₁₈,R₂₂,R₂₃=are absent
R₁₄,R₂₄,R₃₈,R₄₀,R₄₁,R₅₇,R₇₂=are independently A or absent;
R₅₉,R₆₀,R₆₂,R₆₅=are independently A,C or absent;
R₆,R₄₅,R₆₇=are independently A,C,G or absent;
R₄=N or absent;
R₂₁,R₄₂=are independently A,C,U or absent;
R₁,R₉,R₂₇,R₂₉,R₃₂,R₄₆,R₅₁=are independently A,G or absent;
R₁₇,R₄₉,R₅₃,R₅₆,R₅₈=are independently A,G,U or absent;
R₆₃=A,U or absent;
R₃,R₁₃,R₃₇=are independently C or absent;
R₄₈,R₅₅,R₆₄,R₇₀=are independently C,G or absent;
R₂,R₅,R₆₆,R₆₈=are independently C,G,U or absent;
R₁₁,R₁₆,R₂₆,R₂₈,R₃₀,R₃₁,R₃₅,R₃₆,R₄₃,R₄₄,R₆₁,R₇₁=are independently C,U or absent;
R₁₀,R₁₂,R₁₅,R₁₉,R₂₀,R₂₅,R₃₃,R₅₂,R₆₉=are independently G or absent;
R₇,R₃₄,R₅₀=are independently G,U or absent;
R₈,R₃₉,R₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{MET},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Met is:
R₀,R₁₈,R₂₂,R₂₃=are absent
R₁₄,R₂₄,R₃₈,R₄₀,R₄₁,R₅₇,R₇₂=are independently A or absent;
R₅₉,R₆₂,R₆₅=are independently A,C or absent;
R₆,R₆₇=are independently A,C,G or absent;
R₄,R₂₁=are independently A,C,U or absent;
R₁,R₉,R₂₇,R₂₉,R₃₂,R₄₅,R₄₆,R₅₁=are independently A,G or absent;
R₁₇,R₅₆,R₅₈=are independently A,G,U or absent;
R₄₉,R₅₃,R₆₃=are independently A,U or absent;
R₃,R₁₃,R₂₆,R₃₇,R₄₃,R₆₀=are independently C or absent;
R₂,R₄₈,R₅₅,R₆₄,R₇₀=are independently C,G or absent;
R₅,R₆₆=are independently C,G,U or absent;
R₁₁,R₁₆,R₂₈,R₃₀,R₃₁,R₃₅,R₃₆,R₄₂,R₄₄,R₆₁,R₇₁=are independently C,U or absent;
R₁₀,R₁₂,R₁₅,R₁₉,R₂₀,R₂₅,R₃₃,R₅₂,R₆₉=are independently G or absent;
R₇,R₃₄,R₅₀,R₆₈=are independently G,U or absent;
R₈,R₃₉,R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Leucine TREM Consensus Sequence

In an embodiment, a TREM disclosed herein comprises the sequence of Formula I _{LEU},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Leu is:
Rₒ=absent;
R₃₈,R₅₇=are independently A or absent;
R₆₀=A,C or absent;
R₁,R₁₃,R₂₇,R₄₈,R₅₁,R₅₆=are independently A,C,G or absent;
R₂,R₃,R₄,R₅,R₆,R₇,R₉,R₁₀,R₁₁,R₁₂,R₁₆,R₂₃,R₂,R₂₈,R₂₉,R₃,R₃₁,R₃₂,R₃₃,R₃₄,R₃₇,R₄₁,R₄₂,R₄₃,R₄₄, R₄₅,R₄₆,R₄₉,R₅₀,R₅₈,R₆₂,R₆₃,R₆₅,R₆₆,R₆₇,R₆₈,R₆₉,R₇₀=are independently N or absent;
R₁₇,R₁₈,R₂₁,R₂₂,R₂₅,R₃₅,R₅₅=are independently A,C,U or absent;
R₁₄,R₁₅,R₃₉,R₇₂=are independently A,G or absent;
R₂₄,R₄₀=are independently A,G,U or absent;
R₅₂,R₆₁,R₆₄,R₇₁=are independently C,G,U or absent;
R₃₆,R₅₃,R₅₉=are independently C,U or absent;
R₁₉=G or absent; R₂₀=G,U or absent;
R₈,R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{LEU},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Leu is:
R₀=absent
R₃₈,R₅₇,R₇₂=are independently A or absent;
R₆₀=A,C or absent;
R₄,R₅,R₄₈,R₅₀,R₅₆,R₆₉=are independently A,C,G or absent;
R₆,R₃₃,R₄₁,R₄₃,R₄₆,R₄₉,R₅₈,R₆₃,R₆₆,R₇₀=are independently N or absent;
R₁₁,R₁₂,R₁₇,R₂₁,R₂₂,R₂₈,R₃₁,R₃₇,R₄₄,R₅₅=are independently A,C,U or absent;
R₁,R₉,R₁₄,R₁₅,R₂₄,R₂₇,R₃₄,R₃₉=are independently A,G or absent;
R₇,R₂₉,R₃₂,R₄₀,R₄₅=are independently A,G,U or absent;
R₂₅=A,U or absent;
R₁₃=C,G or absent;
R₂,R₃,R₁₆,R₂₆,R₃₀,R₅₂,R₆₂,R₆₄,R₆₅,R₆₇,R₆₈=are independently C,G,U or absent;
R₁₈,R₃₅,R₄₂,R₅₃,R₅₉,R₆₁,R₇₁=are independently C,U or absent;
R₁₉,R₅₁=are independently G or absent;
R₁₀,R₂₀=are independently G,U or absent;
R₈,R₂₃,R₃₆,R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{LEU},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Leu is:
R₀=absent
R₃₈,R₅₇,R₇₂=are independently A or absent;
R₆₀=A,C or absent;
R₄,R₅,R₄₈,R₅₀,R₅₆,R₅₈,R₆₉=are independently A,C,G or absent;
R₆,R₃₃,R₄₃,R₄₆,R₄₉,R₆₃,R₆₆,R₇₀=are independently N or absent;
R₁₁,R₁₂,R₁₇,R₂₁,R₂₂,R₂₈,R₃₁,R₃₇,R₄₁,R₄₄,R₅₅=are independently A,C,U or absent;
R₁,R₉,R₁₄,R₁₅,R₂₄,R₂₇,R₃₄,R₃₉=are independently A,G or absent;
R₇,R₂₉,R₃₂,R₄₀,R₄₅=are independently A,G,U or absent;
R₂₅=A,U or absent;
R₁₃=C,G or absent;
R₂,R₃,R₁₆,R₃₀,R₅₂,R₆₂,R₆₄,R₆₇,R₆₈=are independently C,G,U or absent;
R₁₈,R₃₅,R₄₂,R₅₃,R₅₉,R₆₁,R₆₅,R₇₁=are independently C,U or absent;
R₁₉,R₅₁=are independently G or absent;
R₁₀,R₂₀,R₂₆=are independently G,U or absent;
R₈,R₂₃,R₃₆,R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Lysine TREM Consensus Sequence

In an embodiment, a TREM disclosed herein comprises the sequence of Formula I _{LYS},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]Xi-R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Lys is:
R₀=absent
R₁₄=A or absent;
R₄₀,R₄₁=are independently A,C or absent;
R₃₄,R₄₃,R₅₁=are independently A,C,G or absent;
R₁,R₂,R₃,R₄,R₅,R₆,R₇,R₁₁,R₁₂,R₁₆,R₂₁,R₂₆,R₃₀,R₃₁,R₃₂,R₄₄,R₄₅,R₄₆,R₄₈,R₄₉,R₅₀,R₅₈,R₆₂,R₆₃,R₆₅, R₆₆,R₆₇,R₆₈,R₆₉,R₇₀=are independently N or absent;
R₁₃,R₁₇,R₅₉,R₇₁=are independently A,C,U or absent;
R₉,R₁₅,R₁₉,R₂₀,R₂₅,R₂₇,R₅₂,R₅₆=are independently A,G or absent;
R₂₄,R₂₉,R₇₂=are independently A,G,U or absent;
R₁₈,R₅₇=are independently A,U or absent;
R₁₀,R₃₃=are independently C,G or absent;
R₄₂,R₆₁,R₆₄=are independently C,G,U or absent;
R₂₈,R₃₅,R₃₆,R₃₇,R₅₃,R₅₅,R₆₀=are independently C,U or absent;
R₈,R₂₂,R₂₃,R₃₈,R₃₉,R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{LYS},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Lys is:
R₀,R₁₈,R₂₃=are absent
R₁₄=A or absent;
R₄₀,R₄₁,R₄₃=are independently A,C or absent;
R₃,R₇=are independently A,C,G or absent;
R₁,R₆,R₁₁,R₃₁,R₄₅,R₄₈,R₄₉,R₆₃,R₆₅,R₆₆,R₆₈=are independently N or absent;
R₂,R₁₂,R₁₃,R₁₇,R₄₄,R₆₇,R₇₁=are independently A,C,U or absent;
R₉,R₁₅,R₁₉,R₂₀,R₂₅,R₂₇,R₃₄,R₅₀,R₅₂,R₅₆,R₇₀,R₇₂=are independently A,G or absent;
R₅,R₂₄,R₂₆,R₂₉,R₃₂,R₄₆,R₆₉=are independently A,G,U or absent;
R₅₇=A,U or absent;
R₁₀,R₆₁=are independently C,G or absent;
R₄,R₁₆,R₂₁,R₃₀,R₅₈,R₆₄=are independently C,G,U or absent;
R₂₈,R₃₅,R₃₆,R₃₇,R₄₂,R₅₃,R₅₅,R₅₉,R₆₀,R₆₂=are independently C,U or absent;
R₃₃,R₅₁=are independently G or absent;
R₈=G,U or absent;
R₂₂,R₃₈,R₃₉,R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{LYS},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Lys is:
R₀,R₁₈,R₂₃=absent
R₉,R₁₄,R₃₄,R₄₁=are independently A or absent;
R₄₀=A,C or absent;
R₁,R₃,R₇,R₃₁=are independently A,C,G or absent;
R₄₈,R₆₅,R₆₈=are independently N or absent;
R₂,R₁₃,R₁₇,R₄₄,R₆₃,R₆₆=are independently A,C,U or absent;
R₅,R₁₅,R₁₉,R₂₀,R₂₅,R₂₇,R₂₉,R₅₀,R₅₂,R₅₆,R₇₀,R₇₂=are independently A,G or absent;
R₆,R₂₄,R₃₂,R₄₉=are independently A,G,U or absent;
R₁₂,R₂₆,R₄₆,R₅₇=are independently A,U or absent;
R₁₁,R₂₈,R₃₅,R₄₃=are independently C or absent;
R₁₀,R₄₅,R₆₁=are independently C,G or absent;
R₄,R₂₁,R₆₄=are independently C,G,U or absent;
R₃₇,R₅₃,R₅₅,R₅₉,R₆₀,R₆₂,R₆₇,R₇₁=are independently C,U or absent;
R₃₃,R₅₁=are independently G or absent;
R₈,R₃₀,R₅₈,R₆₉=are independently G,U or absent;
R₁₆,R₂₂,R₃₆,R₃₈,R₃₉,R₄₂,R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Phenylalanine TREM Consensus Sequence

In an embodiment, a TREM disclosed herein comprises the sequence of Formula I _{PHE},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Phe is:
R₀,R₂₃=are absent
R₉,R₁₄,R₃₈,R₃₉,R₅₇,R₇₂=are independently A or absent;
R₇₁=A,C or absent;
R₄₁,R₇₀=are independently A,C,G or absent;
R₄,R₅,R₆,R₃₀,R₃₁,R₃₂,R₃₄,R₄₂,R₄₄,R₄₅,R₄₆,R₄₈,R₄₉,R₅₈,R₆₂,R₆₃,R₆₆,R₆₇,R₆₈,R₆₉=are independently N or absent;
R₁₆,R₆₁,R₆₅=are independently A,C,U or absent;
R₁₅,R₂₆,R₂₇,R₂₉,R₄₀,R₅₆=are independently A,G or absent;
R₇,R₅₁=are independently A,G,U or absent;
R₂₂,R₂₄=are independently A,U or absent;
R₅₅,R₆₀=are independently C or absent;
R₂,R₃,R₂₁,R₃₃,R₄₃,R₅₀,R₆₄=are independently C,G,U or absent;
R₁₁,R₁₂,R₁₃,R₁₇,R₂₈,R₃₅,R₃₆,R₅₉=are independently C,U or absent;
R₁₀,R₁₉,R₂₀,R₂₅,R₃₇,R₅₂=are independently G or absent;
R₁=G,U or absent;
R₈,R₁₈,R₅₃,R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{PHE},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]Xi-R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Phe is:
R₀,R₁₈,R₂₃=absent
R₁₄,R₂₄,R₃₈,R₃₉,R₅₇,R₇₂=are independently A or absent;
R₄₆,R₇₁=are independently A,C or absent;
R₄,R₇₀=are independently A,C,G or absent;
R₄₅=A,C,U or absent;
R₆,R₇,R₁₅,R₂₆,R₂₇,R₃₂,R₃₄,R₄₀,R₄₁,R₅₆,R₆₉=are independently A,G or absent;
R₂₉=A,G,U or absent;
R₅,R₉,R₆₇=are independently A,U or absent;
R₃₅,R₄₉,R₅₅,R₆₀=are independently C or absent;
R₂₁,R₄₃,R₆₂=are independently C,G or absent;
R₂,R₃₃,R₆₈=are independently C,G,U or N or absent;
R₃,R₁₁,R₁₂,R₁₃,R₂₈,R₃₀,R₃₆,R₄₂,R₄₄,R₄₈,R₅₈,R₅₉,R₆₁,R₆₆=are independently C,U or absent;
R₁₀,R₁₉,R₂₀,R₂₅,R₃₇,R₅₁,R₅₂,R₆₃,R₆₄=are independently G or absent;
R₁,R₃₁,R₅₀=are independently G,U or absent;
R₈,R₁₆,R₁₇,R₂₂,R₅₃,R₅₄,R₆₅=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{PHE},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]Xi-R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Phe is:
R₀,R₁₈,R₂₂,R₂₃=absent
R₅,R₇,R₁₄,R₂₄,R₂₆,R₃₂,R₃₄,R₃₈,R₃₉,R₄₁,R₅₇,R₇₂=are independently A or absent;
R₄₆=A,C or absent;
R₇₀=A,C,G or absent;
R₄,R₆,R₁₅,R₅₆,R₆₉=are independently A,G or absent;
R₉,R₄₅=are independently A,U or absent;
R₂,R₁₁,R₁₃,R₃₅,R₄₃,R₄₉,R₅₅,R₆₀,R₆₈,R₇₁=are independently C or absent;
R₃₃=C,G or absent;
R₃,R₂₈,R₃₆,R₄₈,R₅₈,R₅₉,R₆₁=are independently C,U or absent;
R₁,R₁₀,R₁₉,R₂₀,R₂₁,R₂₅,R₂₇,R₂₉,R₃₇,R₄₀,R₅₁,R₅₂,R₆₂,R₆₃,R₆₄=are independently G or absent;
R₈,R₁₂,R₁₆,R₁₇,R₃₀,R₃₁,R₄₂,R₄₄,R₅₀,R₅₃,R₅₄,R₆₅,R₆₆,R₆₇=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Proline TREM Consensus Sequence

In an embodiment, a TREM disclosed herein comprises the sequence of Formula I _{PRO},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Pro is:
R₀=absent
R₁₄,R₅₇=are independently A or absent;
R₇₀,R₇₂=are independently A,C or absent;
R₉,R₂₆,R₂₇=are independently A,C,G or absent;
R₄,R₅,R₆,R₁₆,R₂₁,R₂₉,R₃₀,R₃₁,R₃₂,R₃₃,R₃₄,R₃₇,R₄₁,R₄₂,R₄₃,R₄₄,R₄₅,R₄₆,R₄₈,R₄₉,R₅₀,R₅₈,R₆₁,R₂, R₆₃,R₆₄,R₆₆,R₆₇,R₆₈=are independently N or absent;
R₃₅,R₆₅=are independently A,C,U or absent;
R₂₄,R₄₀,R₅₆=are independently A,G or absent;
R₇,R₂₅,R₅₁=are independently A,G,U or absent;
R₅₅,R₆₀=are independently C or absent;
R₁,R₃,R₇₁=are independently C,G or absent;
R₁₁,R₁₂,R₂₀,R₆₉=are independently C,G,U or absent;
R₁₃,R₁₇,R₁₈,R₂₂,R₂₃,R₂₈,R₅₉=are independently C,U or absent;
R₁₀,R₁₅,R₁₉,R₃₈,R₃₉,R₅₂=are independently G or absent;
R₂=are independently G,U or absent;
R₈,R₃₆,R₅₃,R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{PRO},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]Xi-R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Pro is:
R₀,R₁₇,R₁₈,R₂₂,R₂₃=absent;
R₁₄,R₄₅,R₅₆,R₅₇,R₅₈,R₆₅,R₆₈=are independently A or absent;
R₆₁=A,C,G or absent;
R₄₃=N or absent;
R₃₇=A, C,U or absent;
R₂₄,R₂₇,R₃₃,R₄₀,R₄₄,R₆₃=are independently A,G or absent;
R₃,R₁₂,R₃₀,R₃₂,R₄₈,R₅₅,R₆₀,R₇₀,R₇₁,R₇₂=are independently C or absent;
R₅,R₃₄,R₄₂,R₆₆=are independently C,G or absent;
R₂₀=C,G,U or absent;
R₃₅,R₄₁,R₄₉,R₆₂=are independently C,U or absent;
R₁,R₂,R₆,R₉,R₁₀,R₁₅,R₁₉,R₂₆,R₃₈,R₃₉,R₄₆,R₅₀,R₅₁,R₅₂,R₆₄,R₆₇,R₆₉=are independently G or absent;
R₁₁,R₁₆=are independently G,U or absent;
R₄,R₇,R₈,R₁₃,R₂₁,R₂₅,R₂₈,R₂₉,R₃₁,R₃₆,R₅₃,R₅₄,R₅₉=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{PRO},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]Xi-R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Pro is:
R₀,R₁₇,R₁₈,R₂₂,R₂₃=absent
R₁₄,R₄₅,R₅₆,R₅₇,R₅₈,R₆₅,R₆₈=are independently A or absent;
R₃₇=A,C,U or absent;
R₂₄,R₂₇,R₄₀=are independently A,G or absent;
R₃,R₅,R₁₂,R₃₀,R₃₂,R₄₈,R₄₉,R₅₅,R₆₀,R₆₁,R₆₂,R₆₆,R₇₀,R₇₁,R₇₂=are independently C or absent;
R₃₄,R₄₂=are independently C,G or absent;
R₄₃=C,G,U or absent;
R₄₁=C,U or absent;
R₁,R₂,R₆,R₉,R₁₀,R₁₅,R₁₉,R₂₀,R₂₆,R₃₃,R₃₈,R₃₉,R₄₄,R₄₆,R₅₀,R₅₁,R₅₂,R₆₃,R₆₄,R₆₇,R₆₉=are independently G or absent;
R₁₆=G,U or absent;
R₄,R₇,R₈,R₁₁,R₁₃,R₂₁,R₂₅,R₂₈,R₂₉,R₃₁,R₃₅,R₃₆,R₅₃,R₅₄,R₅₉=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Serine TREM Consensus Sequence

In an embodiment, a TREM disclosed herein comprises the sequence of Formula I _{SER},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₃—R₄—R₆₅—R₆₆—R₆₇—R₆₈—R₆₉—R₇₀—R₇₁—R₇₂

wherein the consensus for Ser is:
Rₒ=absent;
R₁₄,R₂₄,R₅₇=are independently A or absent;
R₄₁=A,C or absent;
R₂,R₃,R₄,R₅,R₆,R₇,R₉,R₁₀,R₁₁,R₁₂,R₁₃,R₁₆,R₂₁,R₂₅,R₂,R₂₇,R₂₈,R₃₀,R₃₁,R₃₂,R₃₃,R₃₄,R₃₇,R₄₂,R₄₃, R₄₄,R₄₅,R₄₆,R₄₈,R₄₉,R₅₀,R₆₂,R₆₃,R₆₄,R₆₅,R₆₆,R₆₇,R₆₈,R₆₉,R₇₀=are independently N or absent;
R₁₈=A,C,U or absent;
R₁₅,R₄₀,R₅₁,R₅₆=are independently A,G or absent;
R₁,R₂₉,R₅₅,R₇₂=are independently A,G,U or absent;
R₃₉=A,U or absent;
R₆₀=C or absent;
R₃₈=C,G or absent;
R₁₇,R₂₂,R₂₃,R₇₁=are independently C,G,U or absent;
R₈,R₃₅,R₃₆,R₅₅,R₅₉,R₆₁=are independently C,U or absent;
R₁₉,R₂₀=are independently G or absent;
R₅₂=G,U or absent;
R₅₃,R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{SER},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Ser is:
R₀,R₂₃=absent
R₁₄,R₂₄,R₄₁,R₅₇=are independently A or absent;
R₄₄=A,C or absent;
R₂₅,R₄₅,R₄₈=are independently A,C,G or absent;
R₂,R₃,R₄,R₅,R₃₇,R₅₀,R₆₂,R₆₆,R₆₇,R₆₉,R₇₀=are independently N or absent;
R₁₂,R₂₈,R₆₅=are independently A,C,U or absent;
R₉,R₁₅,R₂₉,R₃₄,R₄₀,R₅₆,R₆₃=are independently A,G or absent;
R₇,R₂₆,R₃₀,R₃₃,R₄₆,R₅₈,R₇₂=are independently A,G,U or absent;
R₃₉=A,U or absent;
R₁₁,R₃₅,R₆₀,R₆₁=are independently C or absent;
R₁₃,R₃₈=are independently C,G or absent;
R₆,R₁₇,R₃₁,R₄₃,R₆₄,R₆₈=are independently C,G,U or absent;
R₃₆,R₄₂,R₄₉,R₅₅,R₅₉,R₇₁=are independently C,U or absent;
R₁₀,R₁₉,R₂₀,R₂₇,R₅₁=are independently G or absent;
R₁,R₁₆,R₃₂,R₅₂=are independently G,U or absent;
R₈,R₁₈,R₂₁,R₂₂,R₅₃,R₅₄=are independently U or absent; [R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{SER},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Ser is:
R₀,R₂₃=absent
R₁₄,R₂₄,R₄₁,R₅₇,R₅₈=are independently A or absent;
R₄₄=A,C or absent;
R₂₅,R₄₈=are independently A,C,G or absent;
R₂,R₃,R₅,R₃₇,R₆₆,R₆₇,R₆₉,R₇₀=are independently N or absent;
R₁₂,R₂₈,R₆₂=are independently A,C,U or absent;
R₇,R₉,R₁₅,R₂₉,R₃₃,R₃₄,R₄₀,R₄₅,R₅₆,R₆₃=are independently A,G or absent;
R₄,R₂₆,R₄₆,R₅₀=are independently A,G,U or absent;
R₃₀,R₃₉=are independently A,U or absent;
R₁,R₁₇,R₃₅,R₆₀,R₆₁=are independently C or absent;
R₁₃,R₃₈=are independently C,G or absent;
R₆,R₆₄=are independently C,G,U or absent;
R₃₁,R₄₂,R₄₃,R₄₉,R₅₅,R₅₉,R₆₅,R₆₈,R₇₁=are independently C,U or absent;
R₁₀,R₁₉,R₂₀,R₂₇,R₅₁,R₅₂=are independently G or absent;
R₁,R₁₆,R₃₂,R₇₂=are independently G,U or absent;
R₅,R₁₈,R₂₁,R₂₂,R₃₆,R₅₃,R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Threonine TREM Consensus Sequence

In an embodiment, a TREM disclosed herein comprises the sequence of Formula I _{THR},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Thr is:
R₀,R₂₃=absent
R₁₄,R₄₁,R₅₇=are independently A or absent;
R₅₆,R₇₀=are independently A,C,G or absent;
R₄,R₅,R₆,R₇,R₁₂,R₁₆,R₂₆,R₃₀,R₃₁,R₃₂,R₃₄,R₃₇,R₄₂,R₄₄,R₄₅,R₄₆,R₄₈,R₄₉,R₅₀,R₅₈,R₆₂,R₆₃,R₆₄,R₆₅,R₆₆,R₆₇,R₆₈,R₇₂=are independently N or absent;
R₁₃,R₁₇,R₂₁,R₃₅,R₆₁=are independently A,C,U or absent;
R₁,R₉,R₂₄,R₂₇,R₂₉,R₆₉=are independently A,G or absent;
R₁₅,R₂₅,R₅₁=are independently A,G,U or absent;
R₄₀,R₅₃=are independently A,U or absent;
R₃₃,R₄₃=are independently C,G or absent;
R₂,R₃,R₅₉=are independently C,G,U or absent;
R₁₁,R₁₈,R₂₂,R₂₈,R₃₆,R₅₄,R₅₅,R₆₀,R₇₁=are independently C,U or absent;
R₁₀,R₂₀,R₃₈,R₅₂=are independently G or absent;
R₁₉=G,U or absent;
R₈,R₃₉=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{THR},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Thr is:
R₀,R₁₈,R₂₃=absent
R₁₄,R₄₁,R₅₇=are independently A or absent;
R₉,R₄₂,R₄₄,R₄₈,R₅₆,R₇₀=are independently A,C,G or absent;
R₄,R₆,R₁₂,R₂₆,R₄₉,R₅₈,R₆₃,R₆₄,R₆₆,R₆₈=are independently N or absent;
R₁₃,R₂₁,R₃₁,R₃₇,R₆₂=are independently A,C,U or absent;
R₁,R₁₅,R₂₄,R₂₇,R₂₉,R₄₆,R₅₁,R₆₉=are independently A,G or absent;
R₇,R₂₅,R₄₅,R₅₀,R₆₇=are independently A,G,U or absent;
R₄₀,R₅₃=are independently A,U or absent;
R₃₅=C or absent;
R₃₃,R₄₃=are independently C,G or absent;
R₂,R₃,R₅,R₁₆,R₃₂,R₃₄,R₅₉,R₆₅,R₇₂=are independently C,G,U or absent; R₁₁,R₁₇,R₂₂,R₂₈,R₃₀,R₃₆,R₅₅,R₆₀,R₆₁,R₇₁=are independently C,U or absent;
R₁₀,R₁₉,R₂₀,R₃₈,R₅₂=are independently G or absent;
R₈,R₃₉,R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{THR},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]Xi-R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Thr is:
R₀,R₁₈,R₂₃=absent
R₁₄,R₄₀,R₄₁,R₅₇=are independently A or absent;
R₄₄=A,C or absent;
R₉,R₄₂,R₄₈,R₅₆=are independently A,C,G or absent;
R₄,R₆,R₁₂,R₂₆,R₅₈,R₆₄,R₆₆,R₆₈=are independently N or absent;
R₁₃,R₂₁,R₃₁,R₃₇,R₄₉,R₆₂=are independently A,C,U or absent;
R₁,R₁₅,R₂₄,R₂₇,R₂₉,R₄₆,R₅₁,R₆₉=are independently A,G or absent;
R₇,R₂₅,R₄₅,R₅₀,R₆₃,R₆₇=are independently A,G,U or absent;
R₅₃=A,U or absent;
R₃₅=C or absent;
R₂,R₃₃,R₄₃,R₇₀=are independently C,G or absent;
R₅,R₁₆,R₃₄,R₅₉,R₆₅=are independently C,G,U or absent;
R₃,R₁₁,R₂₂,R₂₈,R₃₀,R₃₆,R₅₅,R₆₀,R₆₁,R₇₁=are independently C,U or absent;
R₁₀,R₁₉,R₂₀,R₃₈,R₅₂=are independently G or absent;
R₃₂=G,U or absent;
R₈,R₁₇,R₃₉,R₅₄,R₇₂=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Tryptophan TREM Consensus Sequence

In an embodiment, a TREM disclosed herein comprises the sequence of Formula I _{TRP},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]Xi-R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Trp is:
Rₒ=absent;
R₂₄,R₃₉,R₄₁,R₅₇=are independently A or absent;
R₂,R₃,R₂₆,R₂₇,R₄₀,R₄₈=are independently A,C,G or absent;
R₄,R₅,R₆,R₂₉,R₃₀,R₃₁,R₃₂,R₃₄,R₄₂,R₄₄,R₄₅,R₄₆,R₄₉,R₅₁,R₅₈,R₆₃,R₆₆,R₆₇,R₆₈=are independently N or absent;
R₁₃,R₁₄,R₁₆,R₁₈,R₂₁,R₆₁,R₆₅,R₇₁=are independently A,C,U or absent;
R₁,R₉,R₁₀,R₁₅,R₃₃,R₅₀,R₅₆=are independently A,G or absent;
R₇,R₂₅,R₇₂=are independently A,G,U or absent;
R₃₇,R₃₈,R₅₅,R₆₀=are independently C or absent;
R₁₂,R₃₅,R₄₃,R₆₄,R₆₉,R₇₀=are independently C,G,U or absent;
R₁₁,R₁₇,R₂₂,R₂₈,R₅₉,R₆₂=are independently C,U or absent;
R₁₉,R₂₀,R₅₂=are independently G or absent;
R₈,R₂₃,R₃₆,R₅₃,R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{TRP},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]Xi-R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Trp is:
R₀,R₁₈,R₂₂,R₂₃=absent
R₁₄,R₂₄,R₃₉,R₄₁,R₅₇,R₇₂=are independently A or absent;
R₃,R₄,R₁₃,R₆₁,R₇₁=are independently A,C or absent;
R₆,R₄₄=are independently A,C,G or absent;
R₂₁=A,C,U or absent;
R₂,R₇,R₁₅,R₂₅,R₃₃,R₃₄,R₄₅,R₅₆,R₆₃=are independently A,G or absent;
R₅₈=A,G,U or absent;
R₄₆=A,U or absent;
R₃₇,R₃₈,R₅₅,R₆₀,R₆₂=are independently C or absent;
R₁₂,R₂₆,R₂₇,R₃₅,R₄₀,R₄₈,R₆₇=are independently C,G or absent;
R₃₂,R₄₃,R₆₈=are independently C,G,U or absent;
R₁₁,R₁₆,R₂₈,R₃₁,R₄₉,R₅₉,R₆₅,R₇₀=are independently C,U or absent;
R₁,R₉,R₁₀,R₁₉,R₂₀,R₅₀,R₅₂,R₆₉=are independently G or absent;
R₅,R₈,R₂₉,R₃₀,R₄₂,R₅₁,R₆₄,R₆₆=are independently G,U or absent;
R₁₇,R₃₆,R₅₃,R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{TRP},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Trp is:
R₀,R₁₈,R₂₂,R₂₃=absent
R₁₄,R₂₄,R₃₉,R₄₁,R₅₇,R₇₂=are independently A or absent;
R₃,R₄,R₁₃,R₆₁,R₇₁=are independently A,C or absent;
R₆,R₄₄=are independently A,C,G or absent;
R₂₁=A,C,U or absent;
R₂,R₇,R₁₅,R₂₅,R₃₃,R₃₄,R₄₅,R₅₆,R₆₃=are independently A,G or absent;
R₅₈=A,G,U or absent;
R₄₆=A,U or absent;
R₃₇,R₃₈,R₅₅,R₆₀,R₆₂=are independently C or absent;
R₁₂,R₂₆,R₂₇,R₃₅,R₄₀,R₄₈,R₆₇=are independently C,G or absent;
R₃₂,R₄₃,R₆₈=are independently C,G,U or absent;
R₁₁,R₁₆,R₂₈,R₃₁,R₄₉,R₅₉,R₆₅,R₇₀=are independently C,U or absent;
R₁,R₉,R₁₀,R₁₉,R₂₀,R₅₀,R₅₂,R₆₉=are independently G or absent;
R₅,R₈,R₂₉,R₃₀,R₄₂,R₅₁,R₆₄,R₆₆=are independently G,U or absent;
R₁₇,R₃₆,R₅₃,R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Tyrosine TREM Consensus Sequence

In an embodiment, a TREM disclosed herein comprises the sequence of Formula I _{TYR},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Tyr is:
R₀=absent
R₁₄,R₃₉,R₅₇=are independently A or absent;
R₄₁,R₄₈,R₅₁,R₇₁=are independently A,C,G or absent;
R₃,R₄,R₅,R₆,R₉,R₁₀,R₁₂,R₁₃,R₁₆,R₂₅,R₂₆,R₃₀,R₃₁,R₃₂,R₄₂,R₄₄,R₄₅,R₄₆,R₄₉,R₅₀,R₅₈,R₆₂,R₆₃,R₆₆,
R₆₇,R₆₈,R₆₉,R₇₀=are independently N or absent;
R₂₂,R₆₅=are independently A,C,U or absent;
R₁₅,R₂₄,R₇,R₃₃,R₃₇,R₄₀,R₅₆=are independently A,G or absent;
R₇,R₂₉,R₃₄,R₇₂=are independently A,G,U or absent;
R₂₃,R₅₃=are independently A,U or absent;
   R₃₅,R₆₀=are independently C or absent;
R₂₀=C,G or absent;
R₁,R₂,R₂₈,R₆₁,R₆₄=are independently C,G,U or absent;
R₁₁,R₁₇,R₂₁,R₄₃,R₅₅=are independently C,U or absent;
R₁₉,R₅₂=are independently G or absent;
R₈,R₁₈,R₃₆,R₃₈,R₅₄,R₅₉=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{TYR},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Tyr is:
R₀,R₁₈,R₂₃=absent
R₇,R₉,R₁₄,R₂₄,R₂₆,R₃₄,R₃₉,R₅₇=are independently A or absent;
R₄₄,R₆₉=are independently A,C or absent;
R₇₁=A,C,G or absent;
R₆₈=N or absent;
R₅₈=A,C,U or absent;
R₃₃,R₃₇,R₄₁,R₅₆,R₆₂,R₆₃=are independently A,G or absent;
R₆,R₂₉,R₇₂=are independently A,G,U or absent;
R₃₁,R₄₅,R₅₃=are independently A,U or absent;
R₁₃,R₃₅,R₄₉,R₆₀=are independently C or absent;
R₂₀,R₄₈,R₆₄,R₆₇,R₇₀=are independently C,G or absent;
R₁,R₂,R₅,R₁₆,R₆₆=are independently C,G,U or absent;
R₁₁,R₂₁,R₂₈,R₄₃,R₅₅,R₆₁=are independently C,U or absent;
R₁₀,R₁₅,R₁₉,R₂₅,R₂₇,R₄₀,R₅₁,R₅₂=are independently G or absent;
R₃,R₄,R₃₀,R₃₂,R₄₂,R₄₆=are independently G,U or absent;
R₈,R₁₂,R₁₇,R₂₂,R₃₆,R₃₈,R₅₀,R₅₄,R₅₉,R₆₅=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{TYR},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Tyr is:
R₀,R₁₈,R₂₃=absent
R₇,R₉,R₁₄,R₂₄,R₂₆,R₃₄,R₃₉,R₅₇,R₇₂=are independently A or absent;
R₄₄,R₆₉=are independently A,C or absent;
R₇₁=A,C,G or absent;
R₃₇,R₄₁,R₅₆,R₆₂,R₆₃=are independently A,G or absent;
R₆,R₂₉,R₆₈=are independently A,G,U or absent;
R₃₁,R₄₅,R₅₈=are independently A,U or absent;
R₁₃,R₂₈,R₃₅,R₄₉,R₆₀,R₆₁=are independently C or absent;
R₅,R₄₈,R₆₄,R₆₇,R₇₀=are independently C,G or absent;
R₁,R₂=are independently C,G,U or absent;
R₁₁,R₁₆,R₂₁,R₄₃,R₅₅,R₆₆=are independently C,U or absent;
R₁₀,R₁₅,R₁₉,R₂₀,R₂₅,R₂₇,R₃₃,R₄₀,R₅₁,R₅₂=are independently G or absent;
R₃,R₄,R₃₀,R₃₂,R₄₂,R₄₆=are independently G,U or absent;
R₈,R₁₂,R₁₇,R₂₂,R₃₆,R₃₈,R₅₀,R₅₃,R₅₄,R₅₉,R₆₅=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Valine TREM Consensus Sequence

In an embodiment, a TREM disclosed herein comprises the sequence of Formula I _{VAL},

R₀—R₁—R₂—R₃—R₄—R₅—R₆—R₇—R₈—R₉—R₁₀—R₁₁—R₁₂—R₃—R₄—R₁₅—R₁₆—R₇—R₁₈—R₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Val is:
Rₒ,R₂₃=absent;
R₂₄,R₃₈,R₅₇=are independently A or absent;
R₉,R₇₂=are independently A,C,G or absent;
R₂,R₄,R₅,R₆,R₇,R₁₂,R₁₅,R₁₆,R₂₁,R₂₅,R₂₆,R₂₉,R₃₁,R₃₂,R₃₃,R₃₄,R₃₇,R₄₁,R₄₂,R₄₃,R₄₄,R₄₅,R₄₆,R₄₈,R₄₉, R₅₀,R₅₈,R₆₁,R₆₂,R₆₃,R₆₄,R₆₅,R₆₆,R₆₇,R₆₈,R₆₉,R₇₀=are independently N or absent;
R₁₇,R₃₅,R₅₉=are independently A,C,U or absent;
R₁₀,R₁₄,R₂₇,R₄₀,R₅₂,R₅₆=are independently A,G or absent;
R₁,R₃,R₅₁,R₅₃=are independently A,G,U or absent;
R₃₉=C or absent;
R₁₃,R₃₀,R₅₅=are independently C,G,U or absent;
R₁₁,R₂₂,R₂₈,R₆₀,R₇₁=are independently C,U or absent;
R₁₉=G or absent;
R₂₀=G,U or absent;
R₈,R₁₈,R₃₆,R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{VAL},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Val is:
R₀,R₁₈,R₂₃=absent;
R₂₄,R₃₈,R₅₇=are independently A or absent;
R₆₄,R₇₀,R₇₂=are independently A,C,G or absent;
R₁₅,R₁₆,R₂₆,R₂₉,R₃₁,R₃₂,R₄₃,R₄₄,R₄₅,R₄₉,R₅₀,R₅₈,R₆₂,R₆₅=are independently N or absent;
R₆,R₁₇,R₃₄,R₃₇,R₄₁,R₅₉=are independently A,C,U or absent;
R₉,R₁₀,R₁₄,R₂₇,R₄₀,R₄₆,R₅₁,R₅₂,R₅₆=are independently A,G or absent;
R₇,R₁₂,R₂₅,R₃₃,R₅₃,R₆₃,R₆₆,R₆₈=are independently A,G,U or absent;
R₆₉=A,U or absent;
R₃₉=C or absent;
R₅,R₆₇=are independently C,G or absent;
R₂,R₄,R₁₃,R₄₈,R₅₅,R₆₁=are independently C,G,U or absent;
R₁₁,R₂₂,R₂₈,R₃,R₃₅,R₆₀,R₇₁=are independently C,U or absent;
R₁₉=G or absent;
R₁,R₃,R₂₀,R₄₂=are independently G,U or absent;
R₈,R₂₁,R₃₆,R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{VAL},

R₀—R₁—R₂—R₃—R₄—R₅—R₆—R₇—R₈—R₉—R₁₀—R₁₁—R₁₂—R₁₃—R₁₄—R₁₅—R₁₆—R₁₇—R₁₈—R₁₉—R₂₀—R₂₁—R₂₂—R₂₃—R₂₄—R₂₅—R₂₆—R₂₇—R₂₈—R₂₉—R₃₀—R₃₁—R₃₂—R₃₃—R₃₄—R₃₅—R₃₆—R₃₇—R₃₈—R₃₉—R₄₀—R₄₁—R₄₂—R₄₃—R₄₄—R₄₅—R₄₆—[R₄₇]ₓ₁—R₄₈—R₄₉—R₅₀—R₅₁—R₅₂—R₅₃—R₅₄—R₅₅—R₅₆—R₅₇—R₅₈—R₅₉—R₆₀—R₆₁—R₆₂—R₆₃—R₆₄—R₆₅—R₆₆—R₆₇—R₆₈—R₆₉—R₇₀—R₇₁—R₇₂

wherein the consensus for Val is:
R₀,R₁₈,R₂₃=absent
R₂₄,R₃₈,R₄₀,R₅₇,R₇₂=are independently A or absent;
R₂₉,R₆₄,R₇₀=are independently A,C,G or absent;
R₄₉,R₅₀,R₆₂=are independently N or absent;
R₁₆,R₂₆,R₃₁,R₃₂,R₃₇,R₄₁,R₄₃,R₅₉,R₆₅=are independently A,C,U or absent;
R₉,R₁₄,R₂₇,R₄₆,R₅₂,R₅₆,R₆₆=are independently A,G or absent;
R₇,R₁₂,R₂₅,R₃₃,R₄₄,R₄₅,R₅₃,R₅₈,R₆₃,R₆₈=are independently A,G,U or absent;
R₆₉=A,U or absent;
R₃₉=C or absent;
R₅,R₆₇=are independently C,G or absent;
R₂,R₄,R₁₃,R₁₅,R₄₈,R₅₅=are independently C,G,U or absent;
R₆,R₁₁,R₂₂,R₂₈,R₃₀,R₃₄,R₃₅,R₆₀,R₆₁,R₇₁=are independently C,U or absent;
R₁₀,R₁₉,R₅₁=are independently G or absent;
R₁,R₃,R₂₀,R₄₂=are independently G,U or absent;
R₈,R₁₇,R₂₁,R₃₆,R₅₄=are independently U or absent;
[R₄₇]ₓ₁=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Variable Region Consensus Sequence

In an embodiment, a TREM disclosed herein comprises a variable region at position R₄₇. In an embodiment, the variable region is 1-271 ribonucleotides in length (e.g. 1-250, 1-225, 1-200, 1-175, 1-150, 1-125, 1-100, 1-75, 1-50, 1-40, 1-30, 1-29, 1-28, 1-27, 1-26, 1-25, 1-24, 1-23, 1-22, 1-21, 1-20, 1-19, 1-18, 1-17, 1-16, 1-15, 1-14, 1-13, 1-12, 1-11, 1-10, 10-271, 20-271, 30-271, 40-271, 50-271, 60-271, 70-271, 80-271, 100-271, 125-271, 150-271, 175-271, 200-271, 225-271, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 110, 125, 150, 175, 200, 225, 250, or 271 ribonucleotides). In an embodiment, the variable region comprises any one, all or a combination of Adenine, Cytosine, Guanine or Uracil.

In an embodiment, the variable region comprises a ribonucleic acid (RNA) sequence encoded by a deoxyribonucleic acid (DNA) sequence disclosed in Table 3, e.g., any one of SEQ ID NOs: 452-561 disclosed in Table 3.

TABLE 3
Exemplary variable region sequences.

| | SEQ ID NO | SEQUENCE |
|---|---|---|
| 1 | 452 | AAAATATAAATATATTTC |
| 2 | 453 | AAGCT |
| 3 | 454 | AAGTT |
| 4 | 455 | AATTCTTCGGAATGT |
| 5 | 456 | AGA |
| 6 | 457 | AGTCC |
| 7 | 458 | CAACC |
| 8 | 459 | CAATC |
| 9 | 460 | CAGC |
| 10 | 461 | CAGGCGGGTTCTGCCCGCGC |
| 11 | 462 | CATACCTGCAAGGGTATC |
| 12 | 463 | CGACCGCAAGGTTGT |
| 13 | 464 | CGACCTTGCGGTCAT |
| 14 | 465 | CGATGCTAATCACATCGT |
| 15 | 466 | CGATGGTGACATCAT |
| 16 | 467 | CGATGGTTTACATCGT |
| 17 | 468 | CGCCGTAAGGTGT |
| 18 | 469 | CGCCTTAGGTGT |
| 19 | 470 | CGCCTTTCGACGCGT |
| 20 | 471 | CGCTTCACGGCGT |
| 21 | 472 | CGGCAGCAATGCTGT |
| 22 | 473 | CGGCTCCGCCTTC |
| 23 | 474 | CGGGTATCACAGGGTC |
| 24 | 475 | CGGTGCGCAAGCGCTGT |
| 25 | 476 | CGTACGGGTGACCGTACC |
| 26 | 477 | CGTCAAAGACTTC |
| 27 | 478 | CGTCGTAAGACTT |
| 28 | 479 | CGTTGAATAAACGT |
| 29 | 480 | CTGTC |
| 30 | 481 | GGCC |
| 31 | 482 | GGGGATT |
| 32 | 483 | GGTC |
| 33 | 484 | GGTTT |
| 34 | 485 | GTAG |
| 35 | 486 | TAACTAGATACTTTCAGAT |
| 36 | 487 | TACTCGTATGGGTGC |
| 37 | 488 | TACTTTGCGGTGT |
| 38 | 489 | TAGGCGAGTAACATCGTGC |
| 39 | 490 | TAGGCGTGAATAGCGCCTC |
| 40 | 491 | TAGGTCGCGAGAGCGGCGC |
| 41 | 492 | TAGGTCGCGTAAGCGGCGC |
| 42 | 493 | TAGGTGGTTATCCACGC |
| 43 | 494 | TAGTC |
| 44 | 495 | TAGTT |
| 45 | 496 | TATACGTGAAAGCGTATC |
| 46 | 497 | TATAGGGTCAAAAACTCTATC |
| 47 | 498 | TATGCAGAAATACCTGCATC |
| 48 | 499 | TCCCCATACGGGGGC |
| 49 | 500 | TCCCGAAGGGGTTC |
| 50 | 501 | TCTACGTATGTGGGC |
| 51 | 502 | TCTCATAGGAGTTC |
| 52 | 503 | TCTCCTCTGGAGGC |
| 53 | 504 | TCTTAGCAATAAGGT |
| 54 | 505 | TCTTGTAGGAGTTC |
| 55 | 506 | TGAACGTAAGTTCGC |
| 56 | 507 | TGAACTGCGAGGTTCC |
| 57 | 508 | TGAC |
| 58 | 509 | TGACCGAAAGGTCGT |
| 59 | 510 | TGACCGCAAGGTCGT |
| 60 | 511 | TGAGCTCTGCTCTC |
| 61 | 512 | TGAGGCCTCACGGCCTAC |
| 62 | 513 | TGAGGGCAACTTCGT |
| 63 | 514 | TGAGGGTCATACCTCC |
| 64 | 515 | TGAGGGTGCAAATCCTCC |
| 65 | 516 | TGCCGAAAGGCGT |
| 66 | 517 | TGCCGTAAGGCGT |
| 67 | 518 | TGCGGTCTCCGCGC |
| 68 | 519 | TGCTAGAGCAT |
| 69 | 520 | TGCTCGTATAGAGCTC |
| 70 | 521 | TGGACAATTGTCTGC |
| 71 | 522 | TGGACAGATGTCCGT |
| 72 | 523 | TGGACAGGTGTCCGC |
| 73 | 524 | TGGACGGTTGTCCGC |
| 74 | 525 | TGGACTTGTGGTC |
| 75 | 526 | TGGAGATTCTCTCCGC |
| 76 | 527 | TGGCATAGGCCTGC |
| 77 | 528 | TGGCTTATGTCTAC |
| 78 | 529 | TGGGAGTTAATCCCGT |
| 79 | 530 | TGGGATCTTCCCGC |
| 80 | 531 | TGGGCAGAAATGTCTC |
| 81 | 532 | TGGGCGTTCGCCCGC |
| 82 | 533 | TGGGCTTCGCCCGC |
| 83 | 534 | TGGGGGATAACCCCGT |
| 84 | 535 | TGGGGGTTTCCCCGT |
| 85 | 536 | TGGT |
| 86 | 537 | TGGTGGCAACACCGT |
| 87 | 538 | TGGTTTATAGCCGT |
| 88 | 539 | TGTACGGTAATACCGTACC |
| 89 | 540 | TGTCCGCAAGGACGT |
| 90 | 541 | TGTCCTAACGGACGT |
| 91 | 542 | TGTCCTATTAACGGACGT |
| 92 | 543 | TGTCCTTCACGGGCGT |
| 93 | 544 | TGTCTTAGGACGT |
| 94 | 545 | TGTGCGTTAACGCGTACC |
| 95 | 546 | TGTGTCGCAAGGCACC |
| 96 | 547 | TGTTCGTAAGGACTT |
| 97 | 548 | TTCACAGAAATGTGTC |
| 98 | 549 | TTCCCTCGTGGAGT |
| 99 | 550 | TTCCCTCTGGGAGC |
| 100 | 551 | TTCCCTTGTGGATC |
| 101 | 552 | TTCCTTCGGGAGC |
| 102 | 553 | TTCTAGCAATAGAGT |
| 103 | 554 | TTCTCCACTGGGGAGC |
| 104 | 555 | TTCTCGAGAGGGAGC |
| 105 | 556 | TTCTCGTATGAGAGC |
| 106 | 557 | TTTAAGGTTTTCCCTTAAC |
| 107 | 558 | TTTCATTGTGGAGT |
| 108 | 559 | TTTCGAAGGAATCC |
| 109 | 560 | tttcttcggaagc |
| 110 | 561 | TTTGGGGCAACTCAAC |

### Method of Making TREMs

Methods for designing and constructing expression vectors and modifying a host cell for production of a target (e.g., a TREM or an enzyme disclosed herein) use techniques known in the art. For example, a cell is genetically modified to express an exogenous TREM using cultured mammalian cells (e.g., cultured human cells), insect cells, yeast, bacteria, or other cells under the control of appropriate promoters. Generally, recombinant methods may be used. See, in general, Pharmaceutical Biotechnology: Fundamentals and Applications, Springer (2013); Green and Sambrook (Eds.), Molecular Cloning: A Laboratory Manual (Fourth Edition), Cold Spring Harbor Laboratory Press (2012). For example, mammalian expression vectors may comprise non-transcribed elements such as an origin of replication, a suitable promoter and enhancer, and other 5′ or 3′ flanking non-transcribed sequences. DNA sequences derived from the SV40 viral genome, for example, SV40 origin, early promoter, enhancer, splice, and polyadenylation sites may be used to provide the other genetic elements required for expression of a heterologous DNA sequence.

A method of making a TREM or TREM composition disclosed herein comprises use of a host cell, e.g., a modified host cell, expressing a TREM.

The modified host cell is cultured under conditions that allow for expression of the TREM. In an embodiment, the culture conditions can be modulated to increase expression of the TREM. The method of making a TREM further comprises purifying the expressed TREM from the host cell culture to produce a TREM composition. In an embodiment the TREM is a TREM fragment, e.g., a fragment of a tRNA encoded by a deoxyribonucleic acid sequence disclosed in Table 1. E.g., the TREM includes less than the full sequence of a tRNA, e.g., less than the full sequence of a tRNA with the same anticodon, from the same species as the subject being treated, or both. In an embodiment, the production of a TREM fragment, e.g., from a full length TREM or a longer fragment, can be catalyzed by an enzyme, e.g., an enzyme having nuclease activity (e.g., endonuclease activity or ribonuclease activity), e.g., RNase A, Dicer, Angiogenin, RNaseP, RNaseZ, Rny1 or PrrC.

In an embodiment, a method of making a TREM described herein comprises contacting (e.g., transducing or transfecting) a host cell (e.g., as described herein, e.g., a modified host cell) with an exogenous nucleic acid described herein, e.g., a DNA or RNA, encoding a TREM under conditions sufficient to express the TREM. In an embodiment, the exogenous nucleic acid comprises an RNA (or DNA encoding an RNA) that comprises a ribonucleic acid (RNA) sequence of an RNA encoded by a DNA sequence disclosed in Table 1. In an embodiment, the exogenous nucleic acid comprises an RNA sequence (or DNA encoding an RNA sequence) that is at least 60%, 65%, 70%, 75%, 80%, 82%, 85%, 87%, 88%, 90%, 92%, 95%, 96%, 97%, 98%, 99% or 100% identical to an RNA sequence encoded by a DNA sequence provided in Table 1. In an embodiment, the exogenous nucleic acid comprises an RNA sequence (or DNA encoding an RNA sequence) that comprises at least 30 consecutive nucleotides of a ribonucleic acid (RNA) sequence encoded by a deoxyribonucleic acid (DNA) sequence disclosed in Table 1. In an embodiment, the exogenous nucleic acid comprises an RNA sequence (or DNA encoding an RNA sequence) that comprises at least 30 consecutive nucleotides of an RNA sequence at least 60%, 65%, 70%, 75%, 80%, 82%, 85%, 87%, 88%, 90%, 92%, 95%, 96%, 97%, 98%, 99% or 100% identical to an RNA sequence encoded by a DNA sequence provided in Table 1.

In an embodiment, the host cell is transduced with a virus (e.g., a lentivirus, adenovirus or retrovirus) expressing a TREM, e.g., as described in Example 2.

The expressed TREM can be purified from the host cell or host cell culture to produce a TREM composition, e.g., as described herein. Purification of the TREM can be performed by affinity purification, e.g., as described in the MACS Isolation of specific tRNA molecules protocol, or other methods known in the art. In an embodiment, a TREM is purified by a method described in Example 1.

In an embodiment, a method of making a TREM, e.g., TREM composition, comprises contacting a TREM with a reagent, e.g., a capture reagent comprising a nucleic acid sequence complimentary with a TREM. A single capture reagent or a plurality of capture reagents can be used to make a TREM, e.g., a TREM composition. When a single capture reagent is used, the capture reagent can have at least 50%, 55%, 60%, 65%, 70%, 75%, 80%, 85%, 90%, or 95% complimentary sequence with the TREM. When a plurality of capture reagents is used, a composition of TREMs having a plurality of different TREMs can be made. In an embodiment, the capture reagent can be conjugated to an agent, e.g., biotin.

In an embodiment, the method comprises denaturing the TREM, e.g., prior to hybridization with the capture reagent. In an embodiment, the method comprises, renaturing the TREM, after hybridization and/or release from the capture reagent.

In an embodiment, a method of making a TREM, e.g., TREM composition, comprises contacting a TREM with a reagent, e.g., a separation reagent, e.g., a chromatography reagent. In an embodiment, a chromatography reagent includes a column chromatography reagent, a planar chromatography reagent, a displacement chromatography reagent, a gas chromatography reagent, a liquid chromatography reagent, an affinity chromatography reagent, an ion-exchange chromatography reagent, or a size-exclusion chromatography reagent.

In an embodiment, a TREM made by any of the methods described herein can be: (i) charged with an amino acid, e.g., a cognate amino acid; (ii) charged with a non-cognate amino acid (e.g., a mischarged TREM (mTREM); or (iii) not charged with an amino acid, e.g., an uncharged TREM (uTREM).

In an embodiment, a TREM made by any of the methods described herein is an uncharged TREM (uTREM). In an embodiment, a method of making a uTREM comprises culturing the host cell in media that has a limited amount of one or more nutrients, e.g., the media is nutrient starved.

In an embodiment, a charged TREM, e.g., a TREM charged with a cognate AA or a non-cognate AA, can be uncharged, e.g., by dissociating the AA, e.g., by incubating the TREM at a high temperature.

### Exogenous Nucleic Acid Encoding a TREM or a TREM Fragment

In an embodiment, an exogenous nucleic acid, e.g., a DNA or RNA, encoding a TREM (e.g., a TREM corresponding to a con-rare codon), comprises a nucleic acid sequence comprising a nucleic acid sequence of one or a plurality of RNA sequences encoded by a DNA sequence disclosed in Table 1, e.g., any one of SEQ ID NOs: 1-451 as disclosed in Table 1. In an embodiment, an exogenous nucleic acid, e.g., a DNA or RNA, encoding a TREM comprises a nucleic acid sequence at least 60%, 65%, 70%, 75%, 80%, 85%, 90%, 95%, 96%, 97%, 98%, or 99% identical to an RNA sequence encoded by a DNA sequence disclosed in Table 1, e.g., any one of SEQ ID NOs: 1-451 as disclosed in Table 1.

In an embodiment, an exogenous nucleic acid, e.g., a DNA or RNA, encoding a TREM (e.g., a TREM corresponding to a con-rare codon),comprises the nucleic acid sequence of an RNA sequence encoded by a DNA sequence disclosed in Table 1, e.g., any one of SEQ ID NOs: 1-451 as disclosed in Table 1. In an embodiment, an exogenous nucleic acid, e.g., a DNA or RNA, encoding a TREM comprises a nucleic acid sequence at least 60%, 65%, 70%, 75%, 80%, 85%, 90%, 95%, 96%, 97%, 98%, 99%, or 100% identical to a plurality of RNA sequences encoded by a DNA sequence disclosed in Table 1, e.g., any one of SEQ ID NOs: 1-451 as disclosed in Table 1. In an embodiment, an exogenous nucleic acid encoding a TREM comprises an RNA sequence encoded by a DNA sequence at least 60%, 65%, 70%, 75%, 80%, 85%, 90%, 95%, 96%, 97%, 98%, or 99% identical to a DNA sequence disclosed in Table 1, e.g., any one of SEQ ID NOs: 1-451 as disclosed in Table 1.

In an embodiment, an exogenous nucleic acid, e.g., a DNA or RNA, encoding a TREM (e.g., a TREM corresponding to a con-rare codon), comprises an RNA sequence of one or a plurality of TREM fragments, e.g., a fragment of an RNA encoded by a DNA sequence disclosed in Table 1, e.g., as described herein, e.g., a fragment of any one of SEQ ID NOs: 1-451 as disclosed in Table 1. In an embodiment, a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of a nucleic acid sequence of an RNA encoded by a DNA sequence provided in Table 1, e.g., any one of SEQ ID NOs: 1-451 as disclosed in Table 1. In an embodiment, a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of a nucleic acid sequence at least 60%, 65%, 70%, 75%, 80%, 85%, 90%, 95%, 96%, 97%, 98%, or 99% identical to an RNA encoded by a DNA sequence provided in Table 1. In an embodiment, a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of a nucleic acid sequence encoded by a DNA sequence at least 60%, 65%, 70%, 75%, 80%, 85%, 90%, 95%, 96%, 97%, 98%, or 99% identical to a DNA sequence provided in Table 1, e.g., any one of SEQ ID NOs: 1-451 as disclosed in Table 1.

In an embodiment, a TREM fragment (e.g., a TREM fragment corresponding to a con-rare codon),comprises at least 5, 6, 7, 8, 9, 10, 11, 12, 13, 14, 15, 16, 17, 18, 19, 20, 21, 22, 23 24, 25, 26, 27, 28, 29 or 30 consecutive nucleotides of an RNA sequence encoded by a DNA sequence disclosed in Table 1 e.g., any one of SEQ ID NOs: 1-451 as disclosed in Table 1. In an embodiment, a TREM fragment comprises at least 5, 6, 7, 8, 9, 10, 11, 12, 13, 14, 15, 16, 17, 18, 19, 20, 21, 22, 23 24, 25, 26, 27, 28, 29 or 30 consecutive nucleotides of an RNA sequence at least 60%, 65%, 70%, 75%, 80%, 82%, 85%, 87%, 88%, 90%, 92%, 95%, 96%, 97%, 98%, or 99% identical to an RNA sequence encoded by a DNA sequence provided in Table 1 e.g., any one of SEQ ID NOs: 1-451 as disclosed in Table 1. In an embodiment, a TREM fragment comprises at least 5, 6, 7, 8, 9, 10, 11, 12, 13, 14, 15, 16, 17, 18, 19, 20, 21, 22, 23 24, 25, 26, 27, 28, 29 or 30 consecutive nucleotides of an RNA sequence encoded by a DNA sequence at least 60%, 65%, 70%, 75%, 80%, 82%, 85%, 87%, 88%, 90%, 92%, 95%, 96%, 97%, 98%, or 99% identical to a DNA sequence provided in Table 1 e.g., any one of SEQ ID NOs: 1-451 as disclosed in Table 1.

In an embodiment, the exogenous nucleic acid comprises a DNA, which upon transcription, expresses a TREM.

In an embodiment, the exogenous nucleic acid comprises an RNA, which upon reverse transcription, results in a DNA which can be transcribed to provide the TREM.

In an embodiment, the exogenous nucleic acid encoding a TREM comprises: (i) a control region sequence; (ii) a sequence encoding a modified TREM; (iii) a sequence encoding more than one TREM; or (iv) a sequence other than a tRNAMET sequence.

In an embodiment, the exogenous nucleic acid encoding a TREM comprises a promoter sequence. In an embodiment, the exogenous nucleic acid comprises an RNA Polymerase III (Pol III) recognition sequence, e.g., a Pol III binding sequence. In an embodiment, the promoter sequence comprises a U6 promoter sequence or fragment thereof. In an embodiment, the nucleic acid sequence comprises a promoter sequence that comprises a mutation, e.g., a promoter-up mutation, e.g., a mutation that increases transcription initiation, e.g., a mutation that increases TFIIIB binding. In an embodiment, the nucleic acid sequence comprises a promoter sequence which increases Pol III binding and results in increased tRNA production, e.g., TREM production.

Also disclosed herein is a plasmid comprising an exogenous nucleic acid encoding a TREM. In an embodiment, the plasmid comprises a promoter sequence, e.g., as described herein.

### TREM Composition

In an embodiment, a TREM composition, e.g., a composition comprising a TREM, e.g., a pharmaceutical composition comprising a TREM, comprises a pharmaceutically acceptable excipient. Exemplary excipients include those provided in the FDA Inactive Ingredient Database (https://www.accessdata.fda.gov/scripts/cder/iig/index.Cfm).

In an embodiment, a TREM composition, e.g., a composition comprising a TREM, e.g., a pharmaceutical composition comprising a TREM, comprises at least 1, 2, 3, 4, 5, 6, 7, 8, 9, 10, 15, 20, 30, 40, 50, 60, 70, 80, 90, 100 or 150 grams of TREM. In an embodiment, a TREM composition, e.g., a composition comprising a TREM, e.g., a pharmaceutical composition comprising a TREM, comprises at least 1, 2, 3, 4, 5, 6, 7, 8, 9, 10, 15, 20, 30, 40, 50 or 100 milligrams of TREM.

In an embodiment, a TREM composition, e.g., a composition comprising a TREM, e.g., a pharmaceutical composition comprising a TREM, is at least 10, 20, 30, 40, 50, 60, 70, 80, 90, 95 or 99% dry weight TREMs.

In an embodiment, a TREM composition, e.g., a composition comprising a TREM produced by any of the methods of making disclosed herein can be charged with an amino acid using an in vitro charging reaction as disclosed in Example 14, or as known in the art.

In an embodiment, a TREM composition, e.g., a composition comprising a TREM comprises at least 1×10⁶ TREM molecules, at least 1×10⁷ TREM molecules, at least 1×10⁸ TREM molecules or at least 1×10⁹ TREM molecules.

### TREM Purification

A TREM composition, e.g., a composition comprising a TREM, e.g., a pharmaceutical composition comprising a TREM, may be purified from host cells by nucleotide purification techniques. In one embodiment, a TREM composition, e.g., a composition comprising a TREM is purified by affinity purification, e.g., as described in the MACS Isolation of specific tRNA molecules protocol, or by a method described in Example 1. In one embodiment, a TREM composition, e.g., a composition comprising a TREM is purified by liquid chromatography, e.g., reverse-phase ion-pair chromatography (IP-RP), ion-exchange chromatography (IE), affinity chromatography (AC), size-exclusion chromatography (SEC), and combinations thereof. See, e.g., Baronti et al. Analytical and Bioanalytical Chemistry (2018) 410:3239-3252.

### TREM Quality Control and Production Assessment

A TREM, or a TREM composition, e.g., a composition comprising a TREM, e.g., a pharmaceutical composition comprising a TREM, produced by any of the methods disclosed herein can be assessed for a characteristic associated with the TREM or the TREM preparation, such as purity, host cell protein or DNA content, endotoxin level, sterility, TREM concentration, TREM structure, or functional activity of the TREM. Any of the above-mentioned characteristics can be evaluated by providing a value for the characteristic, e.g., by evaluating or testing the TREM, the TREM composition, or an intermediate in the production of the composition comprising a TREM. The value can also be compared with a standard or a reference value. Responsive to the evaluation, the TREM composition can be classified, e.g., as ready for release, meets production standard for human trials, complies with ISO standards, complies with cGMP standards, or complies with other pharmaceutical standards. Responsive to the evaluation, the TREM composition can be subjected to further processing, e.g., it can be divided into aliquots, e.g., into single or multi-dosage amounts, disposed in a container, e.g., an end-use vial, packaged, shipped, or put into commerce. In embodiments, in response to the evaluation, one or more of the characteristics can be modulated, processed or re-processed to optimize the TREM composition. For example, the TREM composition can be modulated, processed or re-processed to (i) increase the purity of the TREM composition; (ii) decrease the amount of HCP in the composition; (iii) decrease the amount of DNA in the composition; (iv) decrease the amount of fragments in the composition; (v) decrease the amount of endotoxins in the composition; (vi) increase the in vitro translation activity of the composition; (vii) increase the TREM concentration of the composition; or (viii) inactivate or remove any viral contaminants present in the composition, e.g., by reducing the pH of the composition or by filtration.

In an embodiment, the TREM (e.g., the TREM composition or an intermediate in the production of the TREM composition) has a purity of at least 30%, 40%, 50%, 60%, 70%, 80%, 85%, 90%, 91%, 92%, 93%, 94%, 95%, 96%, 97%, 98%, or 99%, i.e., by mass.

In an embodiment, the TREM (e.g., the TREM composition or an intermediate in the production of the TREM composition) has a host cell protein (HCP) contamination of less than 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, or 500 ng/ml.

In an embodiment, the TREM (e.g., the TREM composition or an intermediate in the production of the TREM composition) has a host cell protein (HCP) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, 100 ng, 200 ng, 300 ng, 400 ng, or 500 ng per milligram (mg) of the composition.

In an embodiment, the TREM (e.g., the TREM composition or an intermediate in the production of the TREM composition) has a DNA content, e.g., host cell DNA content, of less than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, or 500 ng/ml.

In an embodiment, the TREM (e.g., the TREM composition or an intermediate in the production of the TREM composition) has less than 0.1%, 1%, 2%, 3%, 4%, 5%, 6%, 7%, 8%, 9%, 10%, 15%, 20%, 25% TREM fragments.

In an embodiment, the TREM (e.g., the TREM composition or an intermediate in the production of the TREM composition) has low levels or absence of endotoxins, e.g., as measured by the Limulus amebocyte lysate (LAL) test;

In an embodiment, the TREM (e.g., the TREM composition or an intermediate in the production of the TREM composition) has in-vitro translation activity, e.g., as measured by an assay described in Example 9.

In an embodiment, the TREM (e.g., the TREM composition or an intermediate in the production of the TREM composition) has a TREM concentration of at least 0.1 ng/mL, 0.5 ng/mL, 1 ng/mL, 5 ng/mL, 10 ng/mL, 50 ng/mL, 0.1 ug/mL, 0.5 ug/mL, 1 ug/mL, 2 ug/mL, 5 ug/mL, 10 ug/mL, 20 ug/mL, 30 ug/mL, 40 ug/mL, 50 ug/mL, 60 ug/mL, 70 ug/mL, 80 ug/mL, 100 ug/mL, 200 ug/mL, 300 ug/mL, 500 ug/mL, 1000 ug/mL, 5000 ug/mL, 10,000 ug/mL, or 100,000 ug/mL.

In an embodiment, the TREM (e.g., the TREM composition or an intermediate in the production of the TREM composition) is sterile, e.g., the composition or preparation supports the growth of fewer than 100 viable microorganisms as tested under aseptic conditions, the composition or preparation meets the standard of USP <71>, and/or the composition or preparation meets the standard of USP <85>.

In an embodiment, the TREM (e.g., the TREM composition or an intermediate in the production of the TREM composition) has an absence of, or an undetectable level of a viral contaminant, e.g., no viral contaminants. In an embodiment, a viral contaminant, e.g., any residual virus, present in the composition is inactivated or removed. In an embodiment, a viral contaminant, e.g., any residual virus, is inactivated, e.g., by reducing the pH of the composition. In an embodiment, a viral contaminant, e.g., any residual virus, is removed, e.g., by filtration or other methods known in the field.

### TREM Administration

A TREM composition, e.g., a composition comprising a TREM, or a pharmaceutical composition comprising a TREM described herein can be administered to a target cell, tissue or subject (e.g., a target cell or tissue comprising a nucleic acid sequence having a con-rare codon), e.g., by direct administration to a target cell, tissue and/or an organ in vitro, ex-vivo or in vivo. In-vivo administration may be via, e.g., by local, systemic and/or parenteral routes, for example intravenous, subcutaneous, intraperitoneal, intrathecal, intramuscular, ocular, nasal, urogenital, intradermal, dermal, enteral, intravitreal, intracerebral, intrathecal, or epidural.

In an embodiment, a TREM composition, e.g., a composition comprising a TREM, or a pharmaceutical composition comprising a TREM disclosed herein is administered to a subject having a symptom or disorder disclosed herein, e.g., a disorder associated with a con-rare codon. In an embodiment, a TREM composition, e.g., a composition comprising a TREM, or a pharmaceutical composition comprising a TREM disclosed herein is administered to prevent or treat the symptom or disorder, e.g., a disorder associated with a con-rare codon. In an embodiment, administration of the TREM composition, e.g., a composition comprising a TREM or a pharmaceutical composition comprising a TREM results in treatment or prevention of the symptom or disorder. In an embodiment, administration of the TREM composition, e.g., a composition comprising a TREM or a pharmaceutical composition comprising a TREM modulates a tRNA pool in the subject, e.g., resulting in treatment of the symptom or disorder.

In an embodiment, a TREM composition, e.g., a composition comprising a TREM or a pharmaceutical composition comprising a TREM disclosed herein is administered to a cell from a subject having a symptom or disorder disclosed herein, e.g., a disorder associated with a con-rare codon. In an embodiment, administration of the TREM composition, e.g., a composition comprising a TREM, or the pharmaceutical composition comprising a TREM modulates a production parameter of an RNA, or a protein encoded by the RNA, having a con-rare codon. In an embodiment, the TREM composition, e.g., a composition comprising a TREM or pharmaceutical composition comprising a TREM can be administered to the cell in vivo, in vitro or ex vivo.

In an embodiment, a TREM composition or a pharmaceutical composition comprising a TREM disclosed herein is administered to a tissue in a subject having a symptom or disorder disclosed herein, e.g., a disorder associated with a con-rare codon.

### Vectors and Carriers

In some embodiments the TREM, or TREM composition, or pharmaceutical composition comprising a TREM described herein, is delivered to cells, e.g. mammalian cells or human cells, using a vector. The vector may be, e.g., a plasmid or a virus. In some embodiments, delivery is in vivo, in vitro, ex vivo, or in situ. In some embodiments, the virus is an adeno associated virus (AAV), a lentivirus, an adenovirus. In some embodiments, the system or components of the system are delivered to cells with a viral-like particle or a virosome. In some embodiments, the delivery uses more than one virus, viral-like particle or virosome.

### Carriers

A TREM, TREM composition, or a pharmaceutical composition comprising a TREM described herein may comprise, may be formulated with, or may be delivered in, a carrier.

### Viral Vectors

The carrier may be a viral vector (e.g., a viral vector comprising a sequence encoding a TREM). The viral vector may be administered to a cell or to a subject (e.g., a human subject or animal model) to deliver a TREM, a TREM composition or a pharmaceutical composition comprising a TREM. A viral vector may be systemically or locally administered (e.g., injected).

Viral genomes provide a rich source of vectors that can be used for the efficient delivery of exogenous genes into a mammalian cell. Viral genomes are known in the art as useful vectors for delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors include a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus, replication deficient herpes virus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology (Third Edition) Lippincott-Raven, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the teachings of which are incorporated herein by reference. In some embodiments the system or components of the system are delivered to cells with a viral-like particle or a virosome.

### Cell and Vesicle-Based Carriers

A TREM, a TREM composition or a pharmaceutical composition comprising a TREM described herein can be administered to a cell in a vesicle or other membrane-based carrier.

In embodiments, a TREM, TREM composition or pharmaceutical composition comprising a TREM described herein is administered in or via a cell, vesicle or other membrane-based carrier. In one embodiment, the TREM, TREM composition or pharmaceutical composition comprising a TREM can be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Lipid nanoparticles are another example of a carrier that provides a biocompatible and biodegradable delivery system for a TREM, TREM composition or pharmaceutical composition comprising a TREM described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Exosomes can also be used as drug delivery vehicles for a TREM, or TREM composition, or a pharmaceutical composition comprising a TREM described herein. For a review, see Ha et al. July 2016. Acta Pharmaceutica Sinica B. Volume 6, Issue 4, Pages 287-296; https://doi.org/10.1016/j.apsb.2016.02.001.

Ex vivo differentiated red blood cells can also be used as a carrier for a TREM, TREM composition or a pharmaceutical composition comprising a TREM described herein. See, e.g., WO2015073587; WO2017123646; WO2017123644; WO2018102740; wO2016183482; WO2015153102; WO2018151829; WO2018009838; Shi et al. 2014. Proc Natl Acad Sci USA. 111(28): 10131-10136; U.S. Pat. No. 9,644,180; Huang et al. 2017. Nature Communications 8: 423; Shi et al. 2014. Proc Natl Acad Sci USA. 111(28): 10131-10136.

Fusosome compositions, e.g., as described in WO2018208728, can also be used as carriers to deliver a TREM, a TREM composition, or a pharmaceutical composition comprising a TREM described herein.

All references and publications cited herein are hereby incorporated by reference.

The following examples are provided to further illustrate some embodiments of the present invention, but are not intended to limit the scope of the invention; it will be understood by their exemplary nature that other procedures, methodologies, or techniques known to those skilled in the art may alternatively be used.

### EXAMPLES

### Table of Contents for Examples

Con-rare codon identification

| | |
|---|---|
| Example la | Quantitative tRNA profiling by Oxford Nanopore sequencing |
| Example 1b | Quantitative tRNA profiling by next generation sequencing |
| Example 2 | Quantification of protein expression levels across cell lines or tissue types |
| Example 3 | Evaluation of contextual rarity and identification of contextually rare |
| | codons |
| Example 4 | Identification of a nucleic acid sequence having con-rare codons (A) |
| Example 5 | Identification of a nucleic acid sequence having con-rare codons (B) |
| Example 6 | Exemplary nucleic acid sequence having con-rare codons |
| Example 7 | Exemplary computational pipeline for codon modifying a nucleic acid |
| | sequence |

Determining the effect of TREM administration on protein expression

| | |
|---|---|
| Example 8 | Determining that administration of a TREM affects expression of a protein |
| | encoded by a nucleic acid sequence having a con-rare codon |

Manufacturing and preparation of TREMs

| | |
|---|---|
| Example 9 | Manufacture of TREM in a mammalian production host cell, and use |
| | thereof to modulate a cellular function |
| Example 10 | Manufacture of TREM in a mammalian production host cell, and use |
| | thereof to modulate a cellular function |
| Example 11 | Manufacture of TREMs in modified mammalian production host cell |
| | expressing an oncogene |
| Example 12 | Preparation of a TREM production host cell modified to inhibit a repressor |
| | of tRNA synthesis |
| Example 13 | Manufacture of TREMs in modified mammalian production host cell |
| | overexpressing an oncogene and a tRNA modifying enzyme |

Assays to analyze TREM activity

| | |
|---|---|
| Example 14 | TREM translational activity assay |

Production of TREMs

| | |
|---|---|
| Example 15 | Production of a candidate TREM complementary to the con-rare codon |
| | through mammalian cell purification |
| Example 16 | Production of a candidate TREM complementary to the con-rare codon |
| | through bacterial cell purification |
| Example 17 | Production of a candidate TREM complementary to the con-rare codon |
| | through chemical synthesis |
| Example 18 | Production of a candidate TREM complementary to the con-rare codon |
| | through in vitro transcription |

### Example 1a: Quantitative tRNA Profiling by Oxford Nanopore Sequencing

This Example describes the quantification of tRNA levels in a cell line or tissue type which is useful for identifying con-rare codons and candidate con-rare codons. Transfer RNA levels are determined using Oxford Nanopore direct RNA sequencing, as previously described in Sadaoka et al., Nature Communications (2019) 10, 754. Briefly, cells transfected with a tRNA molecule are lysed and total RNA is purified using a method such as phenol chloroform. RNAs smaller than 200 nucleotides are separated from the lysate using a small RNA isolation kit per manufacturer's instructions, to generate a small RNA (sRNA) fraction.

The sRNA fraction is de-acylated using 100 mM Tris-HCl (pH 9.0) at 37° C. for 30 minutes. The solution is neutralized by the addition of an equal volume of 100 mM Na-acetate/acetic acid (pH 4.8) and 100 mM NaCl, followed by ethanol precipitation. Deacylated sRNA is dissolved in water, and its integrity verified by agarose gel electrophoresis. Deacylated sRNA is then polyadenylated using yeast poly(A) tailing kit per manufacturer's instructions to generate a sRNA polyadenylated pool. Following polyadenylation, a reverse transcription reaction is performed to generate cDNA using SuperScript III Reverse Transcriptase (Thermo Fisher Scientific) or a thermostable group II intron RT (TGIRT, InGex LLC) that is less sensitive to RNA structure and modifications. A sequencing adapter is ligated onto the cDNA mixture by incubating the cDNA mixture with RNA adapter, T4 ligase and ligation buffer following the standard protocol for Oxford Nanopore resulting in a cDNA library. Nanopore sequencing is then performed on the libraries and the sequences are mapped to a genomic database, in this example to the genomic tRNA database, GtRNAdb. The methods described in this example can be adopted for use to evaluate the tRNA pool across cell lines or tissue types.

### Example 1b: Quantitative tRNA Profiling by Next Generation Sequencing

This Example describes the quantification of tRNA levels in a cell line or tissue type. Transfer RNA levels are determined using next generation sequencing, as previously described in Pinkard et al., Nature Communications (2020) 11, 4104.

Briefly, cells transfected with a tRNA molecule are lysed and total RNA is purified using a method such as phenol chloroform. RNAs smaller than 200 nucleotides are separated from the lysate using a small RNA isolation kit per manufacturer's instructions, to generate a small RNA (sRNA) fraction.

The sRNA fraction is de-acylated using 100 mM Tris-HCl (pH 9.0) at 37° C. for 45 minutes. The solution is neutralized by the addition of an equal volume of 100 mM Na-acetate/acetic acid (pH 4.8) and 100 mM NaCl, followed by ethanol precipitation. Deacylated sRNA is splint ligated in a reaction with 3′ adapter, a mix of 4 splint strands and annealing buffer at 37° C. for 15 minutes followed by addition of a RNL2 ligase reaction buffer mix at 37° C. for 1h and then at at 4° C. for hr. The deacylated and splint ligated sRNA is precipitated using a method such as phenol chloroform extraction.

The deacylated and splint ligated sRNA is reverse transcribed using an RT enzyme such as Superscript IV at 55° C. for 1 hr. The reaction product is desalted in a micro bio0sepin P30 according to manufacturer directions and sample is run on a denaturing polyacrylamide gel. Gel band from 65-200 nt was excised, and sRNA was extracted. The sRNA was circularized using a circligase and purified. The purified circularized RNA was PCR amplified and product run on a e-gel ex. Bands from 100-250 nt were excised and purified using qiaquick gel extraction kit according to manufacturer directions and RNA was precipitated. Next generation sequencing is then performed on the libraries and the sequences are mapped to a genomic database, in this example to the genomic tRNA database, GtRNAdb. The methods described in this example can be adopted for use to evaluate the tRNA pool across cell lines or tissue types.

### Example 2: Quantification of Protein Expression Levels Across Cell Lines or Tissue Types

This Example describes the quantification of protein expression levels across cell lines or tissue types which is useful for identifying con-rare codons and candidate con-rare codons.

### Cell Culture Sample Preparation

The protein expression levels are monitored using SILAC based mass-spectrometry proteomics, as previously described in Geiger et al., Molecular and Cellular Proteomics (2012) 10, 754.

Briefly, populations of cells are cultured either in media containing isotope-labeled amino acids, such as Lys8 (e.g., 13C615N2-lysine) and Arg10 (e.g., 13C615N4-arginine); or in media containing natural amino acids. The media is further supplemented with 10% dialyzed serum. Cell cultured in media containing isotope-labeled amino acids incorporate the isotope-labeled amino acids into all of the proteins translated after incubation with said isotope-labeled amino acids. For example, all peptides containing a single arginine will be 6 Da heavier in cells cultured in the presence of instead of isotope-labeled amino acid compared to cells cultured with natural amino acids. Cultured are lysed and sonicated. Cell lysates (e.g., about 100 g) are diluted in 8 M urea in 0.1 M Tris-HCl followed by protein digestion with trypsin according to the FASP protocol (Wisniewski, J. R., et al. (2009) Universal sample preparation method for proteome analysis. Nat. Methods 6, 359-362). After an overnight digestion, peptides are eluted from the filters with 25 mM ammonium bicarbonate buffer. From each sample, about 40 ug of peptides are separated into six fractions by strong anion exchange as described previously (Wisniewski, J. R., et al. (2009) Combination of FASP and StageTip-based fractionation allows in-depth analysis of the hippocampal membrane proteome. J. Proteome Res. 8, 5674-5678).

Eluted peptides are concentrated and purified on C18 StageTips, e.g., as described in Rappsilber et al., Nature Protocols (2007).

### LC-MS/MS Analysis

Peptides are separated by reverse-phase chromatography using a nano-flow HPLC (Easy nanoLC, Thermo Fisher Scientific). The high performance liquid chromatography (HPLC) is coupled to an LTQ-Orbitrap Velos mass spectrometer (Thermo Fisher Scientific). Peptides are loaded onto the column with buffer A (0.5% acetic acid) and eluted with a 200 min linear gradient from 2 to 30% buffer B (80% acetonitrile, 0.5% acetic acid). After the gradient the column is washed with 90% buffer B and re-equilibrated with buffer A.

Mass spectra are acquired in a data-dependent manner, with an automatic switch between MS and MS/MS scans using a top 10 method. MS spectra are acquired in the Orbitrap analyzer, with a mass range of 300-1650 Th and a target value of 106 ions. Peptide fragmentation is performed with the HCD method and MS/MS spectra is acquired in the Orbitrap analyzer and with a target value of 40,000 ions. Ion selection threshold is set to 5000 counts. Two of the data sets are acquired with a high field Orbitrap cell in which the resolution is 60,000 instead of 30,000 (at 400 m/z) for the MS scans. In the first of the two replicates with the high field Orbitrap MS/MS scans are acquired with 15,000 resolution, and in the second with 7500 resolution, which is the same as in the standard Orbitrap, but with shorter transients.

### Data Analysis

Raw MS files are analyzed by MaxQuant using standard metrics, e.g., as described in Table 2 of Tyanova S et al. (2016) Nat. Protocols 11(12) pp. 2301-19. Categorical annotation is supplied in the form of Gene Ontology (GO) biological process, molecular function, and cellular component, the TRANSFAC database as well as participation in a KEGG pathway and membership in a protein complex as defined by CORUM.

The methods described in this example can be adopted for use to evaluate the protein expression levels across cell lines or tissue types.

### Example 3: Evaluation of Contextual Rarity and Identification of Contextually Rare Codons

This example describes the method used to determine components of contextual rarity (con-rarity) for con-rare codons or candidate con-rare codons. This method utilizes the cell line or tissue protein expression level determined by proteomics described in Example 2 or taken from literature. This method also utilizes the tRNA profile determined by Nanopore or other tRNA sequencing platform described in the Example 1 or taken from literature.

### Codon Count Per Nucleic Acid Sequence

Using the coding DNA sequence (CDS) defined using National Center for Biotechnology Information (NCBI https://www.ncbi.nlm.nih.gov/) or other database, the protein-coding sequence is segmented into codons and summed per codon to give a codon count per nucleic acid sequence for each codon encoded in the protein-coding sequence.

### Normalized Proteome Codon Count

The codon count per nucleic acid sequence is then multiplied by the corresponding cell line or tissue protein expression level determined by proteomics to give a cell type normalized proteome codon count across the cell line or tissue.

### Con-Rarity

Con-rarity is a function of normalized proteome codon count and the tRNA expression level. In an embodiment, the con-rarity is determined by dividing the normalized proteome codon count by the tRNA expression level determined by Nanopore or other tRNA sequencing experiment. This provides a measure of codon usage that is contextually dependent on the tRNA profile, e.g., tRNA abundance levels. A codon is determined to be contextually rare (con-rare) if the con-rarity meets a reference value, e.g., a pre-determined or pre-selected reference value, e.g., a threshold. In an embodiment, a codon is con-rare if the value of a normalized proteome codon count divided by the tRNA expression level for a particular tRNA meets a pre-determined reference. In an embodiment, the reference value is a value under e.g., 1.5× sigma of the normally fit distribution to that codon frequency. See, for example, FIG. 2

### Example 4: Identification of a Nucleic Acid Sequence Having Con-Rare Codons (A)

This Example describes the identification of a nucleic acid sequence having con-rare codons or candidates for con-rare codons. Con-rare codons are identified as described in Example 3.

### Codon Count Per Nucleic Acid Sequence

Using the coding DNA sequences (CDS) defined using National Center for Biotechnology Information (NCBI https://www.ncbi.nlm.nih.gov/) or other database, all human gene sequences are segmented into codons and summed per codon to give a codon count per nucleic acid sequence, e.g., gene.

### Con-Rare Count Per Nucleic Acid Sequence

Each codon, per nucleic acid sequence, is classified as a con-rare codon or a con-abundant codon. The counts for all con-rare codons, for each nucleic acid sequence, are summed and normalized to the sequence length.

### Determining a Nucleic Acid Sequence Having Con-Rare Codons

The con-rare codon count is fit to a normalized distribution. A nucleic acid sequence that meets a reference value, e.g., a pre-determined reference value, is classified as a nucleic acid sequence having con-rare codons. In an embodiment, a nucleic acid sequence is classified as having con-rare codons if it falls above a reference value, e.g., in the upper 3sigma of the normalized distribution. In an embodiment, a nucleic acid sequence having con-rare codons can have one, two, or more (e.g., 3, 4, 5, 6, 7, 8, 9, 10, 20, 30, 50, 100, 200, 500) of the same con-rare codon or different con-rare codons.

### Example 5: Identification of a Nucleic Acid Sequence Having Con-Rare Codons (B)

This Example describes the identification of a nucleic acid sequence having con-rare codons or candidates con-rare codons. Con-rare codons are identified as described in the Example 3.

### Codon Count Per Nucleic Acid Sequence

Using the coding DNA sequences (CDS) defined using National Center for Biotechnology Information (NCBI https://www.ncbi.nlm.nih.gov/) or other database, all human gene sequences are segmented into codons and summed per codon to give a codon count per nucleic acid sequence, e.g., gene.

### Determining a Nucleic Acid Sequence Having Con-Rare Codons

Each codon, per nucleic acid sequence, is classified as a con-rare codon or a con-abundant codon. For each con-rare codon, the counts per nucleic acid sequence is fit to a normalized distribution. A nucleic acid sequence that meets a reference value, e.g., a pre-determined reference value, is classified as a nucleic acid sequence having con-rare codons. In an embodiment, a nucleic acid sequence is classified as having con-rare codons, e.g., specified con-rare codons, if it falls e.g., in the upper 3sigma of the normalized distribution. In an embodiment, a nucleic acid sequence having con-rare codons can have one, two, or more (e.g., 3, 4, 5, 6, 7, 8, 9, 10, 20, 30, 50, 100, 200, 500) of the same con-rare codon or different con-rare codons.

### Example 6: Exemplary Nucleic Acid Sequence Having Con-Rare Codons

This Example describes an exemplary nucleic acid sequence having con-rare codons or candidates con-rare codons.

The GRK2 nucleic acid sequence encodes the GRK2 protein (G-protein coupled receptor kinase 2). The method of Examples 4 or 5 was used to identify the GRK2 nucleic acid sequence as having con-rare codons. The GRK2 nucleic acid sequence has a coding sequence that has con-rare codons AAG and CTG. The AAG codon codes for lysine and the CTG codon codes for leucine. Under certain cellular conditions, the expression of the GRK2 protein can be affected by the frequency of tRNAs corresponding to one or more con-rare codons in the GRK2 nucleic acid sequence, e.g., CUU-tRNA which corresponds to con-rare codon AAG, and/or CAG-tRNA which corresponds to con-rare codon CTG.

### Example 7: Exemplary Computational Pipeline for Codon Modifying a Nucleic Acid Sequence

This Example describes the computational pipeline that can be utilized to codon modify a nucleic acid sequence.

Mapping con-rare codons Con-rarity (determined using the method described in Example 3) is read into the algorithm. Con-rare codons are identified as described in Example 3. For example, a codon is determined to be contextually rare (con-rare) if the con-rarity meets a reference value, e.g., a pre-determined or pre-selected reference value, e.g., a threshold. A corresponding contextually abundant (con-abundant) codon is identified as the most contextually frequent codon that encodes the same amino acid as the con-rare codon (e.g., an isoacceptor or an isodecoder). In an embodiment, a con-rare codon can have more than one corresponding con-abundant codon. In an embodiment, the corresponding con-abundant codon can be utilized to replace a con-rare codon.

### Con-Rare Codon Modification

Each sequence to be modified is read in and segmented into codons. Each codon is then evaluated to determine if it is a con-rare codon. If the codon is identified as a con-rare codon, the codon is replaced, e.g., with a corresponding con-abundant codon. A con-abundant codon is a codon other than a con-rare codon. This process can be repeated for two, three, four, or a portion of, or all of the con-rare codons found in the sequence. The resultant con-rare modified sequence (e.g., also referred to as contextually modified nucleic acid sequence) is then outputted.

### Example 8: Determining that Administration of a TREM Affects Expression of a Protein Encoded by a Nucleic Acid Sequence Having a Con-Rare Codon

This Example describes administration of a TREM to modulate expression levels of a protein encoded by a nucleic acid sequence having a con-rare codon in its coding sequence (CDS).

To create a system in which to study the effects of TREM administration on protein expression levels of a protein encoded by a nucleic acid sequence having a con-rare codon in its CDS, the sequence for the GRK2 gene (GRK2-CCDS8156.1 sequence) is inserted into a plasmid. The plasmid is transfected in the normal human hepatocyte cell line THLE-3. A TREM is delivered to the CCDS8156.1 containing cells. As a control, a population of cells prior to the delivery of the TREM is set aside. In this example, the tRNA-Lys^{CUU} containing an CUU anticodon, that base pairs to the AAG codon, i.e. with the sequence GCCCGGCUAGCUCAGUCGGUAGAGCAUGGGACUCUUAAUCCCAGGGUCGUGGGUU CGAGCCCCACGUUGGGCG is used. A time course is performed ranging from 30 minutes to 6 hours with hour-long interval time points. At each time point, a population of cells that have been delivered the TREM, and a population of cells that have not been exposed to the TREM are trypsinized, washed and lysed. Cell lysates are analyzed by Western blotting and blots are probed with antibodies against the GRK2 protein. A total protein loading control, such as GAPDH, actin or tubulin, is also used.

The methods described in this example can be adopted to evaluate the expression levels of the GRK2 protein in cells endogenously expressing CCDS8156.1.

### Example 9: Manufacture of TREM in a Mammalian Production Host Cell, and Use Thereof to Modulate a Cellular Function

This example describes the manufacturing of a TREM produced in mammalian host cells.

### Plasmid Generation

To generate a plasmid comprising a TREM which comprises a tRNA gene, in this example, tRNAiMet, a DNA fragment containing the tRNA gene (chr6.tRNA-iMet(CAT) with genomic location 6p22.2 and sequence AGCAGAGTGGCGCAGCGGAAGCGTGCTGGGCCCATAACCCAGAGGTCGATGGATCG AAACCATCCTCTGCTA) is PCR-amplified from human genomic DNA using the following primer pairs: 5′-TGAGTTGGCAACCTGTGGTA and 5′-TTGGGTGTCCATGAAAATCA. This fragment is cloned into the pLKO.1 puro backbone plasmid with a U6 promoter (or any other RNA polymerase III recruiting promoter) following the manufacturer's instructions.

### Transfection

1 mg of plasmid described above is used to transfect a 1 L culture of suspension-adapted HEK293T cells (Freestyle 293-F cells) at 1×10⁵ cells/mL. Cells are harvested at 24, 48, 72, or 96 hours post-transfection to determine the optimized timepoint for TREM expression as determined by Northern blot, or by quantitative PCR (q-PCR).

### Purification

At the optimized harvest cell density point, the TREM is purified as previously described in Cayama et al., Nucleic Acids Research. 28 (12), e64 (2000). Briefly, short RNAs (e.g., tRNAs) are recovered from cells by phenol extraction and concentrated by ethanol precipitation. The total tRNA in the precipitate is then separated from larger nucleic acids (including rRNA and DNA) under high salt conditions by a stepwise isopropanol precipitation. The elution fraction containing the TREM is further purified through probe binding. The TREM fraction is incubated with annealing buffer and the biotinylated capture probe corresponding to a DNA probe or a 2′-OMe nucleic acid that is complementary to a unique region of the TREM being purified, in this example, a probe conjugated to biotin at the 3′ end with the sequence UAGCAGAGGAUGGUUUCGAUCCAUCA, is used to purify the TREM comprising tRNA-Lys-UUU. The mixture is incubated at 90° C. for 2-3 minutes and quickly cooled down to 45° C. and incubated overnight at 45° C. The admixture is then incubated with binding buffer previously heated to 45° C. and streptavidin-conjugated RNase-free magnetic beads for 3 hours to allow binding of the DNA-tRNA complexes to the beads. The mixture is then added to a pre-equilibrated column in a magnetic field separator rack and washed 4 times. The TREM retained on the beads are eluted three times by adding elution buffer pre-heated to 80° C. and then admixed with a pharmaceutically acceptable excipient to make a test TREM product.

### Use

One microgram of the test TREM preparation and a control agent are contacted by transfection, electroporation or liposomal delivery, with a cultured cell line, such as a HEP-3B or HEK293T, a tissue or a subject, for a time sufficient for the TREM preparation to modulate a translation level or activity of the cell, relative to the control agent.

### Example 10: Manufacture of TREM in a Mammalian Production Host Cell, and Use Thereof to Modulate a Cellular Function

This example describes the manufacturing of a TREM produced in mammalian host cells.

### Plasmid Generation

To generate a plasmid comprising a TREM which comprises a tRNA gene, in this example, tRNA-iMet-CAT, a DNA fragment containing at least one copy of the tRNA gene with the sequence AGCAGAGTGGCGCAGCGGAAGCGTGCTGGGCCCATAACCCAGAGGTCGATGGATCG AAACCATCCTCTGCTA is synthesized and cloned into the pLKO.1 puro backbone plasmid with a U6 promoter (or any other RNA polymerase III recruiting promoter) following the manufacturer's instructions and standard molecular cloning techniques.

### Transfection

1 mg of plasmid described above is used to transfect a 1 L culture of suspension-adapted HEK293T cells (Freestyle 293-F cells) at 1×10⁵ cells/mL. Cells are harvested at 24, 48, 72, or 96 hours post-transfection to determine the optimized timepoint for TREM expression as determined by Northern blot, or by quantitative PCR (q-PCR) or Nanopore sequencing.

### Purification

At the optimized harvest timepoint, the cells are lysed and separation from the lysate of RNAs smaller than 200 nucleotides is performed using a small RNA isolation kit per manufacturer's instructions, to generate a small RNA (sRNA) fraction.

To prepare the affinity purification reagents, streptavidin-conjugated RNase-free magnetic beads are incubated at room temperature for 30 min with 200 mM of biotinylated oligonucleotides corresponding to a DNA probe or a 2′-OMe nucleic acid that is complementary to a unique region of the TREM being purified. In this example, a probe with the sequence 5′biotin-TAGCAGAGGATGGTTTCGATCCATCA is used to purify the TREM comprising tRNA-iMet (CAT). The beads are washed and heated for 10 min at 75° C.

The sRNA fraction is heated for 10 min at 75° C. and then mixed with the affinity purification reagent described above. The admixture is incubated at room temperature for 3 hours to allow binding of the TREMs to the bead-bound DNA probe in a sequence specific manner. The beads are then washed until the absorbance of the wash solution at 260 nm is close to zero. Alternatively, the beads are washed three times and the final wash is examined by UV spectroscopy to measure the amount of nucleic acid present in the final wash. The TREM retained on the beads are eluted three times using RNase-free water which can be pre-heated to 80° C., and then admixed with a pharmaceutically acceptable excipient to make a test TREM product.

### Use

One microgram of the test TREM preparation and a control agent are contacted by transfection, electroporation or liposomal delivery, with a cultured cell line, such as HeLa, HEP-3B or HEK293T, a tissue or a subject, for a time sufficient for the TREM preparation to modulate a translation level or activity of the cell, relative to the control agent.

### Example 11: Manufacture of TREMs in Modified Mammalian Production Host Cell Expressing an Oncogene

This example describes the manufacturing of a TREM in mammalian host cells modified to overexpress myc.

### Plasmid Generation and Host Cell Modification

To make the production host cells for this example, HeLa cells (ATCC® CCL-2™) or HEP-3B cells (ATCC® HB-8064™) are transfected with a plasmid containing the gene sequence coding for the c-myc oncogene protein (e.g., pcDNA3-cmyc (Addgene plasmid #16011)) using routine molecular biology techniques. The resulting cell line is referred to herein as HeLamyc+ host cells or HEP-3Bmyc+ host cells.

### Preparation of TREM Expressing Lentivirus

To prepare a TREM expressing lentivirus, HEK293T cells are co-transfected with 3 μg of each packaging vector (pRSV-Rev, pCMV-VSVG-G and pCgpV) and 9 μg of the plasmid comprising a TREM as described in Example 9, using Lipofectamine 2000 according to manufacturer's instructions. After 24 hours, the media is replaced with fresh antibiotic-free media and after 48 hours, virus-containing supernatant is collected and centrifuged for 10 min at 2000 rpm before being filtered through a 0.45 m filter.

Transduction of Host Cells with TREM Expressing Lentivirus

2 mL of virus prepared as described above is used to transduce 100,000 HeLamyc+ host cells or HEP-3Bmyc+ host cells, in the presence of 8 μg/mL polybrene. Forty-eight hours after transduction, puromycin (at 2 μg/mL) antibiotic selection is performed for 2-7 days alongside a population of untransduced control cells.

The TREMs are isolated, purified, and formulated as described in Example 9 or 10 to result in a composition comprising a TREM or preparation comprising a TREM.

### Example 12: Preparation of a TREM Production Host Cell Modified to Inhibit a Repressor of tRNA Synthesis

This example describes the preparation of Hek293Maf-/TRM1 cells for the production of a TREM.

Maf1 is a repressor of tRNA synthesis. A Maf1 knockout HEK293T cell line is generated using standard CRISPR/Cas knockout techniques, e.g., a CRISPR/Cas system can be designed to introduce a frameshift mutation in a coding exon of Maf1 to reduce the expression of Maf1 or knockout Maf1 expression, to generate a Hek293Maf-cell line that has reduced expression level and/or activity of Maf1. This cell line is then transfected with an expression plasmid for modifying enzyme Trm1 (tRNA (guanine26-N2)-dimethyltransferase) such as pCMV6-XL4-Trm1, and selected with a selection marker, e.g., neomycin, to generate a stable cell line overexpressing Trm1 (Hek293Maf-/TRM1 cells).

Hek293Maf-/TRM1 cells can be used as production host cells for the preparation of a TREM as described in any of Examples 9-11.

### Example 13: Manufacture of TREM in Modified Mammalian Production Host Cell Overexpressing an Oncogene and a tRNA Modifying Enzyme

This Example describes the manufacturing of a TREM in mammalian host cells modified to overexpress Myc and Trm1.

### Plasmid Generation

In this example, a plasmid comprising a TREM is generated as described in Example 9 or 10.

### Host Cell Modification, Transduction and Purification

A human cell line, such as HEK293T, stably overexpressing Myc oncogene is generated by transduction of retrovirus expressing the myc oncogene from the pBABEpuro-c-myc^{T58A} plasmid into HEK293T cells. To generate myc-expressing retrovirus, HEK293T cells are transfected using the calcium phosphate method with the human c-myc retroviral vector, pBABEpuro-c-myc^{T58A} and the packaging vector, W2 vector. After 6 hours, transfection media is removed and replaced with fresh media. After a 24-hour incubation, media is collected and filtered through a 0.45 um filter. For the retroviral infection, HEK293T cells are infected with retrovirus and polybrene (8 ug/ml) using spin infection at 18° C. for 1 hour at 2500 rpm. After 24 hours, the cell culture medium is replaced with fresh medium and 24 hours later, the cells are selected with 2 μg/mL puromycin. Once cells stably overexpressing the oncogene myc are established, they are transfected with a Trm1 plasmid, such as the pCMV6-XL4-Trm1 plasmid, and selected with a selection marker, in this case with neomycin, to generate a stable cell line overexpressing Trm1, in addition to Myc. In parallel, lentivirus to overexpress TREM is generated as described in Example 3 with HEK293T cells and PLKO.1-tRNA vectors.

1×10⁵ cells overexpressing Myc and Trm1 are transduced with the TREM virus in the presence of 8 μg/mL polybrene. Media is replaced 24 hours later. Forty-eight hours after transduction, antibiotic selection is performed with 2 μg/mL puromycin for 2-7 days alongside a population of untransduced control cells. The TREMs are isolated, purified and formulated using the method described in Example 9 or 10 to produce a TREM preparation.

### Example 14: TREM Translational Activity Assays

This example describes assays to evaluate the ability of a TREM to be incorporated into a nascent polypeptide chain.

### Translation of the FLAG-AA-his Peptide Sequence

A test TREM is assayed in an in-vitro translation reaction with an mRNA encoding the peptide FLAG-XXX-His6x, where XXX are 3 consecutive codons corresponding to the test TREM anticodon.

A tRNA-depleted rabbit reticulocyte lysate or human cell lysate (Jackson et al. 2001. RNA 7:765-773) is incubated 1 hour at 30° C. with 10-25 ug/mL of the test TREM in addition to 10-25 ug/mL of the tRNAs required for the FLAG and His tag translation. A different mammalian lysate such as a HEK293T human cell-derived lysate can also be used in this assay. In this example, the TREM used is tRNA-Ile-GAT, therefore the peptide used is FLAG-LLL-His6x and the tRNAs added are tRNA-Ile-GAT, in addition to the following, which are added for translate the peptide FLAG and HIS tags: tRNA-Asp-GAC, tRNA-Tyr-TAC, tRNA-Lys-AAA, tRNA-Lys-AAAG, tRNA-Asp-GAT, tRNA-His-CAT. To determine if the test TREM is functionally able to be incorporated into a nascent peptide, an ELISA capture assay is performed. Briefly, an immobilized anti-His6X antibody is used to capture the FLAG-LLL-His6x peptide from the reaction mixture. The reaction mixture is then washed off and the peptide is detected with an enzyme-conjugated anti-FLAG antibody, which reacts to a substrate in the ELISA detection step. If the TREM produced is functional, the FLAG-LLL-His6 peptide is produced and detection occurs by the ELISA capture assay. The methods described in this example can be adopted for use to evaluate the functionality of the TREM.

### Translational Suppression Assay

This assay describes a test TREM having translational adaptor molecule function by rescuing a suppression mutation and allowing the full protein to be translated. The test TREM, in this example tRNA-Ile-GAT, is produced such that it contains the sequence of the tRNA-Ile-GAT body but with the anticodon sequence corresponding to CUA instead of GAT. HeLa cells are co-transfected with 50 ng of TREM and with 200 ng of a DNA plasmid encoding a mutant GFP containing a UAG stop codon at the S29 position as described in Geslain et al. 2010. J Mol Biol. 396:821-831. HeLa cells transfected with the GFP plasmid alone serve as a negative control. After 24 hours, cells are collected and analyzed for fluorescence recovery by flow cytometry. The fluorescence is read out with an emission peak at 509 nm (excitation at 395 nm). The methods described in this example can be adopted for use to evaluate the functionality of the TREM, or if the TREM can rescue the stop mutation in the GFP molecule and can produce the full-length fluorescent protein.

### In Vitro Translational Assay

This assay describes a test TREM having translational adaptor molecule function by successfully being incorporated into a nascent polypeptide chain in an in vitro translation reaction. First, a rabbit reticulocyte lysate that is depleted of the endogenous tRNA using an antisense or complimentary oligonucleotide which (i) targets the sequence between the anticodon and variable loop; or (ii) binds the region between the anticodon and variable loop is generated (see, e.g., Cui et al. 2018. Nucleic Acids Res. 46(12):6387-6400). 10-25 ug/mL of the test TREM is added in addition to 2 ug/uL of a GFP-encoding mRNA to the depleted lysate. A non-depleted lysate with the GFP mRNA and with or without test TREM added are used as a positive control. A depleted lysate with the GFP mRNA but without the test TREM added is used as a negative control. The progress of GFP mRNA translation is monitored by fluorescence increase on a microplate reader at 37° C. for 3-5 h using λₑₓ485/λₑₘ528. The methods described in this example can be adopted for use to evaluate if the test TREM can complement the depleted lysate and is thus likely functional.

### Example 15: Production of a Candidate TREM Complementary to the Con-Rare Codon Through Mammalian Cell Purification

This example describes the production of a TREM in mammalian host cells.

### Plasmid Generation

To generate a plasmid comprising a TREM which comprises a tRNA gene, in this example, tRNA-Ser-AGA, a DNA fragment containing at least one copy of the tRNA gene with the sequence GTAGTCGTGGCCGAGTGGTTAAGGCGATGGACTAGAAATCCATTGGGGTTTCCCCGC GCAGGTTCGAATCCTGCCGACTACG is synthesized and cloned into the pLKO.1 puro backbone plasmid with a U6 promoter (or any other RNA polymerase III recruiting promoter) following the manufacturer's instructions and standard molecular cloning techniques.

### Transfection

One (1) mg of plasmid described above is used to transfect a 1 L culture of suspension-adapted HEK293T cells (Freestyle 293-F cells) at 1×10⁵ cells/mL. Cells are harvested at 24, 48, 72, or 96 hours post-transfection to determine the optimized timepoint for TREM expression as determined by a quantitative method such as Northern blot, quantitative PCR (q-PCR) or Nanopore sequencing.

### Purification

At the optimized harvest timepoint, the cells are lysed, and total RNA is purified using a method such as phenol chloroform. RNAs smaller than 200 nucleotides are separated from the lysate using a small RNA isolation kit per manufacturer's instructions, to generate a small RNA (sRNA) fraction.

The sRNA fraction is incubated with annealing buffer and the biotinylated capture probe corresponding to a DNA probe that is complementary to a unique region of the TREM being purified, in this example, a probe with the sequence 3′ biotin-CCAATGGATTTCTATCCATCGCCTTAACCACTCGGCCACGACTACAAAA is used to purify the TREM comprising tRNA-Ser-AGA. The mixture is incubated at 90° C. for 2-3 minutes and quickly cooled down to 45° C. and incubated overnight at 45° C. The admixture is then incubated with binding buffer previously heated to 45° C. and streptavidin-conjugated RNase-free magnetic beads for 3 hours to allow binding of the DNA-tRNA complexes to the beads. The mixture is then added to a pre-equilibrated column in a magnetic field separator rack and washed 4 times. The TREM retained on the beads are eluted three times by adding elution buffer pre-heated to 80° C. and then admixed with a pharmaceutically acceptable excipient to make a test TREM product.

### Example 16: Production of a Candidate TREM Complementary to a Con-Rare Codon Through Bacterial Cell Purification

This example describes the production of a TREM in bacterial host cells.

### Plasmid Generation

To generate a plasmid to produce a TREM in bacteria, a tRNA gene, in this example, a DNA fragment containing at least one copy of the tRNA-Lys-UUU gene with the sequence GCCCGGATAGCTCAGTCGGTAGAGCATCAGACTTTTAATCTGAGGGTCCAGGGTTCA AGTCCCTGTTCGGGCG is synthesized and cloned into a bacterial tRNA expression vector as previously described in Ponchon et al., Nat Protoc 4, 947-959 (2009).

### Transformation

1×10⁹ bacteria grown from TREM expression plasmid transformed competent bacteria will be harvested at different cell density points, in this example OD(600)=0.5, OD(600)=0.7, OD(600)=0.9 to determine the optimal point of TREM expression as determined by a quantitative method such as Northern blot, quantitative PCR (q-PCR) or Nanopore sequencing.

### Purification

At the optimized harvest cell density point, the TREM is purified as previously described in Cayama et al., Nucleic Acids Research. 28 (12), e64 (2000). Briefly, short RNAs (e.g., tRNAs) are recovered from cells by phenol extraction and concentrated by ethanol precipitation. The total tRNA in the precipitate is then separated from larger nucleic acids (including rRNA and DNA) under high salt conditions by a stepwise isopropanol precipitation. The elution fraction containing the TREM is further purified through probe binding. The TREM fraction is incubated with annealing buffer and the biotinylated capture probe corresponding to a DNA probe that is complementary to a unique region of the TREM being purified, in this example, a probe conjugated to biotin at the 3′ end with the sequence CAGAUUAAAAGUCUG, is used to purify the TREM comprising tRNA-Lys-UUU. The mixture is incubated at 90° C. for 2-3 minutes and quickly cooled down to 45° C. and incubated overnight at 45° C. The admixture is then incubated with binding buffer previously heated to 45° C. and streptavidin-conjugated RNase-free magnetic beads for 3 hours to allow binding of the DNA-tRNA complexes to the beads. The mixture is then added to a pre-equilibrated column in a magnetic field separator rack and washed 4 times. The TREM retained on the beads are eluted three times by adding elution buffer pre-heated to 80° C. and then admixed with a pharmaceutically acceptable excipient to make a test TREM product.

### Example 17: Production of a Candidate TREM Complementary to a Con-Rare Codon Through Chemical Synthesis

This example describes production of a TREM using chemical synthesis.

The TREM, in this example, tRNA-Thr-CGT, is chemically synthesized with the sequence GGCUCUAUGGCUUAGUUGGUUAAAGCGCCUGUCUCGUAAACAGGAGAUCCUGGG UUCGACUCCCAGUGGGGCCUCAA. This TREM is produced by solid-phase chemical synthesis using phosphoroamedite chemistry as previously described, for example as in Zlatev et. al. (2012) Current Protocols, 50 (1), 1.28.1-1.28.16. Briefly, protected RNA phorphoroamedites are sequentially added in a desired order to a growing chain immobilized on a solid support (e.g. controlled pore glass). Each cycle of addition has multiple steps, including: (i) deblocking the DMT group protecting the 5′-hydroxyl of the growing chain, (ii) coupling the growing chain to an incoming phosphoramidite building block, (iii) capping any chain molecules still featuring a 5′-hydroxyl, i.e. those that failed to couple with the desired incoming building block, and (iv) oxidation of the newly formed tricoordinated phosphite triester linkage. After the final building block has been coupled and oxidized, the chain is cleaved from the solid support and all protecting groups except for the DMT group protecting the 5′-hydroxyl are removed. The chain is then purified by RP-HPLC (e.g., DMT-on purification) and the fraction containing the chain is subjected to deprotection of the DMT group under acidic conditions, affording the final TREM. The TREM will feature a 5′-phosphate and a 3′-OH. The TREM is then admixed with a pharmaceutically acceptable excipient to make a test TREM product.

If the TREM needs to be charged, the TREM produced by the chemical synthesis reaction is then aminoacylated in vitro using aminoacyl tRNA synthetase, as previously described in Stanley, Methods Enzymol 29:530-547 (1974). Briefly, the TREM is incubated for 30 min at 37° C. with its synthetase and its cognate amino, in this example, with threonyl-tRNA synthetase and threonine, respectively, and then phenol extracted, filtered using a Nuc-trap column, and ethanol precipitated. The TREM is then admixed with a pharmaceutically acceptable excipient to make a test TREM product.

### Example 18: Production of a Candidate TREM Complementary to a Con-Rare Codon Through In Vitro Transcription

This example describes production of a TREM using in vitro transcription (IVT).

The TREM, in this example, tRNA-Leu-CAA, is produced using in vitro transcription with the sequence GUCAGGAUGGCCGAGUGGUCUAAGGCGCCAGACUCAAGUUCUGGUCUCCGUAUG GAGGCGUGGGUUCGAAUCCCACUUCUGACA as previously described in Pestova et al., RNA 7(10):1496-505 (2001). Briefly, a DNA plasmid containing a bacteriophage T7 promoter followed by the tRNA-Leu-CAA gene sequence is linearized and transcribed in vitro with T7 RNA polymerase at 37° C. for 45 min and then phenol extracted, filtered using a Nuc-trap column, and ethanol precipitated. The TREM is then admixed with a pharmaceutically acceptable excipient to make a test TREM product. Optionally, before admixing with a pharmaceutically acceptable excipient, the TREM is heated and cooled to refold the TREM.

If the TREM needs to be charged, the TREM produced by the IVT reaction is then aminoacylated in vitro using aminoacyl tRNA synthetase, as previously described in Stanley, Methods Enzymol 29:530-547 (1974). Briefly, the TREM is incubated for 30 min at 37° C. with its synthetase and its cognate amino, in this example, with leucyl-tRNA synthetase and leucine, respectively, and then phenol extracted, filtered using a Nuc-trap column, and ethanol precipitated. The TREM is then admixed with a pharmaceutically acceptable excipient to make a test TREM product.

What is claimed is:

1. A method of modulating a production parameter of an RNA, or a protein encoded by an RNA, in a target cell or tissue, comprising:
providing, e.g., administering, to the target cell or tissue, or contacting the target cell or tissue with, an effective amount of a tRNA effector molecule (TREM) (e.g., a TREM composition comprising a TREM), which TREM corresponds to a contextually-rare codon (“con-rare codon”) of the RNA,
thereby modulating the production parameter of the RNA, or protein encoded by the RNA in the target cell or tissue.

2. The method of claim 1, wherein the target cell or tissue is obtained from a subject.

3. The method of claim 1, comprising administering the TREM composition to a subject.

4. The method of claim 1, comprising contacting the TREM composition with the target tissue or cell ex vivo.

5. The method of claim 4, comprising introducing the ex vivo-contacted target tissue or cell into a subject, e.g., an allogeneic or autologous subject.

6. The method of any one of the preceding claims, wherein the target cell or tissue is a specific or selected target cell or tissue, e.g., a cell or tissue type in a particular developmental stage; a cell or tissue type in a particular disease state; or a cell present in a particular extracellular milieu.

7. The method of any one of the preceding claims, wherein the production parameter comprises an expression parameter or a signaling parameter, e.g., as described herein.

8. The method of any one of the preceding claims, wherein the production parameter of the RNA is modulated, e.g., an RNA that can be translated into a polypeptide, e.g., a messenger RNA.

9. The method of claim 7, wherein the production parameter of the RNA is increased or decreased.

10. The method of any one of the preceding claims, wherein the production parameter of the protein encoded by the RNA is modulated.

11. The method of claim 10, wherein the production parameter of the protein is increased or decreased.

12. A method of determining the presence of a nucleic acid sequence, e.g., a DNA or RNA, having a contextually-rare codon (“con-rare codon nucleic acid sequence”), comprising:
acquiring knowledge of the presence of the con-rare codon nucleic acid sequence in a sample from a subject, e.g., a target cell or tissue sample,
wherein responsive to the acquisition of knowledge of the presence of the con-rare codon nucleic acid sequence:
(1) the subject is classified as being a candidate to receive administration of an effective amount of a composition comprising a tRNA effector molecule (TREM) which corresponds to a contextually-rare codon (“con-rare codon”) of the nucleic acid sequence; or
(2) the subject is identified as likely to respond to a treatment comprising the composition comprising the TREM.

13. A method of treating a subject having a disease associated with a contextually-rare codon (“con-rare codon”), comprising:
acquiring knowledge of the presence of a nucleic acid sequence, e.g., a DNA or RNA, having the con-rare codon (“con-rare codon nucleic acid sequence”) in a target cell or tissue sample from the subject; and
administering to the subject an effective amount of a composition comprising a tRNA effector molecule (TREM) which corresponds to the con-rare codon of the nucleic acid sequence,
thereby treating the disease in the subject.

14. A method of providing a tRNA effector molecule (TREM) to a subject, comprising:
providing, e.g., administering, to the subject, an effective amount of a TREM, e.g., a TREM composition comprising a TREM, which TREM corresponds to a contextually-rare codon (“con-rare codon”) for a nucleic acid sequence in a target cell or tissue in the subject,
thereby providing a TREM to the subject.

15. A method of manufacturing a tRNA effector molecule (TREM) composition comprising:
identifying a TREM corresponding to a contextually-rare (con-rare) codon;
combining the TREM with a component, e.g., a carrier or excipient.
thereby manufacturing a TREM composition.

16. The method of any one of the preceding claims, wherein the method comprises acquiring a value for a con-rare codon in the nucleic acid sequence, e.g., DNA or RNA, wherein the value is a function of one or more of the following factors, e.g., by evaluating or determining one or more of the following factors:
(1) the sequence of the codon;
(2) the availability of a corresponding tRNA, e.g., charged tRNA, for that con-rare codon in a target cell or tissue, e.g., one or more iso-acceptor tRNA molecules;
(3) the expression profile (or proteomic properties) of the target cell or tissue (e.g., the abundance of expression of other proteins which include the con-rare codon);
(4) the proportion of the tRNAs corresponding to the con-rare codon which are charged; and
(5) the iso-decoder isotype of the tRNA corresponding to the con-rare codon;

17. The method of claim 16, wherein (1) comprises determining the presence or absence of a con-rare codon.

18. The method of claim 17, wherein a determination of the availability of a tRNA comprises acquiring a measure of one, two, three or all of the following parameters:
(a) level of a tRNA corresponding to the con-rare codon (“con-rare codon tRNA”) compared to a tRNA corresponding to a different codon;
(b) function, e.g., polypeptide chain elongation function, of a con-rare codon tRNA compared to a tRNA corresponding to a different codon;
(c) modification, e.g., aminoacylation or post-transcriptional modification, of a con-rare codon tRNA compared to a tRNA corresponding to a different codon; and/or
(d) sequence of a con-rare codon tRNA.

19. The method of claim 18, wherein a measure of availability (e.g., level) of a con-rare codon tRNA comprises a measure of the con-rare codon tRNA that is charged, e.g., aminoacylated, compared to: (1) the proportion of the con-rare codon tRNA that is not charged; or (2) the proportion of charged tRNA corresponding to a different codon.

20. The method of any one of the preceding claims, wherein at least 10%, 20%, 30%, 40%, 50%, 60%, 70%, 80%, 85%, 90%, 95%, 99%, or 100% (by weight or number) of the TREMs in the TREM composition correspond to a con-rare codon.

21. The method of any one of the preceding claims, wherein the TREM composition comprises TREMs that correspond to a plurality of con-rare codons.

22. The method of any one of the preceding claims, wherein the TREM composition comprises: a first TREM which corresponds to a first con-rare codon; and an additional TREM which corresponds to a different con-rare codon.

23. The method of any one of the preceding claims, wherein the TREM composition was made by a method comprising:
(a) providing a host cell, comprising exogenous nucleic acid, e.g., a DNA or RNA, encoding a TREM under conditions sufficient to express the TREM; and
(b) purifying the expressed TREM from the host cell culture to produce a TREM composition, thereby making a TREM composition.

24. The method of any one of the preceding claims, wherein the TREM composition is a pharmaceutical composition comprising a TREM.

25. The method of any one of the preceding claims, wherein the TREM composition comprises a pharmaceutical excipient.

26. The method of any one of the preceding claims, wherein the TREM composition comprises a TREM fragment, e.g., as described herein.

27. The method of any one of the preceding claims, wherein the TREM composition comprises one or more, e.g., a plurality, of TREMs.


SEQUENCE LISTING (631 sequences)

SEQ ID NO: 1 - DNA, 72 bp, Homo sapiens
gggggtatagctcagtggtagagcgcgtgcttagcatgcacgaggtcctgggttcgatccccagtacctcca

SEQ ID NO: 2 - DNA, 73 bp, Homo sapiens
ggggaattagctcaagtggtagagcgcttgcttagcacgcaagaggtagtgggatcgatgcccacattctcca

SEQ ID NO: 3 - DNA, 73 bp, Homo sapiens
ggggaattagctcaaatggtagagcgctcgcttagcatgcgagaggtagcgggatcgatgcccgcattctcca

SEQ ID NO: 4 - DNA, 73 bp, Homo sapiens
ggggaattagctcaagtggtagagcgcttgcttagcatgcaagaggtagtgggatcgatgcccacattctcca

SEQ ID NO: 5 - DNA, 73 bp, Homo sapiens
ggggaattagctcaagcggtagagcgcttgcttagcatgcaagaggtagtgggatcgatgcccacattctcca

SEQ ID NO: 6 - DNA, 73 bp, Homo sapiens
ggggaattagctcaagtggtagagcgcttgcttagcatgcaagaggtagtgggatcaatgcccacattctcca

SEQ ID NO: 7 - DNA, 73 bp, Homo sapiens
ggggaattagctcaagtggtagagcgctcgcttagcatgcgagaggtagtgggatcgatgcccgcattctcca

SEQ ID NO: 8 - DNA, 73 bp, Homo sapiens
ggggaattagcccaagtggtagagcgcttgcttagcatgcaagaggtagtgggatcgatgcccacattctcca

SEQ ID NO: 9 - DNA, 72 bp, Homo sapiens
gggggtgtagctcagtggtagagcgcgtgcttagcatgcacgaggccccgggttcaatccccggcacctcca

SEQ ID NO: 10 - DNA, 72 bp, Homo sapiens
gggggtgtagctcagtggtagagcgcgtgcttagcatgtacgaggtcccgggttcaatccccggcacctcca

SEQ ID NO: 11 - DNA, 72 bp, Homo sapiens
ggggatgtagctcagtggtagagcgcatgcttagcatgcatgaggtcccgggttcgatccccagcatctcca

SEQ ID NO: 12 - DNA, 72 bp, Homo sapiens
gggggtgtagctcagtggtagagcgcgtgcttagcatgcacgaggccctgggttcaatccccagcacctcca

SEQ ID NO: 13 - DNA, 72 bp, Homo sapiens
gggggtatagctcagcggtagagcgcgtgcttagcatgcacgaggtcctgggttcaatccccaatacctcca

SEQ ID NO: 14 - DNA, 72 bp, Homo sapiens
gggggtgtagctcagtggtagagcgcgtgcttagcatgcacgaggccccgggttcaatccctggcacctcca

SEQ ID NO: 15 - DNA, 73 bp, Homo sapiens
gggggattagctcaaatggtagagcgctcgcttagcatgcgagaggtagcgggatcgatgcccgcatcctcca

SEQ ID NO: 16 - DNA, 73 bp, Homo sapiens
ggggaattagctcaggcggtagagcgctcgcttagcatgcgagaggtagcgggatcgacgcccgcattctcca

SEQ ID NO: 17 - DNA, 72 bp, Homo sapiens
ggggatgtagctcagtggtagagcgcatgcttcgcatgtatgaggtcccgggttcgatccccggcatctcca

SEQ ID NO: 18 - DNA, 72 bp, Homo sapiens
ggggatgtagctcagtggtagagcgcatgcttcgcatgtatgaggccccgggttcgatccccggcatctcca

SEQ ID NO: 19 - DNA, 72 bp, Homo sapiens
ggggatgtagctcagtggtagagcgcgcgcttcgcatgtgtgaggtcccgggttcaatccccggcatctcca

SEQ ID NO: 20 - DNA, 72 bp, Homo sapiens
gggggtgtagctcagtggtagagcgcgtgcttcgcatgtacgaggccccgggttcgacccccggctcctcca

SEQ ID NO: 21 - DNA, 72 bp, Homo sapiens
gggggtgtagctcagtggtagagcgcatgctttgcatgtatgaggtcccgggttcgatccccggcacctcca

SEQ ID NO: 22 - DNA, 72 bp, Homo sapiens
ggggatgtagctcagtggtagagcgcatgctttgcatgtatgaggtcccgggttcgatccccggcatctcca

SEQ ID NO: 23 - DNA, 72 bp, Homo sapiens
ggggatgtagctcagtggtagagcgcatgctttgcatgtatgaggccccgggttcgatccccggcatctcca

SEQ ID NO: 24 - DNA, 72 bp, Homo sapiens
ggggatgtagctcagtggtagagcgcatgctttgcacgtatgaggccccgggttcaatccccggcatctcca

SEQ ID NO: 25 - DNA, 72 bp, Homo sapiens
gggggtgtagctcagtggtagagcgcatgctttgcatgtatgaggcctcgggttcgatccccgacacctcca

SEQ ID NO: 26 - DNA, 72 bp, Homo sapiens
gggggtgtagctcagtggtagagcacatgctttgcatgtgtgaggccccgggttcgatccccggcacctcca

SEQ ID NO: 27 - DNA, 71 bp, Homo sapiens
gggggtgtagctcagtggtagagcgcatgctttgcatgtatgaggcctcggttcgatccccgacacctcca

SEQ ID NO: 28 - DNA, 73 bp, Homo sapiens
gggccagtggcgcaatggataacgcgtctgactacggatcagaagattccaggttcgactcctggctggctcg

SEQ ID NO: 29 - DNA, 73 bp, Homo sapiens
gggccagtggcgcaatggataacgcgtctgactacggatcagaagattctaggttcgactcctggctggctcg

SEQ ID NO: 30 - DNA, 73 bp, Homo sapiens
ggccgcgtggcctaatggataaggcgtctgattccggatcagaagattgagggttcgagtcccttcgtggtcg

SEQ ID NO: 31 - DNA, 73 bp, Homo sapiens
gacccagtggcctaatggataaggcatcagcctccggagctggggattgtgggttcgagtcccatctgggtcg

SEQ ID NO: 32 - DNA, 73 bp, Homo sapiens
gccccagtggcctaatggataaggcactggcctcctaagccagggattgtgggttcgagtcccacctggggta

SEQ ID NO: 33 - DNA, 73 bp, Homo sapiens
gccccagtggcctaatggataaggcactggcctcctaagccagggattgtgggttcgagtcccacctggggtg

SEQ ID NO: 34 - DNA, 73 bp, Homo sapiens
gccccggtggcctaatggataaggcattggcctcctaagccagggattgtgggttcgagtcccacccggggta

SEQ ID NO: 35 - DNA, 73 bp, Homo sapiens
gccccagtggcctaatggataaggcattggcctcctaagccagggattgtgggttcgagtcccatctggggtg

SEQ ID NO: 36 - DNA, 73 bp, Homo sapiens
gccccagtggcctgatggataaggtactggcctcctaagccagggattgtgggttcgagttccacctggggta

SEQ ID NO: 37 - DNA, 73 bp, Homo sapiens
ggccgcgtggcctaatggataaggcgtctgacttcggatcagaagattgcaggttcgagtcctgccgcggtcg

SEQ ID NO: 38 - DNA, 73 bp, Homo sapiens
gaccacgtggcctaatggataaggcgtctgacttcggatcagaagattgagggttcgaatccctccgtggtta

SEQ ID NO: 39 - DNA, 73 bp, Homo sapiens
gaccgcgtggcctaatggataaggcgtctgacttcggatcagaagattgagggttcgagtcccttcgtggtcg

SEQ ID NO: 40 - DNA, 73 bp, Homo sapiens
gaccacgtggcctaatggataaggcgtctgacttcggatcagaagattgagggttcgaatcccttcgtggtta

SEQ ID NO: 41 - DNA, 73 bp, Homo sapiens
gaccacgtggcctaatggataaggcgtctgacttcggatcagaagattgagggttcgaatcccttcgtggttg

SEQ ID NO: 42 - DNA, 73 bp, Homo sapiens
ggccgtgtggcctaatggataaggcgtctgacttcggatcaaaagattgcaggtttgagttctgccacggtcg

SEQ ID NO: 43 - DNA, 85 bp, Homo sapiens
ggctccgtggcgcaatggatagcgcattggacttctagaggctgaaggcattcaaaggttccgggttcgagtcccggcggagtcg

SEQ ID NO: 44 - DNA, 88 bp, Homo sapiens
ggctctgtggcgcaatggatagcgcattggacttctagtgacgaatagagcaattcaaaggttgtgggttcgaatcccaccagagtcg

SEQ ID NO: 45 - DNA, 91 bp, Homo sapiens
ggctctgtggcgcaatggatagcgcattggacttctagctgagcctagtgtggtcattcaaaggttgtgggttcgagtcccaccagagtcg

SEQ ID NO: 46 - DNA, 86 bp, Homo sapiens
ggctctgtggcgcaatggatagcgcattggacttctagatagttagagaaattcaaaggttgtgggttcgagtcccaccagagtcg

SEQ ID NO: 47 - DNA, 74 bp, Homo sapiens
gtctctgtggcgcaatggacgagcgcgctggacttctaatccagaggttccgggttcgagtcccggcagagatg

SEQ ID NO: 48 - DNA, 87 bp, Homo sapiens
ggctctgtggcgcaatggatagcgcattggacttctagcctaaatcaagagattcaaaggttgcgggttcgagtccctccagagtcg

SEQ ID NO: 49 - DNA, 74 bp, Homo sapiens
gtctctgtggcgcaatcggttagcgcgttcggctgttaaccgaaaggttggtggttcgatcccacccagggacg

SEQ ID NO: 50 - DNA, 74 bp, Homo sapiens
gtctctgtggcgcaatcggctagcgcgtttggctgttaactaaaaggttggcggttcgaacccacccagaggcg

SEQ ID NO: 51 - DNA, 74 bp, Homo sapiens
gtctctgtggtgcaatcggttagcgcgttccgctgttaaccgaaagcttggtggttcgagcccacccagggatg

SEQ ID NO: 52 - DNA, 74 bp, Homo sapiens
gtctctgtggcgcaatcggctagcgcgtttggctgttaactaaaaagttggtggttcgaacacacccagaggcg

SEQ ID NO: 53 - DNA, 74 bp, Homo sapiens
gtctctgtggcgcaatcggttagcgcgttcggctgttaaccgaaaggttggtggttcgagcccacccagggacg

SEQ ID NO: 54 - DNA, 74 bp, Homo sapiens
gtctctgtggcgcaatcggttagcgcattcggctgttaaccgaaaggttggtggttcgagcccacccagggacg

SEQ ID NO: 55 - DNA, 74 bp, Homo sapiens
gtctctgtggcgcaatcggttagcgcgttcggctgttaaccgaaagattggtggttcgagcccacccagggacg

SEQ ID NO: 56 - DNA, 74 bp, Homo sapiens
gtctctgtggcgcaatcggttagcgcgttcggctgttaactgaaaggttggtggttcgagcccacccagggacg

SEQ ID NO: 57 - DNA, 74 bp, Homo sapiens
gtctctgtggcgcaatgggttagcgcgttcggctgttaaccgaaaggttggtggttcgagcccatccagggacg

SEQ ID NO: 58 - DNA, 74 bp, Homo sapiens
gtctctgtggcgtagtcggttagcgcgttcggctgttaaccgaaaagttggtggttcgagcccacccaggaacg

SEQ ID NO: 59 - DNA, 74 bp, Homo sapiens
gtctctgtggcgcaatcggctagcgcgtttggctgttaactaaaaggttggtggttcgaacccacccagaggcg

SEQ ID NO: 60 - DNA, 74 bp, Homo sapiens
gtctctgtggcgcaatcggttagcgcgttcggctgttaactgaaaggttagtggttcgagcccacccggggacg

SEQ ID NO: 61 - DNA, 72 bp, Homo sapiens
tcctcgttagtatagtggttagtatccccgcctgtcacgcgggagaccggggttcaattccccgacggggag

SEQ ID NO: 62 - DNA, 72 bp, Homo sapiens
tcctcgttagtatagtggtgagtatccccgcctgtcacgcgggagaccggggttcgattccccgacggggag

SEQ ID NO: 63 - DNA, 72 bp, Homo sapiens
tcctcgttagtatagtggtgagtgtccccgtctgtcacgcgggagaccggggttcgattccccgacggggag

SEQ ID NO: 64 - DNA, 72 bp, Homo sapiens
gggggcatagctcagtggtagagcatttgactgcagatcaagaggtccctggttcaaatccaggtgccccct

SEQ ID NO: 65 - DNA, 72 bp, Homo sapiens
gggggtatagctcaggggtagagcatttgactgcagatcaagaggtccctggttcaaatccaggtgcccccc

SEQ ID NO: 66 - DNA, 72 bp, Homo sapiens
gggggtatagcttagcggtagagcatttgactgcagatcaagaggtccccggttcaaatccgggtgccccct

SEQ ID NO: 67 - DNA, 72 bp, Homo sapiens
gggggtatagcttaggggtagagcatttgactgcagatcaaaaggtccctggttcaaatccaggtgcccctt

SEQ ID NO: 68 - DNA, 72 bp, Homo sapiens
gggggtatagctcaggggtagagcatttgactgcagatcaagaggtccccagttcaaatctgggtgccccct

SEQ ID NO: 69 - DNA, 72 bp, Homo sapiens
gggggtatagctcaggggtagagcatttgactgcagatcaagaagtccccggttcaaatccgggtgccccct

SEQ ID NO: 70 - DNA, 72 bp, Homo sapiens
gggggtatagctcaggggtagagcatttgactgcagatcaagaggtctctggttcaaatccaggtgccccct

SEQ ID NO: 71 - DNA, 72 bp, Homo sapiens
gggggtatagctcaggggtagagcacttgactgcagatcaagaagtccttggttcaaatccaggtgccccct

SEQ ID NO: 72 - DNA, 72 bp, Homo sapiens
ggggatatagctcaggggtagagcatttgactgcagatcaagaggtccccggttcaaatccgggtgcccccc

SEQ ID NO: 73 - DNA, 72 bp, Homo sapiens
gggggtatagttcaggggtagagcatttgactgcagatcaagaggtccctggttcaaatccaggtgccccct

SEQ ID NO: 74 - DNA, 72 bp, Homo sapiens
gggggtatagctcaggggtagagcatttgactgcaaatcaagaggtccctgattcaaatccaggtgccccct

SEQ ID NO: 75 - DNA, 72 bp, Homo sapiens
gggggtatagctcagtggtagagcatttgactgcagatcaagaggtccccggttcaaatccgggtgccccct

SEQ ID NO: 76 - DNA, 72 bp, Homo sapiens
gggcgtatagctcaggggtagagcatttgactgcagatcaagaggtccccagttcaaatctgggtgccccct

SEQ ID NO: 77 - DNA, 72 bp, Homo sapiens
gggggtatagctcacaggtagagcatttgactgcagatcaagaggtccccggttcaaatctgggtgccccct

SEQ ID NO: 78 - DNA, 70 bp, Homo sapiens
gggcgtatagctcaggggtagagcatttgactgcagatcaagaggtccccagttcaaatctgggtgccca

SEQ ID NO: 79 - DNA, 72 bp, Homo sapiens
gggggtatagctcacaggtagagcatttgactgcagatcaagaggtccccggttcaaatccggttactccct

SEQ ID NO: 80 - DNA, 72 bp, Homo sapiens
gggggtatagctcaggggtagagcacttgactgcagatcaagaggtccctggttcaaatccaggtgccccct

SEQ ID NO: 81 - DNA, 72 bp, Homo sapiens
gggggtatagctcagtggtagagcatttgactgcagatcaagaggtccctggttcaaatccgggtgccccct

SEQ ID NO: 82 - DNA, 73 bp, Homo sapiens
gggggtatagctcagtgggtagagcatttgactgcagatcaagaggtccccggttcaaatccgggtgccccct

SEQ ID NO: 83 - DNA, 72 bp, Homo sapiens
gggggtgtagctcagtggtagagcatttgactgcagatcaagaggtccctggttcaaatccaggtgccccct

SEQ ID NO: 84 - DNA, 73 bp, Homo sapiens
gggggtatagctcaggtggtagagcatttgactgcagatcaagaggtccccggttcaaatccgggtgccccct

SEQ ID NO: 85 - DNA, 72 bp, Homo sapiens
gggggtatagctcaggggtagagcatttgactgcagatcaagaggtccccggttcaaatccgggtgccccct

SEQ ID NO: 86 - DNA, 72 bp, Homo sapiens
gggggtatagctcaggggtagagcatttgactgcagatcaagaggtccctggttcaaatccaggtgccccct

SEQ ID NO: 87 - DNA, 72 bp, Homo sapiens
ggttccatggtgtaatggttagcactctggactctgaatccagcgatccgagttcaaatctcggtggaacct

SEQ ID NO: 88 - DNA, 72 bp, Homo sapiens
ggttccatggtgtaatggttagcactctggactctgaatccagcgatccgagttcaagtctcggtggaacct

SEQ ID NO: 89 - DNA, 72 bp, Homo sapiens
ggttccatggtgtaatggtgagcactctggactctgaatccagcgatccgagttcgagtctcggtggaacct

SEQ ID NO: 90 - DNA, 72 bp, Homo sapiens
ggttccatggtgtaatggtaagcactctggactctgaatccagcgatccgagttcgagtctcggtggaacct

SEQ ID NO: 91 - DNA, 72 bp, Homo sapiens
ggttccatggtgtaatggttagcactctggactctgaatccggtaatccgagttcaaatctcggtggaacct

SEQ ID NO: 92 - DNA, 72 bp, Homo sapiens
ggccccatggtgtaatggtcagcactctggactctgaatccagcgatccgagttcaaatctcggtgggaccc

SEQ ID NO: 93 - DNA, 72 bp, Homo sapiens
ggttccatggtgtaatggtaagcactctggactctgaatccagccatctgagttcgagtctctgtggaacct

SEQ ID NO: 94 - DNA, 72 bp, Homo sapiens
ggtcccatggtgtaatggttagcactctggactttgaatccagcgatccgagttcaaatctcggtgggacct

SEQ ID NO: 95 - DNA, 72 bp, Homo sapiens
ggtcccatggtgtaatggttagcactctggactttgaatccagcaatccgagttcgaatctcggtgggacct

SEQ ID NO: 96 - DNA, 72 bp, Homo sapiens
ggccccatggtgtaatggttagcactctggactttgaatccagcgatccgagttcaaatctcggtgggacct

SEQ ID NO: 97 - DNA, 72 bp, Homo sapiens
ggtcccatggtgtaatggttagcactctgggctttgaatccagcaatccgagttcgaatcttggtgggacct

SEQ ID NO: 98 - DNA, 72 bp, Homo sapiens
tccctggtggtctagtggttaggattcggcgctctcaccgccgcggcccgggttcgattcccggtcagggaa

SEQ ID NO: 99 - DNA, 72 bp, Homo sapiens
tccctggtggtctagtggttaggattcggcgctctcaccgccgcggcccgggttcgattcccggtcaggaaa

SEQ ID NO: 100 - DNA, 72 bp, Homo sapiens
tcccatatggtctagcggttaggattcctggttttcacccaggtggcccgggttcgactcccggtatgggaa

SEQ ID NO: 101 - DNA, 72 bp, Homo sapiens
tcccacatggtctagcggttaggattcctggttttcacccaggcggcccgggttcgactcccggtgtgggaa

SEQ ID NO: 102 - DNA, 72 bp, Homo sapiens
tccctggtggtctagtggctaggattcggcgctttcaccgccgcggcccgggttcgattcccggccagggaa

SEQ ID NO: 103 - DNA, 72 bp, Homo sapiens
tccctggtggtctagtggctaggattcggcgctttcaccgccgcggcccgggttcgattcccggtcagggaa

SEQ ID NO: 104 - DNA, 71 bp, Homo sapiens
gcattggtggttcagtggtagaattctcgcctcccacgcgggagacccgggttcaattcccggccaatgca

SEQ ID NO: 105 - DNA, 71 bp, Homo sapiens
gcgccgctggtgtagtggtatcatgcaagattcccattcttgcgacccgggttcgattcccgggcggcgca

SEQ ID NO: 106 - DNA, 71 bp, Homo sapiens
gcattggtggttcaatggtagaattctcgcctcccacgcaggagacccaggttcgattcctggccaatgca

SEQ ID NO: 107 - DNA, 71 bp, Homo sapiens
gcatgggtggttcagtggtagaattctcgcctgccacgcgggaggcccgggttcgattcccggcccatgca

SEQ ID NO: 108 - DNA, 71 bp, Homo sapiens
gcattggtggttcagtggtagaattctcgcctgccacgcgggaggcccgggttcgattcccggccaatgca

SEQ ID NO: 109 - DNA, 71 bp, Homo sapiens
gcattggtggttcagtggtagaattctcgcctgccacgcgggaggcccgggtttgattcccggccagtgca

SEQ ID NO: 110 - DNA, 71 bp, Homo sapiens
gcataggtggttcagtggtagaattcttgcctgccacgcaggaggcccaggtttgattcctggcccatgca

SEQ ID NO: 111 - DNA, 71 bp, Homo sapiens
gcattggtggttcagtggtagaattctcgcctgccatgcgggcggccgggcttcgattcctggccaatgca

SEQ ID NO: 112 - DNA, 72 bp, Homo sapiens
gcgttggtggtatagtggttagcatagctgccttccaagcagttgacccgggttcgattcccggccaacgca

SEQ ID NO: 113 - DNA, 72 bp, Homo sapiens
gcgttggtggtatagtggtgagcatagctgccttccaagcagttgacccgggttcgattcccggccaacgca

SEQ ID NO: 114 - DNA, 72 bp, Homo sapiens
gcgttggtggtatagtggtaagcatagctgccttccaagcagttgacccgggttcgattcccggccaacgca

SEQ ID NO: 115 - DNA, 72 bp, Homo sapiens
gcgttggtggtatagtggtgagcatagttgccttccaagcagttgacccgggctcgattcccgcccaacgca

SEQ ID NO: 116 - DNA, 72 bp, Homo sapiens
gccgtgatcgtatagtggttagtactctgcgttgtggccgcagcaacctcggttcgaatccgagtcacggca

SEQ ID NO: 117 - DNA, 72 bp, Homo sapiens
gccatgatcgtatagtggttagtactctgcgctgtggccgcagcaacctcggttcgaatccgagtcacggca

SEQ ID NO: 118 - DNA, 74 bp, Homo sapiens
ggccggttagctcagttggttagagcgtggcgctaataacgccaaggtcgcgggttcgatccccgtacgggcca

SEQ ID NO: 119 - DNA, 74 bp, Homo sapiens
ggccggttagctcagttggttagagcgtggtgctaataacgccaaggtcgcgggttcgatccccgtactggcca

SEQ ID NO: 120 - DNA, 74 bp, Homo sapiens
ggctggttagctcagttggttagagcgtggtgctaataacgccaaggtcgcgggttcgatccccgtactggcca

SEQ ID NO: 121 - DNA, 74 bp, Homo sapiens
ggccggttagctcagttggttagagcgtggtgctaataacgccaaggtcgcgggttcgaaccccgtacgggcca

SEQ ID NO: 122 - DNA, 74 bp, Homo sapiens
ggccggttagctcagttggttagagcgtggtgctaataacgccaaggtcgcgggttcgatccccgtacgggcca

SEQ ID NO: 123 - DNA, 74 bp, Homo sapiens
ggccggttagctcagttggttagagcgtggtgctaataacgctaaggtcgcgggttcgatccccgtactggcca

SEQ ID NO: 124 - DNA, 74 bp, Homo sapiens
ggccggttagctcagttggtcagagcgtggtgctaataacgccaaggtcgcgggttcgatccccgtacgggcca

SEQ ID NO: 125 - DNA, 74 bp, Homo sapiens
ggccggttagctcagtcggctagagcgtggtgctaataacgccaaggtcgcgggttcgatccccgtacgggcca

SEQ ID NO: 126 - DNA, 74 bp, Homo sapiens
ggctggttagttcagttggttagagcgtggtgctaataacgccaaggtcgtgggttcgatccccatatcggcca

SEQ ID NO: 127 - DNA, 74 bp, Homo sapiens
ggccggttagctcagttggtaagagcgtggtgctgataacaccaaggtcgcgggctcgactcccgcaccggcca

SEQ ID NO: 128 - DNA, 93 bp, Homo sapiens
gctccagtggcgcaatcggttagcgcgcggtacttatatgacagtgcgagcggagcaatgccgaggttgtgagttcgatcctcacctggagca

SEQ ID NO: 129 - DNA, 93 bp, Homo sapiens
gctccagtggcgcaatcggttagcgcgcggtacttatacagcagtacatgcagagcaatgccgaggttgtgagttcgagcctcacctggagca

SEQ ID NO: 130 - DNA, 94 bp, Homo sapiens
gctccagtggcgcaatcggttagcgcgcggtacttatatggcagtatgtgtgcgagtgatgccgaggttgtgagttcgagcctcacctggagca

SEQ ID NO: 131 - DNA, 94 bp, Homo sapiens
gctccagtggcgcaatcggttagcgcgcggtacttatacaacagtatatgtgcgggtgatgccgaggttgtgagttcgagcctcacctggagca

SEQ ID NO: 132 - DNA, 94 bp, Homo sapiens
gctccagtggcgcaatcggttagcgcgcggtacttataagacagtgcacctgtgagcaatgccgaggttgtgagttcaagcctcacctggagca

SEQ ID NO: 133 - DNA, 82 bp, Homo sapiens
ggtagcgtggccgagcggtctaaggcgctggattaaggctccagtctcttcggaggcgtgggttcgaatcccaccgctgcca

SEQ ID NO: 134 - DNA, 82 bp, Homo sapiens
ggtagcgtggccgagcggtctaaggcgctggattaaggctccagtctcttcgggggcgtgggttcgaatcccaccgctgcca

SEQ ID NO: 135 - DNA, 82 bp, Homo sapiens
ggtagcgtggccgagcggtctaaggcgctggattaaggctccagtctcttcgggggcgtgggttcaaatcccaccgctgcca

SEQ ID NO: 136 - DNA, 82 bp, Homo sapiens
ggtagcgtggccgagtggtctaagacgctggattaaggctccagtctcttcgggggcgtgggtttgaatcccaccgctgcca

SEQ ID NO: 137 - DNA, 106 bp, Homo sapiens
gtcaggatggccgagtggtctaaggcgccagactcaagctaagcttcctccgcggtggggattctggtctccaatggaggcgtgggttcgaatcccacttctgaca

SEQ ID NO: 138 - DNA, 105 bp, Homo sapiens
gtcaggatggccgagtggtctaaggcgccagactcaagcttggcttcctcgtgttgaggattctggtctccaatggaggcgtgggttcgaatcccacttctgaca

SEQ ID NO: 139 - DNA, 108 bp, Homo sapiens
gtcaggatggccgagtggtctaaggcgccagactcaagcttactgcttcctgtgttcgggtcttctggtctccgtatggaggcgtgggttcgaatcccacttctgaca

SEQ ID NO: 140 - DNA, 107 bp, Homo sapiens
gtcaggatggccgagtggtctaaggcgccagactcaagttgctacttcccaggtttggggcttctggtctccgcatggaggcgtgggttcgaatcccacttctgaca

SEQ ID NO: 141 - DNA, 106 bp, Homo sapiens
gtcaggatggccgagtggtctaaggcgccagactcaaggtaagcaccttgcctgcgggctttctggtctccggatggaggcgtgggttcgaatcccacttctgaca

SEQ ID NO: 142 - DNA, 74 bp, Homo sapiens
gcctccttagtgcagtaggtagcgcatcagtctcaaaatctgaatggtcctgagttcaagcctcagagggggca

SEQ ID NO: 143 - DNA, 84 bp, Homo sapiens
gtcaggatggccgagcagtcttaaggcgctgcgttcaaatcgcaccctccgctggaggcgtgggttcgaatcccacttttgaca

SEQ ID NO: 144 - DNA, 83 bp, Homo sapiens
gtcaggatggccgagcggtctaaggcgctgcgttcaggtcgcagtctcccctggaggcgtgggttcgaatcccactcctgaca

SEQ ID NO: 145 - DNA, 83 bp, Homo sapiens
gtcaggatggccgagcggtctaaggcgctgcgttcaggtcgcagtctcccctggaggcgtgggttcgaatcccacttctgaca

SEQ ID NO: 146 - DNA, 83 bp, Homo sapiens
accaggatggccgagtggttaaggcgttggacttaagatccaatggacatatgtccgcgtgggttcgaaccccactcctggta

SEQ ID NO: 147 - DNA, 83 bp, Homo sapiens
accgggatggccgagtggttaaggcgttggacttaagatccaatgggctggtgcccgcgtgggttcgaaccccactctcggta

SEQ ID NO: 148 - DNA, 83 bp, Homo sapiens
accagaatggccgagtggttaaggcgttggacttaagatccaatggattcatatccgcgtgggttcgaaccccacttctggta

SEQ ID NO: 149 - DNA, 83 bp, Homo sapiens
accgggatggctgagtggttaaggcgttggacttaagatccaatggacaggtgtccgcgtgggttcgagccccactcccggta

SEQ ID NO: 150 - DNA, 82 bp, Homo sapiens
ggtagcgtggccgagcggtctaaggcgctggatttaggctccagtctcttcggaggcgtgggttcgaatcccaccgctgcca

SEQ ID NO: 151 - DNA, 82 bp, Homo sapiens
ggtagtgtggccgagcggtctaaggcgctggatttaggctccagtctcttcgggggcgtgggttcgaatcccaccactgcca

SEQ ID NO: 152 - DNA, 82 bp, Homo sapiens
ggtagcgtggccgagtggtctaaggcgctggatttaggctccagtcatttcgatggcgtgggttcgaatcccaccgctgcca

SEQ ID NO: 153 - DNA, 73 bp, Homo sapiens
gcccggctagctcagtcggtagagcatgggactcttaatcccagggtcgtgggttcgagccccacgttgggcg

SEQ ID NO: 154 - DNA, 73 bp, Homo sapiens
gcccagctagctcagtcggtagagcataagactcttaatctcagggttgtggattcgtgccccatgctgggtg

SEQ ID NO: 155 - DNA, 74 bp, Homo sapiens
gcagctagctcagtcggtagagcatgagactcttaatctcagggtcatgggttcgtgccccatgttgggtgcca

SEQ ID NO: 156 - DNA, 73 bp, Homo sapiens
gcccggctagctcagtcggtagagcatgagactcttaatctcagggtcgtgggttcgagccccacgttgggcg

SEQ ID NO: 157 - DNA, 73 bp, Homo sapiens
gcccggctagctcagtcggtagagcatgagacccttaatctcagggtcgtgggttcgagccccacgttgggcg

SEQ ID NO: 158 - DNA, 73 bp, Homo sapiens
gcccggctagctcagtcggtagagcatgggactcttaatctcagggtcgtgggttcgagccccacgttgggcg

SEQ ID NO: 159 - DNA, 73 bp, Homo sapiens
gcccggctagctcagtcgatagagcatgagactcttaatctcagggtcgtgggttcgagccgcacgttgggcg

SEQ ID NO: 160 - DNA, 73 bp, Homo sapiens
gcccagctagctcagtcggtagagcatgagactcttaatctcagggtcatgggtttgagccccacgtttggtg

SEQ ID NO: 161 - DNA, 73 bp, Homo sapiens
gcctggctagctcagtcggcaaagcatgagactcttaatctcagggtcgtgggctcgagctccatgttgggcg

SEQ ID NO: 162 - DNA, 73 bp, Homo sapiens
gcccgactacctcagtcggtggagcatgggactcttcatcccagggttgtgggttcgagccccacattgggca

SEQ ID NO: 163 - DNA, 73 bp, Homo sapiens
gcctggatagctcagttggtagagcatcagacttttaatctgagggtccagggttcaagtccctgttcaggca

SEQ ID NO: 164 - DNA, 73 bp, Homo sapiens
acccagatagctcagtcagtagagcatcagacttttaatctgagggtccaaggttcatgtccctttttgggtg

SEQ ID NO: 165 - DNA, 73 bp, Homo sapiens
gcctggatagctcagttggtagagcatcagacttttaatctgagggtccagggttcaagtccctgttcaggcg

SEQ ID NO: 166 - DNA, 73 bp, Homo sapiens
gcccggatagctcagtcggtagagcatcagacttttaatctgagggtccagggttcaagtccctgttcgggcg

SEQ ID NO: 167 - DNA, 73 bp, Homo sapiens
gcctggatagctcagtcggtagagcatcagacttttaatctgagggtccagggttcaagtccctgttcaggcg

SEQ ID NO: 168 - DNA, 73 bp, Homo sapiens
gcccggatagctcagtcggtagagcatcagacttttaatctgagggtccggggttcaagtccctgttcgggcg

SEQ ID NO: 169 - DNA, 73 bp, Homo sapiens
gcctgggtagctcagtcggtagagcatcagacttttaatctgagggtccagggttcaagtccctgtccaggcg

SEQ ID NO: 170 - DNA, 73 bp, Homo sapiens
gcctggatagctcagttggtagaacatcagacttttaatctgacggtgcagggttcaagtccctgttcaggcg

SEQ ID NO: 171 - DNA, 73 bp, Homo sapiens
gcctcgttagcgcagtaggtagcgcgtcagtctcataatctgaaggtcgtgagttcgatcctcacacggggca

SEQ ID NO: 172 - DNA, 73 bp, Homo sapiens
gccctcttagcgcagtgggcagcgcgtcagtctcataatctgaaggtcctgagttcgagcctcagagagggca

SEQ ID NO: 173 - DNA, 73 bp, Homo sapiens
gcctccttagcgcagtaggcagcgcgtcagtctcataatctgaaggtcctgagttcgaacctcagagggggca

SEQ ID NO: 174 - DNA, 73 bp, Homo sapiens
gccctcttagcgcagcgggcagcgcgtcagtctcataatctgaaggtcctgagttcgagcctcagagagggca

SEQ ID NO: 175 - DNA, 73 bp, Homo sapiens
gccctcttagcgcagctggcagcgcgtcagtctcataatctgaaggtcctgagttcaagcctcagagagggca

SEQ ID NO: 176 - DNA, 73 bp, Homo sapiens
gcctcgttagcgcagtaggcagcgcgtcagtctcataatctgaaggtcgtgagttcgagcctcacacggggca

SEQ ID NO: 177 - DNA, 73 bp, Homo sapiens
gccctcttagtgcagctggcagcgcgtcagtttcataatctgaaagtcctgagttcaagcctcagagagggca

SEQ ID NO: 178 - DNA, 73 bp, Homo sapiens
gccgaaatagctcagttgggagagcgttagactgaagatctaaaggtccctggttcgatcccgggtttcggca

SEQ ID NO: 179 - DNA, 73 bp, Homo sapiens
gccgaaatagctcagttgggagagcgttagactgaagatctaaaggtccctggttcaatcccgggtttcggca

SEQ ID NO: 180 - DNA, 73 bp, Homo sapiens
gccgagatagctcagttgggagagcgttagactgaagatctaaaggtccctggttcaatcccgggtttcggca

SEQ ID NO: 181 - DNA, 74 bp, Homo sapiens
gccgaaatagctcagttgggagagcgttagaccgaagatcttaaaggtccctggttcaatcccgggtttcggca

SEQ ID NO: 182 - DNA, 74 bp, Homo sapiens
gctgaaatagctcagttgggagagcgttagactgaagatcttaaagttccctggttcaaccctgggtttcagcc

SEQ ID NO: 183 - DNA, 72 bp, Homo sapiens
ggctcgttggtctaggggtatgattctcgcttaggatgcgagaggtcccgggttcaaatcccggacgagccc

SEQ ID NO: 184 - DNA, 72 bp, Homo sapiens
ggctcgttggtctaggggtatgattctcgcttagggtgcgagaggtcccgggttcaaatcccggacgagccc

SEQ ID NO: 185 - DNA, 72 bp, Homo sapiens
ggctcgttggtctaggggtatgattctcgcttcgggtgcgagaggtcccgggttcaaatcccggacgagccc

SEQ ID NO: 186 - DNA, 72 bp, Homo sapiens
ggctcgttggtctaggggtatgattctcgcttcgggtgtgagaggtcccgggttcaaatcccggacgagccc

SEQ ID NO: 187 - DNA, 72 bp, Homo sapiens
ggctcgttggtctagtggtatgattctcgctttgggtgcgagaggtcccgggttcaaatcccggacgagccc

SEQ ID NO: 188 - DNA, 72 bp, Homo sapiens
ggctcgttggtctaggggtatgattctcggtttgggtccgagaggtcccgggttcaaatcccggacgagccc

SEQ ID NO: 189 - DNA, 72 bp, Homo sapiens
ggctcgttggtctaggggtatgattctcgctttgggtgcgagaggtcccgggttcaaatcccggacgagccc

SEQ ID NO: 190 - DNA, 87 bp, Homo sapiens
gcccggatgatcctcagtggtctggggtgcaggcttcaaacctgtagctgtctagcgacagagtggttcaattccacctttcgggcg

SEQ ID NO: 191 - DNA, 84 bp, Homo sapiens
gctcggatgatcctcagtggtctggggtgcaggcttcaaacctgtagctgtctagtgacagagtggttcaattccacctttgta

SEQ ID NO: 192 - DNA, 82 bp, Homo sapiens
gtagtcgtggccgagtggttaaggcgatggactagaaatccattggggtttccccgcgcaggttcgaatcctgccgactacg

SEQ ID NO: 193 - DNA, 82 bp, Homo sapiens
gtagtcgtggccgagtggttaaggcgatggactagaaatccattggggtctccccgcgcaggttcgaatcctgccgactacg

SEQ ID NO: 194 - DNA, 82 bp, Homo sapiens
gtagtcgtggccgagtggttaaggcgatggactagaaatccattggggtttccccacgcaggttcgaatcctgccgactacg

SEQ ID NO: 195 - DNA, 82 bp, Homo sapiens
gtagtcgtggccgagtggttaaggtgatggactagaaacccattggggtctccccgcgcaggttcgaatcctgccgactacg

SEQ ID NO: 196 - DNA, 82 bp, Homo sapiens
gctgtgatggccgagtggttaaggcgttggactcgaaatccaatggggtctccccgcgcaggttcgaatcctgctcacagcg

SEQ ID NO: 197 - DNA, 82 bp, Homo sapiens
gctgtgatggccgagtggttaaggcgttggactcgaaatccaatggggtctccccgcgcaggttcaaatcctgctcacagcg

SEQ ID NO: 198 - DNA, 82 bp, Homo sapiens
gctgtgatggccgagtggttaaggtgttggactcgaaatccaatgggggttccccgcgcaggttcaaatcctgctcacagcg

SEQ ID NO: 199 - DNA, 82 bp, Homo sapiens
gtcacggtggccgagtggttaaggcgttggactcgaaatccaatggggtttccccgcacaggttcgaatcctgttcgtgacg

SEQ ID NO: 200 - DNA, 82 bp, Homo sapiens
gacgaggtggccgagtggttaaggcgatggactgctaatccattgtgctctgcacgcgtgggttcgaatcccaccctcgtcg

SEQ ID NO: 201 - DNA, 82 bp, Homo sapiens
gacgaggtggccgagtggttaaggcgatggactgctaatccattgtgctctgcacgcgtgggttcgaatcccaccttcgtcg

SEQ ID NO: 202 - DNA, 82 bp, Homo sapiens
gacgaggtggccgagtggttaaggcgatggactgctaatccattgtgctttgcacgcgtgggttcgaatcccatcctcgtcg

SEQ ID NO: 203 - DNA, 82 bp, Homo sapiens
gacgaggtggccgagtggttaaggcgatggactgctaatccattgtgctctgcacgcgtgggttcgaatcccatcctcgtcg

SEQ ID NO: 204 - DNA, 82 bp, Homo sapiens
gacgaggtggccgagtggttaaggcgatggactgctaatccattgtgctctgcacacgtgggttcgaatcccatcctcgtcg

SEQ ID NO: 205 - DNA, 84 bp, Homo sapiens
ggagaggcctggccgagtggttaaggcgatggactgctaatccattgtgctctgcacgcgtgggttcgaatcccatcctcgtcg

SEQ ID NO: 206 - DNA, 82 bp, Homo sapiens
gcagcgatggccgagtggttaaggcgttggacttgaaatccaatggggtctccccgcgcaggttcgaaccctgctcgctgcg

SEQ ID NO: 207 - DNA, 82 bp, Homo sapiens
gtagtcgtggccgagtggttaaggcgatggacttgaaatccattggggtttccccgcgcaggttcgaatcctgccgactacg

SEQ ID NO: 208 - DNA, 82 bp, Homo sapiens
gtagtcgtggccgagtggttaaggcgatggacttgaaatccattggggtctccccgcgcaggttcgaatcctgccgactacg

SEQ ID NO: 209 - DNA, 82 bp, Homo sapiens
gtagtcgtggccgagtggttaaggcgatggacttgaaatccattggggtttccccgcgcaggttcgaatcctgtcggctacg

SEQ ID NO: 210 - DNA, 74 bp, Homo sapiens
ggcgccgtggcttagttggttaaagcgcctgtctagtaaacaggagatcctgggttcgaatcccagcggtgcct

SEQ ID NO: 211 - DNA, 74 bp, Homo sapiens
ggctccgtggcttagctggttaaagcgcctgtctagtaaacaggagatcctgggttcgaatcccagcggggcct

SEQ ID NO: 212 - DNA, 74 bp, Homo sapiens
ggctccgtagcttagttggttaaagcgcctgtctagtaaacaggagatcctgggttcgactcccagcggggcct

SEQ ID NO: 213 - DNA, 74 bp, Homo sapiens
ggcttcgtggcttagctggttaaagcgcctgtctagtaaacaggagatcctgggttcgaatcccagcgaggcct

SEQ ID NO: 214 - DNA, 74 bp, Homo sapiens
ggcgccgtggcttagctggttaaagcgcctgtctagtaaacaggagatcctgggttcgaatcccagcggtgcct

SEQ ID NO: 215 - DNA, 74 bp, Homo sapiens
ggccctgtggcttagctggtcaaagcgcctgtctagtaaacaggagatcctgggttcgaatcccagcggggcct

SEQ ID NO: 216 - DNA, 74 bp, Homo sapiens
ggctctatggcttagttggttaaagcgcctgtctcgtaaacaggagatcctgggttcgactcccagtggggcct

SEQ ID NO: 217 - DNA, 72 bp, Homo sapiens
ggcgcggtggccaagtggtaaggcgtcggtctcgtaaaccgaagatcacgggttcgaaccccgtccgtgcct

SEQ ID NO: 218 - DNA, 74 bp, Homo sapiens
ggctctgtggcttagttggctaaagcgcctgtctcgtaaacaggagatcctgggttcgaatcccagcggggcct

SEQ ID NO: 219 - DNA, 72 bp, Homo sapiens
ggcgcggtggccaagtggtaaggcgtcggtctcgtaaaccgaagatcgcgggttcgaaccccgtccgtgcct

SEQ ID NO: 220 - DNA, 74 bp, Homo sapiens
ggccctgtagctcagcggttggagcgctggtctcgtaaacctaggggtcgtgagttcaaatctcaccagggcct

SEQ ID NO: 221 - DNA, 74 bp, Homo sapiens
ggctctatggcttagttggttaaagcgcctgtcttgtaaacaggagatcctgggttcgaatcccagtagagcct

SEQ ID NO: 222 - DNA, 73 bp, Homo sapiens
ggctccatagctcagtggttagagcactggtcttgtaaaccaggggtcgcgagttcgatcctcgctggggcct

SEQ ID NO: 223 - DNA, 73 bp, Homo sapiens
ggctccatagctcaggggttagagcgctggtcttgtaaaccaggggtcgcgagttcaattctcgctggggcct

SEQ ID NO: 224 - DNA, 73 bp, Homo sapiens
ggctccatagctcaggggttagagcactggtcttgtaaaccaggggtcgcgagttcaaatctcgctggggcct

SEQ ID NO: 225 - DNA, 73 bp, Homo sapiens
ggccctatagctcaggggttagagcactggtcttgtaaaccaggggtcgcgagttcaaatctcgctggggcct

SEQ ID NO: 226 - DNA, 72 bp, Homo sapiens
ggctccatagctcaggggttagagcactggtcttgtaaaccagggtcgcgagttcaaatctcgctggggcct

SEQ ID NO: 227 - DNA, 72 bp, Homo sapiens
ggcctcgtggcgcaacggtagcgcgtctgactccagatcagaaggttgcgtgttcaaatcacgtcggggtca

SEQ ID NO: 228 - DNA, 72 bp, Homo sapiens
gacctcgtggcgcaatggtagcgcgtctgactccagatcagaaggttgcgtgttcaagtcacgtcggggtca

SEQ ID NO: 229 - DNA, 72 bp, Homo sapiens
gacctcgtggcgcaacggtagcgcgtctgactccagatcagaaggttgcgtgttcaaatcacgtcggggtca

SEQ ID NO: 230 - DNA, 72 bp, Homo sapiens
gacctcgtggcgcaacggtagcgcgtctgactccagatcagaaggctgcgtgttcgaatcacgtcggggtca

SEQ ID NO: 231 - DNA, 72 bp, Homo sapiens
gacctcgtggcgcaacggcagcgcgtctgactccagatcagaaggttgcgtgttcaaatcacgtcggggtca

SEQ ID NO: 232 - DNA, 93 bp, Homo sapiens
ccttcaatagttcagctggtagagcagaggactatagctacttcctcagtaggagacgtccttaggttgctggttcgattccagcttgaagga

SEQ ID NO: 233 - DNA, 91 bp, Homo sapiens
ccttcgatagctcagttggtagagcggaggactgtagttggctgtgtccttagacatccttaggtcgctggttcgaatccggctcgaagga

SEQ ID NO: 234 - DNA, 89 bp, Homo sapiens
ccttcgatagctcagttggtagagcggaggactgtagtggatagggcgtggcaatccttaggtcgctggttcgattccggctcgaagga

SEQ ID NO: 235 - DNA, 89 bp, Homo sapiens
ccttcgatagctcagttggtagagcggaggactgtaggctcattaagcaaggtatccttaggtcgctggttcgaatccggctcggagga

SEQ ID NO: 236 - DNA, 94 bp, Homo sapiens
ccttcgatagctcagctggtagagcggaggactgtagattgtatagacatttgcggacatccttaggtcgctggttcgattccagctcgaagga

SEQ ID NO: 237 - DNA, 93 bp, Homo sapiens
ccttcgatagctcagctggtagagcggaggactgtagctacttcctcagcaggagacatccttaggtcgctggttcgattccggctcgaagga

SEQ ID NO: 238 - DNA, 89 bp, Homo sapiens
ccttcgatagctcagctggtagagcggaggactgtaggcgcgcgcccgtggccatccttaggtcgctggttcgattccggctcgaagga

SEQ ID NO: 239 - DNA, 94 bp, Homo sapiens
ccttcgatagctcagctggtagagcggaggactgtagcctgtagaaacatttgtggacatccttaggtcgctggttcgattccggctcgaagga

SEQ ID NO: 240 - DNA, 94 bp, Homo sapiens
ccttcgatagctcagctggtagagcggaggactgtagattgtacagacatttgcggacatccttaggtcgctggttcgattccggctcgaagga

SEQ ID NO: 241 - DNA, 89 bp, Homo sapiens
ccttcgatagctcagctggtagagcggaggactgtagtacttaatgtgtggtcatccttaggtcgctggttcgattccggctcgaagga

SEQ ID NO: 242 - DNA, 89 bp, Homo sapiens
ccttcgatagctcagctggtagagcggaggactgtaggggtttgaatgtggtcatccttaggtcgctggttcgaatccggctcggagga

SEQ ID NO: 243 - DNA, 94 bp, Homo sapiens
ccttcgatagctcagctggtagagcggaggactgtagactgcggaaacgtttgtggacatccttaggtcgctggttcaattccggctcgaagga

SEQ ID NO: 244 - DNA, 90 bp, Homo sapiens
ctttcgatagctcagttggtagagcggaggactgtaggttcattaaactaaggcatccttaggtcgctggttcgaatccggctcgaagga

SEQ ID NO: 245 - DNA, 88 bp, Homo sapiens
tcttcaatagctcagctggtagagcggaggactgtaggtgcacgcccgtggccattcttaggtgctggtttgattccgacttggagag

SEQ ID NO: 246 - DNA, 73 bp, Homo sapiens
gtttccgtagtgtagtggttatcacgttcgcctaacacgcgaaaggtccccggttcgaaaccgggcggaaaca

SEQ ID NO: 247 - DNA, 73 bp, Homo sapiens
gtttccgtagtgtagtggtcatcacgttcgcctaacacgcgaaaggtccccggttcgaaaccgggcggaaaca

SEQ ID NO: 248 - DNA, 73 bp, Homo sapiens
gtttccgtagtgtagtggttatcacgttcgcctaacacgcgaaaggtccctggatcaaaaccaggcggaaaca

SEQ ID NO: 249 - DNA, 73 bp, Homo sapiens
gtttccgtagtgtagtggttatcacgttcgcctaacacgcgaaaggtccgcggttcgaaaccgggcggaaaca

SEQ ID NO: 250 - DNA, 73 bp, Homo sapiens
gtttccgtagtgtagtggttatcacgtttgcctaacacgcgaaaggtccccggttcgaaaccgggcagaaaca

SEQ ID NO: 251 - DNA, 72 bp, Homo sapiens
gggggtgtagctcagtggtagagcgtatgcttaacattcatgaggctctgggttcgatccccagcacttcca

SEQ ID NO: 252 - DNA, 73 bp, Homo sapiens
gtttccgtagtgtagtggttatcacgttcgcctcacacgcgaaaggtccccggttcgaaaccgggcggaaaca

SEQ ID NO: 253 - DNA, 73 bp, Homo sapiens
gcttctgtagtgtagtggttatcacgttcgcctcacacgcgaaaggtccccggttcgaaaccgggcagaagca

SEQ ID NO: 254 - DNA, 73 bp, Homo sapiens
gtttccgtagtgtagcggttatcacattcgcctcacacgcgaaaggtccccggttcgatcccgggcggaaaca

SEQ ID NO: 255 - DNA, 73 bp, Homo sapiens
gtttccgtagtgtagtggttatcacgttcgcctcacacgcgaaaggtccccggttcgaaactgggcggaaaca

SEQ ID NO: 256 - DNA, 74 bp, Homo sapiens
gtttccgtagtgtagtggttatcacgttcgcctcacacgcgtaaaggtccccggttcgaaaccgggcggaaaca

SEQ ID NO: 257 - DNA, 73 bp, Homo sapiens
gtttccgtagtggagtggttatcacgttcgcctcacacgcgaaaggtccccggtttgaaaccaggcggaaaca

SEQ ID NO: 258 - DNA, 73 bp, Homo sapiens
ggttccatagtgtagtggttatcacgtctgctttacacgcagaaggtcctgggttcgagccccagtggaacca

SEQ ID NO: 259 - DNA, 73 bp, Homo sapiens
ggttccatagtgtagcggttatcacgtctgctttacacgcagaaggtcctgggttcgagccccagtggaacca

SEQ ID NO: 260 - DNA, 73 bp, Homo sapiens
ggttccatagtgtagtggttatcacatctgctttacacgcagaaggtcctgggttcaagccccagtggaacca

SEQ ID NO: 261 - DNA, 73 bp, Homo sapiens
gtttccgtggtgtagtggttatcacattcgccttacacgcgaaaggtcctcgggtcgaaaccgagcggaaaca

SEQ ID NO: 262 - DNA, 72 bp, Homo sapiens
agcagagtggcgcagcggaagcgtgctgggcccataacccagaggtcgatggatcgaaaccatcctctgcta

SEQ ID NO: 263 - DNA, 72 bp, Homo sapiens
agcagagtggcgcagcggaagcgtgctgggcccataacccagaggtcgatggatctaaaccatcctctgcta

SEQ ID NO: 264 - DNA, 73 bp, Homo sapiens
tccctggtggtctagtggctaggattcggcgctttcaccgccgcggcccgggttcgattcccggtcagggaat

SEQ ID NO: 265 - DNA, 72 bp, Homo sapiens
gcgttggtggtttagtggtagaattctcgcctcccatgcgggagacccgggttcaattcccggccactgcac

SEQ ID NO: 266 - DNA, 72 bp, Homo sapiens
ggccttggtggtgcagtggtagaattctcgcctcccacgtgggagacccgggttcaattcccggccaatgca

SEQ ID NO: 267 - DNA, 73 bp, Homo sapiens
gtccctggtggtctagtggctaggattcggcgctttcaccgccgcggcccgggttcgattcccggccagggaa

SEQ ID NO: 268 - DNA, 75 bp, Homo sapiens
tgtctctgtggcgcaatcggttagcgcgttcggctgttaaccgaaagattggtggttcgagcccacccagggacg

SEQ ID NO: 269 - DNA, 86 bp, Homo sapiens
tggctccgtggcgcaatggatagcgcattggacttctagaggctgaaggcattcaaaggttccgggttcgagtcccggcggagtcg

SEQ ID NO: 270 - DNA, 74 bp, Homo sapiens
gcccggctagctcagtcggtagagcatgagactcttaatctcagggtcgtgggttcgagccccacgttgggcgc

SEQ ID NO: 271 - DNA, 73 bp, Homo sapiens
gccgtgatcgtatagtggttagtactctgcgttgtggccgcagcaacctcggttcgaatccgagtcacggcag

SEQ ID NO: 272 - DNA, 73 bp, Homo sapiens
gcgttggtggtatagtggtgagcatagctgccttccaagcagttgacccgggttcgattcccggccaacgcag

SEQ ID NO: 273 - DNA, 73 bp, Homo sapiens
tccctggtggtctagtggttaggattcggcgctctcaccgccgcggcccgggttcgattcccggtcagggaaa

SEQ ID NO: 274 - DNA, 73 bp, Homo sapiens
aggttccatggtgtaatggtgagcactctggactctgaatccagcgatccgagttcgagtctcggtggaacct

SEQ ID NO: 275 - DNA, 75 bp, Homo sapiens
tgtctctgtggcgtagtcggttagcgcgttcggctgttaaccgaaaagttggtggttcgagcccacccaggaacg

SEQ ID NO: 276 - DNA, 75 bp, Homo sapiens
tgtctctgtggcgcaatcggttagcgcgttcggctgttaaccgaaaggttggtggttcgagcccacccagggacg

SEQ ID NO: 277 - DNA, 75 bp, Homo sapiens
gtctctgtggcgcaatcggttagcgcattcggctgttaaccgaaaggttggtggttcgagcccacccagggacgc

SEQ ID NO: 278 - DNA, 75 bp, Homo sapiens
gtctctgtggcgcaatgggttagcgcgttcggctgttaaccgaaaggttggtggttcgagcccatccagggacgc

SEQ ID NO: 279 - DNA, 72 bp, Homo sapiens
gcactggtggttcagtggtagaattctcgcctcacacgcgggacacccgggttcaattcccggtcaaggcaa

SEQ ID NO: 280 - DNA, 74 bp, Homo sapiens
gtttccgtagtgtagtggttatcacgttcgcctcacacgcgaaaggtccccggttcgaaactgggcggaaacag

SEQ ID NO: 281 - DNA, 72 bp, Homo sapiens
gcactggtggttcagtggtagaattctcgcctcccacgcgggagacccgggtttaattcccggtcaagataa

SEQ ID NO: 282 - DNA, 75 bp, Homo sapiens
gtttccgtagtgtagtggttatcacgttcgcctcacacgcgtaaaggtccccggttcgaaaccgggcggaaacat

SEQ ID NO: 283 - DNA, 73 bp, Homo sapiens
tagcagagtggcgcagcggaagcgtgctgggcccataacccagaggtcgatggatcgaaaccatcctctgcta

SEQ ID NO: 284 - DNA, 74 bp, Homo sapiens
gtttccgtagtgtagtggttatcacgttcgcctcacacgcgaaaggtccccggttcgaaaccgggcggaaacaa

SEQ ID NO: 285 - DNA, 73 bp, Homo sapiens
tcctcgttagtatagtggtgagtatccccgcctgtcacgcgggagaccggggttcgattccccgacggggagg

SEQ ID NO: 286 - DNA, 72 bp, Homo sapiens
tgcatgggtggttcagtggtagaattctcgcctgccacgcgggaggcccgggttcgattcccggcccatgca

SEQ ID NO: 287 - DNA, 73 bp, Homo sapiens
tccctggtggtctagtggttaggattcggcgctctcaccgccgcggcccgggttcgattcccggtcagggaag

SEQ ID NO: 288 - DNA, 73 bp, Homo sapiens
atccttgttactatagtggtgagtatctctgcctgtcatgcgtgagagagggggtcgattccccgacggggag

SEQ ID NO: 289 - DNA, 72 bp, Homo sapiens
gcattggtggttcagtggtagaattctcgcctgccacgcgggaggcccgggttcgattcccggccaatgcac

SEQ ID NO: 290 - DNA, 84 bp, Homo sapiens
gtcaggatggccgagcggtctaaggcgctgcgttcaggtcgcagtctcccctggaggcgtgggttcgaatcccactcctgacaa

SEQ ID NO: 291 - DNA, 73 bp, Homo sapiens
cgcgttggtggtatagtggtgagcatagctgccttccaagcagttgacccgggttcgattcccggccaacgca

SEQ ID NO: 292 - DNA, 75 bp, Homo sapiens
cgtctctgtggcgcaatcggttagcgcgttcggctgttaaccgaaaggttggtggttcgatcccacccagggacg

SEQ ID NO: 293 - DNA, 73 bp, Homo sapiens
cgcgttggtggtgtagtggtgagcacagctgcctttcaagcagttaacgcgggttcgattcccgggtaacgaa

SEQ ID NO: 294 - DNA, 73 bp, Homo sapiens
cggctcgttggtctaggggtatgattctcgcttcgggtgcgagaggtcccgggttcaaatcccggacgagccc

SEQ ID NO: 295 - DNA, 73 bp, Homo sapiens
ggctcgttggtctaggggtatgattctcgcttagggtgcgagaggtcccgggttcaaatcccggacgagccct

SEQ ID NO: 296 - DNA, 74 bp, Homo sapiens
cgcccggatagctcagtcggtagagcatcagacttttaatctgagggtccagggttcaagtccctgttcgggcg

SEQ ID NO: 297 - DNA, 74 bp, Homo sapiens
gcccggatagctcagtcggtagagcatcagacttttaatctgagggtccagggttcaagtccctgttcgggcgt

SEQ ID NO: 298 - DNA, 107 bp, Homo sapiens
tgtcaggatggccgagtggtctaaggcgccagactcaaggtaagcaccttgcctgcgggctttctggtctccggatggaggcgtgggttcgaatcccacttctgaca

SEQ ID NO: 299 - DNA, 73 bp, Homo sapiens
ttccctggtggtctagtggttaggattcggcgctctcaccgccgcggcccgggttcgattcccggtcaggaaa

SEQ ID NO: 300 - DNA, 90 bp, Homo sapiens
gccttcgatagctcagttggtagagcggaggactgtagtggatagggcgtggcaatccttaggtcgctggttcgattccggctcgaagga

SEQ ID NO: 301 - DNA, 74 bp, Homo sapiens
cgggggattagctcaaatggtagagcgctcgcttagcatgcgagaggtagcgggatcgatgcccgcatcctcca

SEQ ID NO: 302 - DNA, 94 bp, Homo sapiens
agctccagtggcgcaatcggttagcgcgcggtacttatacagcagtacatgcagagcaatgccgaggttgtgagttcgagcctcacctggagca

SEQ ID NO: 303 - DNA, 72 bp, Homo sapiens
gcgccgctggtgtagtggtatcatgcaagattcccattcttgcgacccgggttcgattcccgggcggcgcat

SEQ ID NO: 304 - DNA, 73 bp, Homo sapiens
tcccatatggtctagcggttaggattcctggttttcacccaggtggcccgggttcgactcccggtatgggaac

SEQ ID NO: 305 - DNA, 73 bp, Homo sapiens
gggggatgtagctcagtggtagagcgcgcgcttcgcatgtgtgaggtcccgggttcaatccccggcatctcca

SEQ ID NO: 306 - DNA, 72 bp, Homo sapiens
gcattggtggttcagtggtagaattctcgcctgccacgcgggaggcccgggttcgattcccggccaatgcaa

SEQ ID NO: 307 - DNA, 74 bp, Homo sapiens
gggccagtggcgcaatggataacgcgtctgactacggatcagaagattctaggttcgactcctggctggctcgc

SEQ ID NO: 308 - DNA, 74 bp, Homo sapiens
ggtttccgtagtgtagtggttatcacgttcgcctaacacgcgaaaggtccccggttcgaaaccgggcggaaaca

SEQ ID NO: 309 - DNA, 74 bp, Homo sapiens
agtttccgtagtgtagtggttatcacgttcgcctaacacgcgaaaggtccccggttcgaaaccgggcggaaaca

SEQ ID NO: 310 - DNA, 83 bp, Homo sapiens
aggtagcgtggccgagcggtctaaggcgctggattaaggctccagtctcttcgggggcgtgggttcgaatcccaccgctgcca

SEQ ID NO: 311 - DNA, 74 bp, Homo sapiens
gtttccgtagtgtagtggtcatcacgttcgcctaacacgcgaaaggtccccggttcgaaaccgggcggaaacat

SEQ ID NO: 312 - DNA, 73 bp, Homo sapiens
ggctcgttggtctaggggtatgattctcgctttgggtgcgagaggtcccgggttcaaatcccggacgagccca

SEQ ID NO: 313 - DNA, 73 bp, Homo sapiens
ggctccatagctcaggggttagagcactggtcttgtaaaccagggtcgcgagttcaaatctcgctggggcctg

SEQ ID NO: 314 - DNA, 73 bp, Homo sapiens
tggggatgtagctcagtggtagagcgcatgctttgcatgtatgaggccccgggttcgatccccggcatctcca

SEQ ID NO: 315 - DNA, 74 bp, Homo sapiens
cgcccggctagctcagtcggtagagcatgagactcttaatctcagggtcgtgggttcgagccccacgttgggcg

SEQ ID NO: 316 - DNA, 74 bp, Homo sapiens
gtttccgtagtgtagtggttatcacgttcgcctaacacgcgaaaggtccccggttcgaaaccgggcggaaacaa

SEQ ID NO: 317 - DNA, 74 bp, Homo sapiens
gcccggctagctcagtcggtagagcatgagactcttaatctcagggtcgtgggttcgagccccacgttgggcgt

SEQ ID NO: 318 - DNA, 74 bp, Homo sapiens
gtttccgtagtgtagtggttatcacgttcgcctcacacgcgaaaggtccccggttcgaaaccgggcggaaacac

SEQ ID NO: 319 - DNA, 73 bp, Homo sapiens
cagcagagtggcgcagcggaagcgtgctgggcccataacccagaggtcgatggatcgaaaccatcctctgcta

SEQ ID NO: 320 - DNA, 85 bp, Homo sapiens
ggagaggcctggccgagtggttaaggcgatggactgctaatccattgtgctctgcacgcgtgggttcgaatcccatcctcgtcgc

SEQ ID NO: 321 - DNA, 73 bp, Homo sapiens
ggccccatggtgtaatggttagcactctggactttgaatccagcgatccgagttcaaatctcggtgggacctg

SEQ ID NO: 322 - DNA, 73 bp, Homo sapiens
ggccccatggtgtaatggttagcactctggactttgaatccagcgatccgagttcaaatctcggtgggaccta

SEQ ID NO: 323 - DNA, 83 bp, Homo sapiens
gtagtcgtggccgagtggttaaggcgatggacttgaaatccattggggtctccccgcgcaggttcgaatcctgccgactacgg

SEQ ID NO: 324 - DNA, 73 bp, Homo sapiens
agcagagtggcgcagcggaagcgtgctgggcccataacccagaggtcgatggatcgaaaccatcctctgctat

SEQ ID NO: 325 - DNA, 74 bp, Homo sapiens
ggaccacgtggcctaatggataaggcgtctgacttcggatcagaagattgagggttcgaatccctccgtggtta

SEQ ID NO: 326 - DNA, 83 bp, Homo sapiens
tgtagtcgtggccgagtggttaaggcgatggactagaaatccattggggtctccccgcgcaggttcgaatcctgccgactacg

SEQ ID NO: 327 - DNA, 73 bp, Homo sapiens
agcagagtggcgcagcggaagcgtgctgggcccataacccagaggtcgatggatcgaaaccatcctctgctag

SEQ ID NO: 328 - DNA, 84 bp, Homo sapiens
cgtcaggatggccgagcggtctaaggcgctgcgttcaggtcgcagtctcccctggaggcgtgggttcgaatcccactcctgaca

SEQ ID NO: 329 - DNA, 75 bp, Homo sapiens
ggctccgtggcttagctggttaaagcgcctgtctagtaaacaggagatcctgggttcgaatcccagcggggcctg

SEQ ID NO: 330 - DNA, 74 bp, Homo sapiens
agggccagtggcgcaatggataacgcgtctgactacggatcagaagattccaggttcgactcctggctggctcg

SEQ ID NO: 331 - DNA, 74 bp, Homo sapiens
ggtttccgtagtgtagtggttatcacgttcgcctcacacgcgaaaggtccccggttcgaaaccgggcggaaaca

SEQ ID NO: 332 - DNA, 73 bp, Homo sapiens
aggggatgtagctcagtggtagagcgcatgcttcgcatgtatgaggtcccgggttcgatccccggcatctcca

SEQ ID NO: 333 - DNA, 75 bp, Homo sapiens
tggccggttagctcagttggttagagcgtggtgctaataacgccaaggtcgcgggttcgatccccgtacgggcca

SEQ ID NO: 334 - DNA, 73 bp, Homo sapiens
cggctcgttggtctaggggtatgattctcgcttagggtgcgagaggtcccgggttcaaatcccggacgagccc

SEQ ID NO: 335 - DNA, 74 bp, Homo sapiens
agcccggctagctcagtcggtagagcatgagactcttaatctcagggtcgtgggttcgagccccacgttgggcg

SEQ ID NO: 336 - DNA, 92 bp, Homo sapiens
tccttcgatagctcagttggtagagcggaggactgtagttggctgtgtccttagacatccttaggtcgctggttcgaatccggctcgaagga

SEQ ID NO: 337 - DNA, 74 bp, Homo sapiens
ggggaattagctcaaatggtagagcgctcgcttagcatgcgagaggtagcgggatcgatgcccgcattctccag

SEQ ID NO: 338 - DNA, 74 bp, Homo sapiens
cgccctcttagcgcagcgggcagcgcgtcagtctcataatctgaaggtcctgagttcgagcctcagagagggca

SEQ ID NO: 339 - DNA, 95 bp, Homo sapiens
tgctccagtggcgcaatcggttagcgcgcggtacttatatggcagtatgtgtgcgagtgatgccgaggttgtgagttcgagcctcacctggagca

SEQ ID NO: 340 - DNA, 73 bp, Homo sapiens
tgccgtgatcgtatagtggttagtactctgcgttgtggccgcagcaacctcggttcgaatccgagtcacggca

SEQ ID NO: 341 - DNA, 75 bp, Homo sapiens
ggccggttagctcagttggttagagcgtggtgctaataacgccaaggtcgcgggttcgatccccgtacgggccac

SEQ ID NO: 342 - DNA, 74 bp, Homo sapiens
agtttccgtagtgtagtggttatcacgtttgcctaacacgcgaaaggtccccggttcgaaaccgggcagaaaca

SEQ ID NO: 343 - DNA, 74 bp, Homo sapiens
gcttctgtagtgtagtggttatcacgttcgcctcacacgcgaaaggtccccggttcgaaaccgggcagaagcaa

SEQ ID NO: 344 - DNA, 73 bp, Homo sapiens
ttcctcgttagtatagtggtgagtatccccgcctgtcacgcgggagaccggggttcgattccccgacggggag

SEQ ID NO: 345 - DNA, 83 bp, Homo sapiens
gtagtcgtggccgagtggttaaggcgatggacttgaaatccattggggtttccccgcgcaggttcgaatcctgtcggctacgg

SEQ ID NO: 346 - DNA, 73 bp, Homo sapiens
aggttccatggtgtaatggttagcactctggactctgaatccagcgatccgagttcaaatctcggtggaacct

SEQ ID NO: 347 - DNA, 73 bp, Homo sapiens
tcctcgttagtatagtggtgagtgtccccgtctgtcacgcgggagaccggggttcgattccccgacggggaga

SEQ ID NO: 348 - DNA, 74 bp, Homo sapiens
gtttccgtagtgtagtggttatcacgttcgcctaacacgcgaaaggtccctggatcaaaaccaggcggaaacaa

SEQ ID NO: 349 - DNA, 75 bp, Homo sapiens
cggccggttagctcagttggttagagcgtggtgctaataacgccaaggtcgcgggttcgatccccgtactggcca

SEQ ID NO: 350 - DNA, 73 bp, Homo sapiens
ggccccatggtgtaatggtcagcactctggactctgaatccagcgatccgagttcaaatctcggtgggaccca

SEQ ID NO: 351 - DNA, 73 bp, Homo sapiens
ggccccatggtgtaatggttagcactctggactttgaatccagcgatccgagttcaaatctcggtgggacctt

SEQ ID NO: 352 - DNA, 73 bp, Homo sapiens
tgggggtgtagctcagtggtagagcgcgtgcttagcatgtacgaggtcccgggttcaatccccggcacctcca

SEQ ID NO: 353 - DNA, 73 bp, Homo sapiens
ggggatgtagctcagtggtagagcgcatgcttagcatgcatgaggtcccgggttcgatccccagcatctccag

SEQ ID NO: 354 - DNA, 73 bp, Homo sapiens
agggggtgtagctcagtggtagagcgcgtgcttcgcatgtacgaggccccgggttcgacccccggctcctcca

SEQ ID NO: 355 - DNA, 73 bp, Homo sapiens
gggggtgtagctcagtggtagagcgcgtgcttagcatgcacgaggccccgggttcaatccccggcacctccat

SEQ ID NO: 356 - DNA, 73 bp, Homo sapiens
gggggtgtagctcagtggtagagcgcgtgcttagcatgcacgaggccccgggttcaatccccggcacctccag

SEQ ID NO: 357 - DNA, 107 bp, Homo sapiens
gtcaggatggccgagtggtctaaggcgccagactcaagctaagcttcctccgcggtggggattctggtctccaatggaggcgtgggttcgaatcccacttctgacac

SEQ ID NO: 358 - DNA, 106 bp, Homo sapiens
tgtcaggatggccgagtggtctaaggcgccagactcaagcttggcttcctcgtgttgaggattctggtctccaatggaggcgtgggttcgaatcccacttctgaca

SEQ ID NO: 359 - DNA, 73 bp, Homo sapiens
ggttccatggtgtaatggttagcactctggactctgaatccagcgatccgagttcaaatctcggtggaacctt

SEQ ID NO: 360 - DNA, 83 bp, Homo sapiens
ggtagcgtggccgagcggtctaaggcgctggattaaggctccagtctcttcgggggcgtgggttcgaatcccaccgctgccag

SEQ ID NO: 361 - DNA, 74 bp, Homo sapiens
tgcctccttagcgcagtaggcagcgcgtcagtctcataatctgaaggtcctgagttcgaacctcagagggggca

SEQ ID NO: 362 - DNA, 74 bp, Homo sapiens
agcccggatagctcagtcggtagagcatcagacttttaatctgagggtccagggttcaagtccctgttcgggcg

SEQ ID NO: 363 - DNA, 74 bp, Homo sapiens
gcctccttagcgcagtaggcagcgcgtcagtctcataatctgaaggtcctgagttcgaacctcagagggggcag

SEQ ID NO: 364 - DNA, 73 bp, Homo sapiens
ttccctggtggtctagtggttaggattcggcgctctcaccgccgcggcccgggttcgattcccggtcagggaa

SEQ ID NO: 365 - DNA, 84 bp, Homo sapiens
caccaggatggccgagtggttaaggcgttggacttaagatccaatggacatatgtccgcgtgggttcgaaccccactcctggta

SEQ ID NO: 366 - DNA, 73 bp, Homo sapiens
tggctcgttggtctaggggtatgattctcgcttagggtgcgagaggtcccgggttcaaatcccggacgagccc

SEQ ID NO: 367 - DNA, 74 bp, Homo sapiens
agccccagtggcctaatggataaggcattggcctcctaagccagggattgtgggttcgagtcccatctggggtg

SEQ ID NO: 368 - DNA, 73 bp, Homo sapiens
ggggatatagctcaggggtagagcatttgactgcagatcaagaggtccccggttcaaatccgggtgccccccc

SEQ ID NO: 369 - DNA, 94 bp, Homo sapiens
cccttcgatagctcagctggtagagcggaggactgtagctacttcctcagcaggagacatccttaggtcgctggttcgattccggctcgaagga

SEQ ID NO: 370 - DNA, 90 bp, Homo sapiens
cccttcgatagctcagctggtagagcggaggactgtaggcgcgcgcccgtggccatccttaggtcgctggttcgattccggctcgaagga

SEQ ID NO: 371 - DNA, 74 bp, Homo sapiens
tgggggattagctcaaatggtagagcgctcgcttagcatgcgagaggtagcgggatcgatgcccgcatcctcca

SEQ ID NO: 372 - DNA, 83 bp, Homo sapiens
gtagtcgtggccgagtggttaaggcgatggactagaaatccattggggtctccccgcgcaggttcgaatcctgccgactacgg

SEQ ID NO: 373 - DNA, 74 bp, Homo sapiens
gcctcgttagcgcagtaggtagcgcgtcagtctcataatctgaaggtcgtgagttcgatcctcacacggggcac

SEQ ID NO: 374 - DNA, 92 bp, Homo sapiens
ggctctgtggcgcaatggatagcgcattggacttctagctgagcctagtgtggtcattcaaaggttgtgggttcgagtcccaccagagtcga

SEQ ID NO: 375 - DNA, 75 bp, Homo sapiens
gtctctgtggcgcaatcggttagcgcgttcggctgttaaccgaaaggttggtggttcgagcccacccagggacgc

SEQ ID NO: 376 - DNA, 83 bp, Homo sapiens
ggcagcgatggccgagtggttaaggcgttggacttgaaatccaatggggtctccccgcgcaggttcgaaccctgctcgctgcg

SEQ ID NO: 377 - DNA, 74 bp, Homo sapiens
ggttccatagtgtagtggttatcacgtctgctttacacgcagaaggtcctgggttcgagccccagtggaaccat

SEQ ID NO: 378 - DNA, 74 bp, Homo sapiens
ggttccatagtgtagcggttatcacgtctgctttacacgcagaaggtcctgggttcgagccccagtggaaccac

SEQ ID NO: 379 - DNA, 87 bp, Homo sapiens
tggctctgtggcgcaatggatagcgcattggacttctagatagttagagaaattcaaaggttgtgggttcgagtcccaccagagtcg

SEQ ID NO: 380 - DNA, 84 bp, Homo sapiens
taccagaatggccgagtggttaaggcgttggacttaagatccaatggattcatatccgcgtgggttcgaaccccacttctggta

SEQ ID NO: 381 - DNA, 74 bp, Homo sapiens
ggcccggatagctcagtcggtagagcatcagacttttaatctgagggtccggggttcaagtccctgttcgggcg

SEQ ID NO: 382 - DNA, 74 bp, Homo sapiens
gccgaaatagctcagttgggagagcgttagactgaagatctaaaggtccctggttcgatcccgggtttcggcag

SEQ ID NO: 383 - DNA, 74 bp, Homo sapiens
gcccggatagctcagtcggtagagcatcagacttttaatctgagggtccagggttcaagtccctgttcgggcgg

SEQ ID NO: 384 - DNA, 74 bp, Homo sapiens
gccgaaatagctcagttgggagagcgttagactgaagatctaaaggtccctggttcaatcccgggtttcggcag

SEQ ID NO: 385 - DNA, 83 bp, Homo sapiens
ggacgaggtggccgagtggttaaggcgatggactgctaatccattgtgctttgcacgcgtgggttcgaatcccatcctcgtcg

SEQ ID NO: 386 - DNA, 73 bp, Homo sapiens
ggctcgttggtctaggggtatgattctcggtttgggtccgagaggtcccgggttcaaatcccggacgagcccc

SEQ ID NO: 387 - DNA, 83 bp, Homo sapiens
agtcacggtggccgagtggttaaggcgttggactcgaaatccaatggggtttccccgcacaggttcgaatcctgttcgtgacg

SEQ ID NO: 388 - DNA, 73 bp, Homo sapiens
ctcctcgttagtatagtggttagtatccccgcctgtcacgcgggagaccggggttcaattccccgacggggag

SEQ ID NO: 389 - DNA, 73 bp, Homo sapiens
ggacctcgtggcgcaacggtagcgcgtctgactccagatcagaaggctgcgtgttcgaatcacgtcggggtca

SEQ ID NO: 390 - DNA, 73 bp, Homo sapiens
ggggatgtagctcagtggtagagcgcatgctttgcatgtatgaggccccgggttcgatccccggcatctccat

SEQ ID NO: 391 - DNA, 74 bp, Homo sapiens
gccgaaatagctcagttgggagagcgttagactgaagatctaaaggtccctggttcgatcccgggtttcggcac

SEQ ID NO: 392 - DNA, 73 bp, Homo sapiens
aggggatgtagctcagtggtagagcgcatgctttgcacgtatgaggccccgggttcaatccccggcatctcca

SEQ ID NO: 393 - DNA, 75 bp, Homo sapiens
gtctctgtggcgcaatcggttagcgcgttcggctgttaaccgaaaggttggtggttcgagcccacccagggacgg

SEQ ID NO: 394 - DNA, 73 bp, Homo sapiens
tcccacatggtctagcggttaggattcctggttttcacccaggcggcccgggttcgactcccggtgtgggaac

SEQ ID NO: 395 - DNA, 74 bp, Homo sapiens
ggctccatagctcaggggttagagcgctggtcttgtaaaccaggggtcgcgagttcaattctcgctggggcctg

SEQ ID NO: 396 - DNA, 83 bp, Homo sapiens
tggtagtgtggccgagcggtctaaggcgctggatttaggctccagtctcttcgggggcgtgggttcgaatcccaccactgcca

SEQ ID NO: 397 - DNA, 74 bp, Homo sapiens
ggctccatagctcaggggttagagcactggtcttgtaaaccaggggtcgcgagttcaaatctcgctggggcctc

SEQ ID NO: 398 - DNA, 73 bp, Homo sapiens
tggctcgttggtctagtggtatgattctcgctttgggtgcgagaggtcccgggttcaaatcccggacgagccc

SEQ ID NO: 399 - DNA, 95 bp, Homo sapiens
ccttcgatagctcagctggtagagcggaggactgtagattgtacagacatttgcggacatccttaggtcgctggttcgattccggctcgaaggaa

SEQ ID NO: 400 - DNA, 74 bp, Homo sapiens
aggccctatagctcaggggttagagcactggtcttgtaaaccaggggtcgcgagttcaaatctcgctggggcct

SEQ ID NO: 401 - DNA, 90 bp, Homo sapiens
tccttcgatagctcagctggtagagcggaggactgtagtacttaatgtgtggtcatccttaggtcgctggttcgattccggctcgaagga

SEQ ID NO: 402 - DNA, 73 bp, Homo sapiens
tggctcgttggtctaggggtatgattctcgctttgggtgcgagaggtcccgggttcaaatcccggacgagccc

SEQ ID NO: 403 - DNA, 74 bp, Homo sapiens
gcccggctagctcagtcggtagagcatgggactcttaatcccagggtcgtgggttcgagccccacgttgggcgc

SEQ ID NO: 404 - DNA, 75 bp, Homo sapiens
cggccggttagctcagttggttagagcgtggtgctaataacgccaaggtcgcgggttcgatccccgtacgggcca

SEQ ID NO: 405 - DNA, 73 bp, Homo sapiens
tcccacatggtctagcggttaggattcctggttttcacccaggcggcccgggttcgactcccggtgtgggaat

SEQ ID NO: 406 - DNA, 83 bp, Homo sapiens
gacgaggtggccgagtggttaaggcgatggactgctaatccattgtgctctgcacgcgtgggttcgaatcccatcctcgtcga

SEQ ID NO: 407 - DNA, 73 bp, Homo sapiens
gccgtgatcgtatagtggttagtactctgcgttgtggccgcagcaacctcggttcgaatccgagtcacggcat

SEQ ID NO: 408 - DNA, 73 bp, Homo sapiens
cgccgtgatcgtatagtggttagtactctgcgttgtggccgcagcaacctcggttcgaatccgagtcacggca

SEQ ID NO: 409 - DNA, 73 bp, Homo sapiens
ggttccatggtgtaatggttagcactctggactctgaatccagcgatccgagttcaaatctcggtggaacctg

SEQ ID NO: 410 - DNA, 74 bp, Homo sapiens
tgcccggctagctcagtcggtagagcatgggactcttaatcccagggtcgtgggttcgagccccacgttgggcg

SEQ ID NO: 411 - DNA, 74 bp, Homo sapiens
gggccgcgtggcctaatggataaggcgtctgacttcggatcagaagattgcaggttcgagtcctgccgcggtcg

SEQ ID NO: 412 - DNA, 72 bp, Homo sapiens
gcgccgctggtgtagtggtatcatgcaagattcccattcttgcgacccgggttcgattcccgggcggcgcac

SEQ ID NO: 413 - DNA, 74 bp, Homo sapiens
gggccgcgtggcctaatggataaggcgtctgattccggatcagaagattgagggttcgagtcccttcgtggtcg

SEQ ID NO: 414 - DNA, 74 bp, Homo sapiens
cgccccggtggcctaatggataaggcattggcctcctaagccagggattgtgggttcgagtcccacccggggta

SEQ ID NO: 415 - DNA, 74 bp, Homo sapiens
gcccggctagctcagtcggtagagcatgagacccttaatctcagggtcgtgggttcgagccccacgttgggcgt

SEQ ID NO: 416 - DNA, 73 bp, Homo sapiens
aggcgcggtggccaagtggtaaggcgtcggtctcgtaaaccgaagatcacgggttcgaaccccgtccgtgcct

SEQ ID NO: 417 - DNA, 83 bp, Homo sapiens
ggtagcgtggccgagtggtctaaggcgctggatttaggctccagtcatttcgatggcgtgggttcgaatcccaccgctgccac

SEQ ID NO: 418 - DNA, 83 bp, Homo sapiens
gggtagcgtggccgagcggtctaaggcgctggattaaggctccagtctcttcgggggcgtgggttcgaatcccaccgctgcca

SEQ ID NO: 419 - DNA, 84 bp, Homo sapiens
agtcaggatggccgagcggtctaaggcgctgcgttcaggtcgcagtctcccctggaggcgtgggttcgaatcccacttctgaca

SEQ ID NO: 420 - DNA, 84 bp, Homo sapiens
gtcaggatggccgagcggtctaaggcgctgcgttcaggtcgcagtctcccctggaggcgtgggttcgaatcccacttctgacag

SEQ ID NO: 421 - DNA, 74 bp, Homo sapiens
gcctcgttagcgcagtaggcagcgcgtcagtctcataatctgaaggtcgtgagttcgagcctcacacggggcag

SEQ ID NO: 422 - DNA, 83 bp, Homo sapiens
ggtagcgtggccgagcggtctaaggcgctggatttaggctccagtctcttcggaggcgtgggttcgaatcccaccgctgccag

SEQ ID NO: 423 - DNA, 89 bp, Homo sapiens
tggctctgtggcgcaatggatagcgcattggacttctagtgacgaatagagcaattcaaaggttgtgggttcgaatcccaccagagtcg

SEQ ID NO: 424 - DNA, 72 bp, Homo sapiens
cgcattggtggttcagtggtagaattctcgcctgccacgcgggaggcccgggttcgattcccggccaatgca

SEQ ID NO: 425 - DNA, 83 bp, Homo sapiens
gctgtgatggccgagtggttaaggcgttggactcgaaatccaatggggtctccccgcgcaggttcgaatcctgctcacagcgt

SEQ ID NO: 426 - DNA, 75 bp, Homo sapiens
ggcgccgtggcttagctggttaaagcgcctgtctagtaaacaggagatcctgggttcgaatcccagcggtgcctg

SEQ ID NO: 427 - DNA, 73 bp, Homo sapiens
cgacctcgtggcgcaacggtagcgcgtctgactccagatcagaaggttgcgtgttcaaatcacgtcggggtca

SEQ ID NO: 428 - DNA, 83 bp, Homo sapiens
agacgaggtggccgagtggttaaggcgatggactgctaatccattgtgctctgcacgcgtgggttcgaatcccatcctcgtcg

SEQ ID NO: 429 - DNA, 75 bp, Homo sapiens
cggcgccgtggcttagttggttaaagcgcctgtctagtaaacaggagatcctgggttcgaatcccagcggtgcct

SEQ ID NO: 430 - DNA, 73 bp, Homo sapiens
ggcctcgtggcgcaacggtagcgcgtctgactccagatcagaaggttgcgtgttcaaatcacgtcggggtcaa

SEQ ID NO: 431 - DNA, 73 bp, Homo sapiens
agcgttggtggtatagtggtaagcatagctgccttccaagcagttgacccgggttcgattcccggccaacgca

SEQ ID NO: 432 - DNA, 73 bp, Homo sapiens
tcctcgttagtatagtggtgagtatccccgcctgtcacgcgggagaccggggttcgattccccgacggggaga

SEQ ID NO: 433 - DNA, 73 bp, Homo sapiens
ggctcgttggtctaggggtatgattctcgcttcgggtgcgagaggtcccgggttcaaatcccggacgagccct

SEQ ID NO: 434 - DNA, 75 bp, Homo sapiens
ggcgccgtggcttagttggttaaagcgcctgtctagtaaacaggagatcctgggttcgaatcccagcggtgcctt

SEQ ID NO: 435 - DNA, 83 bp, Homo sapiens
gtagtcgtggccgagtggttaaggcgatggactagaaatccattggggtctccccgcgcaggttcgaatcctgccgactacgt

SEQ ID NO: 436 - DNA, 73 bp, Homo sapiens
tgacctcgtggcgcaatggtagcgcgtctgactccagatcagaaggttgcgtgttcaagtcacgtcggggtca

SEQ ID NO: 437 - DNA, 73 bp, Homo sapiens
aggcgcggtggccaagtggtaaggcgtcggtctcgtaaaccgaagatcgcgggttcgaaccccgtccgtgcct

SEQ ID NO: 438 - DNA, 73 bp, Homo sapiens
agggggtatagctcagtggtagagcatttgactgcagatcaagaggtccccggttcaaatccgggtgccccct

SEQ ID NO: 439 - DNA, 73 bp, Homo sapiens
gggggtatagctcagtggtagagcatttgactgcagatcaagaggtccctggttcaaatccgggtgccccctc

SEQ ID NO: 440 - DNA, 73 bp, Homo sapiens
gggggtatagctcagtggtagagcatttgactgcagatcaagaggtccccggttcaaatccgggtgccccctc

SEQ ID NO: 441 - DNA, 73 bp, Homo sapiens
aggtcccatggtgtaatggttagcactctggactttgaatccagcgatccgagttcaaatctcggtgggacct

SEQ ID NO: 442 - DNA, 74 bp, Homo sapiens
gacccagtggcctaatggataaggcatcagcctccggagctggggattgtgggttcgagtcccatctgggtcgc

SEQ ID NO: 443 - DNA, 74 bp, Homo sapiens
agccccagtggcctaatggataaggcactggcctcctaagccagggattgtgggttcgagtcccacctggggta

SEQ ID NO: 444 - DNA, 74 bp, Homo sapiens
gccccagtggcctaatggataaggcactggcctcctaagccagggattgtgggttcgagtcccacctggggtgt

SEQ ID NO: 445 - DNA, 74 bp, Homo sapiens
agaccgcgtggcctaatggataaggcgtctgacttcggatcagaagattgagggttcgagtcccttcgtggtcg

SEQ ID NO: 446 - DNA, 75 bp, Homo sapiens
cgtctctgtggcgcaatcggttagcgcgttcggctgttaaccgaaaggttggtggttcgagcccacccagggacg

SEQ ID NO: 447 - DNA, 73 bp, Homo sapiens
ggcgttggtggtatagtggttagcatagctgccttccaagcagttgacccgggttcgattcccggccaacgca

SEQ ID NO: 448 - DNA, 74 bp, Homo sapiens
gtttccgtagtgtagcggttatcacattcgcctcacacgcgaaaggtccccggttcgatcccgggcggaaacag

SEQ ID NO: 449 - DNA, 75 bp, Homo sapiens
tggcgccgtggcttagttggttaaagcgcctgtctagtaaacaggagatcctgggttcgaatcccagcggtgcct

SEQ ID NO: 450 - DNA, 94 bp, Homo sapiens
gctccagtggcgcaatcggttagcgcgcggtacttatatgacagtgcgagcggagcaatgccgaggttgtgagttcgatcctcacctggagcac

SEQ ID NO: 451 - DNA, 72 bp, Homo sapiens
gcatgggtggttcagtggtagaattctcgcctgccacgcgggaggcccgggttcgattcccggcccatgcag

SEQ ID NO: 452 - DNA, 18 bp, Artificial Sequence
  source: /note=“Description of Artificial Sequence Synthetic oligonucleotide”
aaaatataaatatatttc

SEQ ID NO: 453 - DNA, 5 bp, Artificial Sequence
  source: /note=“Description of Artificial Sequence Synthetic oligonucleotide”
aagct

SEQ ID NO: 454 - DNA, 5 bp, Artificial Sequence
  source: /note=“Description of Artificial Sequence Synthetic oligonucleotide”
aagtt

SEQ ID NO: 455 - DNA, 15 bp, Artificial Sequence
  source: /note=“Description of Artificial Sequence Synthetic oligonucleotide”
aattcttcggaatgt

SEQ ID NO: 456 - DNA, 3 bp, Artificial Sequence
  source: /note=“Description of Artificial Sequence Synthetic oligonucleotide”
aga

SEQ ID NO: 457 - DNA, 5 bp, Artificial Sequence
  source: /note=“Description of Artificial Sequence Synthetic oligonucleotide”
agtcc

SEQ ID NO: 458 - DNA, 5 bp, Artificial Sequence
  source: /note=“Description of Artificial Sequence Synthetic oligonucleotide”
caacc

SEQ ID NO: 459 - DNA, 5 bp, Artificial Sequence
  source: /note=“Description of Artificial Sequence Synthetic oligonucleotide”
caatc

SEQ ID NO: 460 - DNA, 4 bp, Artificial Sequence
  source: /note=“Description of Artificial Sequence Synthetic oligonucleotide”
cagc

SEQ ID NO: 461 - DNA, 20 bp, Artificial Sequence
  source: /note=“Description of Artificial Sequence Synthetic oligonucleotide”
caggcgggttctgcccgcgc

SEQ ID NO: 462 - DNA, 18 bp, Artificial Sequence
  source: /note=“Description of Artificial Sequence Synthetic oligonucleotide”
catacctgcaagggtatc

SEQ ID NO: 463 - DNA, 15 bp, Artificial Sequence
  source: /note=“Description of Artificial Sequence Synthetic oligonucleotide”
cgaccgcaaggttgt

SEQ ID NO: 464 - DNA, 15 bp, Artificial Sequence
  source: /note=“Description of Artificial Sequence Synthetic oligonucleotide”
cgaccttgcggtcat

SEQ ID NO: 465 - DNA, 18 bp, Artificial Sequence
  source: /note=“Description of Artificial Sequence Synthetic oligonucleotide”
cgatgctaatcacatcgt

SEQ ID NO: 466 - DNA, 15 bp, Artificial Sequence
  source: /note=“Description of Artificial Sequence Synthetic oligonucleotide”
cgatggtgacatcat

SEQ ID NO: 467 - DNA, 16 bp, Artificial Sequence
  source: /note=“Description of Artificial Sequence Synthetic oligonucleotide”
cgatggtttacatcgt

SEQ ID NO: 468 - DNA, 13 bp, Artificial Sequence
  source: /note=“Description of Artificial Sequence Synthetic oligonucleotide”
cgccgtaaggtgt

SEQ ID NO: 469 - DNA, 12 bp, Artificial Sequence
  source: /note=“Description of Artificial Sequence Synthetic oligonucleotide”
cgccttaggtgt

SEQ ID NO: 470 - DNA, 15 bp, Artificial Sequence
  source: /note=“Description of Artificial Sequence Synthetic oligonucleotide”
cgcctttcgacgcgt

SEQ ID NO: 471 - DNA, 13 bp, Artificial Sequence
  source: /note=“Description of Artificial Sequence Synthetic oligonucleotide”
cgcttcacggcgt

SEQ ID NO: 472 - DNA, 15 bp, Artificial Sequence
  source: /note=“Description of Artificial Sequence Synthetic oligonucleotide”
cggcagcaatgctgt

SEQ ID NO: 473 - DNA, 13 bp, Artificial Sequence
  source: /note=“Description of Artificial Sequence Synthetic oligonucleotide”
cggctccgccttc

SEQ ID NO: 474 - DNA, 16 bp, Artificial Sequence
  source: /note=“Description of Artificial Sequence Synthetic oligonucleotide”
cgggtatcacagggtc

SEQ ID NO: 475 - DNA, 17 bp, Artificial Sequence
  source: /note=“Description of Artificial Sequence Synthetic oligonucleotide”
cggtgcgcaagcgctgt

SEQ ID NO: 476 - DNA, 18 bp, Artificial Sequence
  source: /note=“Description of Artificial Sequence Synthetic oligonucleotide”
cgtacgggtgaccgtacc

SEQ ID NO: 477 - DNA, 13 bp, Artificial Sequence
  source: /note=“Description of Artificial Sequence Synthetic oligonucleotide”
cgtcaaagacttc

SEQ ID NO: 478 - DNA, 13 bp, Artificial Sequence
  source: /note=“Description of Artificial Sequence Synthetic oligonucleotide”
cgtcgtaagactt

SEQ ID NO: 479 - DNA, 14 bp, Artificial Sequence
  source: /note=“Description of Artificial Sequence Synthetic oligonucleotide”
cgttgaataaacgt

SEQ ID NO: 480 - DNA, 5 bp, Artificial Sequence
  source: /note=“Description of Artificial Sequence Synthetic oligonucleotide”
ctgtc

SEQ ID NO: 481 - DNA, 4 bp, Artificial Sequence
  source: /note=“Description of Artificial Sequence Synthetic oligonucleotide”
ggcc

SEQ ID NO: 482 - DNA, 7 bp, Artificial Sequence
  source: /note=“Description of Artificial Sequence Synthetic oligonucleotide”
ggggatt

SEQ ID NO: 483 - DNA, 4 bp, Artificial Sequence
  source: /note=“Description of Artificial Sequence Synthetic oligonucleotide”
ggtc

SEQ ID NO: 484 - DNA, 5 bp, Artificial Sequence
  source: /note=“Description of Artificial Sequence Synthetic oligonucleotide”
ggttt

SEQ ID NO: 485 - DNA, 4 bp, Artificial Sequence
  source: /note=“Description of Artificial Sequence Synthetic oligonucleotide”
gtag

SEQ ID NO: 486 - DNA, 19 bp, Artificial Sequence
  source: /note=“Description of Artificial Sequence Synthetic oligonucleotide”
taactagatactttcagat

SEQ ID NO: 487 - DNA, 15 bp, Artificial Sequence
  source: /note=“Description of Artificial Sequence Synthetic oligonucleotide”
tactcgtatgggtgc

SEQ ID NO: 488 - DNA, 13 bp, Artificial Sequence
  source: /note=“Description of Artificial Sequence Synthetic oligonucleotide”
tactttgcggtgt

SEQ ID NO: 489 - DNA, 19 bp, Artificial Sequence
  source: /note=“Description of Artificial Sequence Synthetic oligonucleotide”
taggcgagtaacatcgtgc

SEQ ID NO: 490 - DNA, 19 bp, Artificial Sequence
  source: /note=“Description of Artificial Sequence Synthetic oligonucleotide”
taggcgtgaatagcgcctc

SEQ ID NO: 491 - DNA, 19 bp, Artificial Sequence
  source: /note=“Description of Artificial Sequence Synthetic oligonucleotide”
taggtcgcgagagcggcgc

SEQ ID NO: 492 - DNA, 19 bp, Artificial Sequence
  source: /note=“Description of Artificial Sequence Synthetic oligonucleotide”
taggtcgcgtaagcggcgc

SEQ ID NO: 493 - DNA, 17 bp, Artificial Sequence
  source: /note=“Description of Artificial Sequence Synthetic oligonucleotide”
taggtggttatccacgc

SEQ ID NO: 494 - DNA, 5 bp, Artificial Sequence
  source: /note=“Description of Artificial Sequence Synthetic oligonucleotide”
tagtc

SEQ ID NO: 495 - DNA, 5 bp, Artificial Sequence
  source: /note=“Description of Artificial Sequence Synthetic oligonucleotide”
tagtt

SEQ ID NO: 496 - DNA, 18 bp, Artificial Sequence
  source: /note=“Description of Artificial Sequence Synthetic oligonucleotide”
tatacgtgaaagcgtatc

SEQ ID NO: 497 - DNA, 21 bp, Artificial Sequence
  source: /note=“Description of Artificial Sequence Synthetic oligonucleotide”
tatagggtcaaaaactctatc

SEQ ID NO: 498 - DNA, 20 bp, Artificial Sequence
  source: /note=“Description of Artificial Sequence Synthetic oligonucleotide”
tatgcagaaatacctgcatc

SEQ ID NO: 499 - DNA, 15 bp, Artificial Sequence
  source: /note=“Description of Artificial Sequence Synthetic oligonucleotide”
tccccatacgggggc

SEQ ID NO: 500 - DNA, 14 bp, Artificial Sequence
  source: /note=“Description of Artificial Sequence Synthetic oligonucleotide”
tcccgaaggggttc

SEQ ID NO: 501 - DNA, 15 bp, Artificial Sequence
  source: /note=“Description of Artificial Sequence Synthetic oligonucleotide”
tctacgtatgtgggc

SEQ ID NO: 502 - DNA, 14 bp, Artificial Sequence
  source: /note=“Description of Artificial Sequence Synthetic oligonucleotide”
tctcataggagttc

SEQ ID NO: 503 - DNA, 14 bp, Artificial Sequence
  source: /note=“Description of Artificial Sequence Synthetic oligonucleotide”
tctcctctggaggc

SEQ ID NO: 504 - DNA, 15 bp, Artificial Sequence
  source: /note=“Description of Artificial Sequence Synthetic oligonucleotide”
tcttagcaataaggt

SEQ ID NO: 505 - DNA, 14 bp, Artificial Sequence
  source: /note=“Description of Artificial Sequence Synthetic oligonucleotide”
tcttgtaggagttc

SEQ ID NO: 506 - DNA, 15 bp, Artificial Sequence
  source: /note=“Description of Artificial Sequence Synthetic oligonucleotide”
tgaacgtaagttcgc

SEQ ID NO: 507 - DNA, 16 bp, Artificial Sequence
  source: /note=“Description of Artificial Sequence Synthetic oligonucleotide”
tgaactgcgaggttcc

SEQ ID NO: 508 - DNA, 4 bp, Artificial Sequence
  source: /note=“Description of Artificial Sequence Synthetic oligonucleotide”
tgac

SEQ ID NO: 509 - DNA, 15 bp, Artificial Sequence
  source: /note=“Description of Artificial Sequence Synthetic oligonucleotide”
tgaccgaaaggtcgt

SEQ ID NO: 510 - DNA, 15 bp, Artificial Sequence
  source: /note=“Description of Artificial Sequence Synthetic oligonucleotide”
tgaccgcaaggtcgt

SEQ ID NO: 511 - DNA, 14 bp, Artificial Sequence
  source: /note=“Description of Artificial Sequence Synthetic oligonucleotide”
tgagctctgctctc

SEQ ID NO: 512 - DNA, 18 bp, Artificial Sequence
  source: /note=“Description of Artificial Sequence Synthetic oligonucleotide”
tgaggcctcacggcctac

SEQ ID NO: 513 - DNA, 15 bp, Artificial Sequence
  source: /note=“Description of Artificial Sequence Synthetic oligonucleotide”
tgagggcaacttcgt

SEQ ID NO: 514 - DNA, 16 bp, Artificial Sequence
  source: /note=“Description of Artificial Sequence Synthetic oligonucleotide”
tgagggtcatacctcc

SEQ ID NO: 515 - DNA, 18 bp, Artificial Sequence
  source: /note=“Description of Artificial Sequence Synthetic oligonucleotide”
tgagggtgcaaatcctcc

SEQ ID NO: 516 - DNA, 13 bp, Artificial Sequence
  source: /note=“Description of Artificial Sequence Synthetic oligonucleotide”
tgccgaaaggcgt

SEQ ID NO: 517 - DNA, 13 bp, Artificial Sequence
  source: /note=“Description of Artificial Sequence Synthetic oligonucleotide”
tgccgtaaggcgt

SEQ ID NO: 518 - DNA, 14 bp, Artificial Sequence
  source: /note=“Description of Artificial Sequence Synthetic oligonucleotide”
tgcggtctccgcgc

SEQ ID NO: 519 - DNA, 11 bp, Artificial Sequence
  source: /note=“Description of Artificial Sequence Synthetic oligonucleotide”
tgctagagcat

SEQ ID NO: 520 - DNA, 16 bp, Artificial Sequence
  source: /note=“Description of Artificial Sequence Synthetic oligonucleotide”
tgctcgtatagagctc

SEQ ID NO: 521 - DNA, 15 bp, Artificial Sequence
  source: /note=“Description of Artificial Sequence Synthetic oligonucleotide”
tggacaattgtctgc

SEQ ID NO: 522 - DNA, 15 bp, Artificial Sequence
  source: /note=“Description of Artificial Sequence Synthetic oligonucleotide”
tggacagatgtccgt

SEQ ID NO: 523 - DNA, 15 bp, Artificial Sequence
  source: /note=“Description of Artificial Sequence Synthetic oligonucleotide”
tggacaggtgtccgc

SEQ ID NO: 524 - DNA, 15 bp, Artificial Sequence
  source: /note=“Description of Artificial Sequence Synthetic oligonucleotide”
tggacggttgtccgc

SEQ ID NO: 525 - DNA, 13 bp, Artificial Sequence
  source: /note=“Description of Artificial Sequence Synthetic oligonucleotide”
tggacttgtggtc

SEQ ID NO: 526 - DNA, 16 bp, Artificial Sequence
  source: /note=“Description of Artificial Sequence Synthetic oligonucleotide”
tggagattctctccgc

SEQ ID NO: 527 - DNA, 14 bp, Artificial Sequence
  source: /note=“Description of Artificial Sequence Synthetic oligonucleotide”
tggcataggcctgc

SEQ ID NO: 528 - DNA, 14 bp, Artificial Sequence
  source: /note=“Description of Artificial Sequence Synthetic oligonucleotide”
tggcttatgtctac

SEQ ID NO: 529 - DNA, 16 bp, Artificial Sequence
  source: /note=“Description of Artificial Sequence Synthetic oligonucleotide”
tgggagttaatcccgt

SEQ ID NO: 530 - DNA, 14 bp, Artificial Sequence
  source: /note=“Description of Artificial Sequence Synthetic oligonucleotide”
tgggatcttcccgc

SEQ ID NO: 531 - DNA, 16 bp, Artificial Sequence
  source: /note=“Description of Artificial Sequence Synthetic oligonucleotide”
tgggcagaaatgtctc

SEQ ID NO: 532 - DNA, 15 bp, Artificial Sequence
  source: /note=“Description of Artificial Sequence Synthetic oligonucleotide”
tgggcgttcgcccgc

SEQ ID NO: 533 - DNA, 14 bp, Artificial Sequence
  source: /note=“Description of Artificial Sequence Synthetic oligonucleotide”
tgggcttcgcccgc

SEQ ID NO: 534 - DNA, 16 bp, Artificial Sequence
  source: /note=“Description of Artificial Sequence Synthetic oligonucleotide”
tgggggataaccccgt

SEQ ID NO: 535 - DNA, 15 bp, Artificial Sequence
  source: /note=“Description of Artificial Sequence Synthetic oligonucleotide”
tgggggtttccccgt

SEQ ID NO: 536 - DNA, 4 bp, Artificial Sequence
  source: /note=“Description of Artificial Sequence Synthetic oligonucleotide”
tggt

SEQ ID NO: 537 - DNA, 15 bp, Artificial Sequence
  source: /note=“Description of Artificial Sequence Synthetic oligonucleotide”
tggtggcaacaccgt

SEQ ID NO: 538 - DNA, 14 bp, Artificial Sequence
  source: /note=“Description of Artificial Sequence Synthetic oligonucleotide”
tggtttatagccgt

SEQ ID NO: 539 - DNA, 19 bp, Artificial Sequence
  source: /note=“Description of Artificial Sequence Synthetic oligonucleotide”
tgtacggtaataccgtacc

SEQ ID NO: 540 - DNA, 15 bp, Artificial Sequence
  source: /note=“Description of Artificial Sequence Synthetic oligonucleotide”
tgtccgcaaggacgt

SEQ ID NO: 541 - DNA, 15 bp, Artificial Sequence
  source: /note=“Description of Artificial Sequence Synthetic oligonucleotide”
tgtcctaacggacgt

SEQ ID NO: 542 - DNA, 18 bp, Artificial Sequence
  source: /note=“Description of Artificial Sequence Synthetic oligonucleotide”
tgtcctattaacggacgt

SEQ ID NO: 543 - DNA, 16 bp, Artificial Sequence
  source: /note=“Description of Artificial Sequence Synthetic oligonucleotide”
tgtccttcacgggcgt

SEQ ID NO: 544 - DNA, 13 bp, Artificial Sequence
  source: /note=“Description of Artificial Sequence Synthetic oligonucleotide”
tgtcttaggacgt

SEQ ID NO: 545 - DNA, 18 bp, Artificial Sequence
  source: /note=“Description of Artificial Sequence Synthetic oligonucleotide”
tgtgcgttaacgcgtacc

SEQ ID NO: 546 - DNA, 16 bp, Artificial Sequence
  source: /note=“Description of Artificial Sequence Synthetic oligonucleotide”
tgtgtcgcaaggcacc

SEQ ID NO: 547 - DNA, 15 bp, Artificial Sequence
  source: /note=“Description of Artificial Sequence Synthetic oligonucleotide”
tgttcgtaaggactt

SEQ ID NO: 548 - DNA, 16 bp, Artificial Sequence
  source: /note=“Description of Artificial Sequence Synthetic oligonucleotide”
ttcacagaaatgtgtc

SEQ ID NO: 549 - DNA, 14 bp, Artificial Sequence
  source: /note=“Description of Artificial Sequence Synthetic oligonucleotide”
ttccctcgtggagt

SEQ ID NO: 550 - DNA, 14 bp, Artificial Sequence
  source: /note=“Description of Artificial Sequence Synthetic oligonucleotide”
ttccctctgggagc

SEQ ID NO: 551 - DNA, 14 bp, Artificial Sequence
  source: /note=“Description of Artificial Sequence Synthetic oligonucleotide”
ttcccttgtggatc

SEQ ID NO: 552 - DNA, 13 bp, Artificial Sequence
  source: /note=“Description of Artificial Sequence Synthetic oligonucleotide”
ttccttcgggagc

SEQ ID NO: 553 - DNA, 15 bp, Artificial Sequence
  source: /note=“Description of Artificial Sequence Synthetic oligonucleotide”
ttctagcaatagagt

SEQ ID NO: 554 - DNA, 16 bp, Artificial Sequence
  source: /note=“Description of Artificial Sequence Synthetic oligonucleotide”
ttctccactggggagc

SEQ ID NO: 555 - DNA, 15 bp, Artificial Sequence
  source: /note=“Description of Artificial Sequence Synthetic oligonucleotide”
ttctcgagagggagc

SEQ ID NO: 556 - DNA, 15 bp, Artificial Sequence
  source: /note=“Description of Artificial Sequence Synthetic oligonucleotide”
ttctcgtatgagagc

SEQ ID NO: 557 - DNA, 19 bp, Artificial Sequence
  source: /note=“Description of Artificial Sequence Synthetic oligonucleotide”
tttaaggttttcccttaac

SEQ ID NO: 558 - DNA, 14 bp, Artificial Sequence
  source: /note=“Description of Artificial Sequence Synthetic oligonucleotide”
tttcattgtggagt

SEQ ID NO: 559 - DNA, 14 bp, Artificial Sequence
  source: /note=“Description of Artificial Sequence Synthetic oligonucleotide”
tttcgaaggaatcc

SEQ ID NO: 560 - DNA, 13 bp, Artificial Sequence
  source: /note=“Description of Artificial Sequence Synthetic oligonucleotide”
tttcttcggaagc

SEQ ID NO: 561 - DNA, 16 bp, Artificial Sequence
  source: /note=“Description of Artificial Sequence Synthetic oligonucleotide”
tttggggcaactcaac

SEQ ID NO: 562 - RNA, 73 nt, Homo sapiens
gcccggcuagcucagucgguagagcaugggacucuuaaucccagggucguggguucgagccccacguugggcg

SEQ ID NO: 563 - DNA, 20 bp, Artificial Sequence
  source: /note=“Description of Artificial Sequence Synthetic primer”
tgagttggcaacctgtggta

SEQ ID NO: 564 - DNA, 20 bp, Artificial Sequence
  source: /note=“Description of Artificial Sequence Synthetic primer”
ttgggtgtccatgaaaatca

SEQ ID NO: 565 - RNA, 26 nt, Artificial Sequence
  source: /note=“Description of Artificial Sequence Synthetic probe”
uagcagaggaugguuucgauccauca

SEQ ID NO: 566 - DNA, 26 bp, Artificial Sequence
  source: /note=“Description of Artificial Sequence Synthetic probe”
tagcagaggatggtttcgatccatca

SEQ ID NO: 567 - DNA, 49 bp, Artificial Sequence
  source: /note=“Description of Artificial Sequence Synthetic probe”
ccaatggatttctatccatcgccttaaccactcggccacgactacaaaa

SEQ ID NO: 568 - RNA, 15 nt, Artificial Sequence
  source: /note=“Description of Artificial Sequence Synthetic probe”
cagauuaaaagucug

SEQ ID NO: 569 - RNA, 77 nt, Artificial Sequence
  source: /note=“Description of Artificial Sequence Synthetic oligonucleotide”
ggcucuauggcuuaguugguuaaagcgccugucucguaaacaggagauccuggguucgacucccaguggggccucaa

SEQ ID NO: 570 - RNA, 84 nt, Artificial Sequence
  source: /note=“Description of Artificial Sequence Synthetic oligonucleotide”
gucaggauggccgaguggucuaaggcgccagacucaaguucuggucuccguauggaggcguggguucgaaucccacuucugaca

SEQ ID NO: 571 - protein, 6 aa, Artificial Sequence
  source: /note=“Description of Artificial Sequence Synthetic 6xHis tag”
HHHHHH

SEQ ID NO: 572 - RNA, 342 nt, Artificial Sequence
  source: /note=“Description of Artificial Sequence Synthetic polynucleotide”
  modified_base (5)..(6): a, c, u, or g
  modified_base (15)..(16): a, c, u, or g
  modified_base (21)..(21): a, c, u, or g
  modified_base (30)..(32): a, c, u, or g
  modified_base (34)..(34): a, c, u, or g
  modified_base (37)..(37): a, c, u, or g
  modified_base (41)..(45): a, c, u, or g
  misc_feature (47)..(317): /note=“This region may encompass 1-271 nucleotides”
  modified_base (47)..(320): a, c, u, or g
  modified_base (328)..(329): a, c, u, or g
  modified_base (333)..(334): a, c, u, or g
  modified_base (336)..(337): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
rkssnndurghbyannyugrndgwdvdydnnnbnhbnryrnnnnndnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnrrduyrannyybnnhnnbwccd

SEQ ID NO: 573 - RNA, 341 nt, Artificial Sequence
  source: /note=“Description of Artificial Sequence Synthetic polynucleotide”
  modified_base (16)..(16): a, c, u, or g
  modified_base (30)..(30): a, c, u, or g
  modified_base (46)..(316): a, c, u, or g
  misc_feature (46)..(316): /note=“This region may encompass 1-271 nucleotides”
  modified_base (319)..(319): a, c, u, or g
  modified_base (328)..(328): a, c, u, or g
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
rksgrwdkrghbyavnyggudgarvrydynhkywbhryrhdhdhrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbhnrgducrayncyydvhhyywccd

SEQ ID NO: 574 - RNA, 341 nt, Artificial Sequence
  source: /note=“Description of Artificial Sequence Synthetic polynucleotide”
  modified_base (16)..(16): a, c, u, or g
  modified_base (30)..(30): a, c, u, or g
  modified_base (46)..(316): a, c, u, or g
  misc_feature (46)..(316): /note=“This region may encompass 1-271 nucleotides”
  modified_base (319)..(319): a, c, u, or g
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
ggggrwdurghbyavnyggudgarvrydynhkywkhryrhdhdhrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbhnrgducraybccydvhhyyucca

SEQ ID NO: 575 - RNA, 343 nt, Artificial Sequence
  source: /note=“Description of Artificial Sequence Synthetic polynucleotide”
  modified_base (2)..(8): a, c, u, or g
  modified_base (12)..(13): a, c, u, or g
  modified_base (17)..(17): a, c, u, or g
  modified_base (22)..(24): a, c, u, or g
  modified_base (26)..(27): a, c, u, or g
  modified_base (30)..(35): a, c, u, or g
  modified_base (38)..(38): a, c, u, or g
  modified_base (43)..(43): a, c, u, or g
  modified_base (45)..(322): a, c, u, or g
  misc_feature (48)..(318): /note=“This region may encompass 1-271 nucleotides”
  modified_base (329)..(329): a, c, u, or g
  modified_base (333)..(342): a, c, u, or g
  variation (1)..(343): /replace=“ ”
  misc_feature (1)..(343): /note=“Variant nucleotides given in the sequence have no preference with respect to those in the annotations for variant positions”
  source: /note=“See specification as filed for detailed description of substitutions and preferred embodiments”
gnnnnnnnkvgnnhddnhwrrnnnrnnvynnnnnnbunckrhnbnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnguuyranyybnnnnnnnnnnd

SEQ ID NO: 576 - RNA, 342 nt, Artificial Sequence
  source: /note=“Description of Artificial Sequence Synthetic polynucleotide”
  modified_base (3)..(3): a, c, u, or g
  modified_base (6)..(7): a, c, u, or g
  modified_base (13)..(13): a, c, u, or g
  modified_base (26)..(26): a, c, u, or g
  modified_base (32)..(32): a, c, u, or g
  modified_base (37)..(37): a, c, u, or g
  modified_base (44)..(44): a, c, u, or g
  modified_base (47)..(317): a, c, u, or g
  misc_feature (47)..(317): /note=“This region may encompass 1-271 nucleotides”
  modified_base (328)..(328): a, c, u, or g
  modified_base (336)..(338): a, c, u, or g
  modified_base (340)..(340): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
grnvbnnvkbgbnydrwurgwygarnrydyynsvyunckrmkbnrrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnshvgguuyranuycbdbynnnbnhr

SEQ ID NO: 577 - RNA, 342 nt, Artificial Sequence
  source: /note=“Description of Artificial Sequence Synthetic polynucleotide”
  modified_base (3)..(3): a, c, u, or g
  modified_base (7)..(7): a, c, u, or g
  modified_base (47)..(317): a, c, u, or g
  misc_feature (47)..(317): /note=“This region may encompass 1-271 nucleotides”
  modified_base (336)..(336): a, c, u, or g
  modified_base (340)..(340): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
grnvyvnskkgssydawuggaygarsgyryykgmyuhckrakymrrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnshrgguuygavuycydbynbdbnyg

SEQ ID NO: 578 - RNA, 341 nt, Artificial Sequence
  source: /note=“Description of Artificial Sequence Synthetic polynucleotide”
  modified_base (2)..(2): a, c, u, or g
  modified_base (4)..(6): a, c, u, or g
  modified_base (12)..(12): a, c, u, or g
  modified_base (17)..(17): a, c, u, or g
  modified_base (25)..(25): a, c, u, or g
  modified_base (28)..(30): a, c, u, or g
  modified_base (43)..(316): a, c, u, or g
  misc_feature (46)..(316): /note=“This region may encompass 1-271 nucleotides”
  modified_base (318)..(319): a, c, u, or g
  modified_base (327)..(327): a, c, u, or g
  modified_base (331)..(332): a, c, u, or g
  modified_base (334)..(337): a, c, u, or g
  modified_base (339)..(340): a, c, u, or g
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
dnynnndurghnhvrynggbhurdnrynnnbsryyruuwahbnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvnnrgukhvwnhcynnbnnnndnnr

SEQ ID NO: 579 - RNA, 341 nt, Artificial Sequence
  source: /note=“Description of Artificial Sequence Synthetic polynucleotide”
  modified_base (17)..(17): a, c, u, or g
  modified_base (28)..(28): a, c, u, or g
  modified_base (46)..(316): a, c, u, or g
  misc_feature (46)..(316): /note=“This region may encompass 1-271 nucleotides”
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
ruyucygurghryvrunggbyuarhgcnuuysryyruuwahbddannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnrgurgukhvwdmcyayysvrrrrbr

SEQ ID NO: 580 - RNA, 341 nt, Artificial Sequence
  source: /note=“Description of Artificial Sequence Synthetic polynucleotide”
  modified_base (46)..(316): a, c, u, or g
  misc_feature (46)..(316): /note=“This region may encompass 1-271 nucleotides”
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
ruyucygurghryvrubggbyuaghgcbuuysryyruuaahyddannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnrgurgukhvwdmcyayysvrrrryg

SEQ ID NO: 581 - RNA, 342 nt, Artificial Sequence
  source: /note=“Description of Artificial Sequence Synthetic polynucleotide”
  modified_base (2)..(6): a, c, u, or g
  modified_base (12)..(12): a, c, u, or g
  modified_base (16)..(16): a, c, u, or g
  modified_base (22)..(22): a, c, u, or g
  modified_base (26)..(26): a, c, u, or g
  modified_base (29)..(29): a, c, u, or g
  modified_base (31)..(32): a, c, u, or g
  modified_base (44)..(44): a, c, u, or g
  misc_feature (47)..(317): /note=“This region may encompass 1-271 nucleotides”
  modified_base (47)..(319): a, c, u, or g
  modified_base (328)..(328): a, c, u, or g
  modified_base (333)..(334): a, c, u, or g
  modified_base (336)..(339): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
knnnnndyrrynhrrnyygrhnumdnrynbnnvhyurucrhdbnbvnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnsrgyubrhnyyysnnhnnnnbmr

SEQ ID NO: 582 - RNA, 339 nt, Artificial Sequence
  source: /note=“Description of Artificial Sequence Synthetic polynucleotide”
  modified_base (2)..(2): a, c, u, or g
  modified_base (6)..(6): a, c, u, or g
  modified_base (44)..(314): a, c, u, or g
  misc_feature (44)..(314): /note=“This region may encompass 1-271 nucleotides”
  modified_base (333)..(333): a, c, u, or g
  variation (1)..(339): /replace=“ ”
  misc_feature (1)..(339): /note=“Variant nucleotides given in the sequence have no preference with respect to those in the annotations for variant positions”
  source: /note=“See specification as filed for detailed description of substitutions and preferred embodiments”
knsbbnwyarywyrguggukmgwrubysyryyurucaygbrsrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnykgrgyubrhkyycchrwnvvksmr

SEQ ID NO: 583 - RNA, 339 nt, Artificial Sequence
  source: /note=“Description of Artificial Sequence Synthetic polynucleotide”
  modified_base (44)..(314): a, c, u, or g
  misc_feature (44)..(314): /note=“This region may encompass 1-271 nucleotides”
  variation (1)..(339): /replace=“ ”
  misc_feature (1)..(339): /note=“Variant nucleotides given in the sequence have no preference with respect to those in the annotations for variant positions”
  source: /note=“See specification as filed for detailed description of substitutions and preferred embodiments”
umcuyruyaguaurguggukmguruyycygycugucaygyggrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnyggrguuyrmkyyccyrasrrggag

SEQ ID NO: 584 - RNA, 342 nt, Artificial Sequence
  source: /note=“Description of Artificial Sequence Synthetic polynucleotide”
  modified_base (3)..(6): a, c, u, or g
  modified_base (12)..(13): a, c, u, or g
  modified_base (16)..(16): a, c, u, or g
  modified_base (24)..(24): a, c, u, or g
  modified_base (26)..(26): a, c, u, or g
  modified_base (29)..(32): a, c, u, or g
  modified_base (34)..(34): a, c, u, or g
  modified_base (42)..(42): a, c, u, or g
  modified_base (44)..(319): a, c, u, or g
  misc_feature (47)..(317): /note=“This region may encompass 1-271 nucleotides”
  modified_base (328)..(328): a, c, u, or g
  modified_base (333)..(334): a, c, u, or g
  modified_base (336)..(340): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
gsnnnndurvynnavnkcgdbyynrnvbnnnnvnyyrcarmnbnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbdruucranyyyvnnhnnnnnyy

SEQ ID NO: 585 - RNA, 340 nt, Artificial Sequence
  source: /note=“Description of Artificial Sequence Synthetic polynucleotide”
  modified_base (16)..(16): a, c, u, or g
  modified_base (45)..(315): a, c, u, or g
  misc_feature (45)..(315): /note=“This region may encompass 1-271 nucleotides”
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
gsgsryrkrgyubasnkgdkuaravyahuugrcyrcaraucmarnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnyyydruucraruyyvgkuryychyy

SEQ ID NO: 586 - RNA, 340 nt, Artificial Sequence
  source: /note=“Description of Artificial Sequence Synthetic polynucleotide”
  modified_base (16)..(16): a, c, u, or g
  modified_base (45)..(315): a, c, u, or g
  misc_feature (45)..(315): /note=“This region may encompass 1-271 nucleotides”
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
gggsryrkrgyuyasnugrkuagagcayuugacugcaraucmarnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnyyyrruucraauyyvgkurcycmyy

SEQ ID NO: 587 - RNA, 341 nt, Artificial Sequence
  source: /note=“Description of Artificial Sequence Synthetic polynucleotide”
  modified_base (2)..(2): a, c, u, or g
  modified_base (4)..(6): a, c, u, or g
  modified_base (12)..(13): a, c, u, or g
  modified_base (16)..(16): a, c, u, or g
  modified_base (20)..(21): a, c, u, or g
  modified_base (24)..(24): a, c, u, or g
  modified_base (28)..(31): a, c, u, or g
  modified_base (33)..(33): a, c, u, or g
  modified_base (40)..(41): a, c, u, or g
  modified_base (43)..(319): a, c, u, or g
  misc_feature (46)..(316): /note=“This region may encompass 1-271 nucleotides”
  modified_base (327)..(327): a, c, u, or g
  modified_base (331)..(332): a, c, u, or g
  modified_base (335)..(339): a, c, u, or g
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
dnbnnnduvgbnnarnhggnnhanvvynnnnvnyubukrnnhnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnrruuyvanybynnbhnnnnnhd

SEQ ID NO: 588 - RNA, 340 nt, Artificial Sequence
  source: /note=“Description of Artificial Sequence Synthetic polynucleotide”
  modified_base (4)..(5): a, c, u, or g
  modified_base (12)..(12): a, c, u, or g
  modified_base (21)..(21): a, c, u, or g
  modified_base (27)..(28): a, c, u, or g
  misc_feature (45)..(315): /note=“This region may encompass 1-271 nucleotides”
  modified_base (45)..(317): a, c, u, or g
  modified_base (331)..(331): a, c, u, or g
  modified_base (335)..(336): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
kdbnnyduggynbarkmggdnavvrynnhbvryubukrrbhvdvnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnndrruuyvadybyhnbhdnnvhmd

SEQ ID NO: 589 - RNA, 340 nt, Artificial Sequence
  source: /note=“Description of Artificial Sequence Synthetic polynucleotide”
  modified_base (4)..(4): a, c, u, or g
  modified_base (21)..(21): a, c, u, or g
  modified_base (27)..(28): a, c, u, or g
  misc_feature (45)..(315): /note=“This region may encompass 1-271 nucleotides”
  modified_base (45)..(317): a, c, u, or g
  modified_base (331)..(331): a, c, u, or g
  modified_base (336)..(336): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
kdbnvyduggydbarumggknavvrynnwsrryubukrabhsdvnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnndrruuyvadysyhnbhdbnvhmk

SEQ ID NO: 590 - RNA, 342 nt, Artificial Sequence
  source: /note=“Description of Artificial Sequence Synthetic polynucleotide”
  modified_base (1)..(2): a, c, u, or g
  modified_base (5)..(6): a, c, u, or g
  modified_base (9)..(9): a, c, u, or g
  modified_base (12)..(12): a, c, u, or g
  modified_base (16)..(16): a, c, u, or g
  modified_base (20)..(21): a, c, u, or g
  modified_base (26)..(27): a, c, u, or g
  modified_base (29)..(33): a, c, u, or g
  modified_base (41)..(41): a, c, u, or g
  modified_base (44)..(318): a, c, u, or g
  misc_feature (47)..(317): /note=“This region may encompass 1-271 nucleotides”
  modified_base (320)..(321): a, c, u, or g
  modified_base (328)..(328): a, c, u, or g
  modified_base (333)..(336): a, c, u, or g
  modified_base (340)..(341): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
nnbbnndunrwnhrdnhygnnbhrdnnynnnnnvyuyusrnbvnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbnnryubrwnyyhbnnnndvvnnd

SEQ ID NO: 591 - RNA, 340 nt, Artificial Sequence
  source: /note=“Description of Artificial Sequence Synthetic polynucleotide”
  modified_base (1)..(2): a, c, u, or g
  modified_base (5)..(5): a, c, u, or g
  modified_base (12)..(12): a, c, u, or g
  modified_base (20)..(20): a, c, u, or g
  modified_base (29)..(29): a, c, u, or g
  modified_base (31)..(31): a, c, u, or g
  modified_base (39)..(39): a, c, u, or g
  modified_base (43)..(43): a, c, u, or g
  misc_feature (45)..(315): /note=“This region may encompass 1-271 nucleotides”
  modified_base (45)..(316): a, c, u, or g
  modified_base (319)..(319): a, c, u, or g
  modified_base (326)..(326): a, c, u, or g
  modified_base (334)..(334): a, c, u, or g
  modified_base (338)..(338): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
nnbynbdyrgunurdyagbnkrrvvywbnbnvyuyusanbvhndnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnydnryubrwnychbrkbndvvnvd

SEQ ID NO: 592 - RNA, 339 nt, Artificial Sequence
  source: /note=“Description of Artificial Sequence Synthetic polynucleotide”
  modified_base (1)..(1): a, c, u, or g
  modified_base (28)..(28): a, c, u, or g
  modified_base (30)..(30): a, c, u, or g
  modified_base (42)..(42): a, c, u, or g
  modified_base (44)..(314): a, c, u, or g
  misc_feature (44)..(314): /note=“This region may encompass 1-271 nucleotides”
  modified_base (318)..(318): a, c, u, or g
  modified_base (333)..(333): a, c, u, or g
  variation (1)..(339): /replace=“ ”
  misc_feature (1)..(339): /note=“Variant nucleotides given in the sequence have no preference with respect to those in the annotations for variant positions”
  source: /note=“See specification as filed for detailed description of substitutions and preferred embodiments”
ncsydbruggusuakygshkrrsaywbnknsyuyucahbvmndnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbyrnryuyrwyucyyrgbnwrsraw

SEQ ID NO: 593 - RNA, 342 nt, Artificial Sequence
  source: /note=“Description of Artificial Sequence Synthetic polynucleotide”
  modified_base (4)..(7): a, c, u, or g
  modified_base (12)..(12): a, c, u, or g
  modified_base (16)..(16): a, c, u, or g
  modified_base (21)..(22): a, c, u, or g
  modified_base (26)..(26): a, c, u, or g
  modified_base (29)..(34): a, c, u, or g
  modified_base (41)..(319): a, c, u, or g
  misc_feature (47)..(317): /note=“This region may encompass 1-271 nucleotides”
  modified_base (328)..(328): a, c, u, or g
  modified_base (333)..(338): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
rbvnnnnuvrynyrrnyubdnnuadnrynnnnnnyybccvnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvvsubbrwnhcbbnnnnnnbbyw

SEQ ID NO: 594 - RNA, 340 nt, Artificial Sequence
  source: /note=“Description of Artificial Sequence Synthetic polynucleotide”
  modified_base (5)..(5): a, c, u, or g
  modified_base (28)..(28): a, c, u, or g
  modified_base (39)..(40): a, c, u, or g
  modified_base (42)..(42): a, c, u, or g
  modified_base (45)..(315): a, c, u, or g
  misc_feature (45)..(315): /note=“This region may encompass 1-271 nucleotides”
  modified_base (317)..(317): a, c, u, or g
  modified_base (335)..(335): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
gbvbnsvurrudyrrdcggkdadmaudnhhrhyubccannynkdnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbnrgsuubrwuuccyksbsnvygca

SEQ ID NO: 595 - RNA, 340 nt, Artificial Sequence
  source: /note=“Description of Artificial Sequence Synthetic polynucleotide”
  modified_base (5)..(5): a, c, u, or g
  modified_base (28)..(28): a, c, u, or g
  modified_base (39)..(39): a, c, u, or g
  modified_base (42)..(42): a, c, u, or g
  modified_base (45)..(315): a, c, u, or g
  misc_feature (45)..(315): /note=“This region may encompass 1-271 nucleotides”
  modified_base (335)..(335): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
gyvynsvurrudyrrwcggkkadmaudnhhrhyubccandynkdnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbvrgsuuyrwuuccyksbsnvygca

SEQ ID NO: 596 - RNA, 342 nt, Artificial Sequence
  source: /note=“Description of Artificial Sequence Synthetic polynucleotide”
  modified_base (4)..(7): a, c, u, or g
  modified_base (13)..(13): a, c, u, or g
  modified_base (25)..(26): a, c, u, or g
  modified_base (29)..(31): a, c, u, or g
  modified_base (34)..(34): a, c, u, or g
  modified_base (42)..(42): a, c, u, or g
  modified_base (45)..(317): a, c, u, or g
  misc_feature (47)..(317): /note=“This region may encompass 1-271 nucleotides”
  modified_base (319)..(320): a, c, u, or g
  modified_base (328)..(328): a, c, u, or g
  modified_base (332)..(333): a, c, u, or g
  modified_base (336)..(338): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
kgynnnnduvgbnhagbyugghyannvynnndbnyugugrhnvhnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvnnrgyucranycynnbhnnnvrym

SEQ ID NO: 597 - RNA, 339 nt, Artificial Sequence
  source: /note=“Description of Artificial Sequence Synthetic polynucleotide”
  modified_base (44)..(314): a, c, u, or g
  misc_feature (44)..(314): /note=“This region may encompass 1-271 nucleotides”
  variation (1)..(339): /replace=“ ”
  misc_feature (1)..(339): /note=“Variant nucleotides given in the sequence have no preference with respect to those in the annotations for variant positions”
  source: /note=“See specification as filed for detailed description of substitutions and preferred embodiments”
gchrugaybguayagkgguuasyacucugygyuguggccrcagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncwyggyucraaucygagucaygrca

SEQ ID NO: 598 - RNA, 339 nt, Artificial Sequence
  source: /note=“Description of Artificial Sequence Synthetic polynucleotide”
  modified_base (44)..(314): a, c, u, or g
  misc_feature (44)..(314): /note=“This region may encompass 1-271 nucleotides”
  variation (1)..(339): /replace=“ ”
  misc_feature (1)..(339): /note=“Variant nucleotides given in the sequence have no preference with respect to those in the annotations for variant positions”
  source: /note=“See specification as filed for detailed description of substitutions and preferred embodiments”
gcmrugayyguauagugguuagyacucugygyuguggccrcagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncwygguucraaucygagucaygrca

SEQ ID NO: 599 - RNA, 342 nt, Artificial Sequence
  source: /note=“Description of Artificial Sequence Synthetic polynucleotide”
  modified_base (1)..(1): a, c, u, or g
  modified_base (4)..(5): a, c, u, or g
  modified_base (7)..(7): a, c, u, or g
  modified_base (17)..(17): a, c, u, or g
  modified_base (31)..(32): a, c, u, or g
  modified_base (34)..(34): a, c, u, or g
  modified_base (37)..(37): a, c, u, or g
  modified_base (42)..(320): a, c, u, or g
  misc_feature (47)..(317): /note=“This region may encompass 1-271 nucleotides”
  modified_base (328)..(329): a, c, u, or g
  modified_base (332)..(334): a, c, u, or g
  modified_base (336)..(339): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
nvsnnbndurgybyrrnywggbhrdvrydbnnbnyunaurannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnndsywydannchnnnhnnnnsba

SEQ ID NO: 600 - RNA, 340 nt, Artificial Sequence
  source: /note=“Description of Artificial Sequence Synthetic polynucleotide”
  modified_base (45)..(315): a, c, u, or g
  misc_feature (45)..(315): /note=“This region may encompass 1-271 nucleotides”
  modified_base (326)..(327): a, c, u, or g
  modified_base (335)..(335): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
gsyybrkurgykyrruyggyhagmgcrygkurcudauaayryyrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnryrrsywydanncycrymyngrsca

SEQ ID NO: 601 - RNA, 340 nt, Artificial Sequence
  source: /note=“Description of Artificial Sequence Synthetic polynucleotide”
  modified_base (45)..(315): a, c, u, or g
  misc_feature (45)..(315): /note=“This region may encompass 1-271 nucleotides”
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
gsyysrkurgykcaruyggyhagmgcgyggurcudauaayrcyrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngygrgyucrabhcycrymybgrsca

SEQ ID NO: 602 - RNA, 341 nt, Artificial Sequence
  source: /note=“Description of Artificial Sequence Synthetic polynucleotide”
  modified_base (1)..(1): a, c, u, or g
  modified_base (3)..(6): a, c, u, or g
  modified_base (11)..(12): a, c, u, or g
  modified_base (16)..(17): a, c, u, or g
  modified_base (21)..(22): a, c, u, or g
  modified_base (25)..(26): a, c, u, or g
  modified_base (28)..(31): a, c, u, or g
  modified_base (41)..(41): a, c, u, or g
  modified_base (43)..(316): a, c, u, or g
  misc_feature (46)..(316): /note=“This region may encompass 1-271 nucleotides”
  modified_base (318)..(319): a, c, u, or g
  modified_base (327)..(327): a, c, u, or g
  modified_base (331)..(332): a, c, u, or g
  modified_base (335)..(338): a, c, u, or g
  modified_base (340)..(340): a, c, u, or g
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
nbnnnndursnnbarnnhggnnddnnbnnnnvdhycauahnbnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvnnrgdusdanhmynnbhnnnnvnw

SEQ ID NO: 603 - RNA, 339 nt, Artificial Sequence
  source: /note=“Description of Artificial Sequence Synthetic polynucleotide”
  modified_base (4)..(4): a, c, u, or g
  modified_base (44)..(314): a, c, u, or g
  misc_feature (44)..(314): /note=“This region may encompass 1-271 nucleotides”
  variation (1)..(339): /replace=“ ”
  misc_feature (1)..(339): /note=“Variant nucleotides given in the sequence have no preference with respect to those in the annotations for variant positions”
  source: /note=“See specification as filed for detailed description of substitutions and preferred embodiments”
rbcnbvkurgygcagydgghagyryryyrgkyycauaahyyvrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnsdkrgdusdadmmymwsmbvbgsya

SEQ ID NO: 604 - RNA, 339 nt, Artificial Sequence
  source: /note=“Description of Artificial Sequence Synthetic polynucleotide”
  modified_base (44)..(314): a, c, u, or g
  misc_feature (44)..(314): /note=“This region may encompass 1-271 nucleotides”
  variation (1)..(339): /replace=“ ”
  misc_feature (1)..(339): /note=“Variant nucleotides given in the sequence have no preference with respect to those in the annotations for variant positions”
  source: /note=“See specification as filed for detailed description of substitutions and preferred embodiments”
rschbvkurgygcagydgghagcryryyrgkyycauaaycyrrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnswkrgwusdadmcymwsmbvkgsya

SEQ ID NO: 605 - RNA, 342 nt, Artificial Sequence
  source: /note=“Description of Artificial Sequence Synthetic polynucleotide”
  modified_base (2)..(7): a, c, u, or g
  modified_base (9)..(12): a, c, u, or g
  modified_base (16)..(16): a, c, u, or g
  modified_base (23)..(23): a, c, u, or g
  modified_base (26)..(26): a, c, u, or g
  modified_base (28)..(34): a, c, u, or g
  modified_base (37)..(37): a, c, u, or g
  modified_base (41)..(317): a, c, u, or g
  misc_feature (47)..(317): /note=“This region may encompass 1-271 nucleotides”
  modified_base (319)..(320): a, c, u, or g
  modified_base (328)..(328): a, c, u, or g
  modified_base (332)..(333): a, c, u, or g
  modified_base (335)..(340): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
vnnnnnnunnnnvrrnhhgkhhndhnvnnnnnnnhynardnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvnnvbyuhvanymbnnbnnnnnnbr

SEQ ID NO: 606 - RNA, 342 nt, Artificial Sequence
  source: /note=“Description of Artificial Sequence Synthetic polynucleotide”
  modified_base (6)..(6): a, c, u, or g
  modified_base (33)..(33): a, c, u, or g
  modified_base (41)..(41): a, c, u, or g
  modified_base (43)..(43): a, c, u, or g
  modified_base (46)..(317): a, c, u, or g
  misc_feature (47)..(317): /note=“This region may encompass 1-271 nucleotides”
  modified_base (319)..(319): a, c, u, or g
  modified_base (328)..(328): a, c, u, or g
  modified_base (333)..(333): a, c, u, or g
  modified_base (336)..(336): a, c, u, or g
  modified_base (340)..(340): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
rbbvvndurkhhsrrbhygkhhurwbrhdbhdnryuhardnynhdnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvnvgbyuhvanymybnbbnbbvnya

SEQ ID NO: 607 - RNA, 342 nt, Artificial Sequence
  source: /note=“Description of Artificial Sequence Synthetic polynucleotide”
  modified_base (6)..(6): a, c, u, or g
  modified_base (33)..(33): a, c, u, or g
  modified_base (43)..(43): a, c, u, or g
  modified_base (46)..(317): a, c, u, or g
  misc_feature (47)..(317): /note=“This region may encompass 1-271 nucleotides”
  modified_base (319)..(319): a, c, u, or g
  modified_base (333)..(333): a, c, u, or g
  modified_base (336)..(336): a, c, u, or g
  modified_base (340)..(340): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
rbbvvndurkhhsrrbhygkhhurwkrhdbhdnryuhardhynhdnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvnvgbyuhvavymybnbynbbvnya

SEQ ID NO: 608 - RNA, 342 nt, Artificial Sequence
  source: /note=“Description of Artificial Sequence Synthetic polynucleotide”
  modified_base (1)..(7): a, c, u, or g
  modified_base (11)..(12): a, c, u, or g
  modified_base (16)..(16): a, c, u, or g
  modified_base (21)..(21): a, c, u, or g
  modified_base (26)..(26): a, c, u, or g
  modified_base (30)..(32): a, c, u, or g
  modified_base (44)..(320): a, c, u, or g
  misc_feature (47)..(317): /note=“This region may encompass 1-271 nucleotides”
  modified_base (328)..(328): a, c, u, or g
  modified_base (332)..(333): a, c, u, or g
  modified_base (335)..(340): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
nnnnnnnursnnharnhwrrnuudrnrydnnnsvyyyuummbvnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvryuyrwnhybnnbnnnnnnhd

SEQ ID NO: 609 - RNA, 340 nt, Artificial Sequence
  source: /note=“Description of Artificial Sequence Synthetic polynucleotide”
  modified_base (1)..(1): a, c, u, or g
  modified_base (6)..(6): a, c, u, or g
  modified_base (11)..(11): a, c, u, or g
  modified_base (29)..(29): a, c, u, or g
  modified_base (43)..(43): a, c, u, or g
  misc_feature (45)..(315): /note=“This region may encompass 1-271 nucleotides”
  modified_base (45)..(317): a, c, u, or g
  modified_base (331)..(331): a, c, u, or g
  modified_base (333)..(334): a, c, u, or g
  modified_base (336)..(336): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
nhvbdnvkrsnhharbhrrbudrdrydbndgryyyuummymhndnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnrgryuyrwbyysynbnnhndrhr

SEQ ID NO: 610 - RNA, 340 nt, Artificial Sequence
  source: /note=“Description of Artificial Sequence Synthetic polynucleotide”
  misc_feature (45)..(315): /note=“This region may encompass 1-271 nucleotides”
  modified_base (45)..(316): a, c, u, or g
  modified_base (333)..(333): a, c, u, or g
  modified_base (336)..(336): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
vhvbrdvkascwharuhrrbudrwrcrkvdgacuyuumauchswnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnndrgryuyrwkyysyhbnhynkryr

SEQ ID NO: 611 - RNA, 341 nt, Artificial Sequence
  source: /note=“Description of Artificial Sequence Synthetic polynucleotide”
  modified_base (4)..(6): a, c, u, or g
  modified_base (29)..(31): a, c, u, or g
  modified_base (33)..(33): a, c, u, or g
  modified_base (41)..(41): a, c, u, or g
  modified_base (43)..(318): a, c, u, or g
  misc_feature (46)..(316): /note=“This region may encompass 1-271 nucleotides”
  modified_base (327)..(327): a, c, u, or g
  modified_base (331)..(332): a, c, u, or g
  modified_base (335)..(338): a, c, u, or g
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
kbbnnnduagyyyarhyuggbwwgrryrnnnbnyygaarvnbnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbdguucranychnnbhnnnnvma

SEQ ID NO: 612 - RNA, 340 nt, Artificial Sequence
  source: /note=“Description of Artificial Sequence Synthetic polynucleotide”
  modified_base (45)..(315): a, c, u, or g
  misc_feature (45)..(315): /note=“This region may encompass 1-271 nucleotides”
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
kbyvwrruwgyyyaruuggsuagrrydykrbrcygaarrysyhmnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnyckgguucrayycysgguywbrvma

SEQ ID NO: 613 - RNA, 339 nt, Artificial Sequence
  source: /note=“Description of Artificial Sequence Synthetic polynucleotide”
  modified_base (44)..(314): a, c, u, or g
  misc_feature (44)..(314): /note=“This region may encompass 1-271 nucleotides”
  variation (1)..(339): /replace=“ ”
  misc_feature (1)..(339): /note=“Variant nucleotides given in the sequence have no preference with respect to those in the annotations for variant positions”
  source: /note=“See specification as filed for detailed description of substitutions and preferred embodiments”
gcyrarauwgcucaruugggagagyguuasacygaagaucuwmnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnycugguucrayycyggguuucrvca

SEQ ID NO: 614 - RNA, 342 nt, Artificial Sequence
  source: /note=“Description of Artificial Sequence Synthetic polynucleotide”
  modified_base (4)..(6): a, c, u, or g
  modified_base (16)..(16): a, c, u, or g
  modified_base (21)..(21): a, c, u, or g
  modified_base (29)..(34): a, c, u, or g
  modified_base (37)..(37): a, c, u, or g
  modified_base (41)..(320): a, c, u, or g
  misc_feature (47)..(317): /note=“This region may encompass 1-271 nucleotides”
  modified_base (328)..(328): a, c, u, or g
  modified_base (331)..(334): a, c, u, or g
  modified_base (336)..(338): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
sksnnnduvgbbyagnyygbnyyrdvvynnnnnnhunggrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnndguucranycnnnnhnnnbmsm

SEQ ID NO: 615 - RNA, 338 nt, Artificial Sequence
  source: /note=“Description of Artificial Sequence Synthetic polynucleotide”
  modified_base (39)..(39): a, c, u, or g
  modified_base (43)..(313): a, c, u, or g
  misc_feature (43)..(313): /note=“This region may encompass 1-271 nucleotides”
  variation (1)..(338): /replace=“ ”
  misc_feature (1)..(338): /note=“Variant nucleotides given in the sequence have no preference with respect to those in the annotations for variant positions”
  source: /note=“See specification as filed for detailed description of substitutions and preferred embodiments”
ggcusguuggkcuagkgburugruucucrsyuhggrysnragnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncyggguucaaaucvyrgasgagccc

SEQ ID NO: 616 - RNA, 338 nt, Artificial Sequence
  source: /note=“Description of Artificial Sequence Synthetic polynucleotide”
  modified_base (43)..(313): a, c, u, or g
  misc_feature (43)..(313): /note=“This region may encompass 1-271 nucleotides”
  variation (1)..(338): /replace=“ ”
  misc_feature (1)..(338): /note=“Variant nucleotides given in the sequence have no preference with respect to those in the annotations for variant positions”
  source: /note=“See specification as filed for detailed description of substitutions and preferred embodiments”
ggcucguuggucuagkggurugruucucgsuuhggrysbgagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccggguucaaaucccggacgagccc

SEQ ID NO: 617 - RNA, 342 nt, Artificial Sequence
  source: /note=“Description of Artificial Sequence Synthetic polynucleotide”
  modified_base (2)..(7): a, c, u, or g
  modified_base (9)..(13): a, c, u, or g
  modified_base (16)..(16): a, c, u, or g
  modified_base (21)..(21): a, c, u, or g
  modified_base (25)..(28): a, c, u, or g
  modified_base (30)..(34): a, c, u, or g
  modified_base (37)..(37): a, c, u, or g
  modified_base (42)..(320): a, c, u, or g
  misc_feature (47)..(317): /note=“This region may encompass 1-271 nucleotides”
  modified_base (332)..(340): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
dnnnnnnynnnnnarnbhggnbbannnndnnnnnyynswrmnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnrkuuyradycynnnnnnnnnbd

SEQ ID NO: 618 - RNA, 341 nt, Artificial Sequence
  source: /note=“Description of Artificial Sequence Synthetic polynucleotide”
  modified_base (2)..(5): a, c, u, or g
  modified_base (36)..(36): a, c, u, or g
  modified_base (46)..(316): a, c, u, or g
  misc_feature (46)..(316): /note=“This region may encompass 1-271 nucleotides”
  modified_base (319)..(319): a, c, u, or g
  modified_base (331)..(331): a, c, u, or g
  modified_base (335)..(336): a, c, u, or g
  modified_base (338)..(339): a, c, u, or g
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
knnnnbdurgchsarkbugguuavdghrdbkdrcynswraybmvdnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvyngkuuyradyccnrbhnnbnnyd

SEQ ID NO: 619 - RNA, 341 nt, Artificial Sequence
  source: /note=“Description of Artificial Sequence Synthetic polynucleotide”
  modified_base (2)..(3): a, c, u, or g
  modified_base (5)..(5): a, c, u, or g
  modified_base (36)..(36): a, c, u, or g
  modified_base (46)..(316): a, c, u, or g
  misc_feature (46)..(316): /note=“This region may encompass 1-271 nucleotides”
  modified_base (335)..(336): a, c, u, or g
  modified_base (338)..(339): a, c, u, or g
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
knndnbrurgchsarkcugguuavdghrwykrrcunswrayymrdnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvydgguuyraaycchrbynnynnyk

SEQ ID NO: 620 - RNA, 341 nt, Artificial Sequence
  source: /note=“Description of Artificial Sequence Synthetic polynucleotide”
  modified_base (4)..(7): a, c, u, or g
  modified_base (12)..(12): a, c, u, or g
  modified_base (16)..(16): a, c, u, or g
  modified_base (25)..(25): a, c, u, or g
  modified_base (29)..(31): a, c, u, or g
  modified_base (33)..(33): a, c, u, or g
  modified_base (36)..(36): a, c, u, or g
  modified_base (41)..(41): a, c, u, or g
  modified_base (43)..(319): a, c, u, or g
  misc_feature (46)..(316): /note=“This region may encompass 1-271 nucleotides”
  modified_base (327)..(327): a, c, u, or g
  modified_base (331)..(337): a, c, u, or g
  modified_base (341)..(341): a, c, u, or g
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
rbbnnnnurgynhadnhykghyrdnryrnnnsnhynguwansnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnndgwyyvanbyhnnnnnnnrvyn

SEQ ID NO: 621 - RNA, 340 nt, Artificial Sequence
  source: /note=“Description of Artificial Sequence Synthetic polynucleotide”
  modified_base (4)..(4): a, c, u, or g
  modified_base (6)..(6): a, c, u, or g
  modified_base (12)..(12): a, c, u, or g
  modified_base (24)..(24): a, c, u, or g
  modified_base (45)..(315): a, c, u, or g
  misc_feature (45)..(315): /note=“This region may encompass 1-271 nucleotides”
  modified_base (317)..(317): a, c, u, or g
  modified_base (326)..(326): a, c, u, or g
  modified_base (331)..(332): a, c, u, or g
  modified_base (334)..(334): a, c, u, or g
  modified_base (336)..(336): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
rbbnbnduvgynharbygghyrdnryryhbsbcyhguwavsvdrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvndrgwuyvanbyyhnnbndnrvyb

SEQ ID NO: 622 - RNA, 340 nt, Artificial Sequence
  source: /note=“Description of Artificial Sequence Synthetic polynucleotide”
  modified_base (4)..(4): a, c, u, or g
  modified_base (6)..(6): a, c, u, or g
  modified_base (12)..(12): a, c, u, or g
  modified_base (24)..(24): a, c, u, or g
  modified_base (45)..(315): a, c, u, or g
  misc_feature (45)..(315): /note=“This region may encompass 1-271 nucleotides”
  modified_base (326)..(326): a, c, u, or g
  modified_base (332)..(332): a, c, u, or g
  modified_base (334)..(334): a, c, u, or g
  modified_base (336)..(336): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
rsynbnduvgynharbugghyrdnryryhksbcyhguaavsmdrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvhdrgwuyvanbyyhdnbndnrsyu

SEQ ID NO: 623 - RNA, 342 nt, Artificial Sequence
  source: /note=“Description of Artificial Sequence Synthetic polynucleotide”
  modified_base (4)..(6): a, c, u, or g
  modified_base (29)..(32): a, c, u, or g
  modified_base (34)..(34): a, c, u, or g
  modified_base (42)..(42): a, c, u, or g
  modified_base (44)..(317): a, c, u, or g
  misc_feature (47)..(317): /note=“This region may encompass 1-271 nucleotides”
  modified_base (319)..(319): a, c, u, or g
  modified_base (321)..(321): a, c, u, or g
  modified_base (328)..(328): a, c, u, or g
  modified_base (333)..(333): a, c, u, or g
  modified_base (336)..(338): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
rvvnnndurrybhhrhyhgghyuadvvynnnnrnbuccavanbnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvnrnguucranychynbhnnnbbhd

SEQ ID NO: 624 - RNA, 339 nt, Artificial Sequence
  source: /note=“Description of Artificial Sequence Synthetic polynucleotide”
  modified_base (44)..(314): a, c, u, or g
  misc_feature (44)..(314): /note=“This region may encompass 1-271 nucleotides”
  variation (1)..(339): /replace=“ ”
  misc_feature (1)..(339): /note=“Variant nucleotides given in the sequence have no preference with respect to those in the annotations for variant positions”
  source: /note=“See specification as filed for detailed description of substitutions and preferred embodiments”
grmmkvrkggysmaryuggharssykkybrrsuccasakbvrwnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnsygkguucradycmcrkyksbgyma

SEQ ID NO: 625 - RNA, 339 nt, Artificial Sequence; identical to SEQ ID NO: 624
  source: /note=“Description of Artificial Sequence Synthetic polynucleotide”
  modified_base (44)..(314): a, c, u, or g
  misc_feature (44)..(314): /note=“This region may encompass 1-271 nucleotides”
  variation (1)..(339): /replace=“ ”
  misc_feature (1)..(339): /note=“Variant nucleotides given in the sequence have no preference with respect to those in the annotations for variant positions”
  source: /note=“See specification as filed for detailed description of substitutions and preferred embodiments”

SEQ ID NO: 626 - RNA, 342 nt, Artificial Sequence
  source: /note=“Description of Artificial Sequence Synthetic polynucleotide”
  modified_base (3)..(6): a, c, u, or g
  modified_base (9)..(10): a, c, u, or g
  modified_base (12)..(13): a, c, u, or g
  modified_base (16)..(16): a, c, u, or g
  modified_base (25)..(26): a, c, u, or g
  modified_base (30)..(32): a, c, u, or g
  modified_base (42)..(42): a, c, u, or g
  modified_base (44)..(317): a, c, u, or g
  misc_feature (47)..(317): /note=“This region may encompass 1-271 nucleotides”
  modified_base (319)..(320): a, c, u, or g
  modified_base (328)..(328): a, c, u, or g
  modified_base (332)..(333): a, c, u, or g
  modified_base (336)..(340): a, c, u, or g
  variation (1)..(342): /replace=“ ”
  misc_feature (1)..(342): /note=“Variant nucleotides given in the sequence have no preference with respect to those in the annotations for variant positions”
  source: /note=“See specification as filed for detailed description of substitutions and preferred embodiments”
bbnnnndunnynnarnyugsyhwrnnrbdnnnrdcuruarvnynnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvnnvgwuyranucbnnbhnnnnnvd

SEQ ID NO: 627 - RNA, 340 nt, Artificial Sequence
  source: /note=“Description of Artificial Sequence Synthetic polynucleotide”
  modified_base (45)..(315): a, c, u, or g
  misc_feature (45)..(315): /note=“This region may encompass 1-271 nucleotides”
  modified_base (336)..(336): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
bbkkbdauagyucagbugsyuagagydkwkracuruagrkymwknnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnscuggwuyrahucyrrsubsnmsvd

SEQ ID NO: 628 - RNA, 340 nt, Artificial Sequence
  source: /note=“Description of Artificial Sequence Synthetic polynucleotide”
  modified_base (45)..(315): a, c, u, or g
  misc_feature (45)..(315): /note=“This region may encompass 1-271 nucleotides”
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
bbkksdauagyucagyuggyuagagcdkwkgacuruagrkymwknnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnscugguuyrawuccrrsuysdmsva

SEQ ID NO: 629 - RNA, 341 nt, Artificial Sequence
  source: /note=“Description of Artificial Sequence Synthetic polynucleotide”
  modified_base (2)..(2): a, c, u, or g
  modified_base (4)..(7): a, c, u, or g
  modified_base (12)..(12): a, c, u, or g
  modified_base (15)..(16): a, c, u, or g
  modified_base (21)..(21): a, c, u, or g
  modified_base (24)..(25): a, c, u, or g
  modified_base (28)..(28): a, c, u, or g
  modified_base (30)..(33): a, c, u, or g
  modified_base (36)..(36): a, c, u, or g
  modified_base (40)..(319): a, c, u, or g
  misc_feature (46)..(316): /note=“This region may encompass 1-271 nucleotides”
  modified_base (327)..(327): a, c, u, or g
  modified_base (330)..(339): a, c, u, or g
  variation (1)..(341): /replace=“ ”
  misc_feature (1)..(341): /note=“Variant nucleotides given in the sequence have no preference with respect to those in the annotations for variant positions”
  source: /note=“See specification as filed for detailed description of substitutions and preferred embodiments”
dndnnnnuvrynbrnnhugknyannrynbnnnnhunacrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnndrdubranhynnnnnnnnnnyv

SEQ ID NO: 630 - RNA, 340 nt, Artificial Sequence
  source: /note=“Description of Artificial Sequence Synthetic polynucleotide”
  modified_base (15)..(16): a, c, u, or g
  modified_base (24)..(24): a, c, u, or g
  modified_base (27)..(27): a, c, u, or g
  modified_base (29)..(30): a, c, u, or g
  modified_base (41)..(43): a, c, u, or g
  modified_base (45)..(315): a, c, u, or g
  misc_feature (45)..(315): /note=“This region may encompass 1-271 nucleotides”
  modified_base (317)..(318): a, c, u, or g
  modified_base (326)..(326): a, c, u, or g
  modified_base (330)..(330): a, c, u, or g
  modified_base (333)..(333): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
kbkbshdurrydbrnnhgkuyadnrynynndhyuhacrhknnnrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbnnrrdubranhybndvndsdwvyv

SEQ ID NO: 631 - RNA, 340 nt, Artificial Sequence
  source: /note=“Description of Artificial Sequence Synthetic polynucleotide”
  modified_base (45)..(315): a, c, u, or g
  misc_feature (45)..(315): /note=“This region may encompass 1-271 nucleotides”
  modified_base (317)..(318): a, c, u, or g
  modified_base (330)..(330): a, c, u, or g
  variation (1)..(340): /replace=“ ”
  misc_feature (1)..(340): /note=“Variant nucleotides given in the sequence have no preference with respect to those in the annotations for variant positions”
  source: /note=“See specification as filed for detailed description of substitutions and preferred embodiments”
kbkbsydurgydbrbhugkuyadhryvyhhdyyuhacahkhddrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbnngrdubradhyyndvhrsdwvya